(19) United States
(12) Patent Application Publication
(10) Pub. No.: US 2020/0206304 A1
(43) Pub. Date: Jul. 2, 2020

(54) METHODS AND COMPOSITIONS FOR EFFICIENT DELIVERY THROUGH MULTIPLE BIO BARRIERS

(71) Applicant: Cedars-Sinai Medical Center, Los Angeles, CA (US)

(72) Inventors: Eggehard Holler, Los Angeles, CA (US); Julia Y. Ljubimova, Studio City, CA (US); Keith L. Black, Los Angeles, CA (US)

(73) Assignee: Cedars-Sinai Medical Center, Los Angeles, CA (US)

(21) Appl. No.: 16/815,760

(22) Filed: Mar. 11, 2020

Related U.S. Application Data
(63) Continuation-in-part of application No. PCT/US2018/053873, filed on Oct. 2, 2018.
(60) Provisional application No. 62/566,813, filed on Oct. 2, 2017.
(60) Provisional application No. 62/818,890, filed on Mar. 15, 2019.

(51) Int. Cl. A61K 38/10; A61K 47/64; A61K 47/60; C12N 15/113; A61K 47/68; A61P 25/28; A61P 35/00; A61K 49/14

(52) U.S. Cl. CPC A61K 38/10; A61K 47/64; A61K 47/60; A61K 49/14; A61K 47/6811; A61P 25/28; A61P 35/00; C12N 15/1137

(57) ABSTRACT

Mini nanodrugs that include a polymalic-based molecular scaffold with one or more peptides capable of crossing the blood-brain barrier, one or more plaque-binding peptides and one or more therapeutic agents attached to the scaffold are provided. Methods of treating brain diseases or abnormal conditions, and imaging of the same in a subject by administering the mini nanodrugs are described. Methods for reducing formation of amyloid plaques in the brain of a subject are disclosed.

### CROSS REFERENCE TO RELATED APPLICATION

This is a continuation-in-part of international patent application No. PCT/US2018/53873, filed Oct. 2, 2018, which claims the benefit of U.S. provisional application No. 62/566,813, filed Oct. 2, 2017. This application also claims the benefit of U.S. provisional application No. 62/818,890, filed Mar. 15, 2019, all of which are incorporated herein by reference as if fully set forth.

The sequence listing electronically filed with this application titled “Sequence Listing,” which was created on Mar. 11, 2020 and had a size of 3,928 bytes is incorporated by reference herein as if fully set forth.

### STATEMENT REGARDING FEDERALLY SPONSORED RESEARCH OR DEVELOPMENT

The invention was made with government support under Grant Nos. CA188743 and CA209921 awarded by National Institutes of Health. The government has certain rights in the invention.

### FIELD OF INVENTION

The disclosure generally relates to mini nanodrugs that include peptides capable of crossing blood-brain barrier, plaque-binding peptides and/or therapeutic agents conjugated to the polymalic acid-based scaffold. The also disclosure relates to methods for treating brain diseases, including neurological disorders, reducing formation of amyloid plaques in the brains of patients suffering from Alzheimer's disease, and/or imaging the same by administering the mini nanodrugs described herein.

### BACKGROUND

Insufficient delivery across the brain-blood barrier (BBB) prevents many preclinical drugs from reaching their intended targets and results in low efficiencies of conventional drug treatments for neurological disorders (Drean et al. (2016) and Abbot (2013), both of which are incorporated by reference as if fully set forth). Drug delivery across the BBB of healthy subjects is especially challenging because an intact BBB is largely drug impenetrable. Yet, the early treatment of neurological disease is paramount to the success of drug therapies, given that most diseases have a poor prognosis once they reach advanced stages. Moreover, early drug treatment of neuroinflammation and neurodegeneration may prevent the deterioration of the BBB all-together and could maintain its protective ability of excluding infiltrating cytokines and toxins (Alyautdin et al. (2014), which is incorporated herein by reference as if fully set forth).

Attempts to deliver across BBB were used to treat brain tumors by targeting with transcytosis specific peptides. Delivered chemotherapeutics were either direct conjugation of paclitaxel, PTX-Biotin-CPP, or examining αᵥβ₃ integrin chemically attached to PAMAM-G5 dendrimer, peptides targeting paclitaxel-methoxy poly(ethylene glycol)-co-poly(ε-caprolactone)copolymer, polymersomes, or delivery of a suicide gene encapsulated by Angiopep-2-PEG-conjugated nanoparticles of poly (L-lysine)-grafted polyethyleneimine (PEI-PLL) (Regina et al. (2008) Li et al. (2016) Yan et al. (2012); Xinet al. (2012); Lu et al. (2017); and Morales-Zavalaa et al. (2017), all of which are incorporated herein by reference as if fully set forth).

Brain delivery to non-tumor targets were described for the rod-shaped nanoparticles (C. elegans Alzheimer model), PTX (for breast cancer metastases, PET and MRI), electro responsive hydrogel nanoparticles (delivery of anti-seizure Phenytoin), neurotensin (a modulator of nociceptive transmission) O'Sullivan et al. (2016); Gao et al. (2016); Wang et al. (2016); and Demeule et al. (2014), all of which are incorporated herein by reference as if fully set forth).

The examples of targeted delivery across BBB to treat tumors in the brain do not adequately represent the delivery across BBB of healthy brain. In the other examples, small compounds are delivered which readily permeate BBB on their own account.

The penetration of nanodevices across healthy BBB has not been unequivocally accessed by microscopic demonstration.

In addition to delivery of drugs across BBB, another problem is to reduce activity of key markers in Alzheimer diseases such as secretases and Tau protein.

A most advanced example for inhibiting Aβ production is by intravenous injection combined the peptide targeted delivery across BBB and siRNA knockdown of BACE1 β-secretase in neurons (Zheng et al. (2017), which is incorporated herein by reference as if fully set forth). The micellar nanodrug targeted by a specific peptide, selected from a display, for attachment to amyloid peptides, probably including precursor protein (APP) on the surface of neuron cells, was then intracellularly delivered into the neuron endosomal/lysosomal pathway and finally escaped into the cytoplasm to block the secretase mRNA (Zheng et al. (2017), which is incorporated herein by reference as if fully set forth).

The Aβ1-42 targeting D-peptide has been screened using a mirror imaging display selection and has a binding affinity in the sub-micro molar concentration (Wiesehan et al. (2003), which is incorporated herein by reference as if fully set forth).

A study of antisense oligonucleotides (ASO) Tau^{ASO-12} directed against human tau involved the use PS19 mice as tauopathy mouse model that overexpressed a mutant form of tau (DeVos et al. (2017), which is incorporated herein by reference as if fully set forth). The ASO containing fluid was pump-infused into the right lateral ventricle. The ASO application was not targeted and distributed over the brain. Tau mRNA and protein was reduced in the brain spinal cord and cerebrospinal fluid. Mouse survival was extended, and pathological Tau seeding was reversed. While the siRNA knockdown of BACE1 was advanced using systemic injection, that of Tau was in an initial stage, and circumstantial using direct application and prolonged pumping into the brain.

Numerous small molecule inhibitors, peptides and synthetic compounds, have been synthesized, but none passed through clinical trials. Failure could have been lack or impaired BBB penetration, fast clearance from the brain and lack of targeting the diseased neuro cells (Vassar R. (2014), which is incorporated herein by reference as if fully set forth).

Additionally, certain nanoparticles deliver drugs by encapsulation, but they have unfavorable hydrodynamic diameters in the range 30-300 nm and limited BBB penetration. Such particles are also not biodegradable and can result in toxic, insoluble depositions. In addition, nonspecific drug effects may arise due to spontaneous release of drug cargo, via drug diffusion, or via nanoparticle dissolution (Elnegaard et al. (2017), which is incorporated by reference as if fully set forth).

Certain antibody-based drugs, on the other hand, penetrate the BBB and have provided promising results in the laboratory as well as in preclinical treatment trials of neurological disorders, including Alzheimer's disease (Sevigny et al. (2016), which is incorporated as if fully set forth).

However, antibody-based therapeutics, even when humanized, can trigger systemic immune-responses, which complicate long-term treatment perspectives (Borlak et al. (2016), which is incorporated by reference as if fully set forth).

Moreover, antibody molecules are large and limit cargo capacity and hence the delivery of multiple drug cargoes to recipient cells.

### SUMMARY

In an aspect, the invention relates to a mini nanodrug comprising a polymalic acid-based molecular scaffold, at least one peptide capable of crossing the blood-brain barrier, at least one plaque-binding peptide and an endosomolytic ligand. The at least one peptide capable of crossing the blood-brain barrier, the at least one plaque-binding peptide and the endosomolytic ligand are covalently linked to the polymalic acid-based molecular scaffold. The mini nanodrug ranges in size from 1 nm to 10 nm.

In an aspect, the invention relates to a mini nanodrug comprising a polymalic acid-based molecular scaffold, at least one peptide capable of crossing the blood-brain barrier, an endosomolytic ligand and a therapeutic agent. Each of the at least peptide, the endosomolytic ligand and the therapeutic agent are covalently linked to the polymalic acid-based molecular scaffold. The mini nanodrug ranges in size from 1 nm to 10 nm.

In an aspect, the invention relates to a pharmaceutically acceptable composition comprising any one of the mini nanodrugs described herein and a pharmaceutically acceptable carrier or excipient.

In an aspect, the invention relates to a method for treating a disease or abnormal condition in a subject. The method comprises administering a therapeutically effective amount of any one of the mini nanodrugs described herein or any one of the pharmaceutically acceptable compositions described herein to a subject in need thereof.

In an aspect, the invention relates to a method for reducing formation of amyloid plaques in the brain of a subject. The method comprises administering any one of the mini nanodrugs described herein, or any one of the compositions described herein to a subject in need thereof.

In an aspect, the invention relates to a method for treating a proliferative disease in a subject. The method comprises administering a therapeutically effective amount of a mini nanodrug comprising a polymalic acid-based molecular scaffold, at least one peptide capable of crossing the blood-brain barrier, an endosomolytic ligand and an therapeutic agent to a subject in need thereof. Each of the at least peptide, the endosomolytic ligand and the therapeutic agent are covalently linked to the polymalic acid-based molecular scaffold. The mini nanodrug ranges in size from 1 nm to 10 nm.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The following detailed description of the preferred embodiments will be better understood when read in conjunction with the appended drawings.

For the purpose of illustration, there are shown in the drawings embodiments which are presently preferred. It is understood, however, that the invention is not limited to the precise arrangements and instrumentalities shown. In the drawings:

FIG. 1 is a schematic drawing illustrating overview of molecular pathway for the delivery of the mini nanodrugs of the embodiments described herein.

FIG. 2 is a schematic drawing illustrating mini nanodrugs that permeate through multiple bio barriers into targeted tumors.

FIGS. 3A-3B are schematic drawings illustrating advantages of mini nanodrugs for crossing the blood-brain barrier and entering brain parenchyma. FIG. 3A is a schematic drawing illustrating mini nanodrugs carrying AP-2 peptides and tri-leucines (endosomic escape units) entering brain parenchyma. FIG. 3B is a schematic drawing comparing the efficiency of crossing the blood-brain barrier of a mini nanodrug carrying peptides and nanodrugs that carry antibodies.

FIG. 4 is a schematic drawing for synthesis of a mini nanodrug containing a single peptide.

FIGS. 5A-5F illustrate examples of the mini nanodrugs, containing peptides, AONs and antibodies.

FIG. 5A illustrates an example of the mini nanodrugs containing three peptides.

FIG. 5B illustrates an example of the mini nanodrugs containing LLL (40%), BBB-penetrating peptide (2%) and rhodamine dye (1%).

FIG. 5C illustrates an example of the mini nanodrug containing LLL (40%), D peptide (2%), and AON-fluorescein.

FIG. 5D illustrates an example of the mini nanodrug containing LLL (40%), D peptide (2%), rhodamine dye (1%) and AON.

FIG. 5E illustrates an example of the mini nanodrugs containing LLL (40%), BBB-penetrating peptide (2%), IgG (0.2%) and rhodamine dye (1%).

FIG. 5F illustrates an example of the mini nanodrugs containing LLL (40%), ab-TfR or IgG (0.2%) and rhodamine dye (1%).

FIGS. 6A-6C illustrate characterization of synthesized P/LLL/AP-2/ACI-89/rhodamine FIG. 6A illustrates SEC-HPLC top view of scanning A200-A700 nm vs. retention time displaying absorbance of the complete nanoconjugate, FIG. 6B illustrates the scanning profile of the same conjugate as shown on FIG. 6A at 572 nm wavelength indicating the rhodamine component. FIG. 6C illustrates the scanning profile of the same conjugate as shown on FIG. 6A at 220 nm wavelength indicating the P/LLL/AP-2/ACI-89 component.

FIGS. 7A-7C illustrates SEC-HPLC chromatogram of P/LLL/AP-2/D1-peptide/rhodamine at A200-A700 nm vs. retention time displaying absorbance of PMLA/LLL/AP-2/D-peptide/rhodamine complete nanoconjugate. FIG. 7B is a scanning profile of the same nanoconjugate as shown on FIG. 7A at 572 nm indicating the rhodamine component. FIG. 7C is a scanning profile of the same nanoconjugate as shown on FIG. 7A at 220 nm indicating the PMLA/LLL/AP-2/D1-peptide component.

FIGS. 8A-8C illustrate characterization of synthesized P/LLL/AP-2/D3-peptide/rhodamine. FIG. 8A illustrates SEC-HPLC top view displaying A200-A700 nm vs. retention time and absorbance of the P/LLL/AP-2/D3-peptide/rhodamine complete nanoconjugate. FIG. 8B is the scanning profile of the same nanoconjugate as shown on FIG. 8A at 572 nm absorbance of rhodamine. FIG. 8C is the scanning profile of the nanoconjugate recorded at 220 nm wavelength for the P/LLL/AP-2/D3-peptide component.

FIGS. 9A-9G illustrate examples of product verification by HPLC. FIG. 9A illustrates verification of PMLA/LLL/Angiopep-2-PEG3400-MAL/rhodamine. FIG. 9B illustrates verification of PMLA/LLL/“Fe mimetic peptide” (SEQ ID NO: 2) CRTIGPSVC (cyclic)-peptide-PEG2000-Mal/rhodamine. FIG. 9C illustrates verification PMLA/LLL/Miniap-4-PEG2000-Mal/cy 5.5. FIG. 9D illustrates control: PMLA/LLL/rhodamine. FIG. 9E-FIG. 9G illustrate HPLC elutions of the peptide nanoconjugates measured at 220 nm wavelength. FIG. 9E illustrates PMLA/LLL/Angiopep2 (2%)/“Fe Mimetic Peptide” (2%)/rhodamine (1%) dipeptide for targeting. FIG. 9F illustrates PMLA/LLL/angiopep-2 (2%)/miniap-4 (2%)/rhodamine (1%) dipeptide for targeting. FIG. 9G illustrates PMLA/LLL/miniap-4 (2%)/angiopep-2 (2%)/“Fe mimetic peptide” (2%)/rhodamine (1%) tripeptide for targeting. The terms “Fe mimetic peptide” and “cTfRL” are used interchangeably herein

FIGS. 10A-10C illustrate characterization of synthesized P/LLL/AP2. FIG. 10A illustrates SEC-HPLC 3D view of A200-A700 nm vs. retention time and absorbance of the P/LLL/AP2 nanoconjugate constituents. FIG. 10B illustrates SEC-HPLC chromatogram of P/LLL/AP2 recorded at 220 nm wavelength. FIG. 10C illustrates the FTIR (Fourier-transform infrared) spectrum of P/LLL/AP2 nanoconjugate (rhodamine not conjugated; dashed line), AP2 free peptide (solid line) and pre-conjugate (dashed-dotted line).

FIG. 11 illustrates PK for P/LLL/AP-2 (2%)/rhodamine(1%) conjugate measured by fluorescence intensity of the attached dye as a function of time from IV injection into tail vain until blood samples were taken.

FIG. 12 is a photograph of the left hippocampus CA1 examined under fluorescence 2 hours following IV injection of PBS buffer into the tail vain of a mouse

FIG. 13 is a schematic drawing of the brain showing main blood vessels including the superior sagittal sinus (SSS), a large blood vessel that runs along the midline of the brain.

FIGS. 14A-14C illustrate concentration dependent BBB penetration of P/LLL/AP-2/rhodamine. FIG. 14A is a set of photographs illustrating optical imaging data acquired at 120 min after i.v. injection of P/LLL/AP-2/rhodamine at the following concentrations: photograph 1-0.068 μmol/kg; photograph 2-0.173 μmol/kg; photograph 3-0.274 μmol/kg; and photograph 4-0.548 μmol/kg. FIG. 14B is a chart illustrating nanoconjugate fluorescence intensity vs. “distance from vasculature” measurements in brain parenchyma of mice injected with three different concentrations: black: 0.548 μmol/kg; grey: 0.273 μmol/kg; white: 0.068 μmol/kg. FIG. 14C is set of charts: chart 1—Cortex; chart 2—Midbrain and chart 3 Hippocampus, illustrating average nanoconjugate fluorescence in the brain parenchyma measured following injections at four different drug concentrations. The terms “P/LLL/AP-2” and “P/LLL/AP-2/rhodamine” are used interchangeably herein in reference to the mini nanodrugs.

FIGS. 15A-15D illustrate blood vessel diameters, vascular coverage and inter-vessel distances in different brain regions. FIG. 15A is a set of photographs illustrating blood vessels in the cortex, midbrain and hippocampal CA1 cellular layer (outlined). FIG. 15B is a bar graph illustrating vessel diameters. FIG. 15C are bar graphs illustrating vascular coverage. FIG. 15D illustrates the inter vessel distance defined as the shortest (Euclidian) distance between two adjacent blood vessels, comprehensively sampled for all vessels in each image.

FIGS. 16A-16B illustrate that the nanoconjugate composition determines degree and locus of BBB penetration. FIG. 16A is set of photographs illustrating nanoconjugate permeation of the cerebral cortex: photograph 1-P/LLL/AP-2; photograph 2-P/AP-2 and photograph 3-P/LLL at constant injected dose (0.274 μmol/kg). FIG. 16B is a set of bar graphs showing average nanoconjugate fluorescence in the cerebral cortex (1), the midbrain (2) and the hippocampus (2) as a function of nanoconjugate composition and concentration: P/LLL/AP-2 is shown in black, P/AP-2 in grey and P/LLL in white. All nanoconjugates referenced in FIGS. 16A-16B contain rhodamine.

FIGS. 17A-17B illustrate the effect of conjugated LLL residues on nanoconjugate conformation. FIG. 17A is a chemical structure of the conjugate. LLL is indicated with black arrows in the structural scheme. FIG. 17B is a three-dimensional image of short PMLA (16 malic acid residues) with PEG (2 chains of ethylene glycol-hexamer conjugated via maleimide to PMLA), capped sulfhydryl (two moieties) and LLL (4 moieties).

FIGS. 18A-18B illustrate nanoconjugate conformation in the absence of LLL. FIG. 18A illustrates the structural model, and is similar as the one shown in FIG. 18A but lacking LLL. FIG. 18B is a three-dimensional image of the structure shown in FIG. 18A.

FIGS. 19A-19E illustrate nanoconjugate peptide moiety screen. FIG. 19A is a set of photographs illustrating the P/LLL nanoconjugates equipped with different peptides (1—P/LLL/AP-2; 2—P/LLL/M4; and 3—P/LLL/B6) to assess their role in BBB penetration following the injection into mice at the concentration of 0.274 μmol/kg (i.e., at a constant injected dose). FIGS. 19B-19D is a set of bar graphs showing average nanoconjugate fluorescence in the cerebral cortex (FIG. 19B), midbrain (FIG. 19C) and hippocampus (FIG. 19D) as a function of injected concentration.

FIG. 19E illustrates nanoconjugate fluorescence measurements in the cerebral cortex (1), midbrain colliculi (2), hippocampus CA1-3 layers (3) for peptide combinations P/LLL/AP2/rh (three light grey bars on the left side), P/LLL/AP2//M4/rh (light grey bar on the middle right) and P/LLL/AP7/rh (grey bar on the right) injected at concentrations of 0.137 μmol/kg or 0.274 μmol/kg.

FIGS. 20A-20D illustrates pharmacokinetics of nanoconjugate fluorescence in serum and brain tissue. FIG. 20A is a chart illustrating serum clearance analysis was conducted for P/LLL/AP-2 (black) and P/LLL (grey), and optically via imaging of the cerebral vasculature content (black, triangles). FIG. 20B is a set of photographs illustrating optical imaging data of and around the saggital sinus showing drug clearance and parenchyma accumulation over 240 minutes. FIG. 20C illustrates vascular fluorescence intensity profile for the saggital sinus as indicated along the white line in the utmost left panel of FIG. 20B. FIG. 20D is a bar graph illustrating time dependence of nanoconjugate fluorescence intensity in brain tissue for P/LLL/AP-2 (black), P/LLL (grey) and P/AP-2 (white) that are different from the serum PK kinetics. All nanoconjugates referenced in FIGS. 20A-20D contain rhodamine.

FIGS. 21A-21C illustrate concentrations indicated by clouds in different shades of grey of the nanoconjugate (A1-A2) and quantitative in μg/mL in FIG. 21B and FIG. 21C after i.v. injection of P/LLL/AP-2 in the parenchyma of the cerebral cortex. FIG. 21A is set of photographs illustrating optical imaging data showing cortical tissue from mice injected with P/LLL/AP-2 at 0.068 μmol/kg (A1) and 0.274 μmol/kg (A2) and regions (dotted) of interest for comparison of fluorescence intensities in vascular tissue and parenchyma. FIG. 21B illustrates fluorescence ratios in vasculature/cortical brain parenchyma. FIG. 21C illustrates estimated P/LLL/AP-2 concentration in the cortical brain parenchyma as a function of injected dose, based on known concentrations from PK measurements in the vascular and the measured intensity ratios of fluorescence in the vascular to the regions of interest. All nanoconjugates referenced in FIGS. 21A-2C contain rhodamine.

FIGS. 22A-22C illustrate optical imaging data of the normal brain following mice injection with nanoconjugates labeled with rhodamine.

FIG. 22A is a set of photographs illustrating optical imaging data in cortex of normal brain following the injection of mice with 0.274 μmol/kg P/LLL/AP2/rh (left), 0.274 μmol/kg P/LLL/D 1/rh (middle) and 0.274 μmol/kg P/LLL/D1/rh and 21 μmol/kg AP2.

FIG. 22B are bar graphs illustrating the intensity of fluorescence in the samples of the normal brain following injections of mice with 0.274 μmol/kg (4×) of P/LLL/AP2/rh, P/LLL/AP2/D1/rh, P/LLL/D1/rh, P/LLL/AC189/rh, P/LLL/D3/rh or PBS buffer in layers II/III cortex (left), hippocampus CA₁₋₃ (middle) and midbrain colliculi (right).

FIG. 22C are bar graphs illustrating the intensity of fluorescence in the samples of the normal brain following injections of mice with 0.274 μmol/kg of P/LLL/AP2/D1/rh, 0.274 μmol/kg P/LLL/D1/rh and 21 μmol/kg of AP2, or PBS buffer in layers II/III cortex (left), midbrain colliculi (middle) and hippocampus CA₁₋₃ (right).

FIGS. 23A-23C illustrate peptide-dependent labeling of plaques by injected nanoconjugates labeled with rhodamine. FIG. 23A is a photograph illustrating optical imaging data following mice injected with P/LLL/M4. FIG. 23B is a photograph illustrating optical imaging data following mice injected with P/LLL/M4/D 1. FIG. 23C is a bar graph showing fluorescence intensities of Aβ (plaque) binding of nanoconjugates PMLA, P/cTfRL, P/M4, P/LLL, P/LLL/AP-2, P/LLL/M4, P/AP-2/ACI-89, P/LLL/AP-2/D3, P/LLL/AP-2/D1 and P/LLL/M4/D 1 labeled with rhodamine. Plaque vs. background labeling (signal noise) is indicated.

FIG. 24 is a set of photographs illustrating optical imaging data of the brain cortex following the injection of mice with 0.274 μmol/kg of P/LLL/AP2/rh (bottom), or P/LLL/D 1/rh (top).

FIGS. 25A-25B illustrate optical imaging data of brain parenchyma following injection of mice with 0.274 μmol/kg of P/LLL/D 1/rh and 0.274 μmol/kg P/LLL/D1/rh+21 μmol/kg of AP2 (top).

FIG. 25A is a set of photographs illustrating optical imaging data of the brain cortex following the injection of mice with 0.274 μmol/kg of P/LLL/D1/rh (bottom), and 0.274 μmol/kg P/LLL/D1/rh+21 μmol/kg of AP2 (top).

FIG. 25B are bar graphs illustrating the intensity of fluorescence in the samples of the brain parenchyma following injections of mice with 0.274 μmol/kg of P/LLL/D1/rh, P/LLL/D1/rh+21 μmol/kg of AP2 or PBS buffer in layers II/III cortex (left), midbrain colliculi (middle) and hippocampus CA₁₋₃ (right).

FIGS. 26A-26B are scatter plots and line graphs illustrating drug penetration distance through the brain parenchyma extracellular matrix (the intensity of fluorescence vs. distance from the nearest blood vessel) calculated for P/LLL/AP2/rh, P/LLL/AC189/rh, P/LLL/D1/rh and P/LLL/D3/rh in the cortex (FIG. 26A) and hippocampus (FIG. 26B).

FIGS. 27A-27C illustrate fluorescence uptake in the hippocampus and cortex neurons and astroglia.

FIGS. 27A and 27B are set of photographs of neurons and astroglia in hippocampus (FIG. 27A) and cortex (FIG. 27B) of animals that were injected with PBS and P/LLL/ACI89.

FIG. 27C is a set of photographs showing the drug fluorescence (left) and merged (right) only for P/LLL/ACI89 nanoconjugate.

FIG. 28 is a set of photographs showing fluorescence uptake in the cortical layer II/III (B) neurons and astroglia in cortical layers II/III of animals that were injected with P/LLL/D 1/rh, P/LLL/ACI89/rh, P/LLL/D3/rh and PBS.

FIGS. 29A-29D illustrate intracellular fluorescence of mini nanodrugs.

FIG. 29A is an image of the P/LLL/D1 conjugate which demonstrates the method: 20*20 μm² ROIs were placed randomly however away from vessels for each image. Each ROI was converted to binary (black and white) image and the area and number of particles were quantified. 3 images per brain area were tested for 3 mice per group.

FIGS. 29B-29D are bar graphs illustrating intracellular accumulation of measured ROI as average area per particle in samples of the brain following injections of mice with P/LLL/AP2/rh, P/LLL/D1/rh, P/LLL/AC189/rh, P/LLL/D3/rh, or PBS in cortex (FIG. 29B), midbrain (FIG. 29C) or hippocampus (FIG. 29D).

FIGS. 30A-30B illustrate fluorescence in neurons following mice injection with mini nanodrugs.

FIG. 30A is a set of photographs of neuron staining and optical imaging of the brain following injections of mice with 0.274 μmol/Kg of P/LLL/D3/rh: neuron nucleus (yellow, Neun) surrounded with ROIs (top left), drug (gray, rhodamine channel) and ROI's (yellow) (top right) and drug only (grey) (bottom).

FIG. 30B are bar graphs illustrating average fluorescence per neuron nucleus, after PBS deduction of P/LLL/D3/rh (0.274 μmol/Kg), P/LLL/D1 (0.274 μmol/kg), and P/LLL/ACI89 (0274 μmol/Kg). All statistical tests were conducted as a one-way ANOVA with Tukey t-tests conducted between experimental conditions in each brain regions. Statistical significance is indicated as follows: *=p<0.01, **=p<0.001, and ***=p<0.0001.

FIGS. 31A-31C are optical imaging data following mice injections with mini nanodrugs that carry AONs.

FIG. 31A is a set of photographs showing optical imaging data in the samples of the brain cortex following mice injection with P/LLL/D1/AON-F, P/LLL/D3/AON-F and P/LLL/AON-F. Combined images on the left show lectin stained vessels in red, labeled nanoconjugate in green, and DAPI in blue. The correlating binary image used to calculate particulate fluorescence is shown to the right.

FIG. 31B are bar graphs showing data of the diffused fluorescence measurements in the cortex following mice injection with P/LLL/AON-F, P/LLL/D1/AON-F, and P/LLL/D3/AON-F.

FIG. 31C are bar graphs showing data of the particulate fluorescence analysis (area per particle, μm²) in the cortex following mice injection with P/LLL/AON-F, P/LLL/D1/AON-F, and P/LLL/D3/AON-F. All statistical tests were conducted as a one-way ANOVA with post-hoc Tukey t-tests. Statistical significance is indicated as follows: *=p<0.01, **=p<0.001, and ***=p<0.0001.

FIGS. 32A-32D show the effect of doubling the injected dosage for P/LLL/D3/rh/AON on the level of fluorescence in the parenchyma (diffusible nanoconjugate) and the area of fluorescence emitted by the particles after internalization into the brain cells.

FIG. 32A is a set of photographs showing optical imaging data in the brain cortex following injection of the mice with 0.274 μmol/Kg of P/LLL/D3/AON/rh.

FIG. 32B is a set of photographs showing optical imaging data in the brain cortex following injection of the mice with 0.55 μmol/Kg of P/LLL/D3/AON/rh.

FIG. 32C are bar graphs showing data of the diffused fluorescence measurements in the cortex and dose dependence following injection of the mice with P/LLL/D3/AON/rh, P/LLL/D3/rh or PBS.

FIG. 32D are bar graphs showing data of the particulate fluorescence analysis (area per particle, μm²) in the cortex following mice injection with P/LLL/D3/AON/rh, P/LLL/D3/rh or PBS.

FIG. 33 is set of photographs illustrating optical imaging data of the midbrain following the injection of mice with P/LLL/AP-2/IgG, in which P (or polymalic acid backbone) is labeled with rhodamine for fluorescence (top row) and P/LLL/AP-2/IgG, in which IgG is labeled with rhodamine for fluorescence (bottom row). 1× is the dose 0.068 μmol/kg

FIG. 34 are bar graphs illustrating the intensity of fluorescence in the samples of the brain following injections of mice with P/LLL (40%/AP-2/IgG-rh (0.2%), P/LLL/IgG-rh (0.2%) or PBS buffer in cortex (left graph) and midbrain (right graph).

FIGS. 35A-35C illustrate optical imaging data of the brain tissue following mice injections with P/LLL/AP2/IgG/rh, P/LLL/AP2/IgG-rh, and P/LLL/AP2/rh mini nanodrugs.

FIG. 35A is a set of photographs illustrating optical imaging data of the brain following the injection of mice with 2× (0.137 μmol/kg) of P/LLL/AP2/IgG/rh (left), P/LLL/AP2/IgG-rh (middle), P/LLL/AP2/rh (right).

FIG. 35B are bar graphs illustrating the intensity of fluorescence in the cortex layer II/III, midbrain colliculi and hippocampus following 2 hours post injections of mice with P/LLL/AP2/IgG/rh, P/LLL/AP2/IgG-rh, P/LLL/AP2/rh, or PBS buffer.

FIG. 35C are bar graphs illustrating the intensity of fluorescence in the cortex layer II/III, midbrain colliculi and hippocampus CA1-3 layer following 30, 60, 120, 240, or 480 minutes post injections of mice with P/LLL/AP2/IgG/rh or PBS buffer.

FIGS. 36A-36F are bar graphs illustrating optical data quantification 2 hours post injection for IgG and non-IgG mini nanodrugs at 0.274 μmol/kg (4×). From left to right: Cortex dark grey/black, midbrain (light grey), hippocampus (dark grey).

FIG. 36A are bar graphs illustrating the intensity of fluorescence in the brain following injections of mice with P/LLL/AP2/rh, P/LLL/AP2/IgG/rh, or PBS buffer.

FIG. 36B are bar graphs illustrating the intensity of fluorescence in the brain following injections of mice with P/LLL/B6/rh, P/LLL/B6/IgG/rh, or PBS buffer.

FIG. 36C are bar graphs illustrating the intensity of fluorescence in the brain following injections of mice with P/LLL/AD1/rh, P/LLL/D1/IgG/rh, or PBS buffer.

FIG. 36D are bar graphs illustrating the intensity of fluorescence in the brain following injections of mice with P/LLL/D3/rh, P/LLL/D3/IgG/rh, or PBS buffer.

FIG. 36E are bar graphs illustrating the intensity of fluorescence in the brain following injections of mice with P/LLL/M4/rh, P/LLL/M4/IgG/rh, or PBS buffer.

FIG. 36F are bar graphs illustrating the intensity of fluorescence in the brain following injections of mice with P/LLL/TfR-ab/rh, P/LLL/IgG/rh, P/IgG/rh or PBS buffer. Midbrain 3 groups (middle), hippocampus 3 groups (extreme right side)

FIGS. 37A-37E illustrate the BBB permeation efficacies following injections of mice with P/LLL/D1/rh, P/LLL/D3/rh, P/LLL/B6/rh, P/LLL/rh, P/LLL/M4/rh, P/LLL/AP2/rh

FIG. 37A is a set of photographs illustrating optical imaging data of the cortex of the AD brain following the injection of mice with 8× [0.548 μmol/kg] of each of P/LLL/D3/rh (top left), P/LLL/B6/rh (top middle), P/LLL/AP2/rh (top right), P/LLL/rh (bottom left), P/LLL/D 1/rh (bottom middle), and P/LLL/M4/rh (bottom right) in the tumor (left) and the other hemisphere (brain; right).

FIG. 37B are bar graphs illustrating the intensity of fluorescence in the hippocampus of AD brain following injections of mice P/LLL/D 1/rh, P/LLL/D3/rh, P/LLL/B6/rh, P/LLL/rh, P/LLL/M4/rh, P/LLL/AP2/rh or PBS buffer.

FIG. 37C are bar graphs illustrating the intensity of fluorescence in the cortex of AD brain following injections of mice with nanodrug P/LLL/D 1/rh, P/LLL/D3/rh, P/LLL/B6/rh, P/LLL/rh, P/LLL/M4/rh, P/LLL/AP2/rh or PBS buffer.

FIG. 37D are bar graphs illustrating the intensity of fluorescence in AD brain parenchyma following injections of mice with P/LLL/D3/rh or PBS buffer at 2×, 4×, 6×, or 8× dose in the cortex or hippocampus.

FIG. 37E is a photographs illustrating optical imaging data of Aβ plaque in the AD brain parenchyma surrounded by astrocytes (in green) following the injection of mice with P/LLL/D3/rh.

FIGS. 38A-38B are bar graphs illustrating the mean intensity of fluorescence (after PBS deduction) in the normal, AD and tumor (FIG. 38A) or normal and AD brain (FIG. 38B) following injections of mice with 8× of P/LLL/AP2/rh, P/LLL/D3/rh, P/LLL/B6/rh, P/LLL/AP2/rh, P/LLL/D3/rh, P/LLL/B6/rh P/LLL//rh, and 4× of P/LLL/AP2/rh, P/LLL/D3/rh, P/LLL/B6/rh.

FIGS. 39A-39C illustrate optical imaging data the tumor area and the corresponding non-tumor symmetrically positioned in the other brain hemisphere following mice injections with the mini nanodrugs.

FIG. 39A is a set of photographs illustrating optical imaging data in cortex of tumor bearing brain following the injection of mice with 1× (0.0685 μmol/kg) or 4× (0.274 μmol/kg) of P/LLL/B6/rh (bottom), P/LLL/AP2/rh (middle) and P/LLL/rh in the tumor (left) and the other hemisphere (brain; right).

FIG. 39B is a set of photographs illustrating optical imaging data in cortex of tumor bearing brain following the injection of mice with 4× (0.274 μmol/kg) of P/LLL/D3/rh (left), P/LLL/M4/rh (middle left), P/LLL/D 1/rh (middle right) and P/LLL/AC189/rh (right).

FIG. 39C are bar graphs illustrating the intensity of fluorescence in the tumor following injections of mice with 1× of P/LLL/B6/rh, P/LLL/AP2/rh P/LLL/rh, and 4× of P/LLL/B6/rh, P/LLL/rh, P/LLL/AP2/rh, P/LLL/D 1/rh, P/LLL/AC189/rh, P/LLL/D3/rh, P/LLL/M4/rh or PBS buffer.

FIGS. 40A-40B are schematic representations of the mini nanodrugs binding via two pathways mechanism (FIG. 40A) and via the allosteric mechanism (FIG. 40B).

FIGS. 41A-41F illustrate factorial study data for P/LLL/AP2/B6/rh matrix (FIGS. 41A, 41C and 41E) and P/LLL/AP2/rh matrix (FIGS. 41B, 41D and 41F).

FIGS. 41A and 41B illustrate 2D contour plots for the response tumor/brain (T/B) (axis: Z-T/B ratio, Y-% of AP2 loading and X-dose).

FIGS. 41C and 41D illustrate the pareto charts for standardized effects for tumor fluorescence intensity response.

FIGS. 41E and 41F illustrate interaction plots for T/B ratio response.

### DETAILED DESCRIPTION OF THE PREFERRED EMBODIMENTS

Certain terminology is used in the following description for convenience only and is not limiting. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims Further, unless otherwise required by context, singular terms shall include pluralities and plural terms shall include the singular.

The singular terms “a,” “an,” and “the” include plural referents unless context clearly indicates otherwise. Similarly, the word “or” is intended to include “and” unless the context clearly indicates otherwise.

The phrase “at least one” followed by a list of two or more items, such as “A, B, or C,” means any individual one of A, B or C as well as any combination thereof.

The words “right,” “left,” “top,” and “bottom” designate directions in the drawings to which reference is made.

Although methods and materials similar or equivalent to those described herein can be used in the practice or testing of this disclosure, suitable methods and materials are described below.

The term “peptide” refers to a contiguous and relatively short sequence of amino acids linked by peptidyl bonds. The terms “peptide” and “polypeptide” are used interchangeably.” The peptide may have a length of about 2 to 10 amino acids, 8 to 20 amino acids or 6 to 25 amino acids.

The terms “amino acid” and “amino acid residue” are used interchangeably herein.

An “abnormal condition” refers to a function in the cells and tissues in a body of a patient that deviates from the normal function in the body. An abnormal condition may refer to a disease. Abnormal condition may include brain disorders. Brain disorders may be but are not limited to Alzheimer's disease, Multiple sclerosis, Parkinson's disease, Huntington's disease, schizophrenia, anxiety, dementia, mental retardation, and anxiety. Abnormal condition may include proliferative disorders. The terms “proliferative disorder” and “proliferative disease” refer to disorders associated with abnormal cell proliferation. Proliferative disorders may be, but are not limited to, cancer, vasculogenesis, psoriasis, and fibrotic disorders.

An embodiment provides a mini nanodrug comprising a polymalic acid-based molecular scaffold, one or more peptides capable of crossing the blood-brain barrier, an endosomolytic ligand and a therapeutic agent. Each of the peptides capable of crossing the blood-brain barrier, endosomolytic ligand and therapeutic agent may be covalently linked to the polymalic acid-based molecular scaffold.

As used herein, the term “peptide capable of crossing blood-brain barrier” refers to any peptide that can bind to receptors responsible for maintaining the integrity of the brain-blood barrier and brain homeostasis. One or more peptides capable of crossing blood-brain barrier may be an LRP-1 ligand, or a transferrin receptor ligand. One or more peptides capable of crossing blood-brain barrier may be a peptide that may bind the low density lipoprotein (LDL) receptor-related protein (LPR), which possesses the ability to mediate transport of ligands across endothelial cells of the brain-blood barrier. One or more peptides capable of crossing blood-brain barrier may be Angiopep-2, an aprotinine-derived peptide, capable of binding lipoprotein receptor-related protein-1 (LRP-1) and promoting drug delivery in the CNS (Demeule et al., 2008, which is incorporated herein by reference as if fully set forth). The terms “Angiopep-2” and “AP-2” are used herein interchangeably. The Angiopep-2 may be the cysteine-modified Angiopep-2. The cysteine-modified Angiopep-2 peptide may be a peptide comprising the amino acid sequence TFFYGGSRGKRNNFKTEEYC (SEQ ID NO: 1). The Angiopep-2 peptide may be a variant of Angiopep-2 peptide. The variant of the Angiopep-2 peptide may be a peptide comprising an amino acid with at least 70, 72, 75, 80, 85, 90, 91, 92, 93, 94, 95, 96, 97, 98, 99 or 100% sequence identity to a sequence of SEQ ID NO: 1. The variant of the Angiopep-2 peptide may be any variant of the sequence of SEQ ID NO: 1, in which lysine residue at the positions 10 and/or 15 remain invariant.

One or more peptides may be any other peptide capable of binding LPR, crossing blood-brain barrier, and promoting delivery of the mini nanodrug in the CNS.

In an embodiment, one or more peptides may be a peptide that enhances penetration of any one of the mini nanodrugs described herein across the blood-brain barrier via the transferrin receptor (TfR) pathway. The TfR pathway imports iron (complexed to transferrin, Tf) into the brain and is involved in cerebral iron homeostasis. One or more peptides capable of crossing the blood-brain barrier may be a ligand binding to TfR or a ligand binding to transferrin (Tf). The transferrin ligand may be a Fe mimetic peptide, also referred to herein as cTfRL. The Fe mimetic peptide may be a peptide comprising the amino acid sequence CRTIGPSVC (SEQ ID NO: 2). The variant of the Fe mimetic peptide may be a peptide comprising an amino acid with at least 70, 72, 75, 80, 85, 90, 91, 92, 93, 94, 95, 96, 97, 98, 99 or 100% sequence identity to a sequence of SEQ ID NO: 2. The variant of the Fe mimetic peptide may be any variant of the sequence of SEQ ID NO: 2, which is capable to bind its target and penetrate the blood-brain barrier. For example, the variant binding to the immobilized transferrin (Tf) which further binds the transferrin receptor (TfR) may be tested by the surface plasmon resonance (SPR) method (Ding et al. (2016), which is incorporated herein by reference as if fully set forth). The Fe mimetic peptide or a variant thereof may be cyclic, may comprise disulfide bonds (—S—S—), or may comprise any other modifications known in the art. The Fe mimetic peptide or a variant thereof may be linked to PMLA via an appropriate linker at its terminal —NH₂ group when the sulfhydryls forms a disulfide (—SS—)-cyclic variant, or in the linear version at one of the thio groups as thioether.

In an embodiment, the transferrin receptor ligand may be a B6 peptide. The B6 peptide may be a peptide comprising the amino acid sequence CGHKAKGPRK (SEQ ID NO: 8). The B6 peptide may be a peptide comprising an amino acid with at least 70, 72, 75, 80, 85, 90, 91, 92, 93, 94, 95, 96, 97, 98, 99 or 100% sequence identity to an amino acid sequence of SEQ ID NO: 8. The variant of the B6 peptide may be any variant of the amino acid sequence of SEQ ID NO: 8, which is capable to bind its target TfR and penetrate the blood-brain barrier. Binding of the variant of the B6 peptide to a transferrin receptor (TfR) can be tested, for example, by the surface plasmon resonance (SPR) method (Ding et al. (2016), which is incorporated herein by reference as if fully set forth).

One or more peptides capable of crossing the blood-brain barrier may be the MiniAp-4 peptide. MiniAp-4 is a peptide derived from the bee venom, and is capable of penetrating the blood-brain barrier (Oller-Salvia et al. 2010, which is incorporated herein by reference as if fully set forth). The MiniAp-4 peptide may be a peptide comprising the amino acid sequence KAPETAL D (SEQ ID NO: 3). The MiniAp-4 peptide may comprise an amino acid sequence with at least 70, 72, 75, 80, 85, 90, 91, 92, 93, 94, 95, 96, 97, 98, 99 or 100% identity to a sequence of SEQ ID NO: 3. The variant of the MiniAp-4 peptide may be any variant of the sequence of SEQ ID NO: 3, which is capable of penetrating the blood-brain barrier (BBB). Assays for measuring BBB permeation activity are known in the art. For example, BBB permeation of mini nanodrugs can be assayed ex vivo using fluorescence imaging as described in Example 4 herein.

In an embodiment, one or more peptides capable of crossing the blood-brain barrier may be two or more peptides. Two or more peptides may be similar peptides. Two or more peptides may be selected independently.

The mini nanodrug may comprise Angiopep-2, Fe mimetic peptide, B6 peptide, and Miniap-4 peptide in any combination. The mini nanodrug may comprise any other peptides capable of crossing the blood-brain barrier.

In an embodiment, the mini nanodrug may comprise a therapeutic agent. The therapeutic agent may be an antisense oligonucleotide, an siRNA oligonucleotide, a peptide, or a low molecular weight drug. The therapeutic agent may be a combination of two or more therapeutic agents. The therapeutic agent may be an antisense oligonucleotide or an siRNA. The antisense oligonucleotide may be a Morpholino antisense oligonucleotide.

In an embodiment, the therapeutic agent may inhibit the synthesis or activity of the β-secretase or γ-secretase for amyloid 8 (Aβ) production. The antisense oligonucleotide or the siRNA may comprise a sequence complementary to a sequence contained in an mRNA transcript of β-secretase or γ-secretase. The antisense oligonucleotide may comprise a nucleic acid sequence with at least 70, 72, 75, 80, 85, 90, 91, 92, 93, 94, 95, 96, 97, 98, 99 or 100% identity to a sequence of SEQ ID NO: 4. β-secretase and γ-secretase are proteolytic enzymes that cleave the amyloid precursor protein (APP) or its proteolytic fragments at substrate specific amino acid sites and generate the amyloid-β (Aβ) peptide that accumulates in brain tissue and causes Alzheimer's disease (AD). Inhibition β- or γ-secretase activity may have therapeutic potential in the treatment of AD.

In an embodiment, the therapeutic agent may be an oligonucleotide capable of targeting a messenger RNA transcribed from a target gene. The target gene may encode β-secretase enzyme 1 or BACE1. The oligonucleotide may comprise a nucleic acid sequence with at least 70, 72, 75, 80, 85, 90, 91, 92, 93, 94, 95, 96, 97, 98, 99 or 100% identity to SEQ ID NO: 14.

In an embodiment, the mini nanodrug may comprise Angiopep-2, Fe mimetic peptide, B6 peptide, or Miniap-4 peptide, or any combination thereof, and the antisense oligonucleotide or the siRNA comprising a nucleic acid sequence complementary to the sequence contained in an mRNA transcript of β-secretase or γ-secretase.

In an embodiment, the therapeutic agent may be a therapeutic peptide, for example, for AD treatment. The therapeutic peptides may be a peptide that may target the amyloid plagues and induce the degradation activity of the mini nanodrugs to the Alzheimer disease (AD) lesions. The therapeutic peptide may be a β-sheet breaker peptide. As used herein, the term “β-sheet breaker peptide” refers to a peptide that disrupts β-sheet elements and the self-recognition motif of Aβ by inhibiting the interconnection of β-sheet Aβ1-42, so as to prevent misfolding and aggregation of Aβ (Lin et al. (2014), which is incorporated herein by reference as if fully set forth).

The β-sheet breaker peptide may be H102 peptide. The 102 peptide may be a peptide comprising the amino acid sequence HKQLPFFEED (SEQ ID NO: 6). The 102 peptide may be a peptide comprising an amino acid with at least 70, 72, 75, 80, 85, 90, 91, 92, 93, 94, 95, 96, 97, 98, 99 or 100% sequence identity to an amino acid sequence of SEQ ID NO: 6. The variant of the H102 peptide may be any variant of the sequence of SEQ ID NO: 6, which is capable of inhibiting formation of β-sheet Aβ1-4 and by “misfolding” and aggregation of Aβ. Thus, the variant of the H102 peptide may be any variant that is capable of solubilizing plaques. The ability to solubilize plaques may be measured. For example, the number and the size of plaques in treated and referenced animals can be measured ex vivo by optical imaging as described in Example 4 herein. In vivo assays, for example, positron emission tomography (PET), near-infrared spectroscopy (NIR), or infra-red (IR) imaging are known in the art, and can be used for imaging amyloid plaques (Nordberg (2008), Kung et al. (2012), and Cheng et al. (2018), all of which are incorporated herein by reference as if fully set forth).

In an embodiment, the mini nanodrug may comprise one or more peptides capable of crossing the blood-brain barrier, and a β-sheet breaker peptide. The mini nanodrug may comprise Angiopep-2, Fe mimetic peptide, B6 peptide, or Miniap-4 peptide, or any combination thereof, and the H102 peptide. The mini nanodrug may further carry any of the antisense oligonucleotides described herein.

In an embodiment, the therapeutic peptide for AD treatment may be a plaque-binding peptide. As used herein, the term “plaque-binding peptide” refers to a peptide that binds to or labels neuritic plaques that consists of amyloid peptide β (Aβ), the characteristic pathological hallmark of AD. The plaque-binding peptide may be a β-sheet breaker peptide(s) described herein. The plaque-binding peptide may be a D-enantiomeric peptide that specifically binds to amyloid β1-42 (Aβ42). The D-enantiomeric peptide may bind to or label plaques that contain Aβ42 in the brain.

In an embodiment, the D-enantiomeric peptide may be one or more of a D1-peptide, a D3-peptide and an ACI-89-peptide, or variants thereof. The D-enantiomeric peptide may be the D1-peptide comprising an amino acid sequence QSHYRHISPAQVC (SEQ ID NO: 9). The D1-peptide may be a peptide comprising an amino acid with at least 70, 72, 75, 80, 85, 90, 91, 92, 93, 94, 95, 96, 97, 98, 99 or 100% sequence identity to an amino acid sequence of SEQ ID NO: 9. The variant of the D1-peptide may be any variant of the sequence of SEQ ID NO: 9, which is capable to of binding or labeling plaques that contain Aβ42. For example, assaying plaques ex vivo may include binding of reagent molecules to structural units (amino acid domains) of the amyloids, and measuring changes in fluorescence properties of the reagent-amyloid formations, e.g., by solubilization of the plaque material in these formations. Different D-peptides may recognize different amino acid sequences in β-amyloids as they are exposed in plaques. By virtue of efficacy of binding, these reagents may destabilize amyloid interactions forming free amyloid species, which can involve further binding to the reagent. The overall efficacy of the reagents may depend on the strength of binding to plaque domains. In case of plaque dissolution, morphometric analysis can be used to compare treated and referenced mice of similar stage of disease.

The D-enantiomeric peptide may be a D3-peptide comprising an amino acid sequence RPRTRLHTHRNRC (SEQ ID NO: 10). The D3-peptide may be a peptide comprising an amino acid with at least 70, 72, 75, 80, 85, 90, 91, 92, 93, 94, 95, 96, 97, 98, 99 or 100% sequence identity to an amino acid sequence of SEQ ID NO: 10.

The variant of the D3-peptide may be any variant of the sequence of SEQ ID NO: 10, which is capable of binding or labeling plaques that contain Aβ42.

The D-enantiomeric peptide may be ACI-89-peptide comprising an amino acid sequence PSHYRHISPAQKC (SEQ ID NO: 11). The ACI-89 peptide may be a peptide comprising an amino acid with at least 70, 72, 75, 80, 85, 90, 91, 92, 93, 94, 95, 96, 97, 98, 99 or 100% sequence identity to an amino acid sequence of SEQ ID NO: 11. The variant of the ACI-89-peptide may be any variant of the sequence of SEQ ID NO: 11, which is capable of binding or labeling plaques that contain Aβ42.

In an embodiment, the mini nanodrug may comprise one or more peptides capable of crossing the blood-brain barrier, and one or more plaque-binding peptides. The mini nanodrug may comprise Angiopep-2, Fe mimetic peptide, B6 peptide, or Miniap-4 peptide, or any combination thereof, and the D1-peptide, D3-peptides or ACI-89 peptide, or any combination thereof. The mini nanodrug may further comprise a β-sheet breaker peptide. The mini nanodrug may further carry any of the antisense oligonucleotides. The mini nanodrug may comprise peptides described herein and therapeutic agents in any combination.

In an embodiment, any one of the mini nanodrugs described herein may comprise an antibody. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody includes any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. Antibodies can be naked or conjugated to other molecules such as toxins, radioisotopes, etc. In other embodiments an antibody is a fusion antibody.

As used herein, the term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multispecific antibodies formed from antibody fragments.

An “Fv antibody” refers to the minimal antibody fragment that contains a complete antigen-recognition and-binding site either as two-chains, in which one heavy and one light chain variable domain form a non-covalent dimer, or as a single-chain (scFv), in which one heavy and one light chain variable domain are covalently linked by a flexible peptide linker so that the two chains associate in a similar dimeric structure. In this configuration the complementarity determining regions (CDRs) of each variable domain interact to define the antigen-binding specificity of the Fv dimer. Alternatively a single variable domain (or half of an Fv) can be used to recognize and bind antigen, although generally with lower affinity.

A “monoclonal antibody” as used herein refers to homogenous antibody population involved in specific recognition and binding of a single antigenic determinant, or epitope. Polyclonal antibodies include a population of antibody species each directed to a different antigenic determinant. The term “monoclonal antibody” encompasses both and full-length monoclonal antibodies and antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to those obtained without limitation by methods including and not limited to hybridoma expression, phage selection, recombinant expression, and by transgenic animals.

In an embodiment, the antibody may be an IgG antibody. The antibody of the invention may be a full-length antibody, for example, of an IgG1, IgG2 IgG3 or IgG4 isotype. The IgG antibody may be a single chain antibody, or consists of IgG antibody fragments. The fragments may be Fab or Fab′2 fragments. The antibody may be a single chain antibody (scFv), and may be produced by acquiring cDNA encoding the variable domains of the heavy (VH) and light chain (VL) from hybridoma producing a monoclonal antibody of the present invention, constructing a scFv expression vector, and causing expression by introducing the expression vector into E. coli, yeast or animal cell. The antibody may be a single chain engineered antibody.

In an embodiment, the antibody may be an antibody specific to at least vasculature protein in a cell. In an embodiment, the vasculature protein may be a transferrin receptor protein. An antibody specific to the transferrin receptor protein (TfR-Ab) may bind the transferrin receptor protein and thereby achieve transcytosis through endothelium associated with BBB. Without limitations, the antibody specific to the vasculature protein may be a monoclonal or polyclonal antibody. Further, the antibody may be a humanized antibody or a chimeric antibody.

Determining percent identity of two amino acid sequences or two nucleic acid sequences may include aligning and comparing the amino acid residues or nucleotides at corresponding positions in the two sequences. If all positions in two sequences are occupied by identical amino acid residues or nucleotides then the sequences are said to be 100% identical. Percent identity is measured by the Smith Waterman algorithm (Smith T F, Waterman M S 1981 “Identification of Common Molecular Subsequences,” J Mol Biol 147: 195-197, which is incorporated herein by reference as if fully set forth).

As used herein, “variant,” or “variant peptide” refers to a peptide that retains a biological activity that is the same or substantially similar to that of the original sequence. The variant may have a sequence that is similar to, but not identical to, the original sequence of the peptide or a fragment thereof. The variant may include one or more amino acid substitutions, deletions, insertions of amino acid residues, or any combination thereof. The variant may be from the same or different species or be a synthetic sequence based on a natural or prior sequence. The variant peptide may have the same length as the specified sequence of the peptide or may have additional amino acid residues at either or both termini of the peptide. The variant may be a fragment of the peptide. A fragment of the original sequence is a continuous or contiguous portion of the original sequences. For example, the length of the fragment of the original peptide 20 amino acid-long may vary in be any 2 to 19 contiguous amino acids within the original peptide.

An embodiment comprises amino acid sequences, peptides or polypeptides having a portion of the sequence as set forth in any one of the amino acids listed herein or the complement thereof. These amino acid sequences, peptides or polypeptides may have a length in the range from 2 to full length, 4 to 6, 6 to 8, 8 to 10, 10 to 12, 12 to 14, 14 to 16, or 7 to 13, or 7, 8, 9, 10, 13, 20 or 21 amino acids. An amino acid sequence, peptide or polypeptide having a length within one of the above ranges may have any specific length within the range recited, endpoints inclusive. The recited length of amino acids may start at any single position within a reference sequence (i.e., any one of the amino acids herein) where enough amino acids follow the single position to accommodate the recited length. The recited length may be full length of a reference sequence.

The variant or fragment of any one the peptides described herein capable of crossing the BBB are biologically active when the variant or fragment retains some or all activity of the original peptide, and is capable of transporting the mini nanodrug to which it is attached across the BBB. The variant or fragment of any one the plaque-binding peptides described herein are biologically active when the variant or fragment retains some or all activity of the original peptide, and is capable of binding or labeling neuritic plaques that consists of amyloid peptide β (Aβ).

The activity of the variants and fragments may be determined in an assay. The assay may involve testing variant's ability to bind to a receptor, or traverse BBB. For example, the assay may test binding or labeling neuritic plaques that consists of amyloid peptide β (Aβ). The variants and fragments of the original peptide may be more or less active compared to the original peptide. The variants of fragments may have lower activity compared to the original peptide as long as they are capable of achieving the desirable result.

The peptide or a variant thereof may have additional components or groups. For example, the sequence of the peptide or its variant may be linked to —NH₂ group at the C-terminus. The sequence of the peptide or a variant thereof may be linked to diaminopimelic acid (DAP) or hydroxyl diaminopimelic acid (H-DAP) at the N-terminus. The peptide or a variant thereof may contain bonds to increase stability and folding of the peptide. For instance, the peptide or a variant thereof may comprise disulfide bonds (—S—S—) forming an exocyclic structure that improves resistance to cleavage by peptidases. The sequence of the peptide or a variant thereof may be linked to any other moiety or group.

Without limitations, the peptide may be of any desired molecular weight. In an embodiment, the peptide may have a molecular weight of about 1,000 kDa, about 1,500 kDa, about 2,000 kDa, about 2,500 kDa, about 3,000 kDa, about 3,500 kDa, about 4,000 kDa, about 4,500 kDa, about 5,000 kDa, about 10,000 kDa, or about 15,000 Da. In an embodiment, the peptide may have a molecular weight of about 1 kDa to about 15 kDa. In an embodiment the peptide may have a molecular weight of 15 kDa, or less.

In an embodiment, each of peptides described herein may be conjugated to the polymalic acid-based molecular scaffold by a linker. As used herein, the term “linker” means an organic moiety that connects two parts of a compound.

In an embodiment, the linker may comprise a polyethylene glycol (PEG). Without limitations, the PEG may be of any desired molecular weight. In an embodiment, the PEG may have a molecular weight of about 1,000 Da, about 1,500 Da, about 1,000 Da, about 2,500 Da, about 3,000 Da, about 3,500 Da, about 4,000 Da, about 4,500 Da, about 5,000 Da, about 10,000 Da, about 15,000 Da, about 20,000 Da, about 25,000 Da, or about 30,000 Da. In an embodiment, the PEG may have a molecular weight of about 3,400 Da.

In an embodiment, the mini nanodrug may include an endosomolytic ligand. The endosomolytic ligand may be covalently linked with the polymalic acid-based molecular scaffold. As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), golgi apparatus, microtubule, peroxisome, or other vesicular bodies within the cell, to the cytoplasm of the cell. The endosomolytic ligands may be, but are not limited to, imidazoles, poly or oligoimidazoles, linear or branched polyethyleneimines (PEIs), linear or branched polyamines, e.g. spermine, cationic linear or branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural or synthetic fusogenic lipids, natural or synthetic cationic lipids.

In an embodiment, the endosomolytic ligand may include a plurality of leucine, isoleucine, valine, tryptophan, or phenylalanine residues The endosomolytic ligand may be Trp-Trp-Trp (WWW), Phe-Phe-Phe (FFF), Leu-Leu-Leu (LLL), or Ile-Ile-Ile (I-I-I). The WWW, FFF, LLL or III may enhance the ability of the mini nanodrug to cross the blood-brain barrier.

In an embodiment, the polymalic acid-based molecular scaffold may be polymalic acid. As used herein, the term “polymalic acid” refers to a polymer, e.g., a homopolymer, a copolymer or a blockpolymer that contains a main chain ester linkage. The polymalic acid may be at least one of biodegradable and of a high molecular flexibility, soluble in water (when ionized) and organic solvents (in its acid form), non-toxic, or non-immunogenic (Lee B et al., Water-soluble aliphatic polyesters: poly(malic acid)s, in: Biopolymers, vol. 3a (Doi Y, Steinbuchel A eds., pp 75-103, Wiley-VCH, New York 2002, which is incorporated herein by reference as if fully set forth). In an embodiment, the polymalic acid may be poly(β-L-malic acid), herein referred to as poly-β-L-malic acid or PMLA.

Without limitations, the polymalic acid may be of any length and of any molecular mass. The polymalic acid may have a molecular mass of 10, 20, 30, 40, 50, 60, 70, 80, 90, 100, 110, 120, 130, 140, 150, 160, 170, 180, 190, or 200 kDa. The polymalic acid may have a molecular mass of 10, 20, 30, 40, 50, or 60 kDa.

In an embodiment, the polymalic acid may have a molecular mass in a range between any two of the following molecular masses: 10, 20, 30, 40, 50, 60, 70, 80, 90, 100, 110, 120, 130, 140, 150, 160, 170, 180, 190, or 200 kDa. In an embodiment, the polymalic acid may have a molecular mass in a range between any two of the following masses: 40, 45, 50, 55, or 60 kDa.

Exemplary polymalic acid-based molecular scaffolds amenable to the imaging nanoagents disclosed herein are described, for example, in PCT Appl. Nos. PCT/US04/40660, filed Dec. 3, 2004, PCT/US09/40252, filed Apr. 10, 2009, and PCT/US10/59919, filed Dec. 10, 2010, PCT/US10/62515, filed Dec. 30, 2010; and U.S. patent application Ser. No. 10/580,999, filed Mar. 12, 2007, and Ser. No. 12/935,110, filed Sep. 28, 2010, contents of all which are incorporated herein by reference as if fully set forth.

The mini nanodrug may be linked to an additional therapeutic agent. The additional therapeutic agent may be a drug for treatment of AD. Additional exemplary drugs for treatment of AD may be but are not limited to cholinesterase inhibitors, muscarinic agonists, anti-oxidants or anti-inflammatories. Galantamine (Reminyl), tacrine (Cognex), selegiline, donepezil, (Aricept), saeluzole, acetyl-L-carnitine, idebenone, ENA-713, memric, quetiapine, or verubecestat (3-imino-1,2,4-thiadiazinane 1,1-dioxidederivative) may be used.

The additional therapeutic agent may be an anti-cancer agent. Additional exemplary anti-cancer agents amenable to the present invention may be, but are not limited to, paclitaxel (taxol); docetaxel; germicitibine; alitretinoin; amifostine; bexarotene bleomycin; calusterone; capecitabine; platinate; chlorambucil; cytarabine; daunorubicin, daunomycin; docetaxel; doxorubicin; dromostanolone propionate; fluorouracil (5-FU); leucovorin; methotrexate; mitomycin C; mitoxantrone; nandrolone pamidronate; mithramycin; porfimer sodium; procarbazine; quinacrine; temozolomide; or topotecan.

In an embodiment, the mini nanodrug may further comprise an imaging agent. The imaging agent may be any fluorescent reporter dye. A wide variety of fluorescent reporter dyes, e.g., fluorophores, are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Suitable fluorescent reporters may include xanthene dyes, such as fluorescein or rhodamine dyes. Fluorophores may be, but are not limited to, 1,5 IAEDANS; 1,8-ANS; 4-Methylumbelliferone; 5-carboxy-2,7-dichlorofluorescein; 5-Carboxy fluorescein (5-FAM); 5-Carboxynapthofluorescein (pH 10); 5-Carboxytetramethyl rhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethyl rhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18 (3)); DiR; DiR (DiIC18 (7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin EBG; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodaminelsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; or YOYO-3. Many suitable forms of these fluorescent compounds are available and may be used.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566, contents of which are incorporated herein by reference as if fully set forth). Specific examples of GFP variants include, but are not limited to, enhanced GFP (EGFP), destabilized EGFP, the GFP variants described in Doan et al, Mol. Microbiol, 55:1767-1781 (2005), the GFP variant described in Crameri et al, Nat. Biotechnol., 14:315319 (1996), the cerulean fluorescent proteins described in Rizzo et al, Nat. Biotechnol, 22:445 (2004) and Tsien, Annu. Rev. Biochem., 67:509 (1998), and the yellow fluorescent protein described in Nagal et al, Nat. Biotechnol., 20:87-90 (2002). DsRed variants are described in, e.g., Shaner et al, Nat. Biotechnol., 22:1567-1572 (2004), and include mStrawberry, mCherry, mOrange, mBanana, mHoneydew, and mTangerine. Additional DsRed variants are described in, e.g., Wang et al, Proc. Natl. Acad. Sci. U.S.A., 101:16745-16749 (2004) and include mRaspberry and mPlum. Further examples of DsRed variants include mRFPmars described in Fischer et al, FEBS Lett., 577:227-232 (2004) and mRFPruby described in Fischer et al, FEBS Lett, 580:2495-2502 (2006).

The imaging agent may be one or more cyanine dyes. The cyanine dye may be but is not limited to indocyanine green (ICG), Cy5, Cy5.5, Cy5.18, Cy7 and Cy7.18, IRDye 78, IRDye 680, IRDye 750, IRDye 800 phosphoramidite, DY-681, DY-731, and DY-781.

The imaging agent may be a fluorescent dye suitable for near-infrared (NIR) fluorescence. The NIR imaging may be used for intraoperative visualization and non-invasive imaging of cells and tissues in a subject. The NIR fluorescence imaging involves administration of a fluorescent contrast agent that can be excited at wavelengths of 780 nm or greater, and has a significant Stoke's shift emitting fluorescence at wavelengths of 800 nm or greater.

The imaging agent may be an agent suitable for imaging by magnetic resonance (MRI). The imaging agents may comprise paramagnetic metal ions such as manganese (MnII), iron (FeIII), or gadolinium (Gd-III). The imaging agent may be DOTA-Gd(III).

The molecular scaffold and the components covalently linked with the polymalic acid-based molecular scaffold may be linked to each other via a linker. Linkers typically comprise a direct bond or an atom such as oxygen or sulfur, a unit such as NR¹, C(O), C(O)OC, C(O)NH, SO, SO₂, SO₂NH, —SS— or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroaryl alkynyl, alkenylheteroarylalkyl, alkenylheteroarylalkenyl, alkenylheteroaryl alkynyl, alkynylheteroarylalkyl, alkynylheteroarylalkenyl, alkynylheteroaryl alkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

In an embodiment, the mini nanodrug may further comprise a PK modulating ligand covalently linked with the polymalic acid-based molecular scaffold. As used herein, the terms “PK modulating ligand” and “PK modulator” refers to molecules which can modulate the pharmacokinetics of the imaging nanoagent. For example, the PK modulator can inhibit or reduce resorption of the imaging nanoagent by the reticuloendothelial system (RES) and/or enzyme degradation.

PEGylation is generally used in drug design to increase the in vivo half-life of conjugated proteins, to prolong the circulation time, and enhance extravasation into targeted solid tumors (Arpicco et al., 2002 Bioconjugate Chem 13:757 and Maruyama et al., 1997 FEBS Letters 413:1771, both of which are incorporated herein by reference as if fully set forth). Thus, in an embodiment, the PK modulator may be a PEG. Without limitations, the PEG may be of any desired molecular weight. In an embodiment, the PEG may have a molecular weight of about 1,000 Da, about 1,500 Da, about 1,000 Da, about 2,500 Da, about 3,000 Da, about 3,500 Da, about 4,000 Da, about 4,500 Da, about 5,000 Da, about 10,000 Da, about 15,000 Da, about 20,000 Da, about 25,000 Da, or about 30,000 Da. In an embodiment, the PK modulator may be PEG of about 2,000 Da. Other molecules known to increase half-life may also be used as PK modulators.

Without limitations, the mini nanodrug may be of any desired size. For example, the mini nanodrug may be of a size that allows the mini nanodrug to cross the blood brain barrier via targeting or via transcytosis. In an embodiment, the mini nanodrug may range in size from about 1 nm to about 10 nm; from about 1 nm to about 2 nm; from about 2 nm to about 3 nm; from about 3 nm to about 4 nm; from about 4 nm to about 5 nm; from about 5 nm to about 6 nm; from about 6 nm to about 7 nm; from about 7 nm to about 8 nm; from about 8 nm to about 9 nm; from about 9 nm to about 10 nm. In an embodiment, the mini nanodrug may be about 5 nm to about 10 nm in size. In an embodiment, the mini nanodrug may be 10 nm or less in size. In an embodiment, the mini nanodrug may be 15 nm in size, or less.

It will be understood by one of ordinary skill in the art that the mini nanodrug may exhibit a distribution of sizes around the indicated “size.” Thus, unless otherwise stated, the term “size” as used herein refers to the mode of a size distribution of mini nanodrugs, i.e., the value that occurs most frequently in the size distribution. Methods for measuring the size are known to a skilled artisan, e.g., by dynamic light scattering (such as photocorrelation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

In an embodiment, a pharmaceutically acceptable composition comprising any one the mini nanodrugs disclosed herein and a pharmaceutically acceptable carrier or excipient is provided.

An embodiment provides a method for treating a brain disease or abnormal condition. The method may comprise administering a therapeutically effective amount of a composition comprising any one of the mini nanodrugs described herein to a subject in need thereof.

In an embodiment, the method for treating the brain disease or abnormal conditions may further comprise providing the composition comprising any one of the mini nanodrug described herein to a subject in need thereof. The brain disease may be Alzheimer's disease (AD). AD is a degenerative disorder of the brain first described by Alios Alzheimer in 1907 after examining one of his patients who suffered drastic reduction in cognitive abilities and had generalized dementia. AD is associated with neuritic plaques measuring up to 200 μm in diameter in the cortex, hippocampus, subiculum, hippocampal gyrus, and amygdala. One of the principal constituents of neuritic plaques is amyloid. The plaques are composed of polypeptide fibrils and are often present around blood vessels, reducing blood supply to various neurons in the brain.

These plaques are made up primarily of the amyloid β peptide (Aβ; sometimes also referred to in the literature as β-amyloid peptide or β-peptide), which is also the primary protein constituent in cerebrovascular amyloid deposits. Following administration, the mini nanodrugs may be monitored for their brain distribution, for example, by ex vivo and in vivo imaging methods described herein. The distribution of the mini nanodrugs may be compared with their efficacy in inhibiting or reducing formation of amyloid plaques determined by methods disclosed herein.

AD treatment may involve administering of drugs effective in decreasing amyloid plaque formation.

In an embodiment, the method for treating cancer may comprise administering a therapeutically effective amount of any one of the mini nanodrug described herein to a subject in need thereof.

In an embodiment, the method for treating the brain disease or abnormal condition may comprise co-administering a therapeutically effective amount of an anti-cancer agent and a therapeutically effective amount of a mini nanodrug to a subject in need thereof, wherein the mini nanodrug comprises a polymalic acid-based molecular scaffold and at least one targeting ligand and at least one anti-cancer agent covalently conjugated or linked to the scaffold.

In an embodiment, the method may further comprise analyzing the plaque formation in the subject affected or suffering from AD. The step of analyzing may include observing more than about 50%, 60%, 70%, 80% or about 90% decrease in the formation of AD plaques in the subject. The step of analyzing may include observing of the dissolution of AD plaques in the subject. The step of analyzing may include observing stabilizing growth of the AD plaques in the subject.

In an embodiment, the method may further comprise analyzing inhibition of tumor growth. The step of analyzing may include observing more than about 60%, 70%, 80% or about 90% inhibition of tumor growth in the subject. In an embodiment, the step of analyzing may include observing the inhibition of HER2/neu receptor signaling by suppression of Akt phosphorylation.

The terms “subject” and “individual” are used interchangeably herein, and mean a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In an embodiment, the subject may be a mammal, e.g., a primate, e.g., a human. The terms, “patient” and “subject” are used interchangeably herein. The terms, “patient” and “subject” are used interchangeably herein.

Preferably, the subject is a mammal. The mammal may be a human, non-human primate, mouse, rat, dog, cat, horse, or cow, but are not limited to these examples. Mammals other than humans may be advantageously used as subjects that represent animal models for Alzheimer's disease. As a non-limiting example, Double or Triple Transgenic Alzheimer's mouse may be used. Mammals other than humans may be advantageously used as subjects that represent animal models of cancer. In addition, the methods described herein may be used to treat domesticated animals and/or pets. A subject may be male or female. A subject may be one who has been previously diagnosed with or identified a suffering from Alzheimer's disease, but need not have already undergone treatment. A subject may be one who has been previously diagnosed with or identified as suffering from cancer, but need not have already undergone treatment.

The phrase “therapeutically-effective amount” in the methods described means that amount of a compound, material, or composition which is effective for producing some desired therapeutic effect in at least a sub-population of cells in an animal at a reasonable benefit/risk ratio applicable to any medical treatment. In connection with treating cancer, the “therapeutically effective amount” is that amount effective for preventing further development of a cancer or transformed growth, and even to effect regression of the cancer or solid tumor.

Determination of a therapeutically effective amount is generally well within the capability of those skilled in the art. Generally, a therapeutically effective amount can vary with the subject's history, age, condition, sex, as well as the severity and type of the medical condition in the subject, and administration of other agents alleviate the disease or disorder to be treated.

Toxicity and therapeutic efficacy may be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred. As used herein, the term ED denotes effective dose and is used in connection with animal models. The term EC denotes effective concentration and is used in connection with in vitro models.

The data obtained from the cell culture assays and animal studies may be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose may be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage may be monitored by a suitable bioassay.

The dosage may be determined by a physician and adjusted, as necessary, to suit observed effects of the treatment. Generally, the compositions may be administered so that the active agent is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

In an embodiment, the compositions may be administered at a dosage so that the active agent has an in vivo concentration of less than 500 nM, less than 400 nM, less than 300 nM, less than 250 nM, less than 200 nM, less than 150 nM, less than 100 nM, less than 50 nM, less than 25 nM, less than 20, nM, less than 10 nM, less than 5 nM, less than 1 nM, less than 0.5 nM, less than 0.1 nM, less than 0.05, less than 0.01, nM, less than 0.005 nM, less than 0.001 nM after 15 mins, 30 mins, 1 hr, 1.5 hrs, 2 hrs, 2.5 hrs, 3 hrs, 4 hrs, 5 hrs, 6 hrs, 7 hrs, 8 hrs, 9 hrs, 10 hrs, 11 hrs, 12 hrs or more of time of administration.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule may vary from once a week to daily depending on a number of clinical factors, such as the subject's sensitivity to the peptides. The desired dose may be administered every day or every third, fourth, fifth, or sixth day. The desired dose may be administered at one time or divided into subdoses, e.g., 2-4 subdoses and administered over a period of time, e.g., at appropriate intervals through the day or other appropriate schedule. Such sub-doses may be administered as unit dosage forms. In an embodiment, administration may be chronic, e.g., one or more doses daily over a period of weeks or months. Examples of dosing schedules may include administration daily, twice daily, three times daily or four or more times daily over a period of 1 week, 2 weeks, 3 weeks, 4 weeks, 1 month, 2 months, 3 months, 4 months, 5 months, or 6 months or more.

As used herein, the term “administer” refers to the placement of a composition into a subject by a method or route which results in at least partial localization of the composition at a desired site such that desired effect is produced. A compound or composition described herein may be administered by any appropriate route known in the art including, but not limited to, oral or parenteral routes, including intravenous, intramuscular, subcutaneous, transdermal, airway (aerosol), pulmonary, nasal, rectal, or topical (including buccal and sublingual) administration.

Exemplary modes of administration include, but are not limited to, injection, infusion, instillation, inhalation, or ingestion. “Injection” include, without limitation, intravenous, intramuscular, intraarterial, intrathecal, intraventricular, intracapsular, intraorbital, intracardiac, intradermal, intraperitoneal, trans tracheal, subcutaneous, subcuticular, intraarticular, sub capsular, subarachnoid, intraspinal, intracerebro spinal, and intrastemal injection and infusion. In an embodiment, the compositions may be administered by intravenous infusion or injection.

For administration to a subject, the mini nanodrug may be provided in pharmaceutically acceptable compositions. Accordingly, an embodiment also provides pharmaceutical compositions comprising the mini nanodrugs as disclosed herein. These pharmaceutically acceptable compositions may comprise a therapeutically-effective amount of one or more of the mini nanodrugs, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, the mini nanodrugs may be implanted into a patient or injected using a drug delivery system.

A variety of known controlled- or extended-release dosage forms, formulations, and devices may be adapted for use with the mini nanodrugs and compositions of the disclosure. Examples include, but are not limited to, those described in U.S. Pat. Nos. 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185 B1, all of which are incorporated herein by reference as if fully set forth. These dosage forms may be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In an embodiment, the pharmaceutically acceptable composition may be formulated in dosage unit form for ease of administration and uniformity of dosage. The expression “dosage unit form” as used herein refers to a physically discrete unit of active agent appropriate for the subject to be treated.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zincstearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which may serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (S) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyllaurate; (13) agar; (14) buffering agents, such as magnesium hydroxide and aluminum hydroxide; (15) alginic acid; (16) pyrogen-free water; (17) isotonic saline; (IS) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants may also be present in the formulation. The terms such as “excipient”, “carrier”, “pharmaceutically acceptable carrier” or the likes are used interchangeably herein.

The following list includes particular embodiments of the present invention. But the list is not limiting and does not exclude alternate embodiments, or embodiments otherwise described herein. Percent identity described in the following embodiments list refers to the identity of the recited sequence along the entire length of the reference sequence.

### Embodiments

1. A mini nanodrug comprising a polymalic acid-based molecular scaffold,

at least one peptide capable of crossing the blood-brain barrier, at least one plaque-binding peptide and an endosomolytic ligand, wherein each of the at least one peptide capable of crossing the blood-brain barrier, the at least one plaque-binding peptide and the endosomolytic ligand are covalently linked to the polymalic acid-based molecular scaffold, and the mini nanodrug ranges in size from 1 nm to 10 nm.
2. The mini nanodrug of embodiment 1, wherein the at least one peptide capable of crossing the blood-brain barrier is an LRP-1 ligand, or a transferrin receptor ligand.
3. The mini nanodrug of one or both embodiments 1 and 2, wherein the at least one peptide capable of crossing the blood-brain barrier is a peptide selected from the group consisting of Angiopep-2, Fe mimetic peptide, B6 peptide, and Miniap-4 peptide, or variants thereof.
4. The mini nanodrug of any one or more of embodiments 1-3, wherein the at least one peptide capable of crossing the blood-brain barrier is Angiopep-2 comprising an amino acid sequence of SEQ ID NO: 1, or a variant thereof.
5. The mini nanodrug of any one or more of embodiments 1-4, wherein the at least one peptide capable of crossing the blood-brain barrier is Fe mimetic peptide comprising an amino acid sequence of SEQ ID NO: 2, or a variant thereof.
6. The mini nanodrug of any one or more of embodiments 1-5 wherein the at least one peptide crossing the blood-brain barrier is B6 peptide comprising an amino acid sequence of SEQ ID NO: 8, or a variant thereof.
7. The mini nanodrug of any one or more of embodiments 1-6, wherein the at least one peptide capable of crossing the blood-brain barrier is a Miniap-4 peptide comprising an amino acid sequence of SEQ ID NO: 3, or a variant thereof.
8. The mini nanodrug of any one or more of embodiments 1-7, wherein the at least one peptide capable of crossing the blood-brain barrier comprises at least two peptides.
9. The mini nanodrug of embodiment 8, wherein each of the at two least peptides is selected independently.
10. The mini nanodrug of embodiments 8, wherein the at least two peptides are similar peptides.
11. The mini nanodrug of any one or more of embodiments 1-10, wherein the at least one peptide is conjugated to the polymalic acid-based molecular scaffold by a linker.
12. The mini nanodrug of embodiment 11, wherein the linker comprises a polyethylene glycol (PEG).
13. The mini nanodrug of any one or more of embodiments 1-12, wherein the endosomolytic ligand comprises a plurality of leucine, isoleucine, valine, tryptophan, or phenylalanine residues.
14. The mini nanodrug of any one or more of embodiments 1-13, wherein the endosomolytic ligand comprises Trp-Trp-Trp (WWW), Phe-Phe-Phe (FFF), Leu-Leu-Leu (LLL), or Ile-Ile-Ile (I-I-I).
15. The mini nanodrug of any one or more of embodiments 1-14, wherein the mini nanodrug further comprises a therapeutic agent.
16. The mini nanodrug of any one or more of embodiments 1-15, wherein the therapeutic agent is selected from the group consisting of: an oligonucleotide, an antisense oligonucleotide, an siRNA oligonucleotide, a peptide, and a low molecular weight drug.
17. The mini nanodrug of any one or more of embodiments 1-16, wherein the therapeutic agent is an antisense oligonucleotide complementary to a β-secretase mRNA sequence or a γ-secretase mRNA sequence.
18. The mini nanodrug of any one or more of embodiments 16-17, wherein the antisense oligonucleotide comprises a nucleic acid sequence with at least 90% identity to SEQ ID NO: 4.
19. The mini nanodrug of any one or more of embodiments 1-16, wherein the therapeutic agent is an oligonucleotide capable of targeting a messenger RNA transcribed from a target gene.
20. The mini nanodrug of embodiment 19, wherein the target gene encodes BACE1, and the oligonucleotide comprises a sequence with at least 90% identity to SEQ ID NO: 14.
21. The mini nanodrug of any one or more of embodiments 1-20, wherein the therapeutic agent comprises a β-sheet breaker peptide.
22. The mini nanodrug of embodiment 22, wherein the β-sheet breaker peptide comprises an amino acid sequence of SEQ ID NO: 6 or a variant thereof.
23. The mini nanodrug of any one or more of embodiments 1-22, wherein the plaque-binding peptide is a D-enantiomeric peptide.
24. The mini nanodrug of any one or more of embodiments 1-23, wherein the D-enantiomeric peptide is selected from the group consisting of: a D1-peptide, a D3-peptide and an ACI-89-peptide, or variants thereof.
25. The mini nanodrug of any one or more of embodiments 1-24, wherein the D-enantiomeric peptide is the D1-peptide comprising an amino acid sequence of SEQ ID NO: 9, or a variant thereof.
26. The mini nanodrug of any one or more of embodiments 1-24, wherein the D-enantiomeric peptide is the D3-peptide comprising an amino acid sequence of SEQ ID NO: 10, or a variant thereof.
27. The mini nanodrug of any one or more of embodiments 1-24, wherein the D-enantiomeric peptide is the ACI-89-peptide comprising an amino acid sequence of SEQ ID NO: 11 or a variant thereof.
28. The mini nanodrug of any one or more of embodiments 1-27, wherein the nanodrug further comprises an imaging agent covalently linked with the polymalic acid-based molecular scaffold.
29. The mini nanodrug of embodiment 28, wherein the imaging agent comprises a fluorescence moiety, a radioisotope moiety, or a magnetic resonance imaging moiety.
30. The mini nanodrug of any one or more of embodiments 1-29, wherein the polymalic acid-based molecular scaffold comprises poly(β-L-malic acid).
31. A mini nanodrug comprising a polymalic acid-based molecular scaffold,

at least one peptide capable of crossing the blood-brain barrier, an endosomolytic ligand and a therapeutic agent, wherein each of the at least peptide capable of crossing the blood-brain barrier, the endosomolytic ligand and the therapeutic agent are covalently linked to the polymalic acid-based molecular scaffold, and the nanodrug ranges in size from 1 nm to 10 nm.
32. The mini nanodrug of embodiment 31, wherein the at least one peptide capable of crossing the blood-brain barrier is a peptide selected from the group consisting of Angiopep-2, Fe mimetic peptide, B6 peptide, and Miniap-4 peptide, or variants thereof.
33. The mini nanodrug of any one or both embodiments 31 and 32, wherein the at least one peptide capable of crossing the blood-brain barrier is Angiopep-2 comprising a sequence of SEQ ID NO: 1, or a variant thereof.
34. The mini nanodrug of any one or more of embodiments 31-32, wherein the at least one peptide capable of crossing the blood-brain barrier is Fe mimetic peptide comprising an amino acid sequence of SEQ ID NO: 2, or a variant.
35. The mini nanodrug of any one or more of embodiments 31-32, wherein the at least one peptide capable of crossing the blood-brain barrier is B6 peptide comprising an amino acid sequence of SEQ ID NO: 8, or a variant thereof.
36. The mini nanodrug of any one or more of embodiments 31-32, wherein the at least one peptide capable of crossing the blood-brain barrier is a Miniap-4 peptide comprising an amino acid sequence of SEQ ID NO: 3, or a variant thereof.
37. The mini nanodrug of any one or more of embodiments 31-36, wherein the at least one peptide capable of crossing the blood-brain barrier comprises at least two peptides, wherein each of the at least two peptides are independently selected peptides or similar peptides.
38. The mini nanodrug of any one or more of embodiments 31-37, wherein the at least one peptide capable of crossing the blood-brain barrier is conjugated to the polymalic acid-based molecular scaffold by a linker.
39. The mini nanodrug of any one or more of embodiments 31-37, wherein the endosomolytic ligand comprises Trp-Trp-Tr (WWW), Phe-Phe-Phe (FFF), Leu-Leu-Leu (LLL), or Ile-Ile-Ile (I-I-I).
40. The mini nanodrug of any one or more of embodiments 31-39, wherein the therapeutic agent is selected from the group consisting of: an antisense oligonucleotide, an siRNA oligonucleotide, a peptide, and a low molecular weight drug.
41. The mini nanodrug of any one or more of embodiments 31-40, wherein the therapeutic agent comprises an antisense oligonucleotide complementary to a β-secretase mRNA sequence or a γ-secretase mRNA sequence.
42. The mini nanodrug of embodiment 41, wherein the antisense oligonucleotide comprises a nucleic acid sequence with at least 90% identity to SEQ ID NO: 4.
43. The mini nanodrug of any one or more of embodiments 31-40, wherein the therapeutic agent is an oligonucleotide capable of targeting a messenger RNA transcribed from a target gene.
44. The mini nanodrug of embodiment 43, wherein the target gene encodes BACE1, and the oligonucleotide comprises a sequence with at least 90%identity to SEQ ID NO: 14.
45. The mini nanodrug of any one or more of embodiments 31-44, wherein the therapeutic agent comprises a β-sheet breaker peptide.
46. The mini nanodrug of embodiment 45, wherein the β-sheet breaker peptide comprises an amino acid sequence of SEQ ID NO: 6, or a variant thereof.
47. The mini nanodrug of any one or more of embodiments 31-46, wherein the mini nanodrug further comprises a plaque-binding peptide.
48. The mini nanodrug of embodiment 47, wherein the plaque-binding peptide is a D-enantiomeric peptide selected from the group consisting of: a D1-peptide, a D3-peptide and an ACI-89-peptide, or variants thereof.
49. The mini nanodrug of any one or both of embodiments 47-48, wherein the plaque-binding peptide comprises two or more plaque-binding peptides independently selected from the group consisting of: a D1-peptide, a D3-peptide and an ACI-89-peptide, or variants thereof.
50. The mini nanodrug of any one or more of embodiments 47-49, wherein the plaque-binding peptide comprises two or more plaque-binding peptides selected from the group consisting of: a D1-peptide, a D3-peptide and an ACI-89-peptide, or variants thereof, and the selected peptides are similar.
51. The mini nanodrug of any one or more of embodiments 47-50, wherein the D-enantiomeric peptide is a peptide comprising an amino acid sequence of SEQ ID NO: 9,10 or 11.
52. The mini nanodrug of any one or more of embodiments 31-51, wherein the mini nanodrug further comprises an imaging agent covalently linked with the polymalic acid-based molecular scaffold.
53. The mini nanodrug of embodiment 52, wherein the imaging agent comprises a fluorescence moiety, a radioisotope moiety, or a magnetic resonance imaging moiety.
54. The mini nanodrug of any one or more of embodiments 31-53, wherein the therapeutic agent comprises an anti-cancer agent.
55. The mini nanodrug of any one or more of embodiments 31-54, wherein the polymalic acid-based molecular scaffold comprises poly(β-L-malic acid).
56. The mini nanodrug of embodiment 31 or 55, wherein the poly(β-L-malic acid) has a molecular mass between 40 kDa and 60 kDa.
57. The mini nanodrug of any one or more of embodiments 1-56, wherein the mini nanodrug has a molecular mass between 75 kDa and 500 kDa.
58. The mini nanodrug of any one or more of embodiments 1-57, wherein the mini nanodrug further comprises an antibody, and wherein the antibody is an IgG isotype, fragment of IgG isotype, single chain antibody of an animal or single chain engineered antibody.
59. A pharmaceutically acceptable composition comprising a mini nanodrug of any one or more of embodiments 1-58, and a pharmaceutically acceptable carrier or excipient.
60. A method for treating a brain disease or abnormal condition in a subject, comprising: administering a therapeutically effective amount of a mini nanodrug of any one or more of embodiments 1-58, or a pharmaceutically acceptable composition of embodiment 59 to a subject in need thereof.
61. The method of embodiment 60, wherein the brain disease or abnormal condition is selected from the group consisting of Alzheimer's disease, multiple sclerosis, Parkinson's disease, Huntington's disease, schizophrenia, anxiety, dementia, mental retardation, and anxiety.
62. The method of any or both of embodiments 60-61, wherein the brain disease is Alzheimer's disease.
63. The method of any or more of embodiments 60-62, wherein the Alzheimer's disease is treated, prevented or ameliorated after administration of the mini nanodrug for a period of time.
64. The method of embodiment 63, wherein the period of time is at least one month.
65. The method of any or more of embodiments 60-64, wherein administration is performed at least once a week, at least once a day, or at least twice a day for a period of at least one month.
66. A method for reducing formation of amyloid plaques in the brain of a subject, comprising administering the mini nanodrug of any one or more of embodiments 1-58, or composition of embodiment 59 to a subject in need thereof.
67. A method of detecting amyloid plaques in the brain of a subject comprising administering the mini nanodrug of any one or more of embodiments 1-27, 30-51, and 54-58, wherein the mini nanodrug further comprises an imaging agent comprising a fluorescence moiety, a radioisotope moiety, or a magnetic resonance imaging moiety; and visualizing the mini nanodrug.
68. The method of embodiment 67, wherein the visualizing includes imaging a tissue in a brain of the subject.
69. A method for treating a proliferative disease in a subject, comprising: administering a therapeutically effective amount of a mini nanodrug of any one or more of embodiments 31-44 or the composition comprising the mini nanodrugs of any one of embodiments 31-44 and a pharmaceutically acceptable carrier or excipient to the subject in need thereof.
70. The method of embodiment 69, wherein the proliferative disease is a cancer.
71. The method of embodiment 70, wherein the cancer is selected from the group consisting of: glioma, glioblastoma, breast cancer metastasized to the brain and lung cancer metastasized to the brain.
72. The method of any one or more of embodiments 69-70, wherein the therapeutic agent is an anti-cancer agent.
73. The method of any one or more of embodiments 69-72, wherein the subject is a mammal.
74. The method of embodiment 73, wherein the mammal is selected from the group consisting of: a rodent, a canine, a primate, an equine, an experimental human-breast tumor-bearing nude mouse, and a human.

Further embodiments herein may be formed by supplementing an embodiment with one or more element from any one or more other embodiment herein, and/or substituting one or more element from one embodiment with one or more element from one or more other embodiment herein.

### EXAMPLES

The following non-limiting examples are provided to illustrate particular embodiments. The embodiments throughout may be supplemented with one or more detail from one or more example below, and/or one or more element from an embodiment may be substituted with one or more detail from one or more example below.

### Example 1

### Design of Mini Nanodrugs for Efficient Crossing Blood-Brain Barrier

One of the major problems facing the treatment of neurological disorders is the poor delivery of therapeutic agents and conventional drugs into the brain. As an alternative to these drug designs, a multifunctional and biodegradable nanoconjugate drug delivery system was developed around the naturally occurring polymeric scaffold, polymalic acid, also referred to herein as PMLA or P. For example, β-poly(L-malic acid) can be used. The nanoconjugate drug delivery system capable of crossing the blood-brain barrier (BBB) to access brain tissues affected by neurological disease has been developed.

The nanoconjugate drug delivery system is also referred to herein as a mini nanodrug. The designed mini nanodrugs are characterized by hydrodynamic diameter 5-8 nm, elongated shape and ability of chemical attachment of drugs and operational groups, e.g. receptor targeting, to a polymer platform. The elongated shape enables the mini nanodrug for rapid diffusion compared to spherical nanodrugs of the same mass, and to pass through pores of narrow diameter. The platform also provided chemical anchorage for various modules designed for endosome disruption, MRI and fluorescence imaging or protection. Cleavable linkers can be used that enable drug activation in response to chemistry in the targeted compartment. In the designed mini nanodrugs, several targeting molecules can be ligated to the platform via multiple attachments, and thus nanodrugs can be designed for programmed delivery through multiple bio barriers. The mini nanodrug has a high degree of internal freedom derived from unlimited rotation around the carbon and carbon-oxygen atoms derived from the ester bonds. The rotational freedom allows the scaffold-attached groups to avoid unfavorable molecular crowding.

Using this design, the mini nanodrugs may be developed for highly efficient treatment of preclinical HER2-positive human breast cancer by replacement of targeting antibodies with HER2-affine peptide. The mini nanodrug may be designed to deliver multiple copies of antisense oligonucleotide or docetaxel to the cytoplasm and arrest tumor growth. Delivery of imaging agents may be achieved across the blood-brain barrier (BBB) with peptides targeting different delivery routes when attached separately or combination of routes when attached simultaneously. Another design may be a targeted mini nanodrug carrying the NIR fluorescent dye ICG that brightly lights up glioblastoma in mice for imaging guided tumor resection. In all the designs, mini nanodrugs are cleared with half-lives of one hour and residing times of several hours inside tumors or other targeted regions.

The design of mini nanodrugs to treat Alzheimer disease (AD) is described herein. Despite multiple attempts to persistently treat Alzheimer disease, a satisfactory prevention of toxic Aβ production is still not in sight. Treatment with a nanosize multi drug delivery platform is described herein that was designed for officious targeted multi-prone inhibition of soluble Aβ production. In applying nanocarrier cascade targeting of multiple BBB crossing transcytosis pathways and of agents/cells in the brain, the treatment exceeds the outcome of existing attempts in efficacy, absence of side effects and improved image guided control.

Design of the Mini Nanodrugs Using PMLA as a Biodegradable Platform:

The focus was thus directed towards the development of a mini nanodrug that crosses the BBB of healthy mice.

PMLA (polymalic acid or P) was selected as platform for mini nanodrug development because PMLA is completely biodegradable to carbon dioxide and water, biologically inert, nontoxic and non-immunogenic. PMLA also carries abundant carboxyl groups that can be conjugated with multiple targeting and therapeutic moieties, ultimately constituting a mini nanodrug platform that can carry any number and type of functional moieties. (Ljubimova et al. (2014), which is incorporated herein by reference as if fully set forth).

Certain molecules are transported across the BBB via highly selective endogenous transport mechanisms. For example, the low-density lipoprotein receptor pathway (LRP-1) enables the bidirectional movement of low density lipoproteins across the BBB (Georgieva et al. (2014); and Dehouck et al. (1997), both of which are incorporated herein by reference as if fully set forth).

LRP-1 mediated blood-to-brain transport occurs when suitable ligands bind to and become internalized by LRP-1 in the vascular endothelium. After internalization, LRP-1 bound ligands are transcytosed into the brain parenchyma. A synthetic LRP-1 peptide ligand, Angiopep-2 (AP-2 or AP2; TFFYGGSRGKRNNFKTEEY (SEQ ID NO: 1)), was identified by Demeule et al. (Demeule et al. (2008), which is incorporated herein by reference as if fully set forth). It was reported that the transport of AP-2 saturates at high concentrations and is inhibited by other LRP-1 ligands, confirming AP-2 transcytosis. AP-2 was selected for initial screening.

Another class of peptides enhances BBB drug penetration via the transferrin receptor (TfR) pathway. The TfR pathway imports iron into the brain and is critically involved in maintaining cerebral iron homeostasis. TfRs are selectively expressed on endothelial cells of brain capillaries and thus provide a conduit for selective drug delivery into the brain (Johnsen et al. (2017) which is incorporated herein by reference as if fully set forth). An iron-mimic peptide ligand for TfR-mediated drug delivery, cTfRL, also referred to herein as Fe mimetic peptide, ((SEQ ID NO: 2), CRTIGPSVC —NH2, cyclic, S—S bonded) was isolated via phage display and has been shown to deliver cargo into brain tumors (Staquicini et al. (2011), which is incorporated herein by reference as if fully set forth). Fe mimetic peptide, or cTfRL was also selected for the design.

Another TfR ligand, B6 (CGHKAKGPRK (SEQ ID NO: 9)), has been described and selected for the design of mini nanodrugs (Yin et al. (2015), which is incorporated herein by reference as if fully set forth).

Another selected peptide was Miniap-4 (also referred to herein as M4; H-[Dap] KAPETAL D-NH₂ (SEQ ID NO: 3), a cyclic peptide that was derived from bee venom. This peptide was reported to be capable of translocating proteins and nanoparticles across a human cell-based BBB model, (Oiler-Salvia et al. (2016), which is incorporated herein by reference as if fully set forth).

None of the above-mentioned BBB penetrating peptides have inherent therapeutic value(s) and they have not been designed to carry reversibly bound cargoes by themselves. These peptides were selected as components of cargo delivery molecules and were examined to determine how conjugation with other peptide or non-peptide moieties influences their BBB penetration abilities.

The mini nanodrugs based on the PMLA backbone conjugated to synthetic peptides that enable BBB penetration were additionally designed to carry tri-leucine (LLL). LLL displays pH-responsive lipophilicity and promotes endosomal escape of PMLA bound agents once they are internalized and part of the endosomal pathway. Endosomal escape for cytoplasmic drug delivery was described for intracellular drug treatment (Ding et al. (2011), which is incorporated herein by reference as if fully set forth).

The mini nanodrugs were also conjugated to rhodamine in order to visualize the compound in brain tissues.

The mini nanodrugs were initially designed to be neutral to test their ability to penetrate BBB and be distributed over all brain regions which could potentially be affected by neurological disorders.

Additionally, the mini nanodrugs were designed for multi targeted systemic delivery of antisense oligo nucleotides (AONs) and of microRNAs for multipronged down- and upregulation of mRNA in protein synthesis and chemotherapeutics across blood brain barrier (BBB) to silence Aβ production.

The antisense oligonucleotide may include a sequence complementary to a sequence contained in an mRNA transcript of β-secretase or γ-secretase, for example, of SEQ ID NO: 4. The mini nanodrug may include a nucleic acid sequence capable of targeting a messenger RNA transcribed from the target gene. The nucleic acid sequence may be a microRNA (miRNA). The miRNA may be a microRNA-186 to downregulate a β-secretase enzyme, BACE1. The miRNA-186 sequence may be a sequence of the Morpholino oligonucleotide of SEQ ID NO: 14 for downregulation of BACE1 in AD-patients.

The mini nanodrugs can be conjugated to β-sheet breaker peptides. Besides blocking syntheses of secretases and tau, β-sheet breaker peptides are o designed to specifically interfere with β-sheets within Aβ preventing the misfolding and deposition of Aβ and decreasing toxicity e.g. H102 (HKQLPFFEED; SEQ ID NO: 7) peptide (Zhang et al. (2014), which is incorporated herein by reference as if fully set forth). These and other functional peptides are included for dissolution of aggregates and plaques.

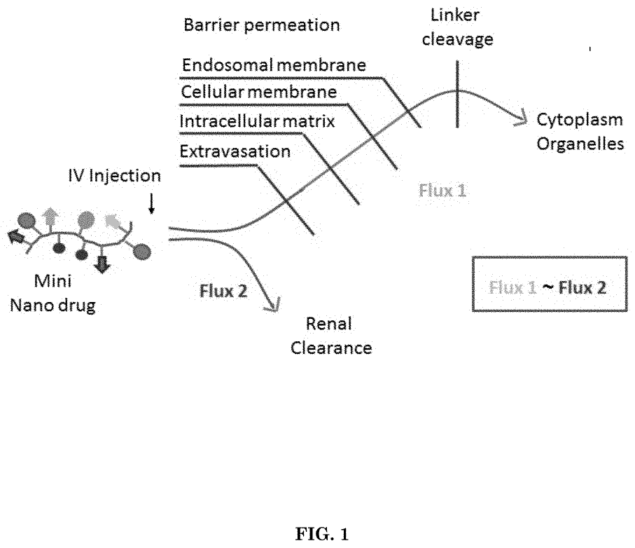

The mini nanodrugs were designed to carry multiple peptides for targeting across BBB and providing a fast and massive flux of delivery into the brain. FIG. 1 is a schematic drawing illustrating overview of molecular pathway of mini nanodrugs. Referring to this figure, the mini nanodrugs are i.v. injected into a subject. The massive flux (flux 1) is maintained by binding of different attached peptides that target specific barriers, such as endosomal membrane, cellular membrane, intracellular matrix, extravasion, along this mini nanodrug pathway. Multiple peptides targeting different pathways to same barriers would increase the overall flux of drug delivery through barriers. At the site of treatment (cytoplasm organelles), the covalent attached drug(s) are cleaved from the nanocarrier by enzymatic reaction or spontaneous reaction with reactant contained only in the targeted site of treatment (i.e. hydrogen ions (pH), or Glutathion-SH for reductive cleavage of disulfide linkers of drug with carrier). Another flux (flux 2) is directed to renal clearance.

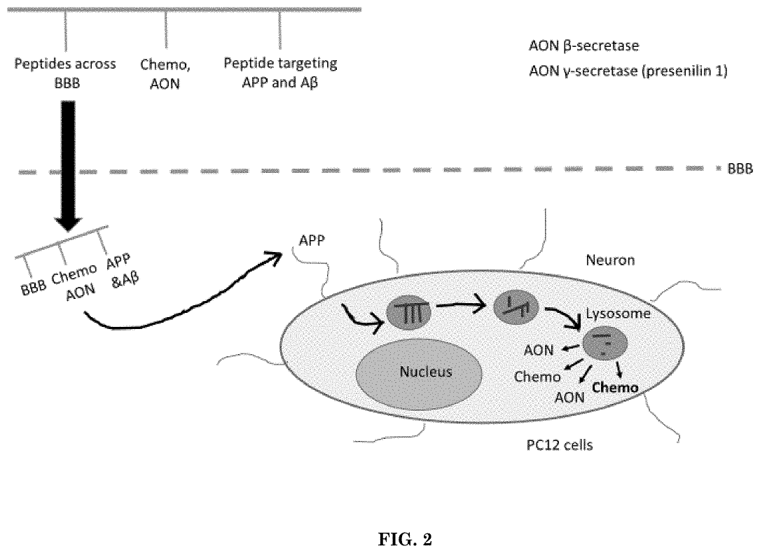

The mini nanodrugs were designed to carry peptides and specifically target to neuron cells which overproduce the Aβ precursor peptides (APPs). The mini nanodrugs were designed to carry antisense oligonucleotides (AONs) to silence mRNAs, and thus, biosynthesis of β-secretase and/or γ-secretase for Aβ production. FIG. 2 is a schematic drawing illustrating mini nanodrugs carrying peptides that permeate through multiple bio barriers into targeted neurons, chemo, AONs, and peptides targeting APP and Aβ. One kind of AON is an AON inhibiting the syntheses of β-secretase and another kind of AON is an AON inhibiting the synthesis of γ-secretase presenilin 1 (the enzyme active) subunit. The delivery of Morpholine-AONs in the form of Morpholino oligonucleotides is advantageous because they do not use electrically charged phosphate groups as building blocks that renders them very resistant against hydrolytic degradation.

To exercise AON-dependent inhibition of protein synthesis, the AON containing platform targets a cell surface located receptor and crosses the cellular membrane by receptor-mediated endocytosis, finally escaping the endosome into the cytoplasm during a late stage of endosome pathway before entering the lysosome. The late stage is characterized by a drop in endosomal pH which activates the LLL-mediated disruption of the endosome membrane allowing the nanoconjugate to enter the cytoplasm. The disruption is accomplished via the platform located tri-leucine (LLL) residues, which display lipophilicity at the low pH.

The synthesis of AON ligated PLMA/LLL platforms containing the amyloid targeted D-peptides, the efficient movement across BBB along the LRP-1 transcytosis pathway, and finally their targeted internalization into neurons comprising deposition as free conjugates and in particles is described herein.

The mini nanodrug further carries drugs (marked as “chemo” on FIG. 2) to inhibit the secretase activities. The mini nanodrug further carries tri-leucine for release of the delivery system across the endosome membrane into the cytoplasm. The mini nanodrug further carries optionally Cy 5.5 (NIR fluorescence), Phalloidin (red fluorescence) or DOTA-Gd(III) for fluorescence imaging or imaging by magnetic resonance (MRI).

Referring to FIG. 2, after IV injection, the mini nanodrug permeates BBB and unfolds inhibition of Aβ-synthesis by blocking β- and γ-secretase protein syntheses and enzyme activities (contained in cytoplasm and/or organelles). The peptides angiopep-2, cyclic MiniAp-4, cyclic CRTIGPSVC (SEQ ID NO: 2)-peptide target the delivery across BBB on parallel routes of transcytosis. Transcytosis of high flux competes successfully with vascular clearance. An amyloid targeting peptide specifically adheres the mini nanodrug to amyloid precursor peptides (APP) on the surface of Aβ overproducing neurons. APP and adhering mini nanodrug are internalized into the endosomal system for cleavage by β-secretase and release of AONs and secretase inhibitory drugs. AONs released into the cytoplasm specifically inhibit the biosynthesis of β-secretase and γ-secretase. The membrane permeable drugs inhibit secretase cleavage of APP and release of Aβ into extracellular space. Absence of Aβ production stops Aβ aggregation, fibril formation and toxic reactions. Dissolution of existing plaques occurs with time or may be accelerated by treatment with aggregate disrupting reagents (e.g., peptides and synthetics).

The mini nanodrugs consisting of degradable non-immunogenic systemic IV-injectable nanoagent is suitable for imaging and treatment of Alzheimer disease. The mini nanodrug can be applied for treatment of other neurological disorder by use of appropriate peptides, chemotherapeutics and antisense oligonucleotides. Because of the multiplicity of attachment sites on the PMLA carrier, the mini nanodrug can be equipped with multiple chemotherapeutics and DNA-antisense drugs for blockage of Alzheimer specific markers. Attachment of chemotherapeutics and oligonucleotides to the mini nanodrug is reversible when responding to local pH or glutathione and suits drug activation inside targeted cells. Reagents carry dyes for NIR or IR image guided space and time resolved analysis.

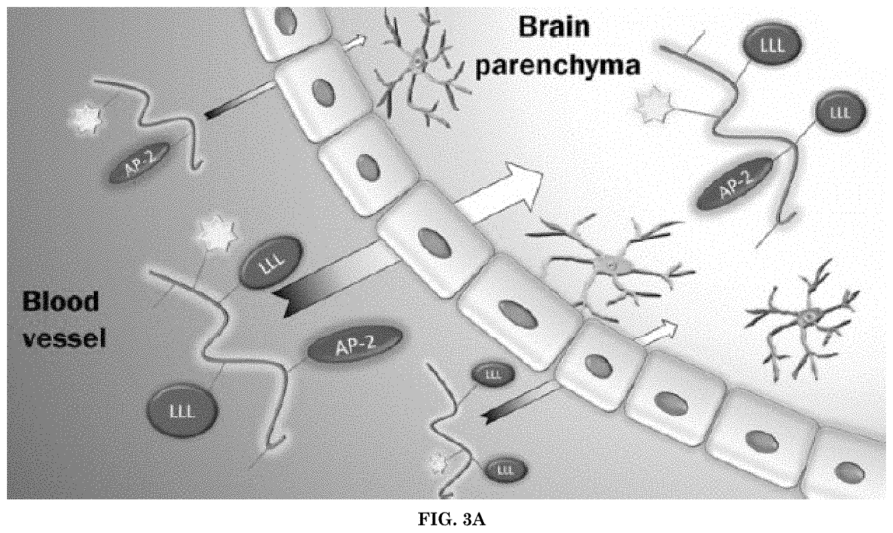

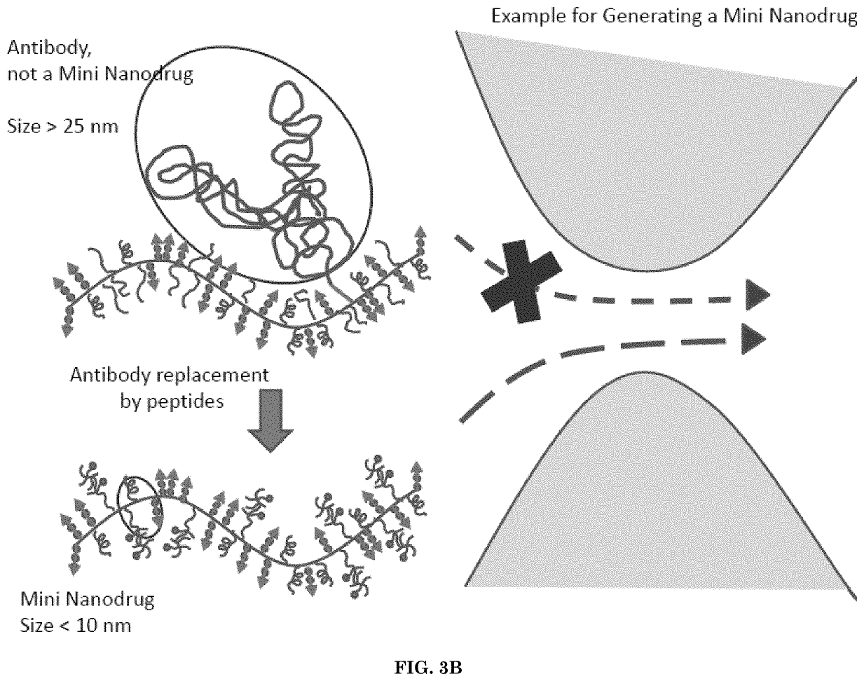

FIGS. 3A-3B are schematic drawings illustrating advantages of mini nanodrugs for crossing the blood-brain barrier and entering brain parenchyma. FIG. 3A is a schematic drawing illustrating mini nanodrugs carrying AP-2 peptides and tri-leucines (endosomic escape units) entering brain parenchyma. The mini nanodrugs for fast delivery and deep penetration were designed to be 6-10 nm size and have an elongated architecture. This was achieved by attaching low molecular targeting peptides to PMLA. FIG. 3B is a schematic drawing comparing the efficiency of crossing the blood-brain barrier of a mini nanodrug carrying peptides and nanodrugs that carry antibodies. However, the mini nanodrugs described herein may contain antibodies if the antibody or of other large molecule's function is required as such and cannot be substituted by peptides because they are not available or if the antibody contains biologically important effector site different from antigen recognition site. This design may widen the application of the mini nanocarriers. The molecular weight of the mini nanodrugs that carry an antibody in addition to peptides may increase and the hydrodynamic diameter may increase to 10-15 nm.

Polymalic acid (PMLA) is an unbranched polymer and macromolecule with multiple pendant carboxylic groups for attachment of a diversity of pharmaceutical functional modules. The linear organization of structurally highly flexible polymalic acid allows enhanced diffusion through interstitial space and optimal accessibility of multiple peptides with interacting sites. The small molecular size on the lower nanoscale and the molecular flexibility provide an optimal penetration in brain.

Favorable high influx from circulating vasculature into brain is obtained by attachment of several different affinity peptides that engage simultaneously in binding to multiple sites and BBB crossing pathways of different specificity. Inside brain, second peptides target specific markers of Alzheimer or of other neurodegenerative diseases. Furthermore, NIR fluorescent dyes are attached for imaging, and chemotherapeutic drugs and antisense oligo nucleotides for treatment. Peptides have low immunogenicity, are robust against denaturation and in an exocyclic form less vulnerable by enzymatic cleavage. Peptides have less affinity and hence favorable release kinetics after receptor binding. Conjugation of targeting peptides with multi attachment sites carried by polymalic acid increases influx of functional groups for inside targeting, imaging and treatment. The enumerated favorable properties make the delivery system surprisingly applicable for efficient and versatile delivery across BBB providing unique advantages over other delivery devices. The mini nanodrugs can be useful in addressing the problem of poor availability of delivery pathways across BBB and their inefficacy to manage large nanoparticles, instability and long circulation times prone for loss of cargo and induction of systemic side effects. The mini nanodrugs can be used for solving additional problems such as expensive production (antibodies), limited shelf life, difficult to manage shipment in solution, and the necessity to apply large volumes for patient application. The mini nanodrugs can be used for solving the problem of incomplete inhibition of secretases and high degree of side effects caused by lack of targeting producer cells, and the need of imaging to control progress of treatment. Alternatively, the mini nanodrugs can carry both peptides and antibodies as described in examples herein.

The nanocarrier's structure is designed for fast diffusion and easy barrier penetration, excellent access of interaction sites, attachment of agents for optical (fluorescence) and magnetic imaging (MRI). Manageable costs by simplified production, storage, shipping, and patient application.

Aβ peptide overproducing cells are peptide targeted. Targeting was also addressed to silence over production of proteins and peptides. Silencing employs antisense oligonucleotides and miRNAs in a multi-pronged initiative and includes inhibition by chemo therapeutics.

### Example 2

### Syntheses of Polymalic Acid (PMLA) Based Nanoconjugates

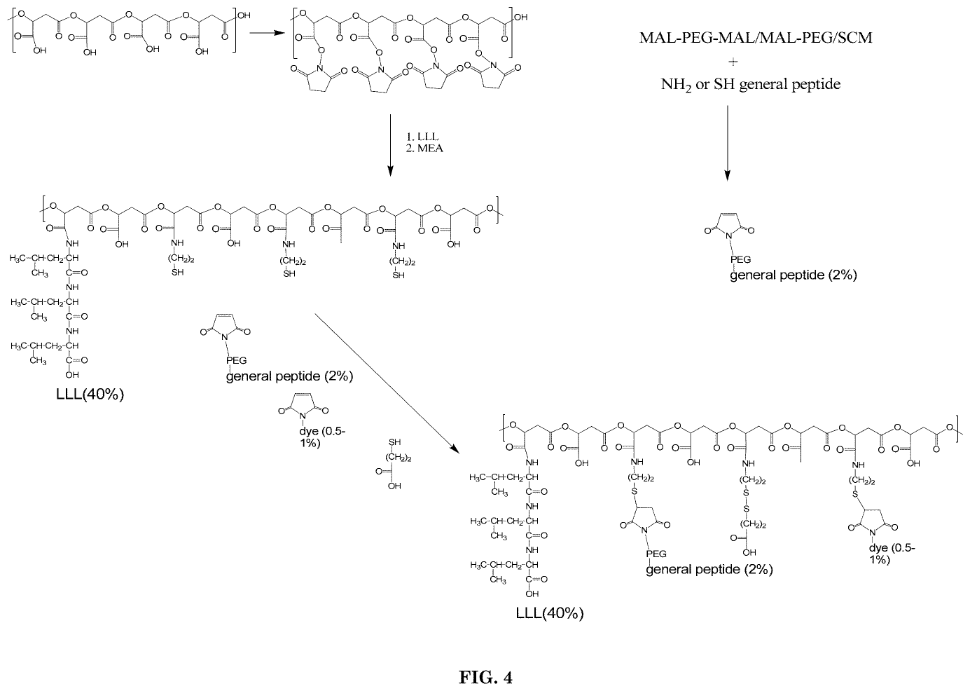

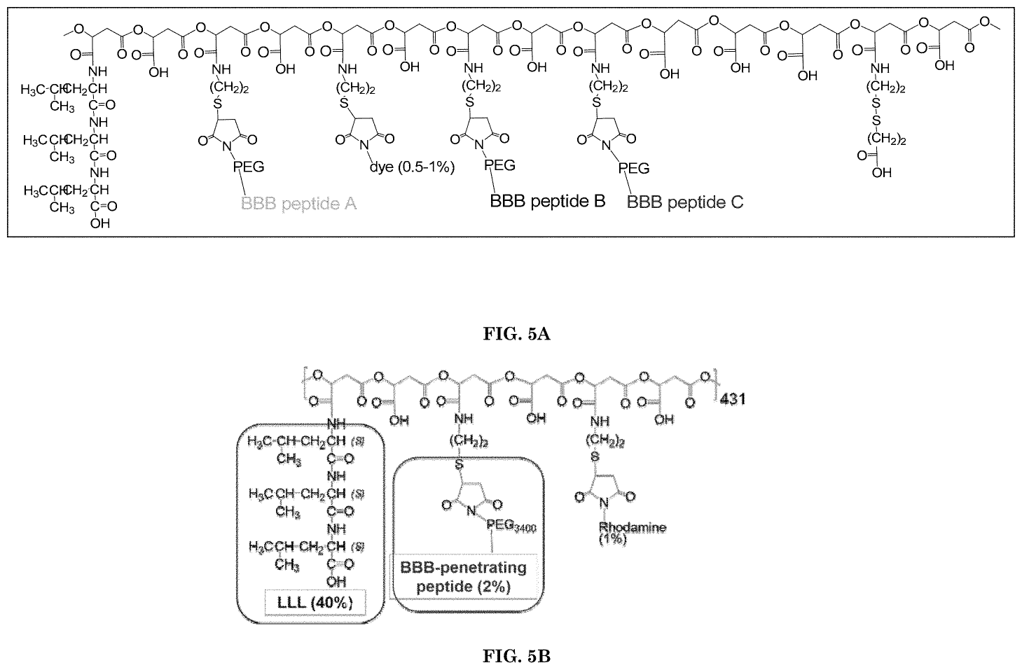

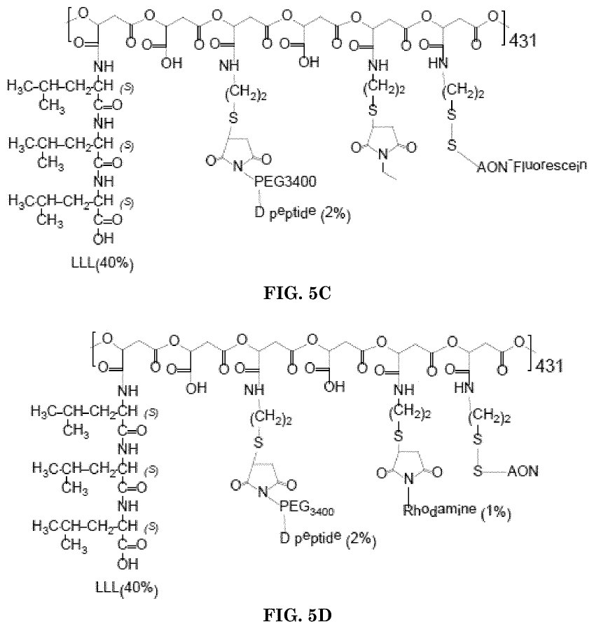

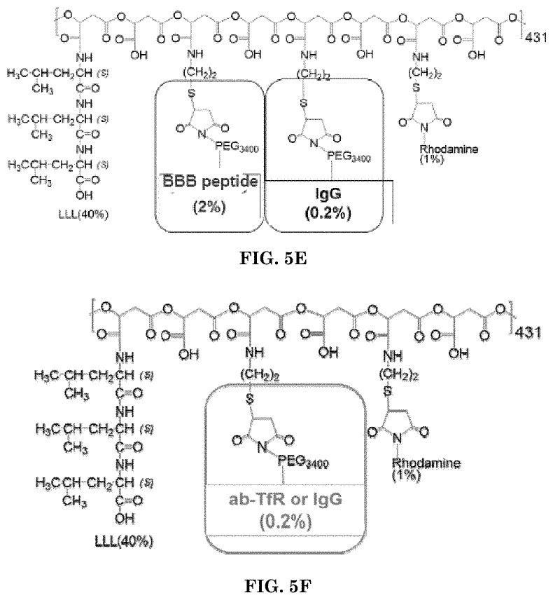

The master schemes depicting representative reactions are illustrated on FIGS. 4 and 5A-5F. FIG. 4 illustrates synthesis of the mini nanodrug with a single peptide. The mini nanodrug has capability for the extension to specific cascade targeting across BBB to addressed brain cells. The flow of synthesis starts on the upper left corner with NHS activation of polymalic acid (PMLA). Activation is followed by amide forming substitution with tri-leucine (LLL) consuming 40% of pendant activated carboxylates, then by amide forming substitution with 2-mercapto ethylamine (MEA) (10% of available carboxylic groups or consuming an optional amount of activated carboxylates) to achieve the intermediate product termed “preconjugate”. The sulfhydryls on the PMLA scaffold react with maleimide tagged peptides and imaging groups forming the corresponding thioether conjugates. The conjugation of peptides to present the maleimide reactive groups employs commercially available bifunctional PEG2000/3400-linkers attached to reactive groups on peptides (and dyes, if required) (see scheme in the upper right corner of the Scheme). Morpholino oligonucleotides (AONs) are loaded by disulfide exchange of preconjugate-SH with 3-pyridyldithiopropionyl-3′-amido-AON (Ljubimova et al. (2014), which is incorporated herein by reference as if fully set forth).

Excess remaining sulfhydryl groups are blocked by exchange reaction with 3-pyridyldithiopropionate (PDP).

FIGS. 5A-5F illustrate examples of the mini nanodrugs, containing peptides, AONs and antibodies. FIG. 5A illustrates an example of the mini nanodrugs containing three peptides. FIG. 5B illustrates an example of the mini nanodrugs containing LLL (40%), BBB-penetrating peptide (2%) and rhodamine dye (1%). FIG. 5C illustrates an example of the mini nanodrug containing LLL (40%), D-peptide (2%), and AON-fluorescein. FIG. 5D illustrates an example of the mini nanodrug containing LLL (40%), D-peptide (2%), rhodamine dye (1%) and AON. FIG. 5E illustrates an example of the mini nanodrugs containing LLL (40%), BBB-penetrating peptide (2%), IgG (0.2%) and rhodamine dye (1%). FIG. 5F illustrates an example of the mini nanodrugs containing LLL (40%), ab-TfR or IgG (0.2%) and rhodamine dye (1%).

Materials: Highly purified poly(β-1-malic acid; 50 kDa) was prepared from the culture broth of Physarum polycephalum as previously described (Ljubimova et al. (2014), which is incorporated herein by reference as if fully set forth). The peptides Angiopep-2-cys (containing an additional C-terminal cysteine group; TFFYGGSRGKRNNFKTEEYCNH₂ (SEQ ID NO: 1)), Angiopep-7-cys (TFFYGGSRGRRNNFRTEEYCNH₂ (SEQ ID NO: 7)), B6 (CGHKAKGPRK (SEQ ID NO: 9)), M4 (H-[Dap] KAPETAL D-NH₂ (SEQ ID NO: 3)), and cTfRL, also referred herein as the Fe mimetic peptide, (CRTIGPSVC-NH2, (SEQ ID NO: 2), S—S bonded) were custom synthesized by AnaSpec (Fremont, Calif., USA). The peptides D1 QSHYRHISPAQVC (SEQ ID NO: 9), D3 RPRTRLHTHRNRC (SEQ ID NO: 10) and ACI89 PSHYRHISPA QKC (SEQ ID NO: 11) all containing an added C-terminal cysteine were custom synthesized by AnaSpec (Fremont, Calif., USA). D-configured peptides were custom-made α-D-amino acids mirror-copies of original phage-displayed peptides in L-configuration are described and commercially available (Wiesehan and Dieter Willbold: Mirror-image Phage Display: Aiming at the Mirror. ChemBioChem 2003, 4, 811-815). Labeled and unlabeled AONs with 5′ primary amine were purchased from Gene Tools (Philomath, Oreg., USA). (3-(2-Pyridyldithio)propionic acid N-hydroxysuccinimide ester (S-PDP) was purchased from ProteoChem (Hurricane, Utah, USA). Rhodamine-maleimide was purchased from ThermoFisher Scientific (Canoga Park, Calif., USA). Mal-PEG3400-Mal or Mal-PEG2000-Mal was purchased from Creative PEGWorks (Durham, N.C., USA). Tri-Leucine was ordered from Bachem (Torrance, Calif., USA) while the antibody used as cargo was IgG2a kappa from murine myeloma, the reagents dimethyl formamide (DMF), acetonitrile (ACN), N-ethylmaleimide and triethylamine (Et₃N), dicyclohexylcarbodiimide (DCC), N-hydroxysuccinimid (NHS), trifluoroacetic acid (TFA), mercaptoethylamine (MEA) and dithiothreitol (DTT) were obtained from Sigma Aldrich (St. Louis, Mo., USA). Sephadex™ G-75 resin and PD-10 columns were purchased from GE Healthcare (Chicago, Ill., USA). Vivaspin centrifuge filter tubes were purchased from sartorius (Stonehouse, UK). PMLA/LLL (40%)/MEA (10%) (“PMLA pre-conjugate”), peptide-PEG3400-maleimide, and peptide-PEG2000-maleimide were synthesized as described herein.

Detailed Syntheses are Shown Below

Products peptides were stored a −20° C. or lyophilized.

Synthesis of PMLA/MEA (10%) (PMLA preconjugate without trileucine): PMLA, 19 mg (116 g/mol monomer, 0.164 μmol) was placed in a glass vial with magnetic stirrer (ambient temperature), and dissolved in 300 μL acetone. N-hydroxy succinimide (NHS, 115 g/mol, 9.6 mg, 0.083 μmol, 50 mole % of PMLA COOH) and N,N′-Dicyclohexylcarbodiimide (DCC, 206 g/mol, 17.7 mg, 0.086 μmol, 50 mol % of PMLA COOH) were dissolved in 500 μL of DMF and added dropwise to the reaction mixture, followed by 15 mg of dithiothreitol (DTT, 154.25 g/mol, 0.097 μmol) in 38 μL of DMF and then cysteamine (MEA, 113.61 g/mol, 1.9 mg, 0.017 μmol, in 7.8 μL DMF) and Et₃N (2.3 μL, 1 eq to MEA). The reaction was monitored using TLC (n-BuOH:H₂O:AcOH 5:1:1, visualization using ninhydrine clip). After reaction termination, 0.8 mL of sodium phosphate buffer (150 nM, pH 6.8) were added and the reaction was stirred for an additional two hours. The mixture was centrifuged to separate from precipitate, and the liquid phase was purified over PD-10 column. Analysis by SEC-HPLC indicated a retention time of 7.0 min (HPLC pump: Hitachi L-2130; detector, Hitachi L-2455; software, EZChrome; Column, Polysep 4000; flow rate: 1 ml/min; buffer, PBS). The final product was lyophilized, and the resulting white fiber solid was stored at −20° C. The general scheme for synthesis of a mini nanodrug containing a single peptide is shown on FIG. 4.

Synthesis of PMLA/LLL (40%)/MEA (10%) (PMLA Pre-Conjugate):

Hereinafter, the percent loading of LLL or of other substituents elsewhere was referred with reference to the total content of malic acid residues in the polymer. PMLA (40 mg of 116 g/mol monomer, 0.345 μmol) was dissolved in 400 μL acetone at ambient temperature. A mixture of N-hydroxy succinimide (NHS, 115 g/mol, 40 mg, 0.345 μmol) and N,N′-dicyclohexylcarbodiimide (DCC, 206 g/mol, 74 mg, 0.36 μmol) dissolved in 400 μL of DMF was added dropwise. After 2 hours, a mixture of tri-leucine (LLL, 357.4 g/mol, 49.3 mg, 0.138 μmol) and tri-fluoro acetic acid (TFA, 114 g/mol, d=1.489 g/mL, 12.7 μL) in 200 μL DMF was added (in portions of 20, 25, 30, 35, 40, 45 and 50 μL in 10 minute intervals). Every addition was followed by Et₃N in DMF (101.2 g/mol, d=0.73 g/mL, 26.65 μL in 200 μL DMF as portions of 15, 20, 25, 30, 35, 40 and 45 μL). The reaction extent was monitored using TLC (n-BuOH:H₂O:AcOH 5:1:1) and ninhydrin reaction. After reaction termination, dithiothreitol (DTT, 7 mg, 154.25 g/mol, 0.045 μmol) in 50 μL of DMF was added, followed by cysteamine (MEA, 113.61 g/mol, 3.92 mg, 0.035 mol, in 10.8 μL DMF) and Et₃N (4.8 μL, 1 eq to MEA) for conjugation of NH—CH₂—CH₂—SH₂. The reaction was monitored using TLC and ninhydrin reaction. After reaction termination, 1.2 mL (identical to reaction volume) of sodium phosphate buffer (150 nM, pH 6.8) were added, the mixture stirred for an additional two hours, and ultimately centrifuged to separate from precipitation. The product was purified over a PD-10 column and characterized by HPLC (7.2 min retention time, 220 nm wavelength, HPLC pump: Hitachi L-2130; detector: Hitachi L-2455; EZChrome software; Polysep 4000 column; flow rate of 1 ml/min; PBS). After lyophilization, the product was stored as a white fibrous material.

SEC-HPLC analysis: The analysis was performed using a Hitachi L-2130 pump with a Hitachi L-2455 detector with EZChrome Software. The SEC-HPLC column was Polysep 4000, at 1 ml/min flow rate, PBS (pH 7.4). For measurements of molecular weight of PMLA nanoconjugates, retention volumes were calibrated with polystyrene sulfonates of known molecular mass. Elution of polymalic acid conjugates was measured at 200-220 nm wavelength.

Syntheses of Mal-Linker-Peptides:

Synthesis of Angiopep-2-PEG3400-Mal: At ambient temperature, Mal-PEG3400-Mal (3400 g/mol, 7.4 mg, 2.2 μmol, 1.05 eq) dissolved in 500 μL of phosphate buffer 100 nM (with 2 mM EDTA) with pH 6.3, received dropwise cysteine modified Angiopep-2 (SEQ ID NO: 1), 2403.7 g/mol, 5 mg, 2.08 μmol, 1 eq, dissolved in 500 μL phosphate buffer pH 6.3. The reaction as monitored by HPLC, was completed after one hour. The lyophilized product (10 mg/mL in phosphate buffer with pH 6.3) was used for the reaction with PMLA preconjugate (SEC-HPLC analysis: retention time 8.2 min at 220 nm wavelength). Angiopep-7-PEG3400-Mal (SEC-HPLC retention 8.25 min at 220 nm) and B6-PEG-Mal (SEC-HPLC retention 7.92 min at 220 nm) were synthesized in the same manner. SEC-HPLC analysis condition as above. Retention 8.2 min at 220 nm wavelength.

Synthesis of “Fe mimetic peptide,” or cTfRL (SEQ ID NO: 2) (cyclic)-peptide-PEG2000-Mal: In a glass vial with magnetic stirrer (ambient temperature), Mal-PEG-SCM 2000 (2000 g/mol, 11.2 mg, 5.6 μmol, 1.05 eq) was dissolved in 500 μL of DMF. “Fe mimetic peptide” (SEQ ID NO: 2): CRTIGPSVC (cyclic)-peptide (932 g/mol, 5 mg, 5.36 μmol, 1 eq) dissolved in 500 μL DMF was added followed by 0.89 μL of Et₃N (101.2 g/mol, d=0.73 g/mL, 6.45 μmol, 1.48 eq, or 8.9 μL of Et₃N solution 10-fold diluted in DMF). The reaction was monitored using HPLC and 0.1 μL of Et₃N were added in case the reaction was not progressing. The reaction ended after an overnight stirring and was purified using PD-10 column, analyzed using HPLC and lyophilized. A solution of 10 mg/mL product in phosphate buffer 6.3 was used for the reaction with PMLA preconjugate.

SEC-HPLC analysis condition as above: Retention 8.3 min at 220 nm wavelength. Mass spectrum at Mw 2817 consistent with expected product.

Synthesis of Miniap-4-PEG2000-Mal: In a glass vial with magnetic stirrer (ambient temperature), Mal-PEG-SCM 2000 (2000 g/mol, 5.5 mg, 2.76 mol, 1.2 eq) was dissolved in 200 μL of DMF. Miniap-4 (SEQ ID NO: 3) (911.1 g/mol, 2.1 mg, 1 eq) dissolved in 200 μL DMF was added followed by 0.45 μL of Et₃N (101.2 g/mol, d=0.73 g/mL, 3.25 μmol, 1.48 eq, or 4.5 μL of Et₃N solution 10-fold diluted in DMF).

The reaction was monitored using HPLC (same conditions mentioned above; Retention 8.1 min at 220 nm wavelength), and 0.3 eq of Mal-PEG2000-SCM (1.32 mg in DMF) and 0.1 μL of Et₃N were added in case the reaction was not progressing. Much excess of Mal-PEG2000-SCM and an overnight reaction were avoided to keep side reactions with lysine at a minimum. The reaction was purified using PD-10 column, analyzed using HPLC and lyophilized. A solution of 10 mg/mL product in phosphate buffer 6.3 was used for the reaction with PMLA preconjugate.

Synthesis of peptide-PEG2000-Mal: At ambient temperature, Mal-PEG2000-SCM (2000 g/mol, 3.5 mg, 1.75 μmol, 1.05 eq) was dissolved in 250 μL of DMF. TfR ligand (932 g/mol, 1.5 mg, 1 eq, 1.6 μmol) dissolved in 250 μL DMF was added followed by 0.34 μL of Et3N (101.2 g/mol, d=0.73 g/mL, 2.4 μmol, 1.5 eq). The reaction was monitored using HPLC (usually overnight), and 0.1 μL of Et3N were added in case the reaction was not progressing. The reaction was purified using a PD-10 column, analyzed using HPLC, and lyophilized. Miniap-4-PEG2000-Mal was synthesized in the same manner, using the N-terminus and the succinimidyl carboxyl methyl ester reaction for attachment.

Synthesis of PMLA/peptide (2%)/dye conjugate from PMLA preconjugate not containing tri-leucine: 2 mg of PMLA/MEA (10%) (127.36 g/mol monomer, 15.7 μmol) were dissolved in 300 μL of phosphate buffer pH 6.3 and were placed in a glass vial with magnetic stirrer at ambient temperature. 2% (0.314 μmol) of peptide-PEG-MAL were added dissolved in phosphate buffer pH 6.3 to 10 mg/mL of optional peptide-linker-Mal: optionally 1.82 mg of angiopep-2-PEG3400-MAL (5802.7 g/mol) or 0.88 mg of “Fe mimetic peptide” (SEQ ID NO: 2): CRTIGPSVC (cyclic)-peptide-PEG2000-Mal (2817 g/mol) or 0.88 mg of Miniap-4-PEG2000-Mal (2796 g/mol), or buffer without peptide (control). The reaction mixture was monitored at 220 nm by HPLC (typically 1 h reaction) and, once completed, Rhodamine C2*) was loaded by thioether formation with the PMLA platform —SH (0.107 mg for 1% loading, 680.79 g/mol, 0.153 μmol, 52.2 μL of 2 mg/mL solution in DMF). The reaction under exclusion of light was monitored using HPLC. Absorbance spectra were recorded to detect dye absorbance in the PMLA conjugate elution peak. After stirring of the reaction mixture for further 1-2 h, 15 μL of 3-(2-pyridyldithiopropionic acid) or PDP (10 mg/mL solution in DMF) was added to cap the free SH groups. The reaction was stirred for an additional hour and purified over PD-10 column, analyzed by HPLC, lyophilized and stored at −20° C. *) Optionally NIR dye Cy5,5 was also used for fluorescence labeling.

Synthesis of PMLA/peptide (2%)/dye conjugate: The reaction was conducted in the same manner as PMLA/LLL/peptide (2%)/dye using PMLA/MEA (10%) conjugate (2 mg, 127.36 g/mol monomer, 0.0157 mmol) and either 1.82 mg, 3.14 μmol, 5802.7 g/mol, of AP2-PEG-MAL or 0.88 mg cTfRL-PEG-Mal, 2817 g/mol, or 0.88 mg M4-PEG-Mal, 2796 g/mol; 0.107 mg, 680.79 g/mol, 0.153 μmol of rhodamine-maleimide (1% loading) was used.

Synthesis of PMLA/LLL (40%)/peptide (2%)/dye (1%): Four milligrams of PMLA/LLL (40%)/MEA (10%) (260.7 g/mol, 15 μmol preconjugate monomer) were dissolved in 350 μL of phosphate buffer pH 6.3 and placed in a glass vial with a magnetic stirrer at ambient temperature. In order to achieve 2% loading, 1.78 mg of Angiopep-2-PEG3400-Mal (5802.7 g/mol), or 2.07 mg of Angiopep-7-PEG3400-Mal (5858.8 g/mol), or 0.87 mg cTfRL-PEG-Mal (2817 g/mol), or 0.86 mg Miniap-4 (M4)-PEG2000-Mal (2796 g/mol) or 1.33 mg B6-PEG2000-Mal (4480 g/mol) or 1.68 mg of ACI89-PEG3400-maleimide (4923 g/mol), or 1.58 mg D3-PEG3400-maleimide (5103 g/mol), or 1.67 mg D1-PEG3400-maleimide (4925 g/mol), or 0.86 mg of “Fe mimetic peptide” (SEQ ID NO: 2): CRTIGPSVC (cyclic)-peptide-PEG2000-Mal (2817 g/mol), or buffer without peptide (control) were all dissolved in phosphate buffer pH 6.3 to 10 mg/mL concentration and were added dropwise. After 1 h, the reactions monitored using SEC-HPLC (220 nm) were completed. Then rhodamine-maleimide (0.104 mg for 1% loading, 680.79 g/mol, 0.149 μmol, 52 μL of 2 mg/mL solution in DMF) was loaded onto the conjugates forming thioethers with the PMLA platform at pendant MEA-SH. The reaction was conducted in the dark and was monitored using HPLC. Success of the conjugation was indicated by the rhodamine absorbance in the PMLA conjugate elution peak. After stirring of the reaction mixture for further 1-2 h, 15 μL of 3-(2-pyridyldithio)propionic acid (10 mg/mL solution in DMF) was added to cap the free SH groups. After stirring the mixture an additional hour, the product was purified over a PD-10 column, analyzed, lyophilized and stored at −20° C.

Synthesis of PMLA/LLL (40%)/peptide (2%)/peptide (2%)/dye (1%): 1 mg of PMLA/LLL (40%)/MEA (10%) (260.7 g/mol monomer, 3.84 μmol) were dissolved in 300 μL of phosphate buffer pH 6.3 and placed in a glass vial with magnetic stirrer at ambient temperature. For 2% loading, (0.077 μmol) of peptide-PEG-MAL were added dissolved in phosphate buffer pH 6.3 to 10 mg/mL concentration: optionally 21.5 μL “Fe mimetic peptide” (SEQ ID NO: 2): CRTIGPSVC (cyclic)-peptide-PEG2000-Mal (2817 g/mol) or 0.215 mg (21.5 μL) of Miniap-4-PEG2000-Mal (2796 g/mol). The reaction is monitored using HPLC. After reaction termination, the second peptide is added: optionally 0.445 mg of angiopep-2-PEG-MAL 3400 (5802.7 g/mol) or 0.215 mg of “Fe mimetic peptide” (SEQ ID NO: 2): CRTIGPSVC (cyclic)-peptide-PEG2000-Mal (2817 g/mol, in case of miniap-4 was the first peptide). The reaction mixture was monitored at 220 nm using HPLC (typically 1 h reaction) and once completed, Rhodamine C2 was added (0.026 mg for 1% loading, 680.79 g/mol, 0.38 μmol, 13.05 μL of 2 mg/mL solution in DMF) and the reaction under exclusion of light was monitored using HPLC. Dye absorbance aside PMLA absorbance were recoded and the reaction stirred for 1 h. Then, 05 μL of 3-(2-pyridyldithiopropionic acid) or PDP (10 mg/mL solution in DMF) was added to cap the free SH groups. The reaction was stirred for an additional hour before purification using PD-10 column, HPLC analysis, lyophilization and storage at −20° C.

Synthesis of PMLA/LLL (40%)/peptide (2%)/peptide (2%)/peptide (2%)/dye (1%): 1 mg of PMLA/LLL (40%)/MEA (10%) (260.7 g/mol monomer, 3.75 μmol) were dissolved in 300 μL of degassed phosphate buffer pH 6.3 and were placed in a glass vial with magnetic stirrer at ambient temperature. For 2% loading, optionally (0.077 μmol) or 0.512 mg of angiopep-2-PEG3400-MAL (5802.7 g/mol) peptide-PEG-MAL were added dissolved in phosphate buffer pH 6.3 to 10 mg/mL concentration. The reaction is monitored at 220 nm and dye absorbance using HPLC, and is typically complete after 1 h. Then, the second peptide is added: optionally 21.5 μL of “Fe mimetic peptide” (SEQ ID NO: 2): CRTIGPSVC (cyclic)-peptide-PEG2000-Mal (2817 g/mol) or 0.215 mg (21.5 μL) of Miniap-4-PEG2000-Mal (2796 g/mol). After addition of the third peptide and reaction completion, the remainder of the conjugate preparation follows the description under S9. After stirring of the reaction mixture for further 1-2 h, 15 μL of 3-(2-pyridyldithio)propionic acid (10 mg/mL solution in DMF) was added to cap the free SH groups. After stirring the mixture an additional hour, the product was purified over a PD-10 column, analyzed, lyophilized and stored at −20° C.

Synthesis of PMLA/LLL/AP2/M4/rhodamine: One milligram of PMLA/LLL (40%)/MEA (10%) (260.7 g/mol , 0.00375 mmol) was dissolved in 300 μL of degassed phosphate buffer (pH 6.3) and was placed in a glass vial with a magnetic stirrer at ambient temperature. Then, 2.3% (0.0862 μmol) or 0.512 mg of Angiopep-2-PEG3400-MAL (5803 g/mol) peptide-PEG-MAL were added dissolved in phosphate buffer pH 6.3 at 10 mg/mL concentration. The reaction was monitored using HPLC, typically for 1 h. Then, 0.215 mg (21.5 μL) of Miniap-4-PEG2000-Mal (2796 g/mol) was added. The reaction mixture was monitored using HPLC (typically 1 h reaction time) and, once completed, the glass vial was covered with aluminum foil and rhodamine C2 was added (0.026 mg for 1% loading, 680.79 g/mol, 0.153 μmol, 13.05 μL of 2 mg/mL solution in DMF) and stirred for 1 h. Then, 15 μL of 3-(2-pyridyldithiopropionic acid (PDP: 10 mg/mL solution in DMF) was added to cap the free SH groups. The reaction was stirred for an additional hour before purification using a PD-10 column (H₂O as solvent), HPLC analysis and lyophilization.

Syntheses of PMLA/LLL/AP2/d-Peptide/Rhodamine Conjugates for Targeting Amyloid Peptides and Plaques Across BBB Involved in Alzheimer's Disease:

Syntheses included the following peptides (of D-amino acid sequences):

D1-peptide (QSHYRHISPAQVC (SEQ ID NO: 10)), all D-amino acids; D3-peptide (RPR TRL HTH RNRC (SEQ ID NO: 11)), all D-amino acids; and ACI-89 (PSHYRHISPAQKC (SEQ ID NO: 12)), all D-amino acids.

These peptides were described in van Groen et al. (2009) and Funke et al. (2012), which are incorporated herein by reference as if fully set forth. Protocols were the same for all the peptides, here described for the synthesis including D-1 peptide:

D1-Peptide Coupling with Mal-PEG-Mal 3400:

In a glass vial with magnetic stirrer (ambient temperature), Mal-PEG-Mal 3400 (3400 g/mol, 9.36 mg, 2.75*10⁻³ mmol, 1.05 eq) was dissolved in 936 μL of phosphate buffer 6.3. D1 peptide (1525.8 g/mol, 4 mg, 1 eq, 2.62*10⁻³ mmol) dissolved in 400 μL phosphate buffer 6.3 was added dropwise.

The reaction was monitored using HPLC and was placed in −20° C. once completed. A solution of 10 mg/mL product in phosphate buffer 6.3 is calculated for the next reaction.

Preparation of PMLA/LLL/Angiopep-2/D 1-PEG-Mal/Rhodamine:

3 mg of PMLA/LLL (40%)/MEA (10%) (260.7 g/mol monomer, 0.0115 mmol) were dissolved in 700 μL of phosphate buffer pH 6.3 and were placed in a glass vial with magnetic stirrer at ambient temperature. D1-Peg-Mal was added (4924.8 g/mol, 1.13 mg/2%, 2.3*10⁻³ mmol, 10 mg/mL solution in phosphate buffer 6.3). The reaction is monitored using HPLC, and is typically 1 h. Then, 1.0 eq of 2% (2.3*10⁻⁴ mmol) or 1.33 mg of angiopep-2-PEG-MAL 3400 (5802.7 g/mol) peptide-PEG-MAL were added dissolved in phosphate buffer pH 6.3 at a concentration of 10 mg/mL. The reaction was monitored with HPLC, and once completed (usually 1 h), the glass vial was covered with aluminum foil and Rhodamine C2 was added (0.0786 mg for 1% loading, 680.79 g/mol, 1.15*10⁻⁴ mmol, 39.3 μL of 2 mg/mL solution in DMF). Mixed view required to see dye absorbance in the PMLA peak. Typically, the reaction should be stirred for 1 h. Then, either 15 μL of 3-(2-pyridyldithiopropionic acid) (PDP, 10 mg/mL solution in DMF) or N-ethylmaleimide (10 mg, 125 g/mol, 0.08 mmol in 50 μL DMF) were added to cap the free SH groups. The reaction was stirred for an additional hour before purification using PD-10 column (eluted with water), HPLC analysis and lyophilization.

Preparation of PMLA/LLL/Miniap-4/D1-PEG-Mal/Rhodamine:

2 mg of PMLA/LLL (40%)/MEA (10%) (260.7 g/mol monomer, 0.0077 mmol) were dissolved in 800 μL of phosphate buffer pH 6.3 and were placed in a glass vial with magnetic stirrer at ambient temperature. 1.15 eq of 2% or 0.867 mg of D1-PEG-MAL 3400 (4924.8 g/mol) were added dissolved in phosphate buffer pH 6.3 to 10 mg/mL concentration. The reaction is monitored using HPLC, and is typically 1 h. Then, miniap-PEG-Mal was added (2796 g/mol, 0.43 mg 2%, 10 mg/mL solution in phosphate buffer 6.3). the reaction was monitored with HPLC. Once completed, the glass vial was covered with aluminum foil and Rhodamine C2 was added (0.0516 mg for 1% loading, 680.79 g/mol, 25.8 μL of 2 mg/mL solution in DMF) and reaction was monitored again using HPLC. Mixed view required to see dye absorbance in the PMLA peak. Typically, the reaction should be stirred for 1 h. Then, 15 μL of 3-(2-pyridyldithiopropionic acid) or PDP (10 mg/mL solution in DMF) was added to cap the free SH groups. The reaction was stirred for an additional hour before purification using PD-10 column, HPLC analysis and freeze drying.

All conjugates and pre-conjugates are kept at −20° C. D-peptides and AP2 loading were quantified using HPLC. Average amount of D-peptides loaded is 1.5%.

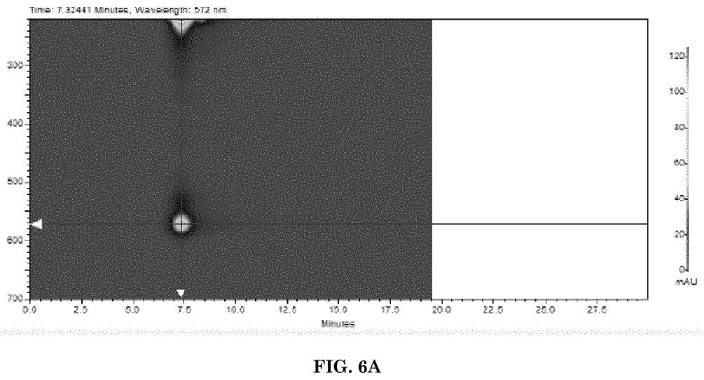

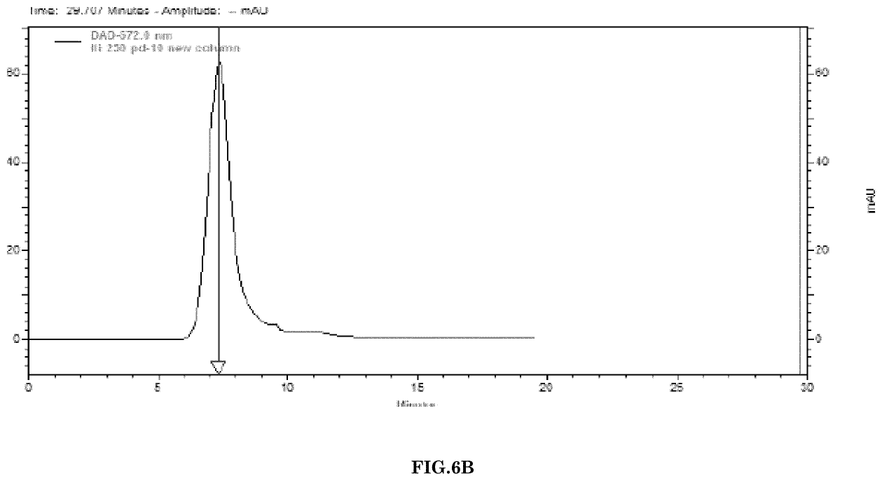

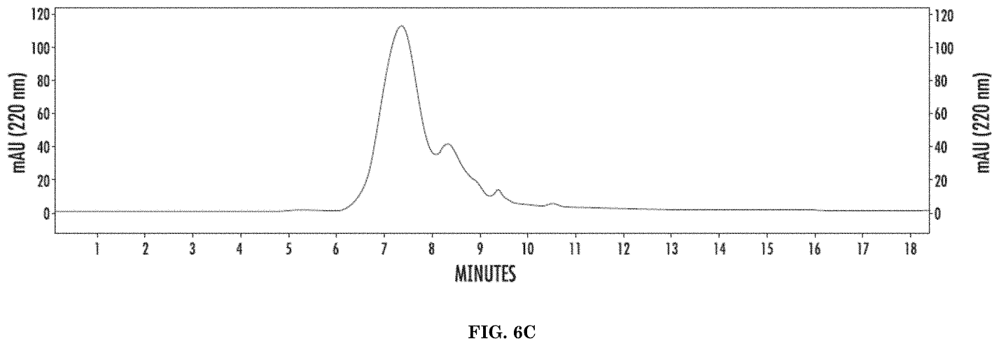

FIGS. 6A-6C illustrate characterization of synthesized P/LLL/AP-2/ACI-89/rhodamine FIG. 6A illustrates SEC-HPLC top view of scanning A200-A700 nm vs. retention time displaying absorbance of the complete nanoconjugate. FIG. 6B illustrates the scanning profile of the same conjugate as shown on FIG. 6A at 572 nm wavelength indicating the rhodamine is part of the physical entity. FIG. 6C illustrates the scanning profile of the same conjugate as shown on FIG. 6A at 220 nm wavelength indicating the P/LLL/AP-2/ACI-89 is part of the physical identity.

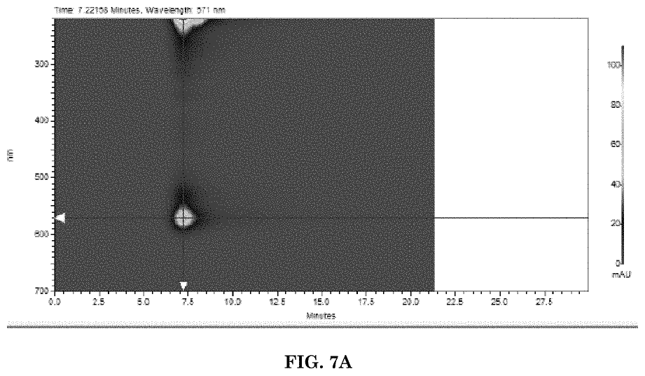

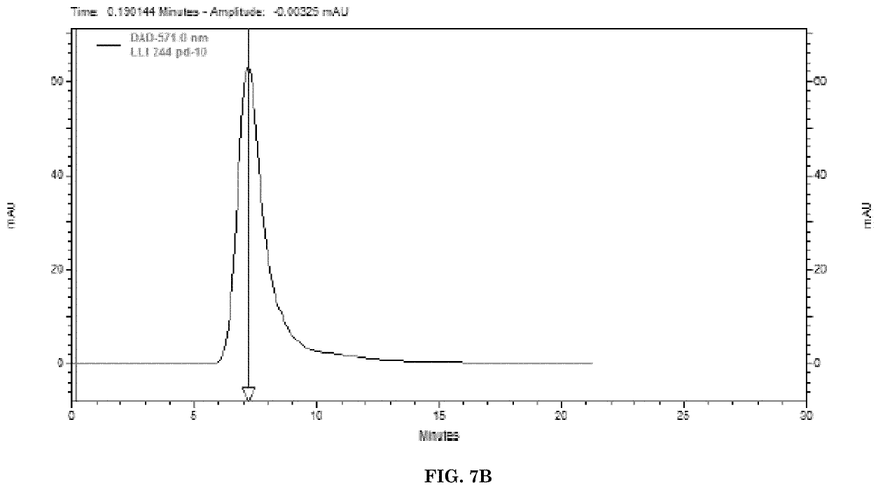

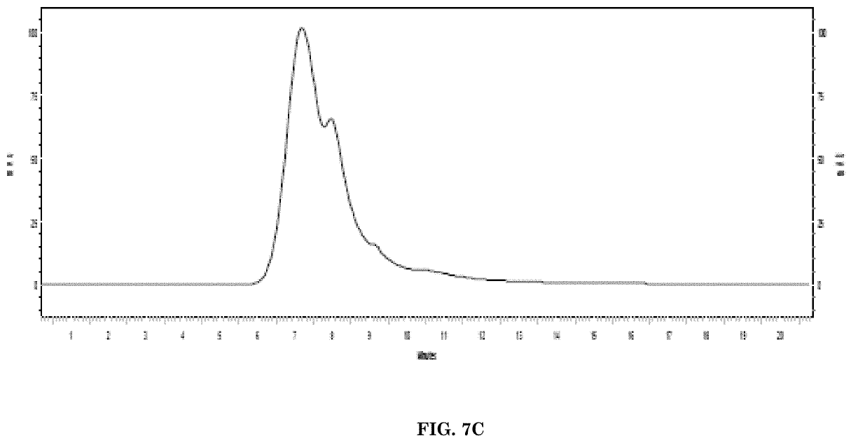

FIGS. 7A-7C illustrates SEC-HPLC chromatogram of P/LLL/AP-2/D1-peptide/rhodamine at A200-A700 nm vs. retention time displaying absorbance of PMLA/LLL/AP-2/D1-peptide/rhodamine complete nanoconjugate. FIG. 7B is a scanning profile of the same nanoconjugate as shown on FIG. 7A at 572 nm indicating the rhodamine component. FIG. 7C is a scanning profile of the same nanoconjugate as shown on FIG. 7A at 220 nm indicating the PMLA/LLL/AP-2/D 1-peptide component.

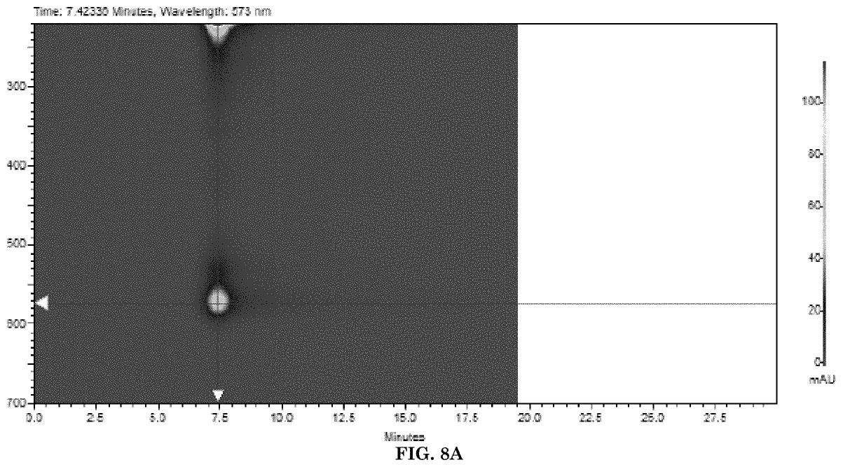

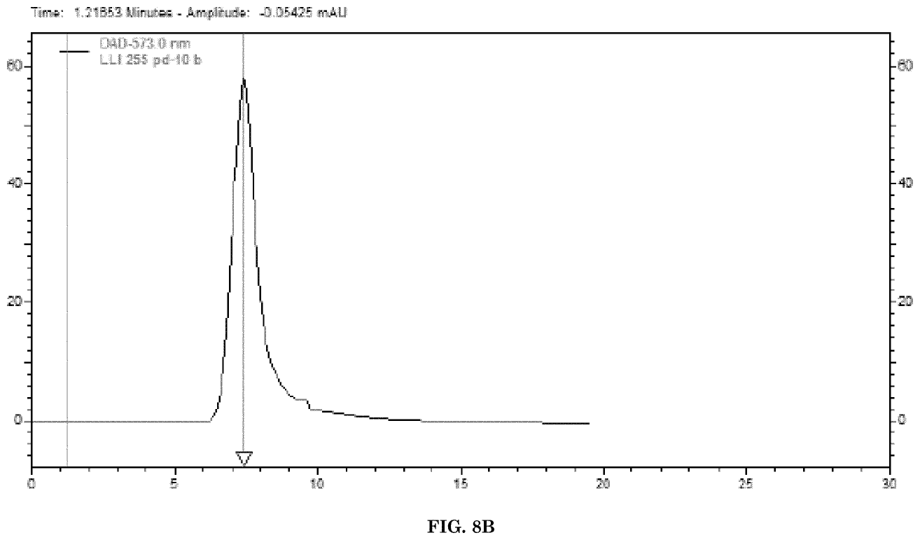

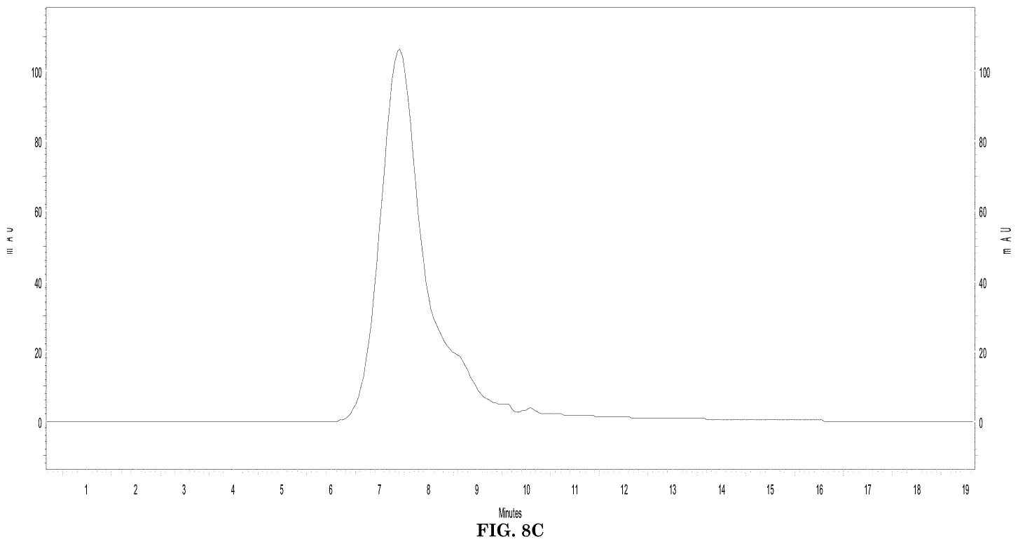

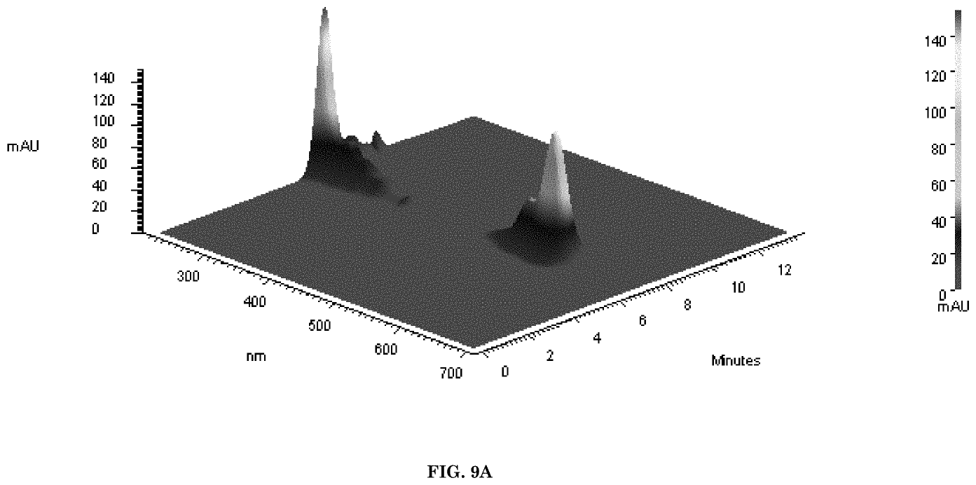

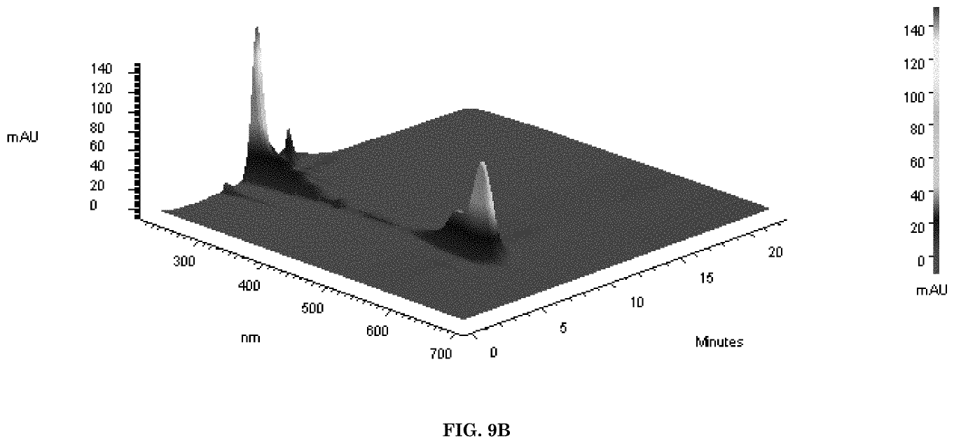

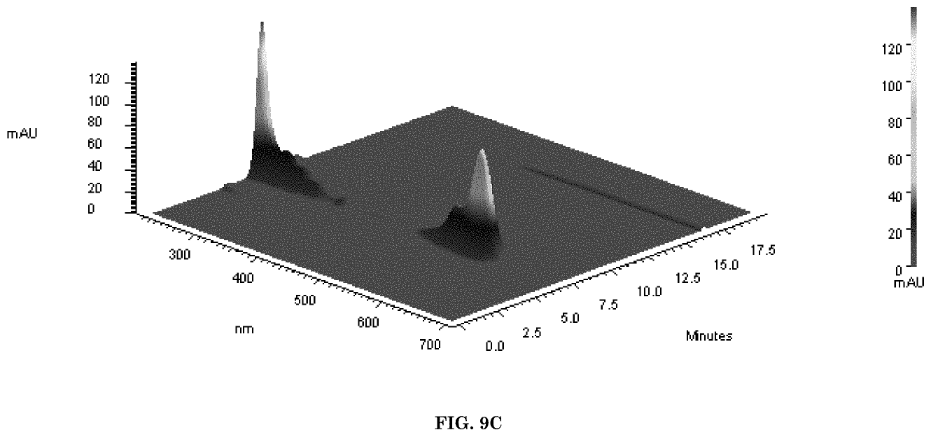

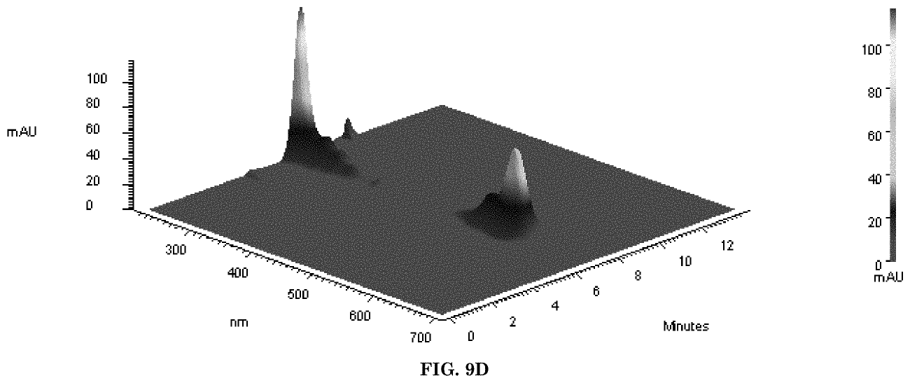

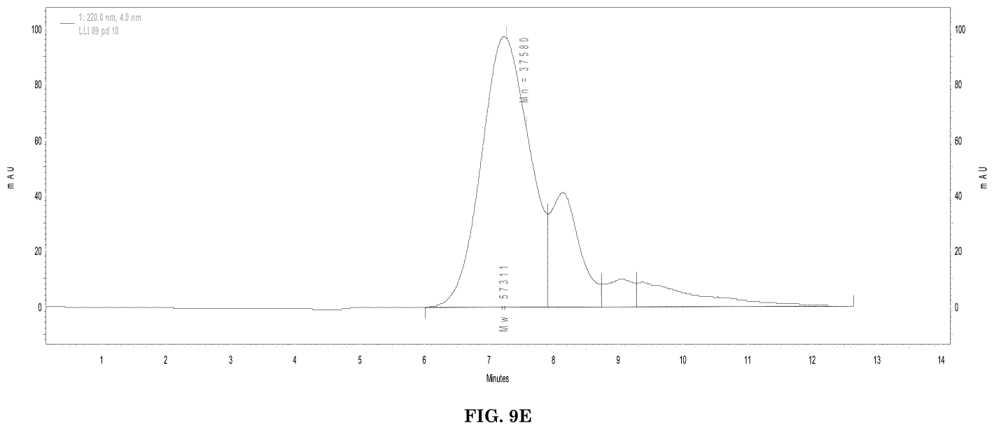

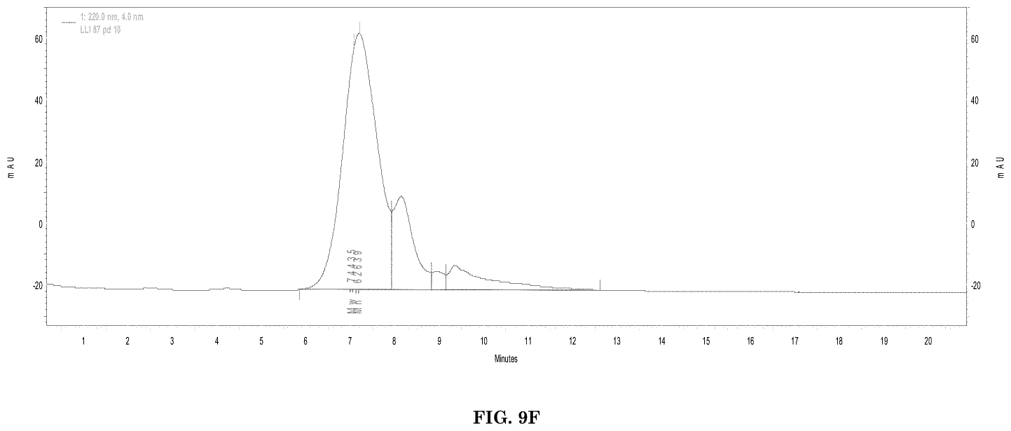

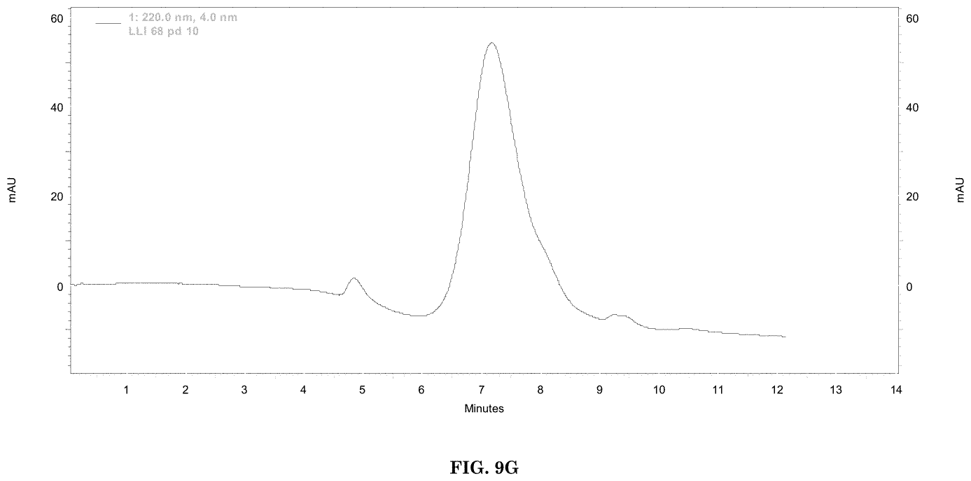

FIGS. 8A-8C illustrate characterization of synthesized P/LLL/AP-2/D3-peptide/rhodamine. FIG. 8A illustrates SEC-HPLC top view displaying A200-A700 nm vs. retention time and absorbance of the P/LLL/AP-2/D3-peptide/rhodamine complete nanoconjugate. FIG. 8B is the scanning profile of the same nanoconjugate as shown on FIG. 8A at 572 nm absorbance of rhodamine. FIG. 8C is the scanning profile of the nanoconjugate recorded at 220 nm wavelength for the P/LLL/AP-2/D3-peptide component. Examples of product verification by HPLC are illustrated on FIGS. 9A-9G.

The iv application of the nanoconjugates including D1, D3 or ACI-89 peptides follow the same protocol as for the nanoconjugates carrying the peptides P/LLL/AP2/Rh or similar.

AON Activation (Addition of SPDP Crosslinker)

28.85 mg of AON (white solid, 8200 g/mol) were dissolved in 0.6 mL of 1:1 v:v PBS:DMF (PBS: Gibco, 1×, pH 7.4,). When dissolved, the clear solution was colorless to light yellow (2-3 minutes with shaking). S-PDP, 10 eq (312 g/mol, 10.98 mg) was dissolved in 0.2 mL DMF and added to the AON. The reaction was shaken at RT for one hour at low speed.

After one hour, the reaction mixture was added dropwise (2 min) into 10 mL of acetone. A white precipitate shows immediately. The dispersion is then centrifuged at 20° C., 4000 rpm for 2-3 minutes. The white precipitation was set as a small pallet at the bottom of the tube. The acetone was removed, while the pallet was added with 10 mL of acetone. The pallet was re-dispersed in the acetone using a bath sonicator (5 min) and a vigorous shaking and vortexing. Following re-dispersion, the mixture was centrifuged again in the same conditions. Acetone was removed from the pellet, and the pellet was dried using air flush for 1-2 minutes or until all acetone is gone. The solid was than dissolved in 1.8 mL of water (milliQ purified), freeze dried, and stored in −20° C. until used. The product was injected to reverse phase HPLC to confirm the activation. In case of a carboxy-fluorescein labeled AON, a peak of the labeled product is also detected using the fluorescence detector (FLD, 19.8 min).

Synthesis of P/LLL (40%)/Peptide (2%)/AON/Rhodamine (1%)

6.6 milligrams of PMLA/LLL (40%)/MEA (10%) (260 g/mol, 24.6 μmol pre-conjugate monomer) were dissolved in 1.2 mL of phosphate buffer pH 6.3 and placed in a glass vial with a magnetic stirrer at ambient temperature. In order to achieve 2% loading, 2.61 mg D3-PEG3400-maleimide (1 eq, 5103 g/mol), or 2.8 mg D1-PEG3400-maleimide (1.1 eq, 4925 g/mol) were dissolved in phosphate buffer pH 6.3 to a 10 mg/mL concentration and were added dropwise. After 1 h, the reactions which were monitored using SEC-HPLC (220 nm) were completed. Rhodamine-maleimide (0.174 mg for 1% loading, 680.79 g/mol, 0.249 μmol, 87 μL of 2 mg/mL solution in DMF) was loaded forming thioethers with the PMLA platform at pendant MEA-SH. The reaction was conducted in the dark and was monitored using SEC-HPLC. Extend of the conjugation was determined via rhodamine absorbance in the PMLA conjugate elution peak. After stirring for a further 1-2 h, 92 μL of citric buffer pH 5.0 were added, followed by 11.1 mg of activated Ms-EGFR AON CTGAGGGTCGCATCTCTGACCG (SEQ ID NO: 13) (10 mg/ml in buffer 6.3, 1.1 mL, 5% of the PMLA malic acids) and after 30 min in RT the reaction mixture was kept at 4° C. overnight. A SEC-HPLC revealed a new absorption at the 260 nm area located at the nanoconjugate retention time which indicated AON attachment, while an absorbance in 570 nm indicated the presence of rhodamine. Then, 10 mg of N-ethylmaleimide in 50 μL of DMF were added to cap the free SH groups at RT. After stirring for an additional 30 minutes, the product was purified over a G-75 column using PBS to elute, analyzed, concentrated to the injected dose using Vivaspin centrifuge filter tubes (30 kDa cutoff, 50 mL), snapped froze and stored at −20° C. prior to use.

P/LLL (40%)/peptide (2%)/Fluorescein-AON conjugates were synthesized in the same manner, without the addition of rhodamine

Carboxy-Fluorescein Quantification of Labelled AON Conjugated to Mini Nanodrugs:

Prior to lyophilization, 20 μl sample of rhodamine labeled nanoconjugate was diluted with 380 μL PBS pH 7.4. Absorbance was scanned at wavelength 500 nm (Flexstation, Molecular Devices, Sunnyvale, Calif., USA). The dye concentration was calculated from A500 measurements using the molar absorbance coefficient 83000 M⁻¹cm⁻¹

Quantification of Conjugated AON Using Reverse Phase HPLC:

50 μL sample of the product after G-75 purification (prior to concentration) was added with 50 μL solution of DTT 1M. the sample was shaken at RT for one hour before HPLC analysis. Then 20 μL were injected to a reverse phase HPLC (Agilent 1260 infinity II, with diode array detector-DAD and fluorescence detector—FLD, with a XB-C-18 100A 100×4.60 mm column, Phenomenex) with a 2-60% ACN (0.1% TFA) gradient in H₂O (2% TFA). The AON retention time was 18.7 min at 260 nm, and in case of labeled AON, the peak was visible using the FLD (19.8 min) as well. The area under the curve (AUC) was then measured, and a calibration curve of AUC Vs. C made using known concentrations of activated AON was used for quantification.

Chemical activation of IgG antibodies: To the solution of mAb (5 mg, ˜33 nmol, Mw ˜150 kDa) dissolved in 1 mL of 2 mM EDTA in PBS to a concentration of 5 mg/mL was added a 50 mM solution in water of tris(2-carboxyethyl) phosphine hydrochloride (TCEP, to a final concentration of 5 mM). The mixture was gently shaken for 30 min at RT. TCEP was removed using Sephadex PD-10 (2 mM EDTA in PBS or 100 mM sodium phosphate buffer 5.5), and the reduced antibody was immediately added dropwise to maleimide-PEG3400-maleimide (25 mg, 1:5 ratio to Ab) dissolved in 200 μL of 2 mM EDTA in PBS. The reaction mixture was stirred at RT for 0.5 h and then concentrated using a centrifuge membrane filter (Vivaspin, cutoff 30 kDa, 20 mL) and purified over Sephadex G-75 pre-equilibrated with 100 mM sodium phosphate buffer pH 6.3. Pure fractions containing antibody were collected, and the Ab concentration was quantified using UV (280 nm, coefficient 1.55). The reaction yield was 70-85%.

Synthesis of PMLA/LLL/Peptide/IgG/Rhodamine Conjugate:

First, 1 mg of pre-conjugate (258.5 g/mol monomer, 3.9×10⁻³ mmol, PMLA/LLL/MEA) was dissolved in 200 μL of sodium phosphate buffer pH 6.3 and then added with 0.026 mg rhodamine-maleimide (680.79 g/mol, 2 mg/mL solution in DMF, 3.9×10⁻⁵ mmol). The reaction mixture was stirred for 1 hour at RT. Meanwhile, the collected activated Ab is concentrated using centrifuge membrane filter (Vivaspin, cutoff 30 kDa, 20 mL) and concentration was adjusted using 100 mM sodium phosphate buffer and 150 mM NaCl, pH 6.3 to 8-10 mg/mL. The Ab amount is calculated to be 0.2% of the malic acid monomers in the pre-conjugate (for PMLA/LLL/MEA each 1.16 mg of Ab should be reacted with 1 mg of pre-conjugate). The Ab is added into the reaction mixture of pre-conjugate and rhodamine and the reaction is monitored using SEC-HPLC (PBS). After 40 min, AP-2-PEG3400-maleimide (0.449 mg, 5803 g/mol, 7.7×10⁻⁵ mmol) or D3 (0.395 mg, 5103 g/mol, 7.7×10⁻⁵ mmol) or D1 (0.381 mg, 4923 g/mol, 7.7×10⁻⁵ mmol) or M4 (0.216 mg, 2796 g/mol, 7.7×10⁻⁵ mmol) or B6 (0.345 mg, 4480 g/mol, 7.7×10⁻⁵ mmol) (each 2 mol % with regard to malic acid in the pre-conjugate and peptide-PEG-maleimide in a solution of 10 mg/mL buffer 6.3) is added and allowed to react at RT. After monitored by HPLC, the mixture is transferred after 30 min to 4° C. overnight. A large access of N-ethylmaleimide is added to cap the remaining SH groups, and the mixture is stirred at RT for 20-30 min. After purification over G-75 column, equilibrated with PBS, the product is concentrated if needed using filter centrifugation.

Rhodamine Labelling of IgG Ab:

To the solution of mAb (3.7 mg, ˜24 nmol, Mw 155 kDa) dissolved in 0.41 mL of 2 mM EDTA in PBS was added a 50 mM solution in water of tris(2-carboxyethyl) phosphine hydrochloride (TCEP, to a final concentration of 5 mM). The mixture was gently shaken for 30 min at RT. Excess TCEP was removed using Sephadex PD-10 (2 mM EDTA in PBS or 100 mM sodium phosphate buffer 5.5), and the reduced antibody was immediately combined with 18 μL of rhodamine-maleimide (2 mg/mL in DMF). The mixture was shaken for 1 minute and then added dropwise to maleimide-PEG3400-maleimide (18.5 mg, 1:5 ratio to Ab) dissolved in 200 μL of 2 mM EDTA in PBS. Stirred at RT for 0. concentrated using Vivaspin, cutoff 30 kDa, 20 mL, and purified over Sephadex G-75 pre-equilibrated with 100 mM sodium phosphate buffer pH 6.3. Pure fractions containing antibody were collected, and the Ab concentration was quantified using UV (280 nm, coefficient 1.55). Rhodamine labeling was confirmed using SEC-HPLA and UV (abs. at 570 nm). The reaction yield was 70-85%. The molecular weight by SEC-HPLC has been published

Synthesis of PMLA/LLL/AP2/IgG-Rhodamine Conjugate:

Synthesis was carried out analogous to the synthesis of PMLA/LLL/peptide/IgG/rhodamine conjugate using the rhodamine-labeled Ab.

Zeta Potential Measurements: Synthesized conjugates were characterized with respect to their ζ potential using a Zetasizer Nano ZS90 (Malvern Instruments, Malvern, UK). Ten microliter aliquots of nanoconjugate samples were diluted in 0.99 mL PBS, and the voltage applied was 150 mV. Data represent the mean of three measurements±their standard deviation.

For mini nanodrugs containing IgG, ζ potential was measured at voltage of 6.92 mV.

Dynamic Light Scattering: Synthesized conjugates were characterized using a Zetasizer Nano ZS90 (Malvern Instruments, Malvern, UK). Ten microliter aliquots of nanoconjugate samples were diluted in 0.99 mL PBS and were measured 3 times using a clear cuvette. Data represent the mean of three measurements and polydispersity index.

Chemical characterization: Copolymers were subjected to hydrolytic cleavage in sealed ampoules containing 2 M HCl for 12 h at 100° C. Malic acid in the hydrolysate was quantified by a colorimetric method based on an enzymatic reaction using malate dehydrogenase (Rozemaet al. (2003) Bioconjugate Chemistry, 14, 51-57, which is incorporated herein by reference as if fully set forth).

FTIR measurements: A dry sample of the materials tested was added to KBr powder and scanned using a Bruker Alpha instrument with a DRIFT module (Bruker, Billerica, Mass., USA). KBr alone was used for the background scan.

3D images and energy calculations: Calculations followed Chem3D Pro 11.0 (CambridgeSoft, Wellesley, Mass., USA).

Rhodamine (rh) quantification of the final nanoconjugates: Prior to lyophilization, 10 μL sample of rhodamine labeled nanoconjugate was diluted with 990 μL PBS pH 7.4. Absorbance was scanned at wavelength 570 nm (Flexstation, Molecular Devices, Sunnyvale, Calif., USA). The dye concentration was calculated from A570 measurements using the molar absorbance coefficient 119000 M⁻¹cm⁻¹. In addition, fluorescence scans (excitation 570 nm/emission 600 nm, cutoff 590 nm) confirmed the presence of rhodamine in the samples.

Synthesis and Characterization of Nanoconjugates

PMLA-based nanoconjugates were synthesized as candidates for trans-BBB drug delivery as shown in Table 1.

TABLE 1
Nomenclature of nanoconjugates, functional components,
analytical properties (ξ potential, SEC-
HPLC retention time (rt) and molecular mass)

| | | ζ | SEC- | Calculated |
|---|---|---|---|---|
| | | po- | HPLC | molecular |
| | | tential | rt | mass |
| Nanoconjugate | Components | [mV] | [min] | [g/mol] |
| P/LLL/AP2^{a} | PMLA/LLL/AP2/ | −11.6 | 7.215/ | 164000/ |
| | rhodamine | | 7.22 | 165000 |
| P/AP2 | PMLA/AP2/ | −11.5 | NA | 108000 |
| | rhodamine | | | |
| P/LLL/AP2-1 | PMLA/LLL/AP2(1%)/ | −2.5 | 7.52 | 139,000 |
| | rhodamine | | | |
| P/LLL | PMLA/LLL/ | −16.5 | NA | 115000 |
| | rhodamine | | | |
| P/LLL/AP7^{b} | PMLA/LLL/AP7/ | −5.48 | NA | 166000 |
| | rhodamine | | | |
| P/Rh | PMLA/rhodamine | −22.9 | NA | 52000 |
| P/LLL/M4^{c} | PMLA/LLL/M4/ | −10.4 | NA | 139000 |
| | rhodamine | | | |
| P/LLL/cTfRL^{d} | PMLA/LLL/cTfRL/ | −9.58 | NA | 139000 |
| | rhodamine | | | |
| P/M4 | PMLA/M4/ | −14.6 | NA | 82000 |
| | rhodamine | | | |
| P/cTfRL | PMLA/cTfRL/ | −15.2 | NA | 82000 |
| | rhodamine | | | |
| P/LLL/AP2/M4 | PMLA/LLL/AP2/ | −5.5 | NA | 189000 |
| | Miniap4/rhodamine | | | |
| P/LLL/B6^{e} | PMLA/LLL/B6/ | −6.1 | NA | 158000 |
| | rhodamine | | | |
| P/LLL/AP2/B6 | PMLA/LLL/ | −6.2 | 7.36 | 159,000 |
| | AP2(1%)/B6(1%)/ | | | |
| | rhodamine | | | |
| P/LLL/ | PMLA/LLL/ | −2.2 | NA | 222000 |
| AP2 (4%) | AP2(4%)/rhodamine | | | |
| P/LLL/D1^{f} | PMLA/LLL/D1/ | −3.06/ | 7.20 | 154,000 |
| | rhodamine | −3.1 | | |
| P/LLL/D3^{g} | PMLA/LLL/D3/ | −7.3/ | 7.20 | 155,000 |
| | rhodamine | −7.34 | | |
| P/LLL/ACI89^{h} | PMLA/LLL/ACI89/ | −13.8 | 7.15 | 154,000 |
| | rhodamine | | | |
| P/LLL/B6 | PMLA/LLL/B6/ | −6.1 | 7.50 | 153,000 |
| | rhodamine | | | |
| P/LLL/B6-1 | PMLA/LLL/B6(1%)/ | −10.8 | 7.52 | 160,000 |
| | rhodamine | | | |
| P/LLL/M4 | PMLA/LLL/M4/ | −10.4 | 7.20 | 138,000 |
| | rhodamine | | | |
| P/LLL/AP2/ | PMLA/LLL/AP2/ | −7.4 | 6.60 | 294,000 |
| IgG | IgG(0.2%)/rhodamine | | | |
| P/LLL/B6/IgG | PMLA/LLL/B6/ | −7.9 | 6.55 | 282,000 |
| | IgG(0.2%)/rhodamine | | | |
| P/LLL/D1/IgG | PMLA/LLL/D1/ | −7.8 | 6.55 | 279,000 |
| | IgG(0.2%)/rhodamine | | | |
| P/LLL/M4/IgG | PMLA/LLL/M4/ | −6.1 | 6.43 | 269,000 |
| | IgG(0.2%)/rhodamine | | | |
| P/LLL/D3/IgG | PMLA/LLL/D3(2%) | −6.5 | 6.46 | 281,000 |
| | IgG(0.2%)/rhodamine | | | |
| P/LLL/IgG | PMLA/LLL/IgG(0.2%)/ | −10.4 | 6.69 | 244,000 |
| | rhodamine | | | |
| P/LLL/AP2/D1 | PMLA/LLL/AP2(2%)/ | −7.44 | 7.05 | 207000 |
| | D1(2%)/rhodamine | | | |
| P/LLL/D1/ | PMLA/LLL/D1/ | −6.98 | 7.45 | 190000 |
| AON-F | AON-Fluorescein(1%) | | | |
| P/LLL/D3/ | PMLA/LLL/D3/ | −7.31 | 7.64 | 194000 |
| AON | AON(1%)/rhodamine | | | |

Peptide sequences: ^{a} TFFYGGSRGKRNNFKTEEY (SEQ ID NO: 15), or TFFYGGSRGKRNNFKTEEYC-NH₂ (SEQ ID NO: 1); ^{b} TFFYGGSRGRRNNFREEYCNH₂ (SEQ ID NO: 7); ^{c} H-[Dap] KAPETAL D-NH2 (SEQ ID NO: 3), cyclic; ^{d} CRTIGPSVC-NH2 (SEQ ID NO: 2), cyclic, S—S bonded; ^{e} CGHKAKGPRK (SEQ ID NO: 8), ^{f} QSHYRHISPAQVC (SEQ ID NO: 9). ^{g} RPRTRLHTHRNRC (SEQ ID NO: 10); and ^{h} PSHYRHISPA QKC (SEQ ID NO: 11).

Unless mentioned otherwise, all PMLA conjugates contained 1% rhodamine within 100% of total pendant PMLA carboxylic groups. Tri-leucine (LLL) was conjugated with 40% of the pendant carboxylates of the PMLA backbone via DDC/NHS chemistry in eight of the nanoconjugates as shown in FIGS. 8A-8D.

Peptide moieties consisting of either B6, AP-2, AP-7, M4, cTfRL, D1, D3 and AC189 were conjugated to the polymer via a maleimide-thiol bond, and either a PEG3400 or PEG2000 linker was used to allow flexible peptide interactions with biological targets. M4 and cTfRL peptides were attached to the PEG linker via their N-terminus since these small cyclic peptides did not contain a terminal cysteine (unlike AP-2, AP-7 and B6). Each of the peptides was conjugated a stoichiometry of 2% of total pendant carboxylates unless indicated otherwise. For one conjugate, a 4% load of AP2 was added to the PMLA backbone (Table 1). The cyclic peptide M4 was attached to a PEG linker (maleimide-PEG2000-succinimidyl carboxymethyl ester (maleimide-PEG2000-SCM) via the N-terminus. The free maleimide remaining on the linker was then conjugated with SH-groups on the pre-conjugate at a loading of 2% of malic acid residues using phosphate buffer pH 6.3. Rhodamine-maleimide was then used to label the conjugate (1% of the malic acid residues). The remaining thiol groups were capped by N-ethylmaleimide to prevent side reactions and aggregation. A schematic structure of the products is shown in FIGS. 5A-5F, while the chemical nomenclature, calculated molecular weight, ζ potential, and SEC-HPLC retention are listed in Table 1. SEC-HPLC diode array detector (DAD) profiles showed the nanoconjugate absorbance at 220 nm and the rhodamine absorbance at 570 nm at the same retention time, and thus confirming the rhodamine labeling.

For the AON delivery testing, the following conjugates were synthesized: P/LLL/peptide/AON-carboxy-fluorescein (or P/LLL/peptide/AON-F, and P/LLL/peptide/AON/rh. The schematic structures of P/LLL/peptide/AON-F and P/LLL/peptide/AON/rh are illustrated on FIGS. 5C and 5D, respectively.

AONs (Ms-EGFR; SEQ ID NO: 13) were purchased with a primary amine 5′ terminal, which was reacted with 3-(2-Pyridyldithio)propionic acid N-hydroxysuccinimide ester (S-PDP). Four molar equivalents of the activated AON were added to the reaction mixture following the Maleimide-PEG₃₄₀₀-D peptide in case of a labelled AON or following rhodamine-Maleimide in case of a non-labeled AON. After purification, the loading of the AON on the final product was quantified using reverse-phase HPLC. First, in a 100 μL sample, dithiotheritol (DTT) was used to reduce the di-sulfide bond between the AON and PMLA backbend and thus release the AON to the solution. The AON was then injected to a reverse phase C-18 HPLC and the area under the curve was measured. The final amount was calculated using a pre-made calibration curve in the same conditions. In addition, when the fluorescein labeled AON was used, a fluorimeter was also used (in a similar manner to the rhodamine labeled nanoconjugates) to confirm HPLC results. Generally, AON loading was measured to be in the range of 1-1.5% of the PMLA malic acid residues.

Nanoconjugates were also modified with an antibody as shown on FIGS. 5E-5F. A non-specific IgG2a Ab was dissolved in PBS pH 7.4, 2 mM in EDTA, and centrifuged using 2× Vivaspin membrane centrifugation to removes stabilizers and excipients. The quantity of Ab was measured using UV, before 1-2 disulfide groups were reduced with tris(2-carboxyethyl) phosphine hydrochloride (TCEP). The clean reduced Ab was single-arm conjugated with maleimide-PEG3400-maleimide. During stepwise addition, the remaining arm of the linker was thiolated with the pre-conjugate-SH in sodium phosphate buffer pH 6.3. After HPLC monitoring (typically 20 min), the selected shuttle peptide-linker-maleimide and rhodamine-maleimide were thiolated with preconjugate-SH. The reaction was maintained at 4° C. overnight before excess thiols were capped with N-ethylmaleimide. The product was purified over G-75 column equilibrated with PBS (Patil et al., 2015, ACS Nano, 9 (5), 5594-5608, which is incorporated herein by reference as if fully set forth). Chemical nomenclature of the mini nanodrugs that carry antibody, SEC-HPLC retention times, calculated molecular weight and ζ potential are listed in Table 1. The negative ζ potential of the mini nanodrugs fall into groups 3 and 4 ranging from −5.5 mV to −11.6 mV previously identified as nanoconjugates most suitable for crossing BBB.

For the syntheses of all conjugates, PMLA pendant carboxylates were activated by the DCC/NHS method to attach LLL and 2-mercapto ethylamine (MEA) (Ding et al. (2010); and Patil et al. (2015), both of which are incorporated as if fully set forth). MEA was then used to form thioethers with peptide-PEG-maleimide and rhodamine-maleimide. The conjugates were characterized by their calculated molecular mass as shown in Table 1, malic acid content, FTIR analysis, SEC-HPLC elution profile and ζ potential.

Conjugates without attached rhodamine were also characterized by the hydrodynamic diameter using dynamic light scattering (DLS) as shown in Table 2.

TABLE 2
Hydrodynamic diameter and PDI for selected
nanoconjugates measured by DLS.

| | Hydrodynamic diameter | |
|---|---|---|
| | (PDI) (volume mode) | |
| Nanomolecule | [nm] | |
| P/LLL/AP2 | 4.45 | (0.39) |
| P/AP2 | 5.93 | (0.79) |
| P/LLL | 2.68 | (0.50) |
| PMLA | 3.68 | 0.89) |

Examples of product verification by HPLC are illustrated on FIGS. 9A-9G. FIG. 9A illustrates verification of PMLA/LLL/angiopep-2-PEG3400-MAL/rhodamine. FIG. 9B illustrates verification of PMLA/LLL/“Fe mimetic peptide” CRTIGPSVC (SEQ ID NO: 2) (cyclic)-peptide-PEG2000-Mal/rhodamine. FIG. 9C illustrates verification PMLA/LLL/Miniap-4-PEG2000-Mal/cy 5.5. FIG. 9D illustrates control: PMLA/LLL/rhodamine. FIG. 9E illustrates PMLA/LLL/angiopep-2 (2%)/“Fe Mimetic Peptide” (2%)/rhodamine (1%) dipeptide for targeting. FIG. 9F illustrates PMLA/LLL/angiopep-2 (2%)/miniap-4 (2%)/rhodamine (1%) dipeptide for targeting. FIG. 9G illustrates PMLA/LLL/miniap-4 (2%)/angiopep-2 (2%)/“Fe mimetic Peptide” (2%)/rhodamine (1%) tripeptide for targeting.

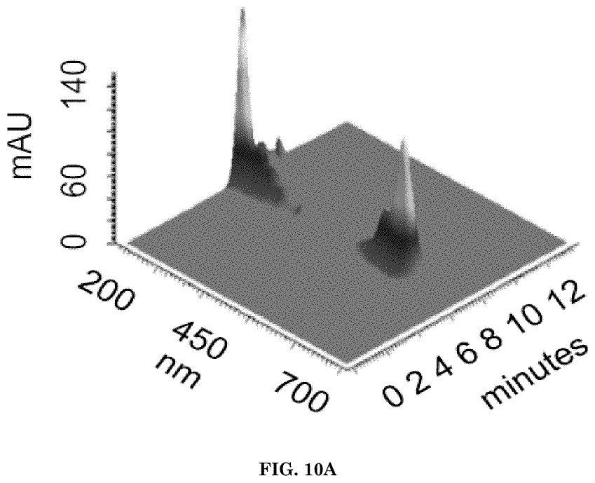

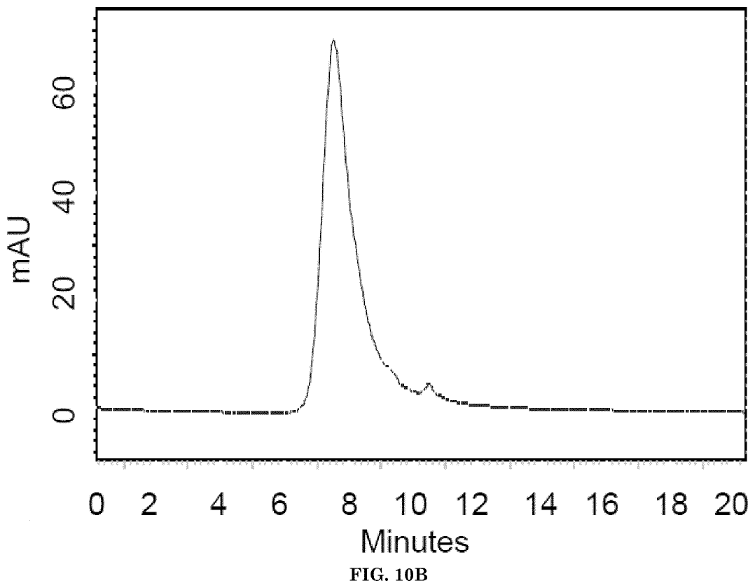

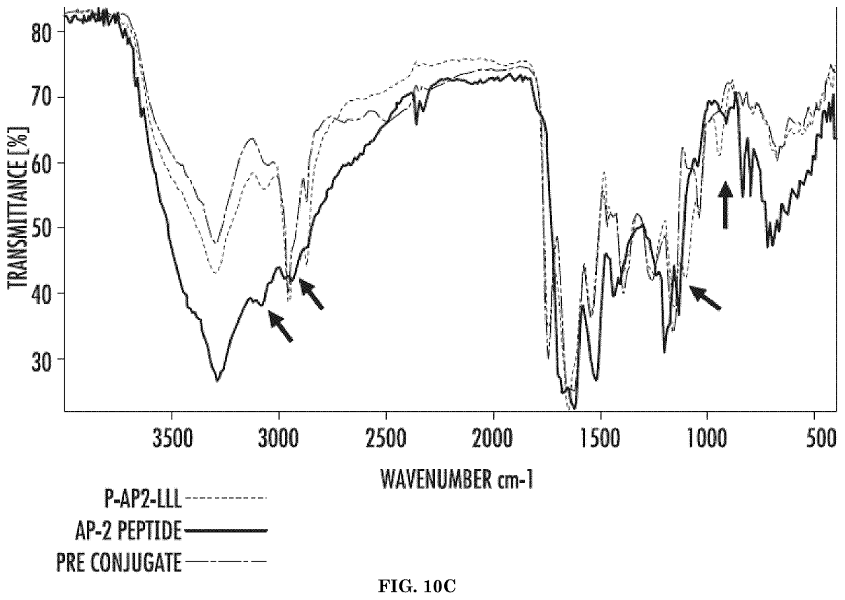

FIGS. 10A-10C illustrate characterization of synthesized P/LLL/AP2. FIG. 10A illustrates SEC-HPLC 3D view of A200-A700 nm vs. retention time and absorbance of all the P/LLL/AP2 nanoconjugate constituents. FIG. 10B illustrates SEC-HPLC chromatogram of P/LLL/AP2 recorded at 220 nm wavelength. FIG. 10C illustrates FTIR spectrum of P/LLL/AP2 nanoconjugate (dashed line), AP2 free peptide (solid lined) and pre-conjugate (dashed-dotted line). Arrows in FIG. 10C indicate peak shifts in the P/LLL/AP2 conjugate in the absence of rhodamine labeling, compared with AP2 peptide and preconjugate.

Referring to FIG. 10C, the FTIR spectrum of P/LLL/AP2 contains several distinctive peaks that can be attributed to both the pre-conjugate and the pristine AP2 peptide, while some peaks were shifted or decreased in intensity. A prominent peak shift is visible from 3050 cm⁻¹ in the pre-conjugate spectrum to 3057 cm^{—1} in the P/LLL/AP2 spectrum as well as other changes in peaks at the lower frequencies of 1040, 1104 and 950 cm⁻¹. The analytic data illustrated in FIG. 10A and FIG. 10B, and especially for material absorbing at wavelength 577 nm in the sec-HPLC eluant indicated that the conjugation of rhodamine (FIG. 10A) and AP2-PEG-Mal with the polymer platform was successful. Referring to FIGS. 10A-10B, these data, the HPLC elution profile (FIG. 10B), and the presence of absorption at 577 nm wavelength indicating the excitation wavelength of rhodamine (FIG. 10A), demonstrate that the conjugation of the rhodamine dye and AP2-PEG-Mal with the polymer platform was successful. In addition, the content of malic acid in P/LLL/AP2 agreed with the 85% of malic acid yield reported for synthesized PMLA conjugates (Ding et al. (2011), which is incorporated herein by reference as if fully set forth).

To ensure that the different conjugate moieties do not have effects on the rhodamine signal, i.e. via electrochemical and electrostatic forces, the fluorescence emission of the nanoconjugates P/LLL/AP2, P/AP2, P/LLL/cTfRL and P/LLL/AP7 was measured in solution. 20-30% higher fluorescence intensity was observed for the LLL-containing nanoconjugates in comparison with P/AP2. It was assumed that this effect reflected the hydrophobicity of LLL side chains, but this was ruled out to affect the outcome of fluorescence measurements in brain tissues.

SEC-HPLC Analysis Data (LLL Present):

PMLA/LLL (40%)/AP2 (2%)/rhodamine (1%) or P/LLL/AP2: retention time (rt)=7.215; PMLA/LLL (40%)/rhodamine (1%) or P/LLL: rt=7.1; PMLA/LLL (40%)/AP 7 (2%)/rhodamine (1%) or P/LLL/AP 7: rt=7.27; PMLA/LLL (40%)/Miniap-4 (2%)/rhodamine (1%) or P/LLL/M4: rt=7.2; PMLA/LLL (40%)/cTfRL (2%)/rhodamine (1%) or P/LLL/cTfRL: rt=7.22; PMLA/LLL (40%)/AP-2 (2%)/rhodamine (1%) or P/LLL/AP2/M4: rt=7.05; PMLA/LLL (40%)/B6 (2%)/rhodamine (1%) or P/LLL/B6: rt=7.5. The ζ-potentials and calculated molecular mass for each of these conjugates are listed in Table 1.

SEC-HPLC analysis data (LLL absent): PMLA/rhodamine (1%) or P/Rh: rt=7.23; PMLA/AP2 (2%)/rhodamine (1%) or P/AP2: rt=7.18; PMLA/Miniap-4 (2%)/rhodamine (1%) or P/LLL: rt=7.1; PMLA/cTfRL (2%)/rhodamine (1%) or P/cTfRL: rt=7.05. The ζ-potentials and calculated molecular mass for each of these conjugates are listed in Table 1. The SEC-HPLC analysis of all conjugates above was performed using a Hitachi L-2130 pump with a Hitachi L-2455 detector with EZChrome Software. The column that was used was a Polysep 4000, and the flow rate lml/min; the buffer was PBS (pH 7.4).

Amylo-Glo Staining:

Slides were air dries and fixed using freshly diluted 2% PFA for 5 minutes. The PFA was washed 3 timed with PBS, and brains were covered with 70% EtOH for 5 minutes before washing with dH2O for 2 minutes. 1× Amylo-Glo (Biosenses, #TR-300-AG 100×, diluted with PBS) was then added for 10 minutes. The slides were then washed with PBS (5 minutes) and dH2O (15 seconds) and mounted (Fluoromount-G, No DAPI, Invitrogen), and stored at 4° C. prior to imaging.

GFAP Staining:

Slides were air-dried and fixed using freshly diluted 2% PFA for 5 minutes. The PFA was washed 3 timed with PBS, and brains blocked for 2 h using a blocking buffer (0.25 mL of triton 10%, 0.2 mL DMSO, 2.5 mL BSA 10%, 0.2 mL Goat serum and 6.85 mL PBS). Then, primary Ab (GFAP, host-chicken, Neuromics #CH23011, diluted in blocking buffer 1:500) and the slides were kept at 4° C. overnight. The slides were then washed with blocking buffer (5 times) and the secondary Ab was added (Goat anti chicken labelled with Alexa 488, Jackson Immuno, diluted 1:750) for 2.5 h. following washing with PBS (7 times) the slides were mounted (Fluoromount-G, Invitrogen), and stored at 4° C. prior to imaging.

Animal Procedures:

Healthy Mice: Eight to nine-week-old BALB/C and BLACK-6 mice were obtained from Charles River Laboratories (Wilmington, Mass., USA). Mouse maintenance and experimental procedures followed the guidelines established by the Cedars Sinai Institutional Animal Care and Use Committee (IACUC Protocol #7416). Three to four mice of each sex were used for each experiment. A total of 126 mice were used to produce the data shown in this publication.

Nanoconjugates were dissolved freshly in PBS (pH 7.4) prior to each experiment and injected intravenously (i.v.) into the lateral tail vein. Mice were anesthetized with isoflurane beforehand, and their tails were briefly warmed to facilitate injections. All conjugates were administered as a single dose, at final concentrations ranging from 0.068 to 0.548 μmol of total nanoconjugate per kg body weight, or as indicated for each experiment. The drug injection volume was kept constant at 150 μL. After each injection, mice were promptly returned to their cages. Eight to nine-week-old B116 mice were obtained from Charles River Laboratories (Wilmington, Mass., USA). Mouse maintenance and experimental procedures followed the guidelines established by the Cedars Sinai Institutional Animal Care and Use Committee (IACUC Protocol #7416). Three to four mice of each sex were used for each experiment. Nanoconjugates were dissolved freshly in PBS (pH 7.4) prior to each experiment and injected intravenously (i.v.) into the lateral tail vein. 15 min before euthanasia mice were injected with tomato lectin to visualize the blood vessels. All mice were euthanized 2 h post drug injection. Mice were anesthetized with isoflurane beforehand and their tails were briefly warmed to facilitate injections. All conjugates were administered as a single dose, as indicated for each experiment (4× or 8×). The drug injection volume was kept constant at 150 μL. After each injection, mice were promptly returned to their cages.

AD-Mouse models: 6-8 month old 5-FAD (B6.Cg-Tg (APPswe/PS1ΔE9) 85Dbo/J hemizygous) mice were obtained from Jackson Laboratories. Mouse maintenance and experimental procedures followed the guidelines established by the Cedars Sinai Institutional Animal Care and Use Committee (IACUC Protocol #7416). Three to four mice of each sex were used for each experiment. Nanoconjugates were dissolved freshly in PBS (pH 7.4) prior to each experiment and injected intravenously (i.v.) into the lateral tail vein. 15 min before euthanasia mice were injected with tomato lectin to visualize the blood vessels. Mice were anesthetized with isoflurane beforehand and their tails were briefly warmed to facilitate injections. The drug injection volume was kept constant at 150 μL. After each injection, mice were promptly returned to their cages. Brains were collected and preserved in OTC for immunostaining.

P/LLL/D3/rh nanoconjugate was injected at the doses of 2× (0.137 μmol/Kg), 4× (0.274 μmol/Kg), 6× (0.411 μmol/Kg) and 8× (0.55 μmol/Kg), while all other nanoconjugates were injected at a dose of 8× (0.55 μmol/Kd) only. The brains were harvested and a 14 μm thick slices which were fixed and stained with DAPI to envision the nuclei

6-8 month old 5-FAD (B6.Cg-Tg (APPswe/PS1ΔE9) 85Dbo/J hemizygous) mice were obtained from Jackson Laboratories (check). Mouse maintenance and experimental procedures followed the guidelines established by the Cedars Sinai Institutional Animal Care and Use Committee (IACUC Protocol #7416). Three to four mice of each sex were used for each experiment. Nanoconjugates were dissolved freshly in PBS (pH 7.4) prior to each experiment and injected intravenously (i.v.) into the lateral tail vein. 15 min before euthanasia mice were injected with tomato lectin to visualize the blood vessels. Mice were anesthetized with isoflurane beforehand and their tails were briefly warmed to facilitate injections. The drug injection volume was kept constant at 150 μL. After each injection, mice were promptly returned to their cages. Brains were collected and preserved in OTC for immunostaining.

P/LLL/D3/rh nanoconjugate was injected at the doses of 2× (0.137 μmol/Kg), 4× (0.274 μmol/Kg), 6× (0.411 μmol/Kg) and 8× (0.55 μmol/Kg), while all other nanoconjugates were injected at a dose of 8× (0.55 μmol/Kg) only. The brains were harvested and a 14 μm thick slices which were fixed and stained with DAPI to envision the nuclei

Brain tumor mouse models of intracranial glioblastoma: Mouse glioblastoma cell line GL261 was a gift from Dr. Bathes lab (UC San Diego, San Diego, Calif.) and was cultured in Dulbecco's modified Eagle medium (DMEM) (ATCC, Manassas, Va.) containing 10% fetal bovine serum with 1% penicillin (10 μg/mL), streptomycin (10 μg/mL), and amphotericin B (0.25 μg/mL) at 37° C. with 5% CO₂. All animal experiments were performed according to the guidelines of the Institutional Animal Care and Use Committee (IACUC) at Cedars-Sinai Medical Center (Los Angeles, Calif., USA).

Twenty thousand GL261 cells in a volume of 2 μL, were implanted intracranially into the right basal ganglia in 45 female 8 weeks old C57BL/6J mice (from Jackson Laboratory, Sacramento, Calif.). 3 mice per nanoconjugate treatment group were randomized into 15 groups and were injected once intravenously in 21 days after cell implantation, with either PBS, PMLA/LLL/Rh (1%), PMLA/LLL/Rh (1%), PMLA/AP2 (2%)/LLL/Rh (1%), PMLA/LLL/AP2 (2%)/Rh (1%), PMLA/AP2 (1%)/Rh (1%), PMLA/LLL/AP2 (1%)/B6 (1%)Rh (1%), PMLA/B6 (1%)/Rh (1%), PMLA/LLL/B6 (2%)/Rh (1%), PMLA/LLL/D 1 (2%)/Rh (1%), PMLA/LLL/ACI89 (2%)/Rh (1%), PMLA/LLL/D 3 (2%)/Rh (1%), PMLA/LLL/amsTfRAb (0.2%)/R (1%) or PMLA/LLL/Miniap4 (2%)/Rh (1%). The nanoconjugates were injected at a dose of 0.067 μmol/Kg-0 .274 μmol/Kg.

Animal Drug Injections. Nanoconjugates were dissolved freshly in PBS (pH 7.4) prior to each experiment and injected intravenously (i.v.) into the lateral tail vein. Mice were anesthetized with isoflurane beforehand, and their tails were briefly warmed to facilitate injections. All conjugates were administered as a single dose, at final concentrations ranging from 0.068 to 0.548 μmol of total nanoconjugate per kg body weight, or as indicated for each experiment. The drug injection volume was kept constant at 150 μL. After each injection, mice were promptly returned to their cages. Fifteen minutes before euthanasia mice were injected with mix of 75 μl Tomato Lectin (DyLight 488 Lycopersicon Esculentum (Tomato) Lectin, catalog #DL-1174 Vector laboratories, 1 mg/ml) and 50 μl Ricin Lectin (Fluorescein Ricinus Communis Agglutinin I (RCA 120), catalog #FL-1081 Vector laboratories, 5 mg/mL) to label tumor and brain vessels. Mice were anesthetized and euthanized by cervical dislocation followed by decapitation in 2 h after the injection of nanoconjugates. Brains were collected, saved in OCT and used for optical analysis.

Tissue Processing and Staining

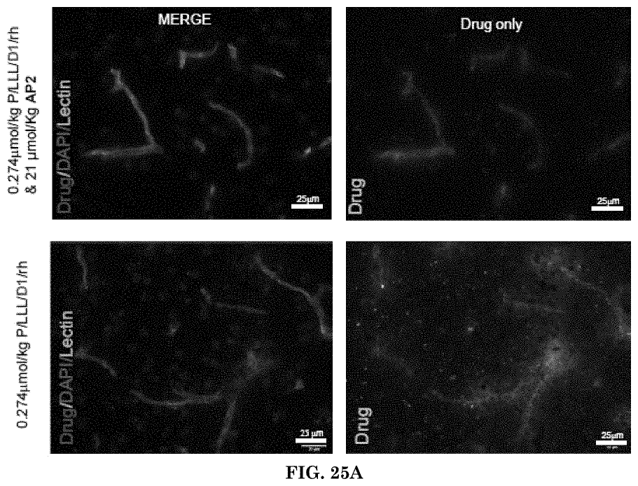

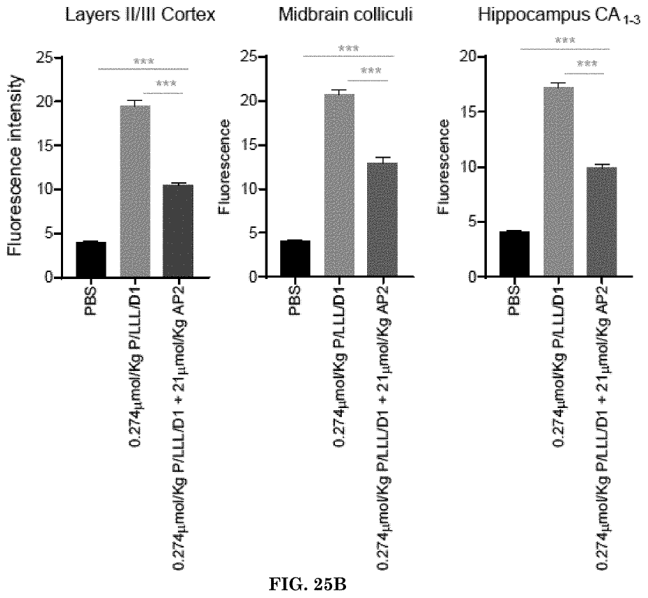

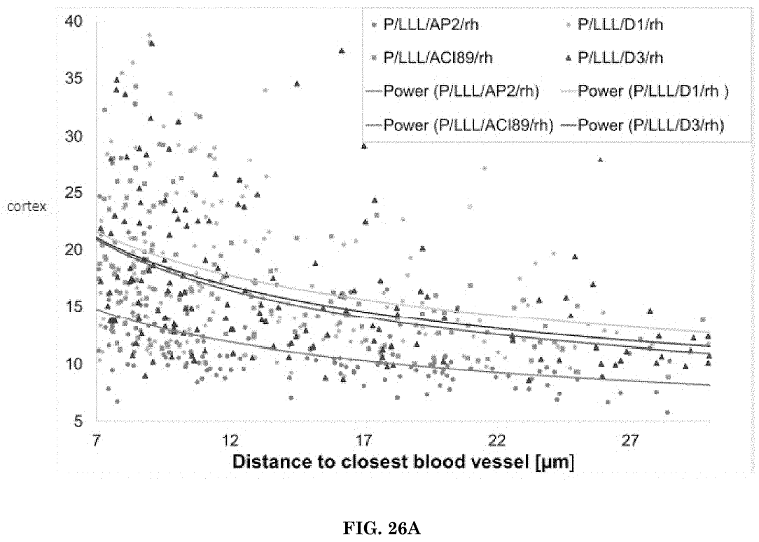

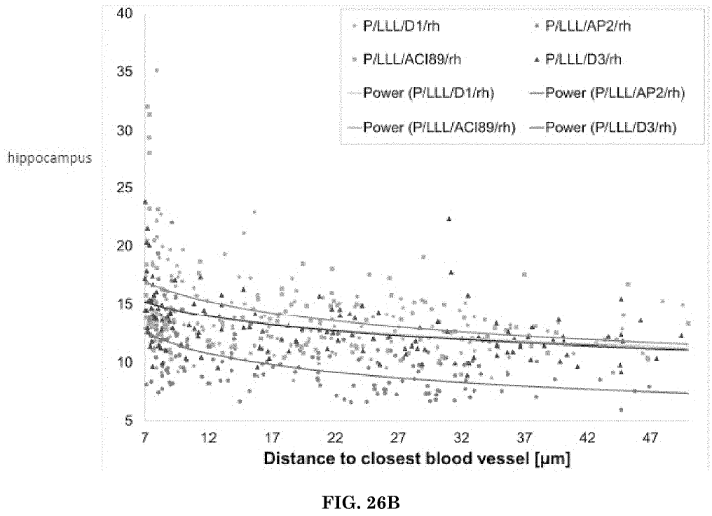

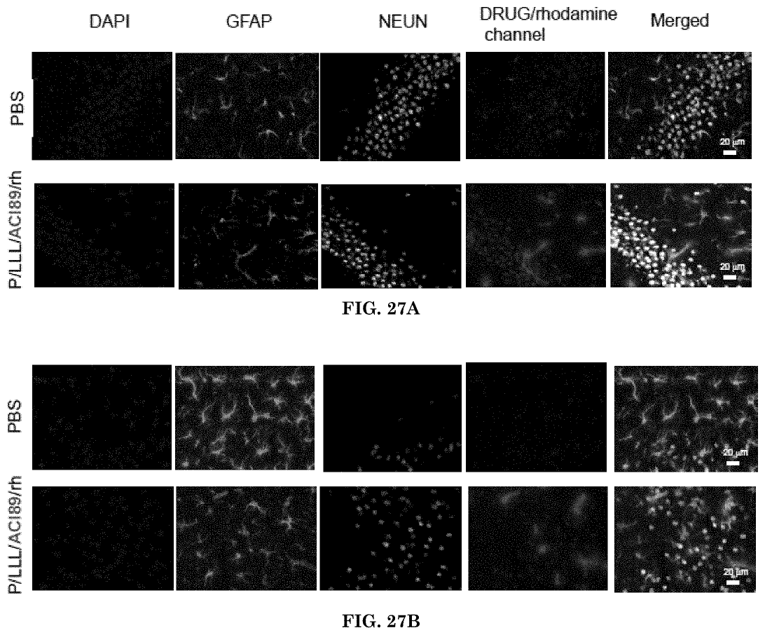

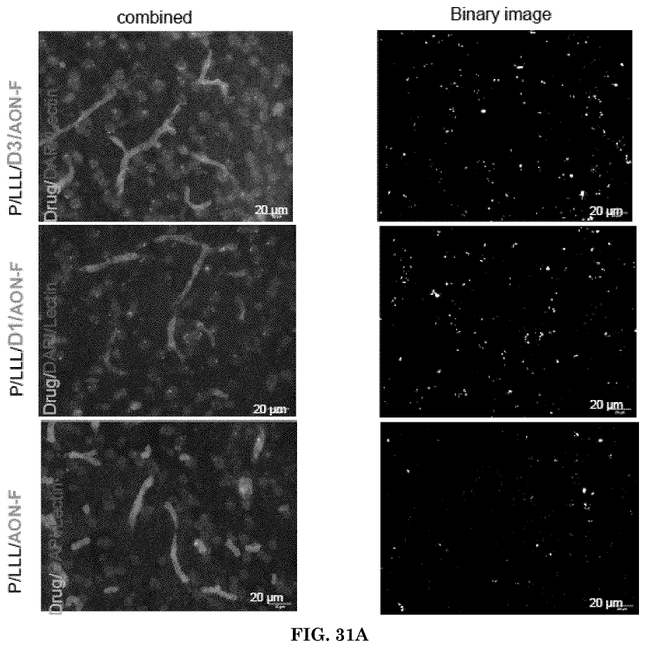

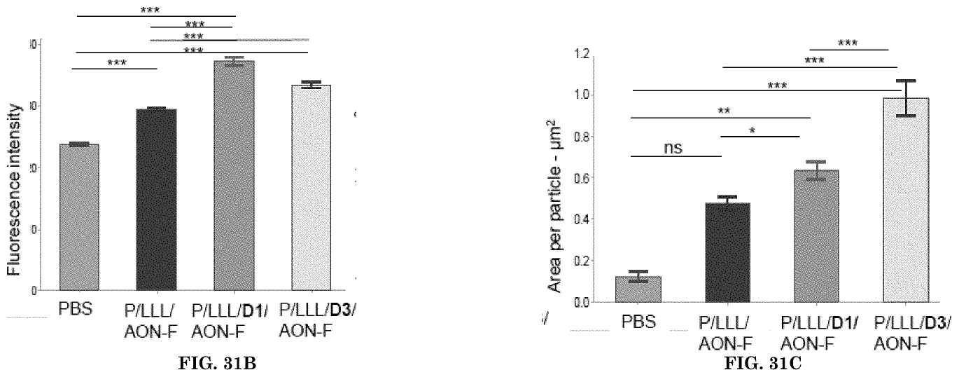

The cerebral vasculature was stained in every experiment as to differentiate blood vessels from brain parenchyma. In experiments in FIGS. 25 and 26A-26B DyLight 488 tomato-lectin (DL-1174; Vector Laboratories, Burlingame, Calif.) was injected as a 150 μL bolus at a 1:2 dilution in saline, 15 minutes prior to euthanasia. For the carboxy-fluoresced labelled groups (FIGS. 31A-31C), DyLight 650 tomato-lectin (150 μL bolus at a 1:1 dilution in saline) was used. This led to widespread and optimal staining of the vasculature. Immunohistochemical staining of the vasculature was performed for tissue shown in FIGS. 27A-27C. This was accomplished in 8-14 μm thick cryosections that were air-dried at room temperature, fixed with 1% paraformaldehyde for 5 min and then rinsed with PBS. the sections were mounted between coverslips in Fluoromount-G with DAPI (Invitrogen, Carlsbad, Calif., USA).

Neuronal and glial counterstaining: In FIGS. 27A-27C, brain tissue from drug-injected mice was counterstained to determine if our nanoconjugates are taken up by neurons or glia. Cryosections (8-14 mm thickness) were air-dried for 10 min, fixed with 2% paraformaldehyde for 5 min, and then rinsed three times with PBS. The sections were then incubated in a humid chamber with blocking buffer containing 5% normal BSA, 0.25% Triton X-100, 2% DMSO and 1% normal goat serum (all from Sigma) in PBS (PBS-T) for 1-2 h at room temperature. The antibodies anti-Neun (Abcam, Cambridge, Mass., USA; AB104225) and anti-GFAP (Neuromics, Edina, Minn., USA; C1122102) were then diluted 1:500 in PBS-T and tissue sections incubated simultaneously with antibody solutions overnight at 4° C. in a humid chamber. Tissue sections were then washed five times with PBS-T and incubated in appropriate secondary antibody diluted 1:250 in PBS-T, for 2-4 h. After five washes in PBS at room temperature, sections were mounted between coverslips in Fluoromount-G with DAPI.

### Example 3

### Pharmacokinetic (PK) Analysis of Nanoconjugates in Serum and Brain

Animal drug administration was performed as described herein using eight to nine week old BALB/C mice. A total of 110 mice were used to produce the data described herein.

Retroorbital blood collection & tissue collection: Blood was drawn from the retroorbital sinus at multiple time points to measure the concentration of drug in the serum. Time points ranged from 30 to 480 minutes and are indicated separately for each experiment. Blood was collected with a microhematocrit capillary tube (I.D. 1.1 mm; Chase Scientific Glass, Rockwood, Tenn., USA) and 150 μl blood was collected per mouse into a BD Microtainer SST and stored at room temperature for 45 min, and then centrifuged at 6000 rpm for 5 min. The serum was then transferred into fresh tubes and stored at −80° C. until further use.

Immediately following blood collection, mice were euthanized at predetermined timepoints. Euthanasia was conducted by spinal dislocation of deeply anesthetized animals; the brain, spleen, liver, heart, lungs and kidneys were promptly removed, flash frozen, and placed into −80° C. storage. All tissue used for microscopic analysis was embedded in optimal cutting temperature compound (OCT; Sakura, Torrance, Calif., USA) and placed on dry ice for freezing.

PK measurements using serum: Fluorescently-labeled nanoconjugates with known concentrations (in mol/mL) were used to obtain standard fluorescence calibration curves, which were used to convert raw fluorescent measurements in collected serum to mol/mL units shown in this paper. Amounts of 20 μL of the processed blood serum containing injected conjugates were placed in 96-well white opaque plates and the fluorescence was measured using a fluorimeter at 570/600 nm excitation/emission with a 590 nm cutoff (Flexstation, Molecular Devices, Sunnyvale, Calif., USA). Results were converted to μg/mL using the calibration curve and plotted as a function of time. PK half-life t_{1/2} values were calculated using Prism (Graphpad, LaJolla, Calif., USA).

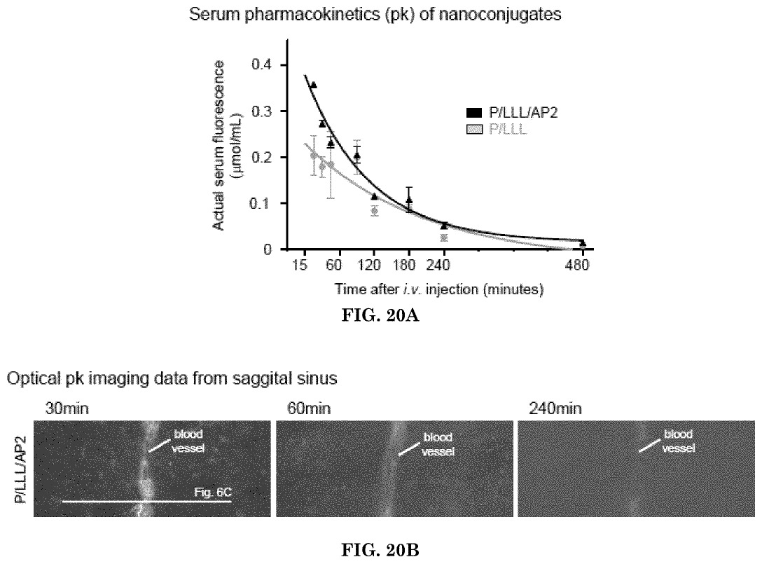

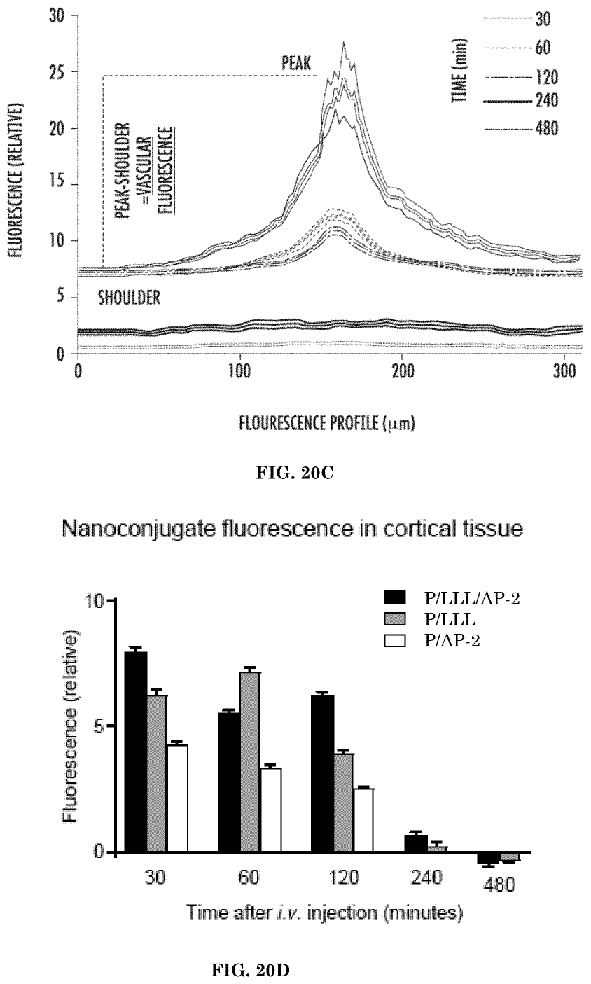

Optical drug clearance measurements (e.g., FIG. 20A, 20C) were obtained from optical imaging data of the saggital sinus blood vessel from mice that were sacrificed at multiple timepoints from 30 to 480 minutes. Vascular fluorescence was defined as the difference between fluorescent peaks and shoulders in a linear profile that was drawn perpendicularly across the blood vessel (see FIG. 20C). The sequential decrease in fluorescence was then converted to mol/mL via calculation with a fluorescent standard with a known concentration, and plotted alongside serum measurements in FIG. 20A.

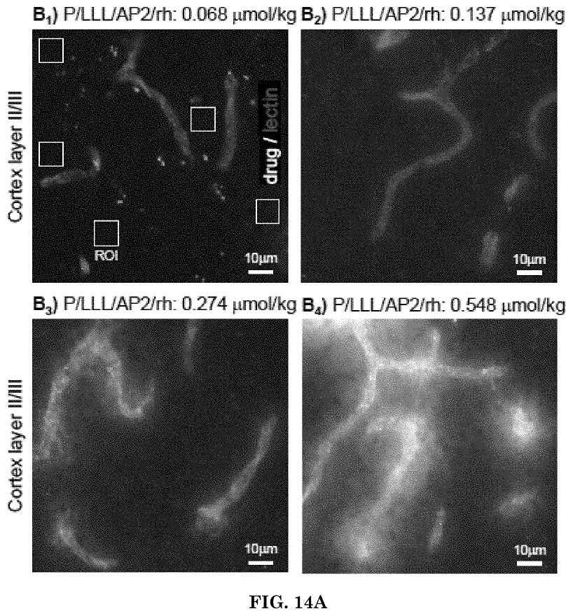

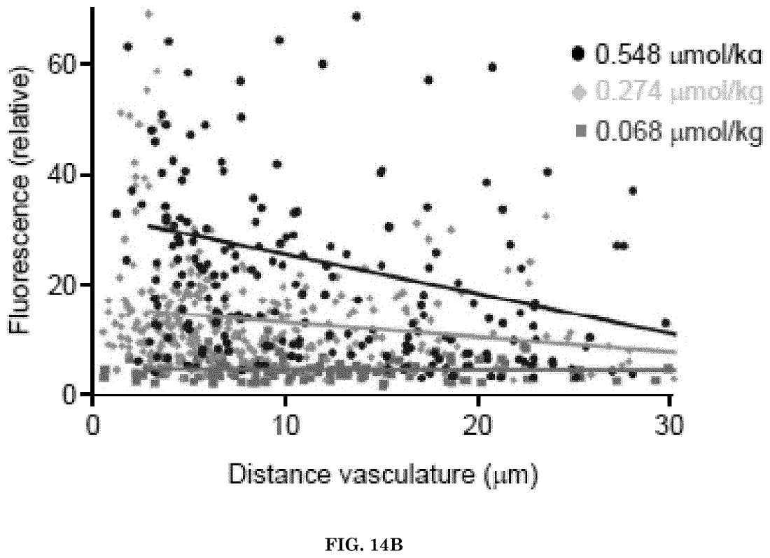

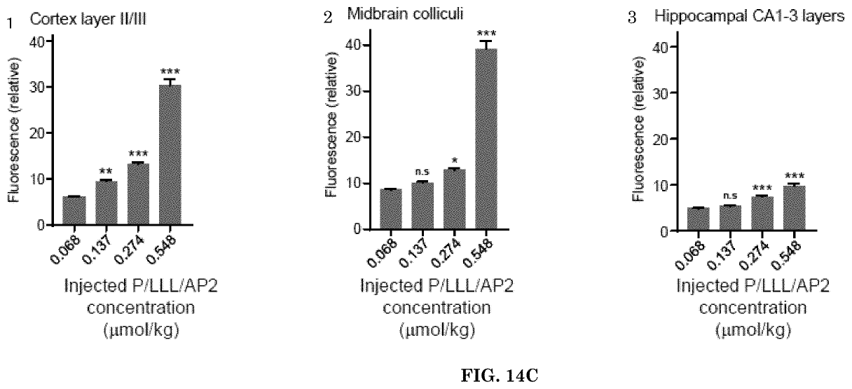

Tissue processing & staining: The cerebral vasculature was stained in every experiment in order to differentiate blood vessels from brain parenchyma. In most experiments (FIGS. 14A-14C, 15A-15B, 16A-16B, 19A-19E, 21A-21C) DyLight 488 tomato-lectin (DL-1174; Vector Laboratories, Burlingame, Calif.) was injected as a 150 μl bolus at a 1:2 dilution in saline, 15 minutes prior to euthanasia. This led to widespread and optimal staining of the vasculature. Immunohistochemical staining of the vasculature was performed for tissue shown in FIGS. 20A-20D. This was accomplished in 8-14 μm thick cryosections that were air-dried at room temperature, fixed with 1% paraformaldehyde for 5 min and then rinsed with PBS. The sections were then incubated in a humid chamber with blocking buffer (5% normal BSA and 0.1% Triton X-100 in PBS) for 1 hr. Sections were stained with anti-von Willebrand Factor (vWF, Abcam, Cambridge, UK) conjugated to AlexaFluor 488 (Thermo-fisher scientific, Canoga Park, Calif., USA). After washing, the sections were mounted as described above.

### Example 4

### Image Acquisition and Optical Analysis

Imaging was performed with a Leica DM 6000B epifluorescence microscope (Leica Microsystems, Wetzlar, Germany). Rhodamine-labeled nanoconjugates were visualized with a 534-558 nm excitation and 560-640 nm emission filter set, viewed with a 20× Leica HC Plan Apo 0.70 N.A. and a 40× Leica HCX Plan Apo 0.85 N.A. lens, and recorded with a Leica DFC 360 FX camera. The camera was controlled with Leica LAS X software and images were acquired with 4.5 sec+2.0 gain exposures for the 20× lens and 3.5 sec+2.0 gain exposures for the 40× lens. These parameters were held constant throughout the imaging experiment to enable accurate image-to-image comparisons across trials and experiments. Other fluorophores (DAPI, tomato-lectin, antibodies) were viewed using complementary standard filter sets and their imaging parameters were also held consistent across experimental trials. For the GFAP and Neun stained slides, nanoconjugates images (rhodamine) were acquired 6.0 sec+3.0 gain exposures with a 40× lens. For the labelled AON conjugates (carboxy fluorescein) images a GFP 430-530 nm emission filter set and a 4.0 sec+2.0 gain exposures.

To identify drug distribution in the vessels, tumor tissue out of vessels and normal brain tissue, to test the drugs penetration through BBB (distance) as well as cellular targeting frozen tissue blocks were sectioned at 12-14 μm thickness using a Leica CM3050 S cryostat (Leica Biosystems, Inc., Buffalo Grove, Ill., USA). Tissue sections were air-dried at room temperature, then mounted with ProLongGold Antifade (Thermo Fisher Scientific) mounting medium containing 4′,6-diamidino-2-phenylindole (DAPI) to counterstain cell nuclei. Images were captured using a Leica DM6000 B microscope (Leica Microsystems, Inc., Buffalo Grove, Ill., USA). For both tumor and brain (other hemisphere) 5 images were quantified per mouse using 20 10*10 μm² ROI per field. Rhodamine-labeled nanoconjugates were visualized with a 534-558 nm excitation and 560-640 nm emission filter set, viewed with a 20× Leica HC Plan Apo 0.70 N.A. and a 40× Leica HCX Plan Apo 0.85 N.A. lens, and recorded with a Leica DFC 360 FX camera. The camera was controlled with Leica LAS X software and images were acquired with 2.0 sec+1.0 gain exposures with the 40× lens. These parameters were held constant to enable image-to-image comparisons across specimens.

Analysis of optical data: Analysis of optical imaging data was performed in Image) FIJI (Schindelin et al. (2012) Nature methods, 9, 676-82, which is incorporated herein by reference as if fully set forth).

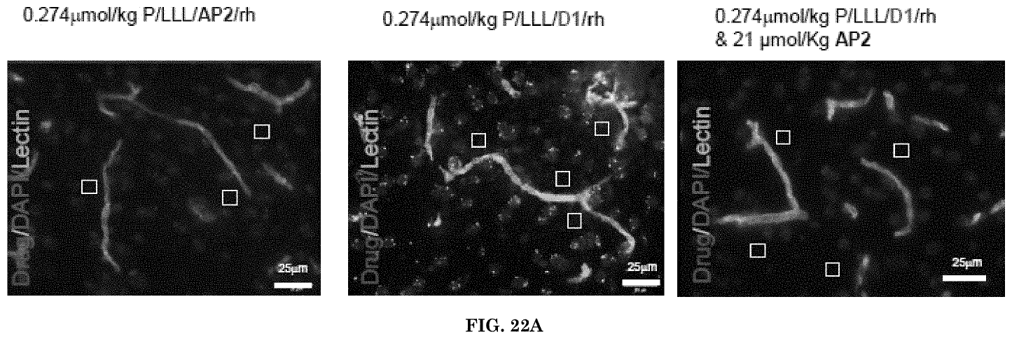

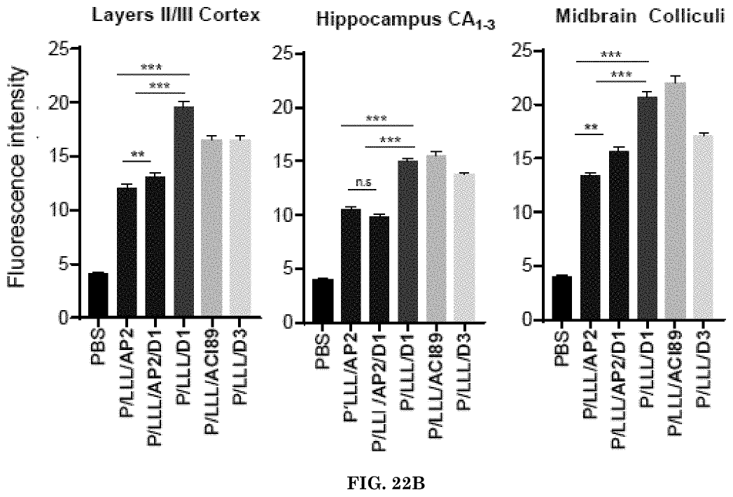

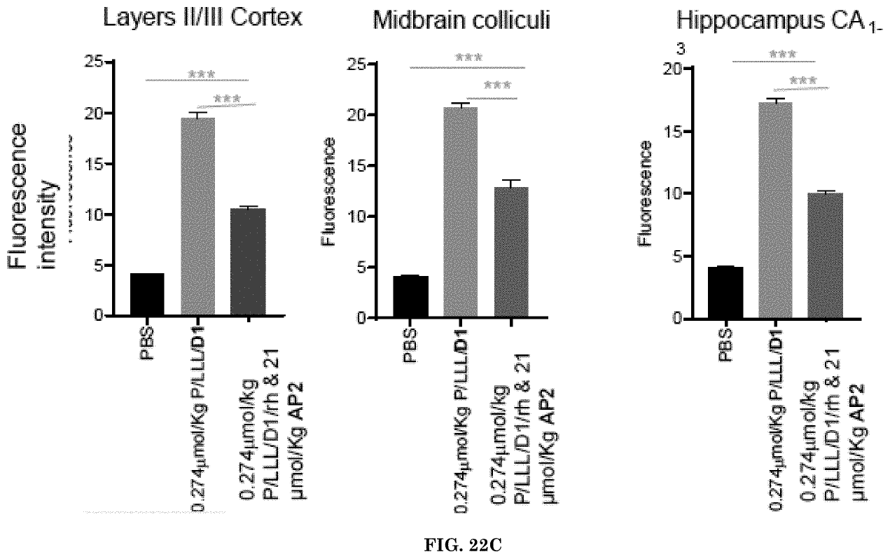

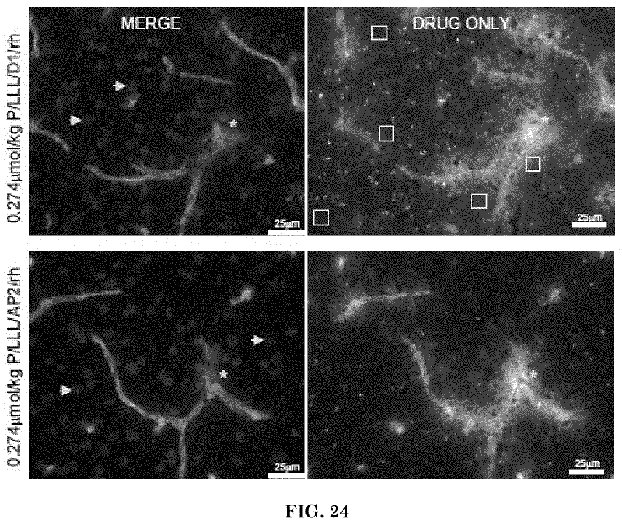

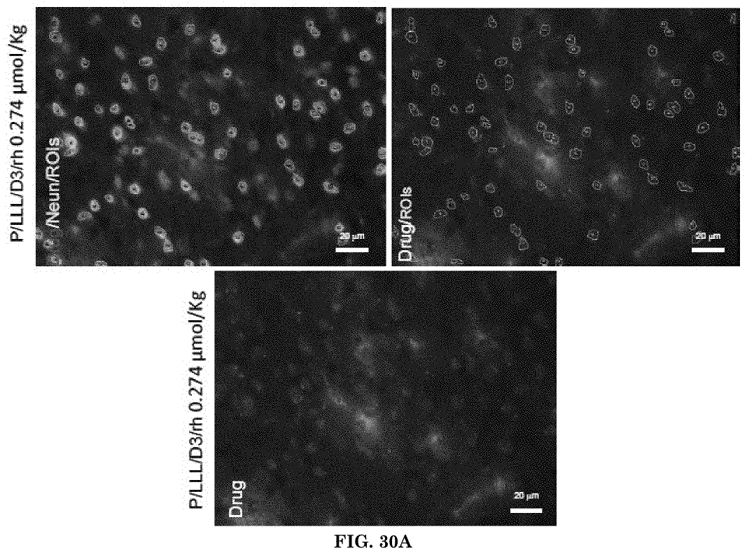

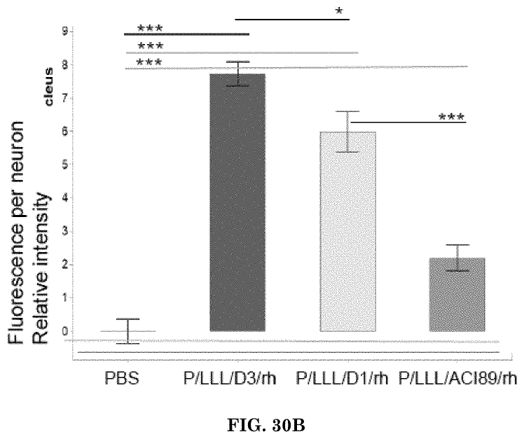

To determine if the nanoconjugates entered the brain parenchyma, an image intensity analysis was performed in regions that did not contain vasculature (i.e., see yellow boxes in FIG. 14A, photograph 1, and FIGS. 22A and 24). In this analysis, 20×10 μm² or 10×10 μm² sized regions of interest (ROI) were randomly overlaid on images showing the vasculature, explicitly avoiding blood vessels. For FIG. 30A, the ROI was drawn around all Neun stained nucleuses detected. Intensity measurements and positions were then obtained for each ROI after it was separately overlaid on the image showing nanoconjugate fluorescence.

Fluorescence measurements were thus based on the anatomy of the cerebral vasculature rather than nanoconjugate labeling of whole sections or extracts of brain parenchyma and were therefore unbiased and intentionally avoiding the nanoconjugate load in the cerebral vasculature. Overall levels of nanoconjugate labeling are shown as means and standard errors for 20 measurements from four separate images of the hippocampal CA1-3 layers, layers II/III of the somatosensory or visual cortices, and the superior and inferior midbrain colliculi (4 mice for each conjugate and brain region).

To determine how nanoconjugate-associated fluorescence relates to the anatomy of the vasculature, the distance of each analyzed ROI was manually measured from the nearest blood vessel wall (using line tool in FIJI). For tumor bearing mice, 5 images per tumor tissue and 5 per brain tissue were analyzed for each mouse. Intensity values were then plotted against the location of the blood vessel wall and summarized in scatterplots (e.g., FIG. 14B).

All nanoconjugate fluorescence measurements in brain parenchyma (e.g., FIGS. 14B-14C (1-3); 16B (1-3); 19B-19E; 20C-20D; and FIG. 22B) are presented as relative fluorescence intensities. Relative fluorescence measurements were obtained by subtracting from each nanoconjugate fluorescence measurement the average intensity of auto fluorescence that was imaged in a corresponding brain region in mice that were injected with PBS (total of 6 mice; 28 images for each brain region). Relative fluorescence measurements thus represent total fluorescence minus representative autofluorescence. Data plots and statistical analysis were conducted in Prism (FIGS. 22A-22C and FIGS. 25A-25B) or in Minitab (FIGS. 30A-30B and 31A-31C). Unless indicated otherwise, fluorescence measurements were compared via a one-way ANOVA combined with pairwise post-hoc comparisons of individual data points; exact parameters and tests are separately indicated for each result. Statistical significance is indicated as follows: *=p<0.01, **=p<0.001, and ***=p<0.0001.

For the particulate fluorescence analysis, images were converted to binary images, and then 5 ROIs 20*20 μm² were drawn per image. The area of each particle on the ROI was measured using the Image) particle analysis tool, where each particle was measured separately. Sizes were then summarized, sorted, and further analyzed using Minitab18 software (Minitab, Inc, State College, Pa., USA). Particulate areas observed between different conditions were compared via a one-way ANOVA combined with post-hoc Tukey tests to test differences between each experimental group.

### Example 5

### In Vivo Data Analysis

Pharmacokinetics for PMLA/angiopep-2 (2%)/rhodamine (1%) conjugate is representative for PKs of the single peptide conjugates.

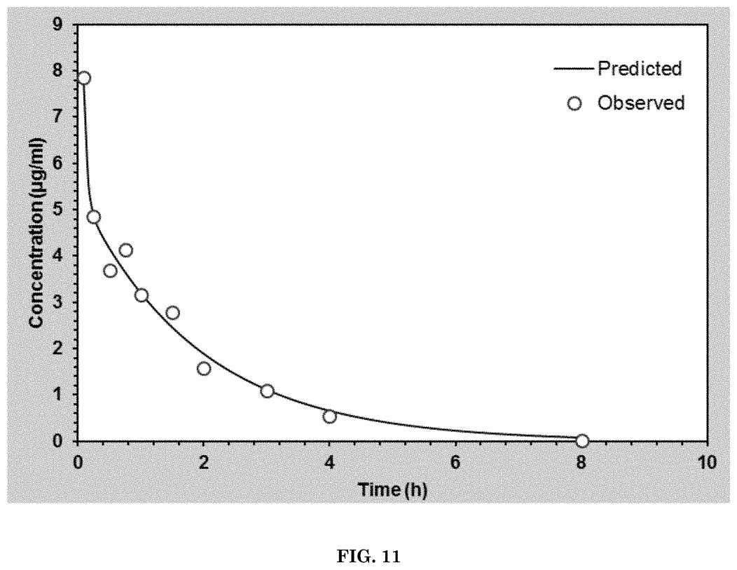

FIG. 11 illustrates PK for PMLA/angiopep-2 (2%)/rhodamine (1%) conjugate measured by fluorescence intensity of the attached dye as a function of time from IV injection into tail vain until blood samples were taken. The sample fluorescence intensity was converted to mg injected nanoconjugate on the basis of standard curves obtained by spiking blood samples with known mg-amounts of conjugate and botting fluorescence intensity as function of mg nanoconjugate. The drawn curve in FIG. 11 was calculated for the obtained best fit to the experimental points. Parameters shown in Table 3 below were calculated on the basis of the curve.

TABLE 3
Calculated Parameters

| Parameter | Unit | Value |
|---|---|---|
| k10 | 1/h | 1.69 |
| k12 | 1/h | 12.0 |
| k21 | 1/h | 6.0 |
| t½Alpha | h | 0.036 |
| t½Beta | h | 1.31 |
| C0 | μg/ml | 18.5 |
| V | (μg)/(μg/ml) | 0.22 |
| CL | (μg)/(μg/ml)/h | 0.366 |
| V2 | (μg)/(μg/ml) | 0.435 |
| CL2 | (μg)/(μg/ml)/h | 2.60 |
| AUC 0-t | μg/ml*h | 10.8 |
| AUC 0-inf | μg/ml*h | 10.9 |
| AUMC | μg/ml*h\hat{ }2 | 19.3 |
| MRT | h | 1.78 |
| Vss | μg/(μg/ml) | 0.65 |

Of the observed two phases, the second phase is considered and follows the half-life of 1.31 h. Residual amount of nanoconjugate after 4 h from injection is less than 6%.

PMLA/LLL (40%)/Angiopep-2 (2%)/rhodamine (1%) nanoconjugate was IV (tail) injected into healthy nude mice. Ex vivo brain slices were examined at 0.5 hours, 1 hour, 2 hours and 4 hours after injection. It was observed that the nanoconjugate was visible around blood vessels for two hours and almost disappeared at 4 hours after injection of the nanoconjugate.

It was observed that the nanoconjugates that do not carry Aβ binding peptide do not show depositions at AD plaques in Alzheimer diseased mice. It was also observed that deposition of dye fluorescence was independent of type of dye at characteristic fluorescence wave lengths.

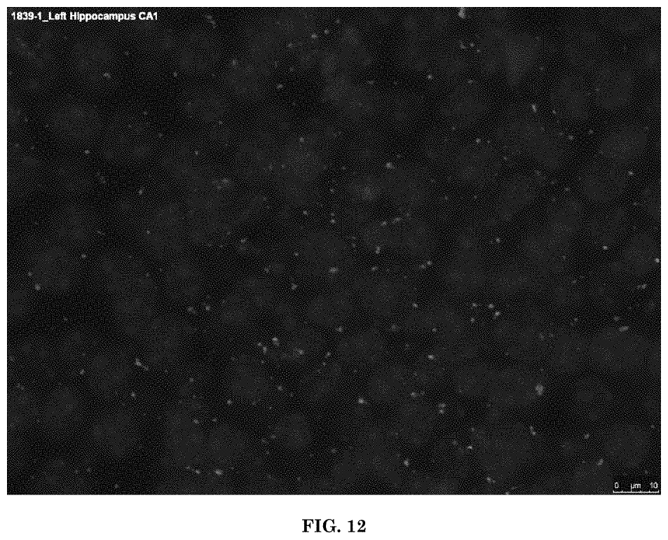

FIG. 12 is an image of the left hippocampus CA1 2 hours after (IV) injection of buffer into the tail vain of a healthy mouse. The location of the fluorescent spots was observed to be next to nuclei, have excitable fluorescence in the green and red wavelength region and have been reported to represent disposed lipophilic material called lipofuscin. These are different from the nanoconjugates, which appear as red “haze,” and are only excitable in the red light range. After applying a filter, the clouds are translated in clouds of shades of white and grey.

### Example 6

### Distribution of Peptides Conjugates as a Function of Time and in Spatial Relation to Blood Vessels

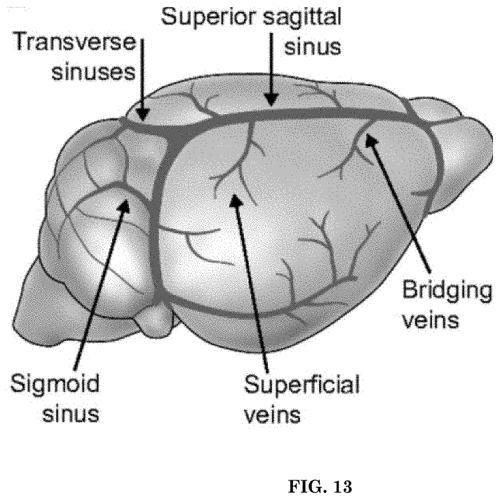

FIG. 13 is a schematic drawing of the brain showing main blood vessels including the Superior Sagittal Sinus (SSS), a large blood vessel that runs along the midline of the brain. Location of the nanoconjugates (also referred to as mini nanodrugs) in the SSS was examined at 60 min and 120 min after injection. Examination of this location as shown on FIGS. 20B-20C provides information about the transfer of the drug from the vasculature into the brain parenchyma and its disappearance after 2-4 hours. This is a qualitative observation (FIG. 20B), but it was found to be convincing when comparing the area surrounding the SSS at 60 min and 110 min after IV injection. At 60 min there is much more drug in the form of a small particle “haze” near the vessel. The haze is almost completely cleared after 120 min. Qualitative analysis can be performed in the form of a fluorescence intensity vs distance from a SSS shown in the profile plot on FIG. 20C. The appearance that peptide nanoconjugates have passed through BBB in the molecular form seen as “haze” or “clouds” indicated that these agents had permeated BBB as solutes. The Morphometric Analysis confirmed that “haze” was generated by fluorescence of the peptide conjugates following subtraction of the fluorescence background caused by lipofuscin shown on FIG. 12.

Similar results have been obtained for all peptide nanoconjugates (or mini nanodrugs) described herein.

### Example 7

### Characterization of Nanoconjugate Fluorescence in Brain Parenchyma

BBB penetration and brain distributions of the nanoconjugates were studied via optical imaging of fluorescence emitted by their rhodamine moiety. All imaging was conducted in fixed cryosections that were obtained from mice at various times after systemic i.v. injections. Two distinct patterns of fluorescence were observed, however only one could be ascribed to the nanoconjugate. One type of fluorescence was attributed to the presence of lipofuscin, which is an intracellular metabolite and waste deposits in neurons (Di Guardo (2015), which is incorporated by reference as if fully set forth). It was hypothesized that nanoconjugate fluorescence may contribute to the lipofuscin signal (i.e., via degradation and accumulation of rhodamine in intracellular organelles), but this type of fluorescence was excluded from the spectral analysis. A distinction between diffuse nanoconjugate fluorescence and lipofuscin has not been reported, even though several studies have shown lip ofuscin-like particulate staining patterns.

This distinction is a precondition to obtaining accurate and reliable optical measurements of nanoconjugate fluorescence.

### Example 8

### Concentration-Dependent BBB Penetration of P/LLL/AP-2

Table 1 lists nanoconjugates that were examined for their ability to penetrate the BBB and distribute in the brain parenchyma. The results indicate that P/LLL/AP2 has the BBB penetration ability.

FIGS. 14A-14C illustrate concentration dependent BBB penetration of P/LLL/AP-2/rhodamine. FIG. 14A is a set of photographs illustrating optical imaging data acquired at 120 min after i.v. injection of P/LLL/AP-2/rhodamine at the following concentrations: photograph 1-0.068 mol/kg; photograph 2-0.173 μmol/kg; photograph 3-0.274 μmol/kg; and photograph 4-0.548 μmol/kg. Drug concentrations are listed with regard to total nanoconjugate content systemically injected. Referring to this figure, the vasculature is shown in red, and the nanoconjugate as whitish diffused clouds. FIG. 14B is a chart illustrating nanoconjugate fluorescence intensity vs. “distance from vasculature” measurements in brain parenchyma of mice injected with three different concentrations: black: 0.548 μmol/kg; grey: 0.273 μmol/kg; white: 0.068 μmol/kg. Referring to FIG. 14B, fluorescence measurements were obtained from 10 μm²-sized regions of interest (ROI) that were randomly overlaid on regions devoid of vasculature shown as yellow squares on photograph 1 of FIG. 14A. Intensity measurements and positions were then obtained for each ROI and plotted against the location of the nearest blood vessel wall. FIG. 14C is set of charts: chart 1—Cortex; chart 2—Midbrain and chart 3 Hippocampus, illustrating average nanoconjugate fluorescence in the brain parenchyma measured following injections at four different drug concentrations. In this figure, fluorescence is shown as relative fluorescence, which is the measured nanoconjugate fluorescence after subtraction of autofluorescence imaged from PBS injected animals using similar acquisition settings. All statistical tests therein were conducted against P/LLL/AP-2/rhodamine at 0.068 μmol/kg; individual test results are indicated with asterisks where *=p<0.01, **=p<0.001, and ***=p<0.0001.

Referring to FIG. 14A, presented are the optical imaging data of mice i.v. tail-injected with different concentrations of P/LLL/AP-2/rhodamine and sacrificed 120 minutes post-injection. The drug concentration is listed as the total concentration of each injected nanoconjugate, where the conjugates contained 40% LLL, 2% peptide and 1% rhodamine, unless indicated otherwise. The tissue shown in FIG. 14A was counterstained with tomato-lectin to show the vasculature (red), while the nanoconjugate is shown in grey/white.

It was observed that injections of P/LLL/AP-2/rhodamine at increasing drug concentrations produced visibly more fluorescence, as is shown for mice injected with 0.068 μmol/kg (Photograph 1), 0.137 μmol/kg (Photograph 2), 0.274 μmol/kg (Photograph 3), and 0.548 μmol/kg (Photograph 4) in FIG. 14A. Referring to Photograph 4 of FIG. 14A, it was also observed that there is much more drug in the form of “haze.” The brain tissue permeation of the nanoconjugate was not uniform, and most of the nanoconjugate fluorescence was concentrated in the perivascular space, between 5-20 μm from the blood vessel wall. Referring to FIG. 14A, this is visible in Photograph 4, as strong nanoconjugate fluorescence (grey “haze”) near the blood vessels, but diminished fluorescence further away from the blood vessels. FIG. 14B explores this relationship in a plot from all of the measurements (for each condition: 4 mice, 3-4 sections with 20 random measurements each). All fluorescence intensity measurements were conducted with 10 μm²-sized regions of interest placed outside of tomato-lectin stained blood vessels (ROI as in Photograph 1 of FIG. 14A); the positions of these ROIs were then measured against the location of the nearest blood vessel wall to produce the scatterplot in FIG. 14B. Fitting the data with a linear regression, indicated a fluorescence intensity decrease (slope) of −0.72±0.15 for the 0.548 μmol/kg drug injection condition, and −0.272±0.07 for the 0.274 μmol/kg drug injection condition. This confirms that nanoconjugate tissue permeation is not uniform and that the drug concentration decreases with distance from the vasculature. However, based on significantly different γ-intercepts, significantly more BBB penetration of P/LLL/AP-2/rhodamine was confirmed following injections at higher drug concentrations. As such, the γ-intercept for the 0.548 μmol/kg drug injection condition was 34.07±2.3; 17.49±0.8 for the 0.274 μmol/kg drug injection, and 6.342±0.34 for drug injected at 0.068 mol/kg.

Referring to FIG. 14C, the results described above are applicable to the cerebral cortex (Chart 1), the midbrain (Chart 2) and the hippocampus (Chart 3). The data shown in on charts 1-3 of FIG. 14C are average nanoconjugate fluorescence intensity values and their standard errors: these were obtained from randomly sampled ROIs, irrespective of their location and distance from the vasculature (4 mice in each condition). Notably, Chart 3 of FIG. 14C shows that fluorescence measurements in the hippocampus were consistently lower than those in the cortex or midbrain. The hippocampus is linked to the formation and maintenance of memories, is affected by neurodegenerative disease, and is thus a crucially important target for potential nanoconjugate therapies (Zeidman and Maguire (2016); which is incorporated by reference as if fully set forth). For example, FIG. 12 shows that the background fluorescence in the hippocampus area was attributed to lipofuscin, which is preexisting autofluorescence and not dependent on injection of the buffer or peptide nanoconjugates. The background fluorescence has been subtracted from the fluorescence intensities illustrated on FIG. 14C.

It was hypothesized that the lower nanoconjugate fluorescence in the hippocampus is due to the comparatively small amount of vascular perfusion of this brain region.

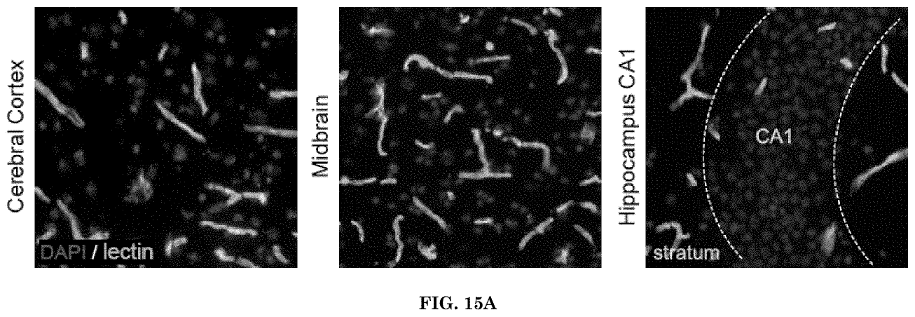

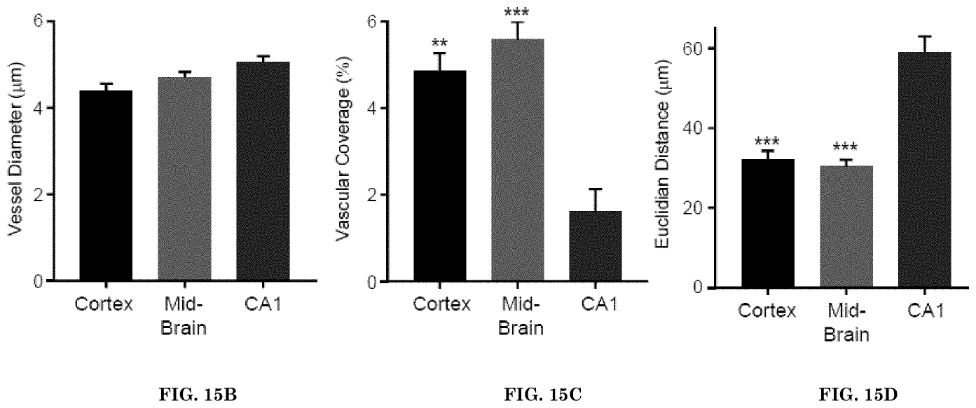

FIGS. 15A-15D illustrate blood vessel diameters, vascular coverage and inter-vessel distances in different brain regions. FIG. 15A is a set of photographs illustrating blood vessels in the cortex, midbrain and hippocampal CA1 cellular layer (outlined). The vessels were stained with tomato-lectin (shown here as white stretches) and nuclei were counterstained with DAPI (grey dots). FIG. 15B are bar graphs illustrating vessel diameters. Referring to FIG. 15B, the vessel diameters were measured as the shortest distance between the vessel walls and were on average 4-5 μm in every brain region. Blood vessels of this diameter were within the range of the cerebral microvasculature. FIG. 15C is a bar graph illustrating vascular coverage. Referring to FIG. 15C, the vascular coverage was defined as the area occupied by tomato-lectin stained blood vessels divided by the total area of each analyzed image. The vascular coverage is similar in the cortex and midbrain but much smaller in the hippocampal CA1 cellular layer (ANOVA: F=22.03; p=0.0003). FIG. 15D illustrates the inter vessel distance defined as the shortest (Euclidian) distance between two adjacent blood vessels, comprehensively sampled for all vessels in each image. Referring to FIG. 15D, it was observed that this distance was largest in the hippocampus (ANOVA: F=36.05; p<0.0001), which confirms that there are the fewest blood vessels in this region. Individual statistical comparisons were conducted against morphological measurements from the hippocampus and are indicated as **=p<0.001 and ***=p<0.0001.

Referring to FIGS. 15B-15C, similar-sized blood vessels were observed in the cortex, midbrain and hippocampus (FIG. 15B), but the area covered by these blood vessels is less in the hippocampus than the cortex or midbrain (FIG. 15C). Referring to FIG. 15D, these results in an inter-vessel distance in the hippocampus of 59 μm, which is almost twice that of the cortex (32 μm) and midbrain (30 μm). By taking into account that P/LLL/AP-2/rhodamine distributes preferentially within ˜30 μm from the microvasculature (i.e., FIG. 14B), it can be argued that the reduced vascular access in the hippocampus may be responsible for its reduced drug perfusion. This issue can be partially resolved through drug injections at higher concentrations, as is observed by a significant dose-dependent increase of hippocampal nanoconjugate fluorescence in FIG. 14C, panel 3 Hippocampus).

### Example 9

### BBB Penetration Depends on Nanoconjugate Composition

Attention was next turned to the effects of individual nanoconjugate moieties on BBB penetration (LLL and AP-2), whereby the concentrations of remaining LLL (40%), AP-2 (2%) and rhodamine (1%) were held constant. The LLL moiety was removed, which resulted in P/AP-2 (with 0% LLL).

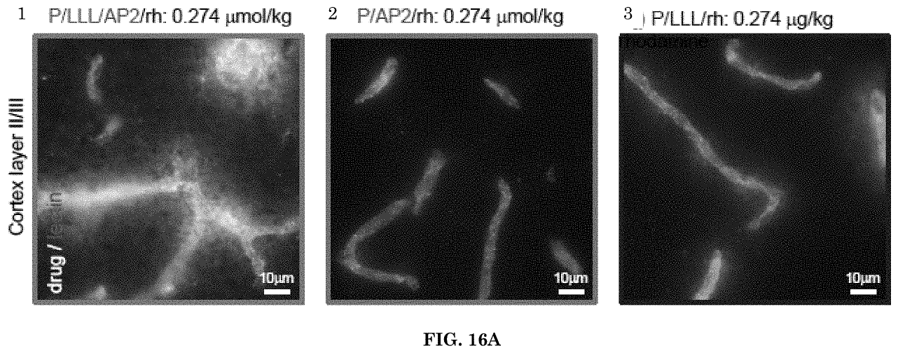

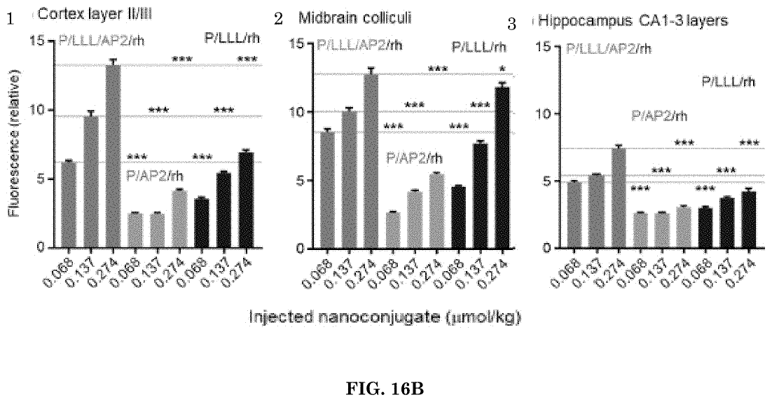

FIGS. 16A-16B illustrate that the nanoconjugate composition determines degree and locus of BBB penetration. FIG. 16A is set of photographs illustrating nanoconjugate permeation of the cerebral cortex: photograph 1—P/LLL/AP2; photograph 2—P/AP-2 and photograph 3—P/LLL. Referring to this figure, optical imaging data showing nanoconjugate permeation of the cerebral cortex: nanoconjugate fluorescence is white “haze” and the vasculature is indicated by red stretches. The most intense “haze” fluorescence was observed for P/LLL/AP-2 as shown on photograph 1. FIG. 16B is a set of bar graphs showing average nanoconjugate fluorescence in the cerebral cortex (1), the midbrain (2) and the hippocampus (2) as a function of nanoconjugate composition and concentration: P/LLL/AP-2 is shown in black, P/AP-2 in grey and P/LLL in white. Average nanoconjugate fluorescence measurements were obtained from 20 randomly sampled regions of interests explicitly outside of the cerebral vasculature (4 mice with 4 images each, for each measurement). Statistical tests were conducted between nanoconjugate types (e.g., black vs grey) within different concentrations. The results are indicated with d asterisks where *=p<0.01, **=p<0.001, and ***=p<0.0001; the dotted lines show the concentration of P/LLL/AP-2 against which each comparison was made.

Referring to FIG. 16A, data shown on photograph 1 vs. photograph 2 show that P/LLL/AP-2 penetrated the brain parenchyma better than P/AP2. This is especially apparent in the perivascular space where much of the diffuse grey or white nanoconjugate fluorescence “haze” can be seen in the P/LLL/AP-2 but not the P/AP-2 condition. Corresponding fluorescence measurements from the cortex are summarized in FIG. 16B, chart 1, (black vs. grey data) and were significantly larger for P/LLL/AP-2 vs. P/AP-2 injected at 0.068 μmol/kg (Tukey: p<0.0001), 0.137 μmol/kg (Tukey: p<0.0001), and 0.274 μmol/kg (Tukey: p<0.0001). Indeed, the fluorescence associated with P/AP2 was invariably lower across all of the cortical tissue that was imaged. Essentially the same observations were made in the midbrain (FIG. 16B, chart 2) and the hippocampus (FIG. 16B, chart 3), and it was concluded that P/AP-2 owns little potential for BBB penetration.

It was examined if the removal of the AP-2 moiety affected the BBB penetration of the nanoconjugate. P/LLL (with 0% AP-2) generated less fluorescence in brain parenchyma than P/LLL/AP2 (FIG. 16A, photograph 1 vs. photograph 3) at all concentrations tested (FIG. 16B, chart 1; black vs. white data). However, brain tissues from mice injected with P/LLL were significantly more fluorescent than tissues from mice injected with P/AP-2 (grey vs. white in FIG. 16B, chart 1): more cortical fluorescence was associated with P/LLL vs. P/AP-2 at 0.068 μmol/kg (Tukey: p<0.01), 0.137 μmol/kg (Tukey: p<0.0001), and 0.274 μmol/kg (Tukey: p<0.0001). This observation was also made in the midbrain (FIG. 16B, chart 2), and in the hippocampus (FIG. 16B, chart 3). Thus, P/LLL penetrates the BBB even without a peptide moiety. The addition of the AP-2 peptide significantly increases BBB penetration, and in combination with LLL, produces the optimal nanoconjugate formula, P/LLL/AP2.

Present results surprisingly indicate that the LLL moiety, in conjugation with PMLA, also contributes to BBB permeation of PMLA, without the need of a BBB penetrating peptide. This mechanism may involve synergistic contributions of PMLA and LLL moieties to introduce a specific hydrophobic/hydrophilic amphiphilic conjugate, which breaks the blood brain barrier.

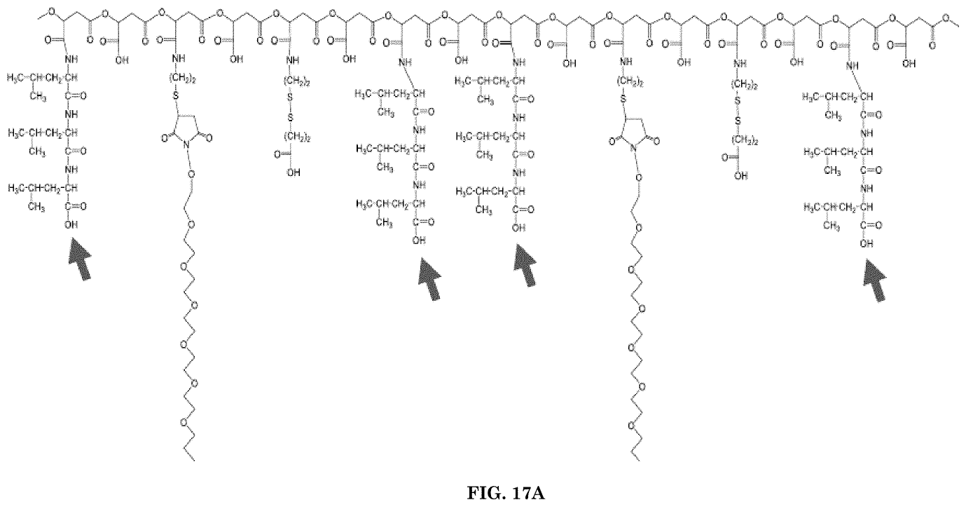

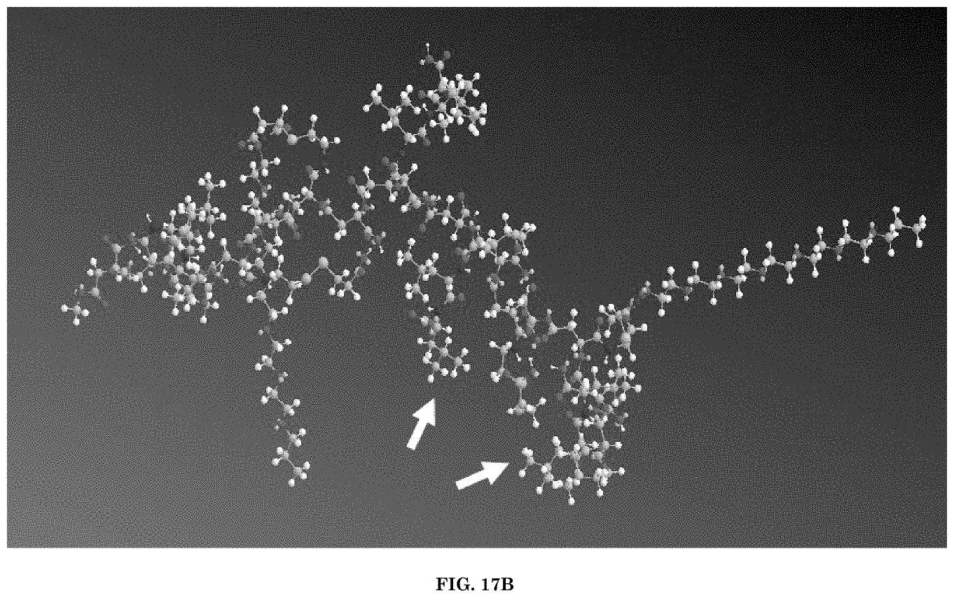

Furthermore, energy calculations show that intra molecular LLL-LLL associations have an impact on the conformation of the nanoconjugate, i.e., which could favor AP-2 or other peptide-independent BBB permeation. FIGS. 17A-17B illustrate the effect of conjugated LLL residues on nanoconjugate conformation. FIG. 17A is a schematic drawing of a chemical structure of the representative conjugate containing LLL and part of the conjugated peptide linker (PEG). LLL is indicated with black arrows in the structural scheme. FIG. 17B is a three-dimensional image of the short representative PMLA structure illustrated in FIG. 17A (16 malic acid residues) with PEG (2 chains of ethylene glycol-hexamer conjugated via maleimide to PMLA), capped sulfhydryl (two moieties) and LLL (4 moieties). Van der Waals interactions between adjacent LLL moieties are indicated with white arrows. The structure shown on FIG. 17B is the result of total energy minimization calculated in vacuum indicated 226 kcal/mol for the analogue with LLL (Chem3D Pro 11.0).

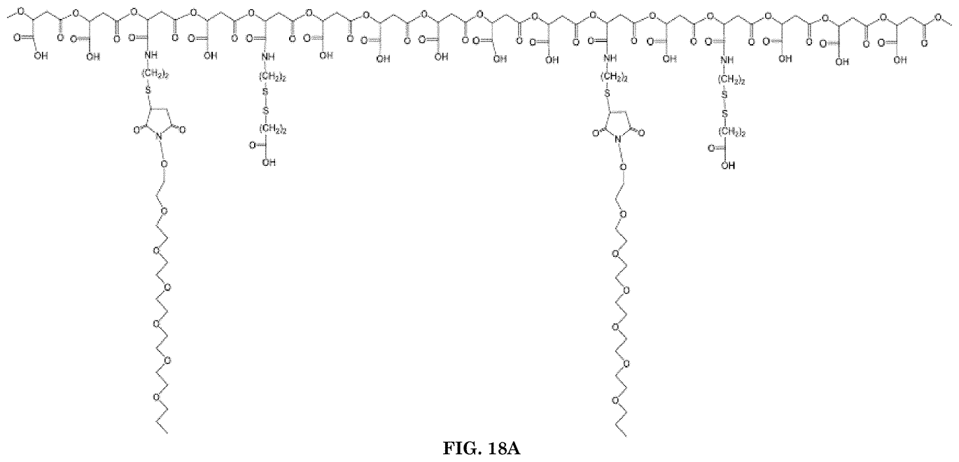

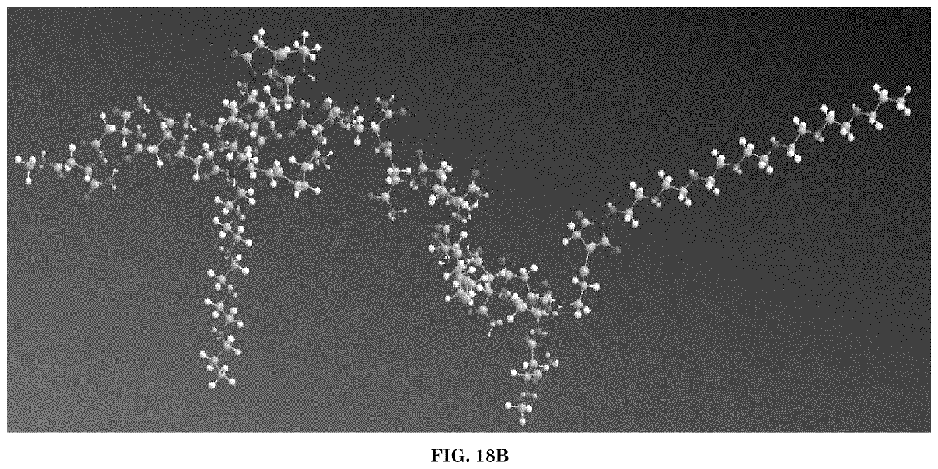

FIGS. 18A-18B illustrate nanoconjugate conformation in the absence of LLL. FIG. 18A illustrate the structural model and is similar as the one shown in FIG. 17A. Because the structure is lacking LLL, the 3-dimensional conformation of the conjugate appears extended in comparison with the one in FIG. 17B. FIG. 18B is a three-dimensional image of the structure shown in FIG. 18A obtained after energy minimum calculation. The total energy is 1194 kcal/mol according to energy minimization calculated for vacuum (Chem3D Pro 11.0). It is known that PMLA is negatively charged (Table 1: ζ potential of P/Rh is −22.9 mV) and therefore hydrophilic; this may increase the distance of its approach and preclude initial interaction with negatively charged endothelial cell membranes. The addition of LLL decreases the negative charge and increases the hydrophobicity of PMLA, which may facilitate interactions with cell membranes. Second, the addition of LLL may hinder the formation of electrostatic contacts between the positively charged AP-2 peptide residues and the negatively charged PMLA backbone. Without LLL, the peptide-linker moieties in the conjugate can fold and attach to the PMLA backbone, ultimately rendering them less available for biological interactions. LLL sterically prevents this interaction so that the AP-2 peptide becomes biologically active by interacting with LRP-1 (or other receptor molecules). Results of dynamic light scattering (DLS, hydrodynamic diameter in a solution) and polydispersity index measurements (PDI, molecular size distributions) agree with this idea and show a diversity of nanoconjugates with different extents of polymer coiling and coil sizes (Table 2).

Energy calculations as shown on FIGS. 17A-17B and 18A-18B indicate that LLL can induce folding of nanoconjugates via LLL-LLL interactions, which ultimately decreases conformations of the free polymer and hence reduces numbers and diameters of conformational variants. Thus, PMLA alone had a measured hydrodynamic diameter of 3.68 nm and a high PDI of 0.89 (Table 2). After formation of the P/LLL conjugate, the average diameter was reduced to 2.68 nm and variant dispersity to 0.50. The measured diameter of P/AP-2 was 5.93 nm and the PDI of 0.79 implicating an increased diversity inferred by irregular attachment of the peptide to the polymer. The effect of conjugating LLL (i.e., P/LLL/AP-2) reduced the size to 4.45 nm and the PDI to 0.39, explained again by the formation of intra conjugate LLL-LLL contacts, even though the conjugate carried more load and molecular weight. Further to this observation, structures and three-dimensional models shown on FIGS. 17A-17B and 18A-18B obtained by energy calculations (in the absence of solvent) show three-dimensional structures of short PMLA analogues which mimic short PMLA conjugates with and without LLL (16 malic acid residues, two hexa ethylene glycol oligomers (282 g/mol) conjugated via maleimide and two sulfhydryl moieties) and conjugated LLL (FIG. 17A; 4 moieties, black arrows) and without conjugated LLL (FIG. 18A). The structural models thus show that LLL moieties can associate to form intramolecular domains, and that LLL-LLL interactions reduce the number of possible confirmations of the PMLA conjugate by increasing rigidity and decreasing the diameter. In summary, conjugation with LLL is favorable for BBB permeation by (i) optimizing the interactions of targeting peptides with receptors of a particular transcytosis pathway, (ii) reducing the diameter of the permeating nanoconjugate, and (iii) increasing the rigidity of the nanoconjugate.

### Example 10

### Screening BBB-Penetrating Peptide Moieties

BBB-penetrating peptides, namely AP-2, M4, B6, and cTfRL were conjugated to P/LLL and screened for their ability to permit or enhance BBB-penetration of the nanoconjugate (Demeule et al. (2008); Staquicini et al. (2011); Yin et al. (2015); Liu et al. (2013); and Oller-Salvia et al. (2016), all of which are incorporated by reference as if fully set forth).

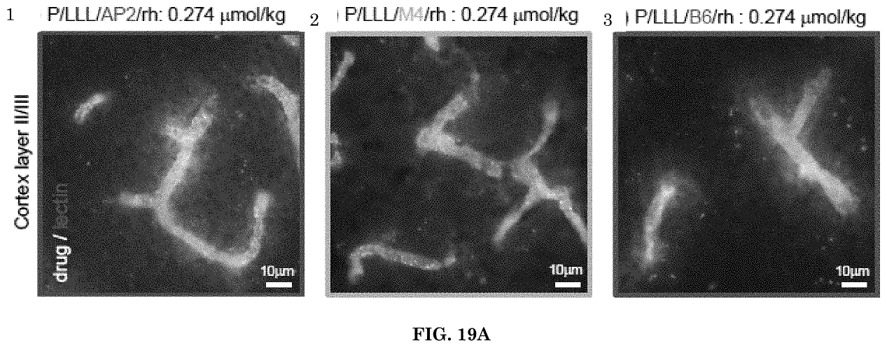

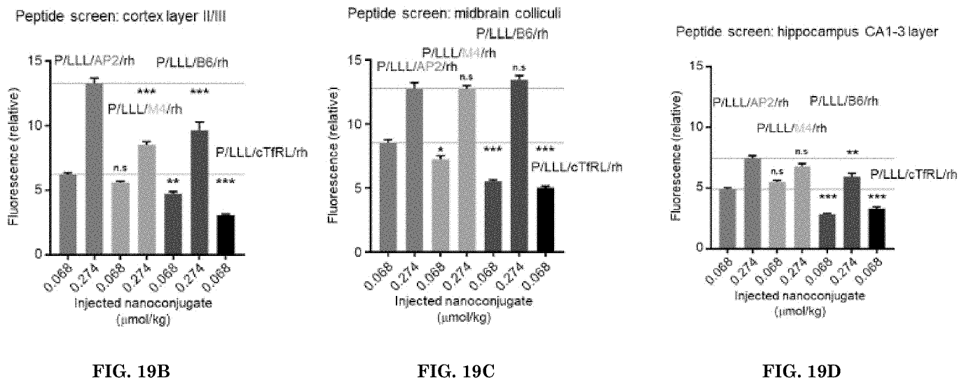

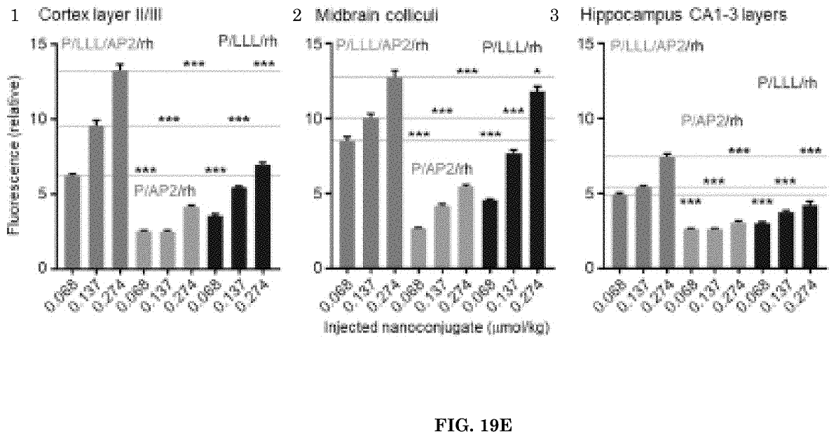

FIGS. 19A-19E illustrate nanoconjugate peptide moiety screen. FIG. 19A is a set of photographs illustrating P/LLL equipped with different peptides (1—P/LLL/AP-2; 2—P/LLL/M4; and 3—P/LLL/B6) to assess their role in BBB penetration. Referring to this figure, optical imaging data of the rhodamine labeled peptide conjugates show permeation of the cerebral cortex by P/LLL conjugated to AP-2 (1), M4 (2) and B6 (3). Nanoconjugate fluorescence is white/grey and the vasculature is red. FIGS. 19B-19D is a set of bar graphs showing average nanoconjugate fluorescence in the cerebral cortex (FIG. 19B), midbrain (FIG. 19C) and hippocampus (FIG. 19D) for P/LLL/AP2/rh (two grey bars on the left), P/LLL//M4/rh (two light grey bars in the middle left) and P/LLL/B6/rh (two dark grey bars in the middle right) and P/LLL/cTfRL/rh (one black bar to the right) injected at concentrations of 0.068 μmol/kg or 0.274 μmol/kg. FIG. 19E illustrates nanoconjugate fluorescence measurements in the cerebral cortex (1), midbrain colliculi (2), hippocampus CA1-3 layers (3) for peptide combinations P/LLL/AP2/rh (three light grey bars on the left side), P/LLL/AP2/M4/rh (light grey bar on the middle right) and P/LLL/AP7/rh (grey bar on the right) injected at concentrations of 0.137 μmol/kg or 0.274 μmol/kg. Statistical tests were conducted against each of the different concentrations of P/LLL/AP2 in each histogram and are indicated with asterisks where *=p<0.01, **=p<0.001, and ***=p<0.0001; the grey lines show the concentration of P/LLL/AP2 against which each comparison was made.

The nanoconjugate with high BBB penetration had the formula P/LLL/AP-2/rhodamine. Referring to FIG. 19A, replacing AP-2 with M4 (photographs 1 and 2; P/LLL/M4) resulted in similar levels of nanoconjugate fluorescence in the cortex of mice injected with 0.068 μmol/kg conjugate (light grey vs. medium grey in FIG. 19B; Sidak: p=0.5749). However, the conjugate P/LLL/M4 injected at 0.274 μmol/kg produced significantly less fluorescence in the cortex than P/LLL/AP-2 (FIG. 19B; Sidak: p<0.0001). Yet, essentially identical levels of P/LLL/M4 and P/LLL/AP-2 fluorescence were measured in both, the midbrain and the hippocampus, regardless of the injected drug concentrations (light grey vs. medium grey in FIGS. 19C and 19D). Hence, P/LLL/M4 and P/LLL/AP-2 appear to permeate the brain tissue with similar efficacies, but P/LLL/M4 shows regional selectivity and poor permeation of the cerebral cortex.

Fluorescence measurements resulting from injections of TfR ligands were generally less than those obtained from injections with P/LLL/AP-2. P/LLL/B6 was almost always less when compared to injections of P/LLL/AP2 in the same brain region (medium grey vs. dark grey in FIGS. 19B-19D). The only exception was for P/LLL/B6 associated fluorescence in the midbrain, which was similar to that measured for P/LLL/AP-2 injected at 0.274 μmol/kg (compare black vs. white in FIG. 19C; Sidak: p s=0.2499). The midbrain contains the highest density of cerebral microvasculature (e.g. FIGS. 15A-15B), and this likely facilitates the drug entry into the brain tissue. This could also explain why P/LLL/AP-2, P/LLL/M4 and P/LLL/B6 show essentially the same levels of nanoconjugate fluorescence in the midbrain if injected at a high enough concentration (0.274 μmol/kg). A nanoconjugate containing the Tf ligand cTfRL at 0.068 μmol/kg (P/LLL/cTfRL), produced fluorescence intensity measurements comparable to B6 in the midbrain and hippocampus (FIGS. 19C and 19D) and low intensities in the cortex (FIG. 19B). Because results for P/LLL/cTfRL were redundant with P/LLL/B6, this nanoconjugate was dismissed from further experiments. As an additional control to experiments shown on FIGS. 16A-16B, P/AP-2/rhodamine (i.e., different peptide omitting LLL) was synthesized. This peptide had poor BBB penetration. Similarly, both P/M4 and P/cTfRL had poor penetration into the brain parenchyma and produced extremely low fluorescence measurements. These results confirm the observation that LLL is required for BBB penetration, regardless of which peptide the conjugate carries.

In another set of experiments, it was evaluated if nanoconjugates with peptide combinations and modified peptide loads traverse the BBB more efficiently (FIG. 19E). A nanoconjugate carrying a combination of AP-2 and M4 (P/LLL/AP-2/M4), each of which was promising on its own, permeated the cortex slightly more than nanoconjugates that contained a single peptide. This is shown in FIG. 19E, where P/LLL/AP-2/M4 injected at 0.137 μmol/kg produced slightly, but not significantly more fluorescence than P/LLL/AP-2 at the same concentration (medium grey vs. dark grey; Sidak: p=0.0617). Thus, P/LLL/AP-2/M4 failed to display a significant sum of effects by each peptide. Moreover, P/LLL/AP-2/M4 has a reduced cargo capacity due to higher occupancy of the polymer platform and thus a reduced number of free ligand attachment sites.

In assessment was made if an increase in the same peptide load on the nanoconjugate could lead to enhanced BBB penetration. Thus far, all of the conjugates carried 2% total peptide content. In FIG. 19E, a doubling of the peptide load was demonstrated, P/LLL/AP-2 (4%) actually resulted in decreased BBB penetration (dark grey vs. light grey; Sidak: p<0.0154). Per these results, it was concluded that 2% peptide was the optimal load for the nanoconjugate delivery system.

The results of injected P/LLL/AP-7 were measured as a control. AP-7 differs from AP-2 by the replacement of two lysine residues in positions 10 and 15 with arginine residues (TFFYGGSRGRRNNFRTEEYCNH₂ (SEQ ID NO: 7)), which reportedly impairs peptide interactions with endothelial LRP-1 receptors (Demeule et al. (2008), which is incorporated by reference herein as if fully set forth).

P/LLL/AP7 permeated cortical brain tissue but produced significantly less fluorescence than P/LLL/AP-2, both injected at 0.137 μmol/kg (dark grey vs medium grey in FIG. 19E; Sidak: p<0.0001). This result confirms a substantial role for authentic AP-2 to enable trans-BBB movement of the nanoconjugate. Together with other findings described herein, it was demonstrated that nanoconjugate transport through the BBB depends on peptide identity, peptide load, and interaction with other nanoconjugate moieties (i.e., LLL).

The results apply to the brain of healthy mice. It is instructive to consider that the performance of certain peptides may differ in pathological conditions in which the BBB is impaired, or trans-BBB receptor expression is altered. For instance, the TfR route may be effective for drug delivery into brain tumors. Gliomas overexpress TfR in their vascular endothelium, and this may aid drug-tumor penetration and delivery via enhanced TfR transport (Meng et al. (2017), which is incorporated herein by reference as if fully set forth). In contrast, the LRP-1 route is linked to less active amyloid 8 protein clearance and effects homeostasis in Alzheimer's disease (Grimmer et al. (2014), which is incorporated herein by reference as if fully set forth).

### Example 11

### Nanoconjugate Pharmacokinetics in Blood and Brain

Fluorescent nanoconjugates were administered via i.v. injections and blood was drawn at 15 to 480 minutes following the injections to measure the blood clearance and pharmacokinetics of P/LLL/AP-2/rhodamine and P/LLL/rhodamine in the serum. FIGS. 20A-20D illustrate pharmacokinetics of nanoconjugate fluorescence in serum and brain tissue. FIG. 20A is a chart illustrating serum clearance analysis that was conducted for P/LLL/AP-2 (black) and P/LLL (grey), and optically via imaging of the cerebral vasculature content (black triangle). FIG. 20B is a set of photographs illustrating optical imaging data showing drug clearance vascular and parenchyma accumulation over 240 minutes. These images show the nanoconjugate P/LLL/AP-2 in whitish “haze” and the vasculature in grey. FIG. 20C illustrates vascular fluorescence intensity profile for the saggital sinus as indicated with a white line in FIG. 20B. Timepoints are indicated in the top right corner of this plot. FIG. 20D is a bar graph illustrating time dependence of nanoconjugate fluorescence intensity in brain tissue for rhodamine P/LLL/AP2 (black), P/LLL (grey) and P/AP2 (white) is different from the serum PK kinetics. Fluorescence has a rapid onset and remains quasi-stable for 120 minutes. Clearance occurs at 240-480 minutes. All data shown are from the cerebral cortex and are relative fluorescence values that were subtracted from background image intensities of representative tissues of PBS injected mice.

The nanoconjugate serum concentrations, as shown in FIG. 20A, were calculated from calibration curves that was previously derived from fluorescence measurements of nanoconjugates with known concentrations. The conjugates P/LLL/AP-2 and P/LLL had serum half-lives of 76.7 min and 119 min, respectively. The half-lives were determined by fitting serum fluorescence measurements with single exponential decay functions: the fluorescence decay associated with P/LLL/AP-2 was a good fit with r²=0.9361, while the decay of P/LLL fit with r²=0.715. The decay functions differed significantly (Extra sum of squares F-test: F=8.281; p=0.0002), thus confirming distinct serum clearances for P/LLL/AP-2 and P/LLL.

Having established the pharmacokinetics of the nanoconjugates in serum, the question next asked was if these measurements could be replicated with optical imaging data of brain slices. To do this, direct optical measurements of vascular P/LLL/AP-2/rhodamine fluorescence in brain tissues was performed. FIG. 20B shows imaging data from a large central blood vessel, the sagittal sinus, from 30 to 240 minutes after i.v. injection. The images show the mini nanodrug (whitish “haze”) and the sinus vasculature (grey). FIG. 20C shows the fluorescence intensity profile for this blood vessel and adjacent brain tissues, as indicated with a white line in FIG. 20B. The fluorescent nanoconjugate is clearly concentrated in the vasculature at 30 minutes post i.v. injection, while subsequent timepoints show a progressive loss of vascular fluorescence (FIG. 20C). The “optical vascular fluorescence” was calculated by measuring the difference between fluorescence peaks and the fluorescence intensity in the surrounding parenchyma (see dashed lines in FIG. 20C) and then plotted the vascular fluorescence over multiple timepoints alongside the actual serum measurements in FIG. 20A (black triangle). The optical vascular fluorescence measurements were converted to mol/mL units via normalization to one time point of serum P/LLL/AP-2 (120 min); the remaining timepoints were then converted using the same ratio (0.115 mol/mL serum concentration for the 0.068 μmol/kg injection at 120 minutes). Remarkably, almost the exact same half-life was obtained for the optically measured serum clearance of P/LLL/AP-2 (optical half-life=73.2 min), and no difference was detected between optical and serum-fitted functions (Extra sum of squares F-test: F=0.3327; p=0.8017). This result confirms the validity of the optical imaging data to understand nanoconjugate pharmacokinetics in the brain.

The decay of nanoconjugate-associated fluorescence in the parenchyma of the cortex is summarized in FIG. 20D. Referring to this figure, fluorescence intensity across saggital sinus (vascular) and adjacent parenchyma at various times (30 min—thin solid line, 60 min—dashed line, 120 min—dashed-dotted line, 240 min—thick solid line and 480 min—dotted line) after injection of P/LLL/AP-2/rhodamine. The conjugate fluorescence was maximal at 30 minutes after the i.v. injection and decreased only slightly until 120 minutes (FIG. 20D; ANOVA: F=531.6; p<0.0001), despite a significant decrease of serum drug (FIG. 20A). At 240 minutes after i.v. injection, nanoconjugate fluorescence could not be distinguished from background fluorescence of the brain parenchyma, suggesting that P/LLL/AP-2/rhodamine is eliminated from the brain within four hours after administration (FIG. 20D). The same observations were made in the midbrain and hippocampus. P/LLL-associated fluorescence in the cortex was lower than that of P/LLL/AP-2 throughout the 30 to 480 minute time period (ANOVA: F=268.5; p<0.0001) but the overall fluorescence buildup and clearance followed the same pattern as seen with P/LLL/AP-2 (FIG. 20D; black). It was also observed that P/AP-2-associated fluorescence was lower than that of other nanoconjugates, but again followed a similar trajectory of fluorescence decay (FIG. 20D; white). While the level in the vascular decreases, the level increases in the parenchyma due to time dependent permeation of the fluorescent conjugate through BBB. After prolonged times (240 min and 480 min), the intensity decreases as is explained by retrograde permeation and accumulation of nanoconjugate back into the vascular (with much lower nanodrug content as before thus inducing the retrograde diffusion).

The analysis of data herein shows that P/LLL/AP-2 associated fluorescence disappears from the serum and brain tissue beginning at 4 hours after i.v. injection. The pharmacokinetic measurements were obtained from tissues of mice injected with 0.068 μmol/kg nanoconjugate concentration. The clearance of drugs injected at higher concentrations was not studied but it could be prolongated; this is likely, considering that more drug accumulation was observed in the parenchyma after administering high drug concentrations (see FIG. 14A-14C).

### Example 12

### Estimating Mini Nanodrug Concentration in the Brain Parenchyma Based on Optical Ratio Measurements

In this analysis, the imaging results were to estimate the actual concentration of P/LLL/AP-2 conjugates in cortical brain parenchyma at 120 minutes after the drug injection. This was accomplished by first measuring P/LLL/AP-2/rhodamine fluorescence in the cerebral vasculature and then the surrounding parenchyma with identical regions of interest, followed by a calculation of the vessel/parenchyma fluorescence ratio

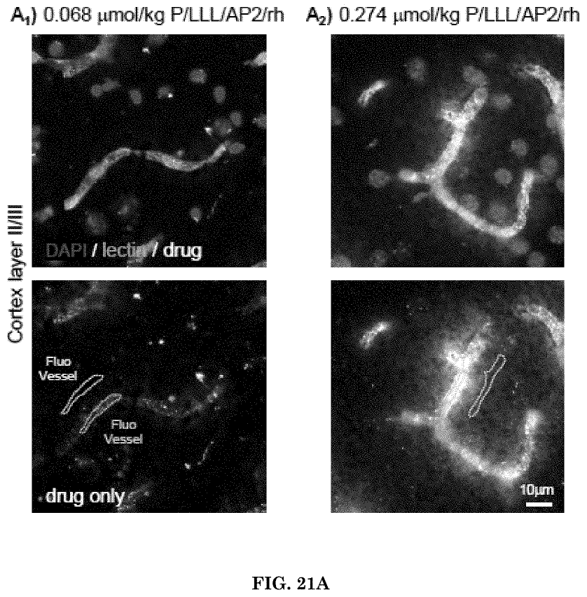

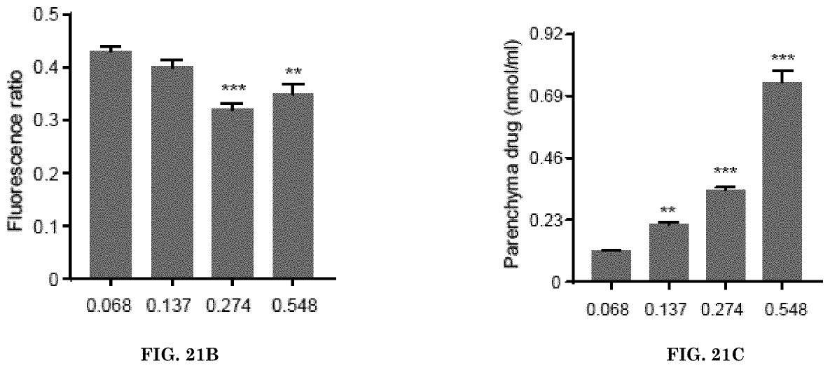

FIGS. 21A-21C illustrate estimation of the nanoconjugate concentration in μg/mL of i.v. injected P/LLL/AP-2 in the parenchyma of the cerebral cortex. (A1-A2). FIG. 21A is set of photographs illustrating optical imaging data showing cortical tissue from mice injected with P/LLL/AP-2/rhodamine at 0.068 μmol/kg (A1) and 0.274 μmol/kg (A2). The top images show cell nuclei (red), vasculature (green stretches) and P/LLL/AP-2 conjugate (white). The lower panels show only P/LLL/AP-2 conjugate-associated fluorescence. Light grey were vessels containing conjugate. Yellow bordered regions of interest selected (ROIs) in vessels, and at indicated distance to vessels ROI were used to quantify vessel-free nanoconjugate and to calculate vasculature/parenchyma fluorescence ratios. The selected ROI were close but not ultimately the regions of highest nanoconjugate staining. FIG. 21B illustrates fluorescence ratios in vasculature/cortical brain parenchyma. Asterisks indicated statistical significance in Tukey test conducted for the 0.068 μmol/kg drug injection condition, where **=p<0.001 and ***=p<0.0001. FIG. 21C illustrates estimated P/LLL/AP-2 concentration in the cortical brain parenchyma. Asterisks indicated statistical significance in Tukey test conducted against the 0.068 μmol/kg drug injection condition, where **=p<0.001 and ***=p<0.0001. Referring to FIG. 21A, data was summarized for 4 mice, 4 sections with 10 measurements for each condition. The images in FIG. 21A (A1 and A2, bottom panel) demonstrate this procedure in two samples from mice injected with 0.068 μmol/kg and 0.274 μmol/kg of P/LLL/AP-2 conjugate, respectively. The fluorescence ratios that resulted from the measurements are summarized in FIG. 21B. A significant reduction in the vasculature/brain parenchyma fluorescence ratio was observed for P/LLL/AP-2/rhodamine injections of 0.274 μmol/kg (ANOVA: F=11.36; p<0.0001; Tukey: p<0.0001) and 0.548 μmol/kg (Tukey: p=0.0018); both concentrations compared to 0.068 μmol/kg. The result indicates somewhat reduced blood-to-brain transport at high concentrations of P/LLL/AP-2, presumably as a consequence of LRP-1 pathway saturation due to high nanoconjugate concentration in the blood.

Using these data, the actual P/LLL/AP-2 concentration was estimated in brain parenchyma by multiplying each of the vasculature/parenchyma ratio measurements with known serum drug concentrations at 120 minutes post injection (0.115 μmol/mL for the 0.068 μmol/kg injection as per FIG. 20A). The resulting drug parenchyma concentrations are plotted in FIG. 21C. A strongly significant overall increase in the drug concentration is observed throughout the cortical parenchyma (ANOVA: F=166.3; p<0.0001). The lowest P/LLL/AP-2 parenchyma concentration is estimated at 0.049±0.001 μmol/ml for the 0.068 μmol/kg injection; the highest parenchyma concentration is 0.32±0.01 μmol/ml for the 0.548 μmol/kg injection. Based on these estimates, the conclusion was made that P/LLL/AP-2 traverses the BBB efficiently and that 40% or higher percentage of free serum drug in the vascular tissue can be detected in the brain within 120 minutes after i.v. administration (% depending on the distance from the vascular tissue).

On that basis, it was tentatively assumed that vascular and proximal parenchymal concentrations are similar (40% and higher vascular concentrations as reference). The similar concentrations could indicate that for P/LLL/AP-2 the blood-brain barrier does not function as a very efficient barrier, at least in the concentration range that was investigated herein. The knowledge of the parenchyma concentration is useful to predict complex formation of the peptide conjugate with receptor molecules in the brain which are intended to be targeted, and thus for the design of cascade targeting.

### Example 13

### BBB Crossing in Normal Brain

The ability of mini nanodrugs carrying D1-, D3- and ACI-89 peptides for BBB penetration was also examined. Comparison was made to mini nanodrugs that carry AP2 peptides. The transfers into cortex of P/LLL/AP2/rh and P/LLL/D 1/rh were chosen which in the first case have been attributed to binding AP2 to LRP1 receptor of the LDL transcytosis pathway and in the second case the D1 which has been shown to bind Aβ₁₋₄₂ (Wiesehan et al., 2003, Chembioche: a European journal of chemical biology, 4 (2003) 748-753, which is incorporated herein by reference as if fully set forth). FIGS. 22A-22C illustrate optical imaging data of the normal brain following mice injection with nanoconjugates labeled with rhodamine. FIG. 22A is a set of photographs illustrating optical imaging data in cortex of normal brain following the injection of mice with 0.274 μmol/kg P/LLL/AP2/rh (left), 0.274 μmol/kg P/LLL/D1/rh (middle) and 0.274 μmol/kg P/LLL/D1/rh and 21 μmol/kg AP2. FIG. 22B are bar graphs illustrating the intensity of fluorescence in the samples of the normal brain following injections of mice with 0.274 μmol/kg (4×) of P/LLL/AP2/rh, P/LLL/AP2/D1/rh, P/LLL/D1/rh, P/LLL/AC189/rh, P/LLL/D3/rh or PBS buffer in layers II/III cortex (left), hippocampus CA₁₋₃ (middle) and midbrain colliculi (right). FIG. 22C are bar graphs illustrating the intensity of fluorescence in the samples of the normal brain following injections of mice with 0.274 μmol/kg of P/LLL/AP2/D1/rh, 0.274 μmol/kg P/LLL/D1/rh and 21 μmol/kg of AP2, or PBS buffer in layers II/III cortex (left), midbrain colliculi (middle) and hippocampus CA₁₋₃ (right).

Briefly, after the nanoconjugates had been injected intravenously at dose 4× (0.274 μmole/Kg) into the tail, BALB/C mice were euthanized after 120 min, the resected brains flash-frozen, and cut slices examined under fluorescence microscope. Referring to FIG. 22A, left panel, the rhodamine-labeled nanoconjugate is recognized by the naked eye in the immediate environment of brain capillaries, and locations farther in the parenchyma when injected at higher doses. Intensities of the diffused nanoconjugate was measured after conversion into gray scale contained in twenty 10×10 μm² ROIs per slice excluding fluorescence in vasculature and in particulates. Particulates are attributed to lipofuscin known as waist deposits in neurons. Referring to FIG. 22B, average intensity values of P/LLL/D1/rh and P/LLL/AP2/rh are displayed in this figure also containing for comparison the results for two other D-peptide nanoconjugates, ACI89 (P/LLL/ACI89/rh) and D3 (P/LLL/D3/rh). The graphs are arranged into panels showing the brain regions Layers II/III Cortex, Hippocampus CA1-3, and Midbrain Colliculi. In comparison with P/LLL/AP2/rh, significantly increased intensities due to BBB permeation are noted for P/LLL/D1/rh, P/LLL/D3/rh, P/LLL/AC189/rh and P/LLL/AP2/rh with maxima in Midbrain. When D1 was combined with AP2 in the two-peptide conjugate P/LLL/AP2/D1/rh, the intensity is significantly less than the sum of contributions P/LLL/AP2/rh+P/LLL/D1/rh. Similarly, as reported previously, the intensities increased as a function of injected dose range 1×-4× (1×=0.068 μmole/Kg) with deviations towards higher efficacy at higher doses (Israel et al., 2019, ACS Nano, 13, 1253-1271, which is incorporated herein by reference as if fully set forth). Taken together, the results for the conjugates containing the D-peptides mirror the results for the AP2-conjugate suggesting their transport through BBB by similar transcytosis pathways. The results seem at variance with the previously assigned adsorptive-mediated transcytosis mechanism for the D3-peptides driven by the contained positive charges. However, the high degree of negative charges in the conjugates with polymalic acid could have overridden here the adsorptive-mediated mechanism for the D-conjugates by a receptor-mediated mechanism.

### Example 14

### The “Boosting Effect” of P/LLL (40%)

The in vivo optical method data have shown a “boosting” effect generated by the P/LLL (40%)-moiety which is present in the P/LLL (40%)/peptide/rh nanoconjugates and enhances the efficacy of BBB permeation due to a property of the shuttle peptide moiety (Israel et al., 2019, ACS Nano, 13, 1253-1271, which is incorporated herein by reference as if fully set forth). The “boosting” effect comprises two ligand sites: a site (A) binding the binding the P/LLL (40%) moiety and (B) shuttle peptide moiety AP2, B6 or M4. Permeation of BBB is observed for different ligands with specificity only for site (A) or only for site (B). After injection of the nanoconjugate containing both kind of ligands forming P/LLL (40%)/AP2, the global permeation efficacy, after subtraction of background, is bigger than the sum of efficacies by the separated ligands (Israel et al., 2019, ACS Nano, 13, 1253-1271, which is incorporated herein by reference as if fully set forth).

This model of subsite (A) and (B) contributing together in a global site (A+B) was, however, dismissed because unlabeled ligand P/LLL (40%) alone in a 70-fold molar excess over the nanoconjugate P/LLL (40%)/AP2/rh consisting of ligands AP2 and P/LLL (40%) did not exhibit competition that would be indicated as a reduction or elimination of the BBB permeation efficacy in mice. In contrast, a slight increase was noted in the mice experiments. It could be concluded that site (A), binding P/LLL (40%), and site (B), binding AP2, were not contiguous in the master binding site which would be active in the LRP1 transcytosis pathway.

Taken into consideration that the LLL and AP2 residues were distributed along the polymer, approximately 150 moieties of LLL (40%) and 8-9 moieties of AP2 (2%) for polymalic acid of an average molecular mass 50000 g/mol, binding and competition is ill defined on the molecular level. Therefore, competition with polymer-free LLL was repeated, expecting that the tri-leucine molecules had less restricted access to receptor binding sites; however, a decrease in permeability was again not observed (data not presented). Since the competition by P/LLL (40%) could not be demonstrated we refer to the reported destabilization of endosomal membranes, which suggests an insertion of tri-leucine containing polymer segments into membranes.

Although binding sites (A) for for P/LLL (40%) and of sites (B) for shuttle peptides did not interact via competition, both contributed to permeation through BBB, and in addition site (A), in a more subtle allosteric interaction, to augment the permeation of the shuttle peptides. A structural and functional interaction with P/LLL may modulate efficacy of membranes involved in transcytosis.

### Example 15

### Mini Nanodrugs that Carry D-Peptides Cross Endothelial Cells Via the LRP1 Transcytosis Pathway

Angiopep-2 (AP2) is a shuttle peptide functioning in the LRP1 transcytosis pathway of vascular brain endothelia (Demeule et al. (2008) J. of neurochemistry, 106, 1534-1544, which is incorporated herein by reference as if fully set forth). The question was asked whether the shuttle peptides D1, D3, and AC189 could also traffic the LRP1 transcytosis pathway. The D-peptides D1, D3 and AC189 were identified by mirror image phage display selection with Aβ₁₋₄₂ as target. Like the original L-peptides, they target Alzheimer's disease-associated Aβ₁₋₄₂ containing plaques (Wiesehan et al., (2003) Chembiochem: a European journal of chemical biology, 4, 748-753; and Zheng et al. (2017) Acta Biomaterialia 49 388-401, both of which are incorporated herein by reference as if fully set forth). Mini nanodrugs that carry the D-peptide were shown to move across BBB via features similar to the pathway of transcytosis found for AP2-nanoconjugates and independently of the previously notified positive charges (Jiang et al., (2016) Biochimica et biophysica acta, 1858, 2717-2724, which is incorporated herein by reference as if fully set forth).

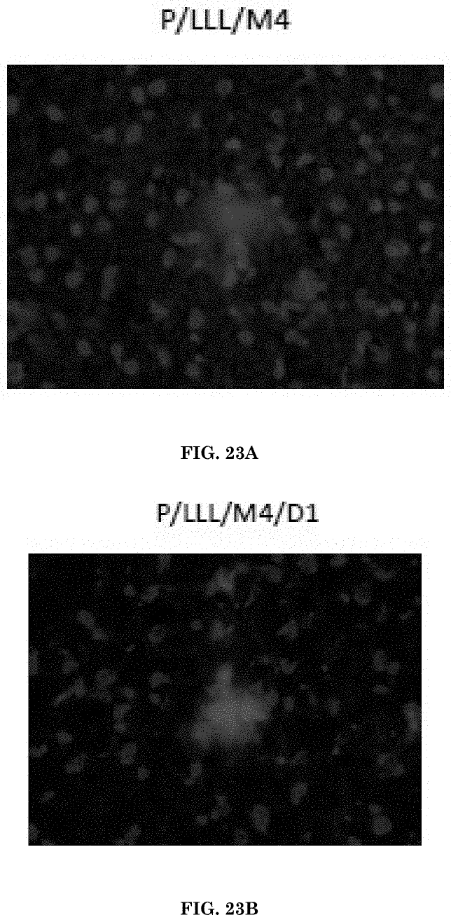

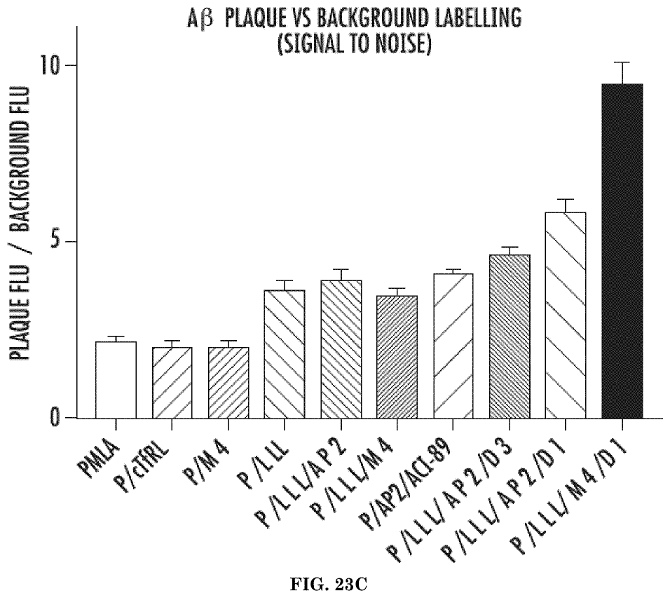

In order to strengthen the AP2-dependent pathway, competition experiments were performed with AP2 as the competitor as shown on FIGS. 23A-23C. AP2 was co-injected in a 70-fold molar excess over the P/LLL/D1 conjugate. In the given excess, AP2 could outcompete P/LLL/D1/rh permeation. However the degree of competition was incomplete although the AP2-concentrations were above the micro molar range typical for phage-display obtained affinity peptides. Referring to FIG. 23C, the average value of the P/LLL/D1/rh fluorescence intensity in the parenchyma dropped by 54.5%±3.0% of the original value for BBB crossing. The competition-resistant fraction of 45.5%-52% permeability is attributed to site (A) occupancy with P/LLL (40%) as discussed above. Referring to FIG. 23B, if the resistant fraction of BBB permeation is attributed to P/LLL (40%), the contribution of D-peptides in site (A) exceeds significantly the one of AP2 in the same site. It remains to be seen whether the improvement reflects the phage-selected binding of Aβ-peptides and/or structural changes inferred by the transition of the L- to D-configuration like the protection against hydrolase-catalyzed cleavage (Jiang et al., (2016) Biochimica et biophysica acta, 1858, 2717-2724, which is incorporated herein by reference as if fully set forth).

### Example 16

### Mini Nanodrugs Targeting Amyloid Plaques

The peptide nanodrugs targeting the carrier to a brain-intern cell or structure were designed. Towards this goal, the nanoconjugates including the D-enantiomeric peptides targeting amyloid and amyloid plaques were used. The efficacy of amyloid targeting peptides D1, D3, ACI-89 was evaluated. FIGS. 23A-23C illustrate peptide-dependent labeling of plagues. FIG. 23A is a photograph illustrating optical imaging data following mice injected with P/LLL/M4. FIG. 23B is a photograph illustrating optical imaging data following mice injected with P/LLL/M4/D1/rhodamine. Referring to FIGS. 23A-23B, plaques staining with the conjugates was observed as “whitish red cloud” in the center of the photographs. Staining by P/LLL/M4/rhodamine (FIG. 23A) was observed to be less intensive than by P/LLL/M4/D1-peptide/rhodamine as was revealed by optical measurement. Referring to these figures, nanodrugs carrying the peptides were iv injected into the mouse tail at doses of 0.548 μmol/kg of P/LLL/M4/rhodamine or P/LLL/M4/D1. The Triple Transgenic Alzheimer's mice (Strain Name: B6;129-sen1^{tm1Mpm}Tg (APPSwe, tauP301L)1Lfa/Mmjax, Short Name: 3xTg) were used. After 8 hours, the mice were euthanized, brain resected and imbedded as described for injection of nanodrugs without intra-brain targeting. Brain was sliced and stained for nuclei. FIG. 23C is a bar graph showing Aβ plaque vs. background labeling (signal noise) shown for PMPLA (P), P/cTfRL, P/M4, P/LLL, P/LLL/AP-2, p/LLL/M4, P/LLL/AP-2/ACI-89, P/LLL/AP-2/D3, P/LLL/AP-2/D1, and P/LLL/M4/D1 mini nanodrugs. All mini nanodrugs shown on FIG. 23C contained fluorescent rhodamine. Experiments were performed essentially as described for FIGS. 23A-23B. All plaques can be labeled by either fluorescent Thioflavine, Amyloglow, fluorescence labeled mAbs against beta-amyloid or were recognized by their autofluorescentce. Plaques have a unique structural appearance like a hairy star of the size of approximately 3 microns or more. The reagents can be also applied applied in vitro to mounted slides after fixation, incubated for 20-30 minutes in the plaque reagent and then washed exhaustively.

In agreement with in vivo obtained labeling, the highest level of plaque labeling was obtained for the P/LLL/M4/D1 mini nanodrug, and the second highest level was obtained for the mini nanodrug P/LLL/AP-2/D1. Background targeting was observed for the nanodrugs lacking D1, D3, ACI-89 peptides.

It was shown that the D 1-peptide nanodrugs containing the D1-peptide in addition to one of the other peptides AP-2, M4 or B6 used for BBB crossing indeed targeted amyloid plaques, after BBB crossing.

Referring to FIGS. 23A-23B, this is shown by the figure showing that plaques are more intensively stained after iv injection of P/LLL/M4/D1-peptide/rhodamine mini nondrug (FIG. 23B; referred to in the figure as P/LLL/M4/D1-peptide) in comparison with staining after iv injection with P/LLL/M4/rhodamine (FIG. 23A; referred to in the figure as P/LLL/M4). The result shows that the nanodrugs, such as P/LLL/M4/rhodamine, can be used for further targeting inside brain by carrying additional peptides, such as D1. The bar-panels of FIG. 23C show quantitatively the effect of increased staining plaques in the presence of conjugated D1 compared to staining in the absence of D1.

### Example 17

### Mini Nanodrugs Targeting Amyloid Plaques Cross Blood-Brain Barrier (BBB)

The localization of two mini nanodrugs, P/LLL/AP2/rh and P/LLL/D1/rh was examined optically as described herein. FIG. 24 illustrate that the BBB-penetrating peptides AP2 vs. D-peptides allow different degrees of trans-BBB nanoconjugate transport.

FIG. 24 is a set of photographs illustrating optical imaging data of the brain cortex following the injection of mice with 0.274 μmol/kg of P/LLL/AP2/rh (bottom), or P/LLL/D1/rh (top). Optical imaging data showing nanoconjugate permeation of the cerebral cortex: nanoconjugate fluorescence is red (left column) and grey (right column) and the vasculature is green. Cell nuclei are blue. Arrows indicate particulate fluorescence and asterisks indicate diffuse, extracellular fluorescence.

The mini nanodrugs are recognized as diffuse staining, the localization being comparable except that the extravascular fluorescence levels in the case of P/LLL/D1/rh is elevated compared to P/LLL/AP2/rh as reference. Referring to FIG. 24, right panel, the difference is especially noticeable in the grey-scale pictures which, to some extent, resolve fluorescence colocalization in cells more so for D 1-conjugated than for AP2-conjugate. Similar distributions were measured when mini nanodrugs carrying the D-peptides ACI89 (P/LLL/ACI89/rh) and D3 (P/LLL/D3/rh) were injected. The mini nanodrug contents in the brain parenchyma was quantified by measuring the ‘diffuse’ nanoconjugate fluorescence, including diffuse fluorescence in the cells, however, excluding fluorescent particulates. Intensity was measured in twenty 10×10 μm-sized regions of interest (ROI) per image (yellow squares in FIG. 24, right panels). These regions of interest were placed away from the vasculature, thus containing only fluorescence in the brain parenchyma. The resulting histograms are similar to data shown in FIG. 23B. They are arranged for comparison in the three brain regions Layers II/III Cortex, Hippocampus CA1-3, and Midbrain Colliculi. In each region, a significant increase in fluorescence was observed for animals that were injected with P/LLL/D1/rh, P/LLL/D3/rh, and P/LLL/AC189/rh in comparison with P/LLL/AP2/rh. The regional distribution indicates a higher presence of mini nanodrugs in Midbrain compared with Cortex and Hippocampus.

### Example 18

### Displacement of P/LLL/D-Peptides/rh by AP2 Indicates Similarity of Binding Sites for AP2 and D-Peptides Involving LRP-1 Mediated Trans BBB Crossing

Available evidence indicates that the shuttle peptides AP2 and D1, D3, and AC189 have in common the LRP-1 transcytosis pathway. The D-peptides D1, D3 and AC189 in their original L-configuration were isolated by phage display using Aβ₁₋₄₂ as target (Funke et al. (2012) PLoS ONE 7 (7): e41457, which is incorporated by reference herein as if fully set forth).

The D-configured peptides were chemically transcribed into D-configured peptides (D-peptides) which, like the original L-peptides, could be used to target Alzheimer's disease-associated Aβ₁₋₄₂ containing plaques in the brain parenchyma and for BBB crossing abilities (Jiang et al, 2016 Biochimica et biophysica acta, 1858 (11), 2717-2724; Wiesehan et al., 2003, Chembiochem: a European journal of chemical biology, 4 (8), 748-53; and Zheng et al, 2017, Acta Biomater, 49, 388-401, all of which are incorporated herein by reference as if fully set forth). As LRP-1 mediates brain homeostasis by shuttling Aβ-peptides from the parenchyma to blood vessels, AP2 and D-peptides function as shuttle peptides to transvers BBB, and AP2 is known to bind LRP-1, it is arguable that AP2, amyloid A13, and D-peptides have in common the LRP-1 pathway (Demeule et al., 2008, Journal of neurochemistry, 106, 1534-1544; Demeule et al., 2008, J Pharm Exp Ther 324,1064-1072; Wiesehan et al., 2003, Chembiochem: a European journal of chemical biology, 4 (8), 748-53; and Herr et al., 2017, Frontiers in molecular neuroscience, 10, 118, all of which are incorporated herein by reference as if fully set forth). By using the optical method, the hypothesis whether AP2 interferes with the D-peptide-guided BBB transfer was investigated. As an example, the AP2 dependent inhibition of P/LLL/D1/rh BBB crossing efficacy was measured. A competition experiment, in which P/LLL/D1/rh was co-injected with a 70-fold molar excess of AP2 peptide was designed. The hypothesis was that excess AP2 would impair trans-BBB movement of P/LLL/D1/rh, if it competed for the binding of the receptor on the transcytosis pathway. FIGS. 25A-25B illustrate optical imaging data of brain parenchyma following injection of mice with 0.274 μmol/kg of P/LLL/D1/rh and 0.274 μmol/kg P/LLL/D1/rh+21 μmol/kg of AP2 (top). FIG. 25A is a set of photographs illustrating optical imaging data of the brain cortex following the injection of mice with 0.274 μmol/kg of P/LLL/D1/rh (bottom), and 0.274 μmol/kg P/LLL/D1/rh+21 μmol/kg of AP2 (top). Optical imaging data showing P/LLL/D1 nanoconjugate permeation of the cerebral cortex when co injected with AP2 peptide (top): Nanoconjugate fluorescence is gray (right image), and the vasculature is green while the nanoconjugate is in red in the merged image (left). For comparison, P/LLL/D1 images of the same dose are shown on the bottom. FIG. 25B are bar graphs illustrating the intensity of fluorescence in the samples of the brain parenchyma following injections of mice with 0.274 μmol/kg of P/LLL/D1/rh, P/LLL/D1/rh+21 μmol/kg of AP2 or PBS buffer in layers II/III cortex (left), midbrain colliculi (middle) and hippocampus CA₁₋₃ (right). Average nanoconjugate fluorescence in layers II/III of the somatosensory cortex, the midbrain colliculi, and the hippocampal CA1-3 cell layers when co injected with AP2, P/LLL/D1 injected alone and compared to PBS. Average nanoconjugate fluorescence measurements were obtained from 20 randomly sampled ROIs explicitly outside of the cerebral vasculature (3 mice with 3 images each, for each measurement). The results are indicated with asterisks where *=p<0.01, **=p<0.001, and ***=p<0.0001.

As shown in FIGS. 25A-25B, the P/LLL/D1/rh fluorescence intensity in the parenchyma dropped significantly, indicating that AP2 peptide blocked approximately 50% of the transcytosis pathway. Since the AP2 route of transcytosis via the LRP-1 mechanism is established, results indicate that the free AP2 decreases P/LLL/D1/rh transcytosis by competing with the binding of the conjugate. An incomplete elimination of the transcytosis is in accordance with the ascribed conjugate P/LLL residue contributing a shuttle peptide-independent BBB “boosting” of BBB permeation. The higher efficacy observed for the D-conjugates compared with the AP2 conjugate (FIG. 24) can be ascribed to several factors: (1) higher affinity of the D-residues to receptor binding, (2) higher stability of D-peptides against hydrolytic cleavage, (3) improved geometrical presentation of the nanoconjugates during transcytosis, and (4) cell located fluorescence (Miller, et al., 1995, Drug Development Research, 35 (1), 20-32; Liu et al., 2016, Chemical record (New York, N.Y.), 16 (4), 1772-86; and Jiang et al, 2016 Biochimica et biophysica acta, 1858 (11), 2717-2724, all of which are incorporated by reference as if fully set forth). Notably, in case AP2 and D1 were conjugated together on the same platform molecule, as in P/LLL/AP2/D1/rh (FIG. 23B), less parenchyma fluorescence was observed compared to injections of P/LLL/D1/rh alone. This indicates an improved geometrical presentation for the all-D1-conjugates.

### Example 19

### Distribution of Mini Nanodrugs Targeting Amyloid Plaques as a Function of Their Distance to Vasculature

Having compared the efficacy of BBB transgression into parenchyma for conjugates of AP2 and D-peptides, their diffusion following the vasculature egression into the parenchyma was investigated. The resulting data were used as a metric to examine how well the mini nanodrug was able to travel from the vessels into the surrounding parenchyma and encounter brain cells. FIGS. 26A-26B are scatter plots and line graphs illustrating drug penetration distance through the brain parenchyma extracellular matrix (the intensity of fluorescence vs. distance from the nearest blood vessel) calculated for P/LLL/AP2/rh, P/LLL/AC189/rh, P/LLL/D1/rh and P/LLL/D3/rh in the cortex (FIG. 26A) and hippocampus (FIG. 26B). Nanoconjugate diffused fluorescence intensity vs. ‘distance from vasculature’ measurements in brain parenchyma of mice injected with nanoconjugates at a dose of 0.274 μmol/Kg. Fluorescence measurements were obtained from 10 mm²-sized regions of interest (ROI) that were randomly overlaid on regions devoid of vasculature (shown by yellow squares in FIG. 22A). Intensity measurements and positions were then obtained for each ROI and plotted against the location of the nearest blood vessel wall. The X axis is set at 5, which is higher than the PBS background level detected (4.6). As shown in FIGS. 26A-26B, both in the cortex (FIG. 26A) and hippocampus (FIG. 26B), fluorescence intensity decreases as a function of distance. Mini nanodrug carrying the D-peptide showed more fluorescence than the same mini nanodrug conjugated with AP2. The higher fluorescence correlated with the high fluorescence proximal to the vasculature, and it showed higher intensity in the cortex than in the hippocampus. Best fits were calculated for exponentials in the case of AP2, D1, AC189, and D3 peptides (cortex). Comparing the power of the fits, showed P/LLL/AP2/rh at r²=0.3636 and P/LLL/ACI89/rh at r²=0.355; these were the closest fits to presenting an exponential decay behavior (a perfect fit will have an r²=1). On the other hand, P/LLL/D3/rh had a value of r²=0.2726 and P/LLL/D3/rh, r²=0.186, showed less of a fit to the exponential behavior. Referring to FIGS. 26A-26B, considering that the curves shown in these figures are nearly parallel, the higher fluorescence of the mini nanodrug in distal brain tissue regions was attributed to enhanced BBB penetration, rather than to the tendency for faster decay. This finding is illustrated by the γ-intercepts of the D-peptide fluorescence curves when compared with curves for the AP2-carrying nanoconjugate (cortex Y intercepts: 14.7 for P/LLL/AP2, 20.9 for P/LLL/ACI89, 21.1 for P/LLL/D3, and 21.6 for P/LLL/D1). The results illustrated on FIGS. 26A-26B indicate that the overall effect of extracellular matrix composition may not greatly vary in the case of the peptide conjugates. However, a high variability of individual peptide localization was noted.

The distribution demonstrates that the mini nanodrugs penetrated many microns deeply into the parenchyma through the extracellular matrix. The interpretation of parenchyma PK studies for P/LLL/AP2/rh and the observation of amyloid peptides crossing BBB from the parenchyma to blood capillaries are in agreement with retrograde movement of mini nanodrugs out of the parenchyma, thus counteracting deep parenchyma distribution. Referring to FIGS. 26A-26B, the deeper distribution of the mini nanodrugs carrying D-peptide vs nanodrugs carrying AP2 in could be due to less retrograde diffusion of the D-peptide nanoconjugates. The concentration could be opportune for interactions with neuron cells, microglia, and astroglia.

### Example 20

### D-Peptide Conjugates Distribute to Brain Cells Through Amyloid Peptides

The D-shuttle peptides had been selected by their molecular interaction with amyloid peptides. The possibility of D-peptide conjugates to distribute into neurons and other cells, which harbor amyloid peptides were considered. FIGS. 27A-27C illustrate fluorescence uptake in the hippocampus and cortex neurons and astroglia. FIGS. 27A and 27B are set of photographs of neurons and astroglia in hippocampus (FIG. 27A) and cortex (FIG. 27B) of animals that were injected with PBS and P/LLL/ACI89. PBS (background) or drug are shown in red, neurons are shown in yellow, nucleus in blue, and astroglia are shown in green. FIG. 27C is a set of photographs showing the drug fluorescence (left) and merged (right) only for P/LLL/ACI89 nanoconjugate. The white arrow points to a non-labeled astroglia, the yellow arrow to a labeled astroglia, and the purple arrow to a labeled neuron. All statistical tests were conducted as a one-way ANOVA with post-hoc Tukey t-tests. Statistical significance is indicated as follows: *=p<0.01, **=p<0.001, and ***=p<0.0001.

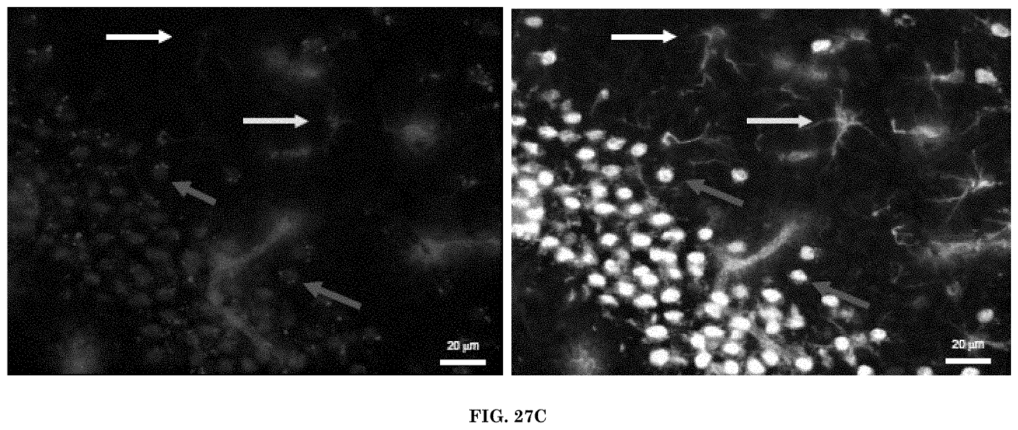

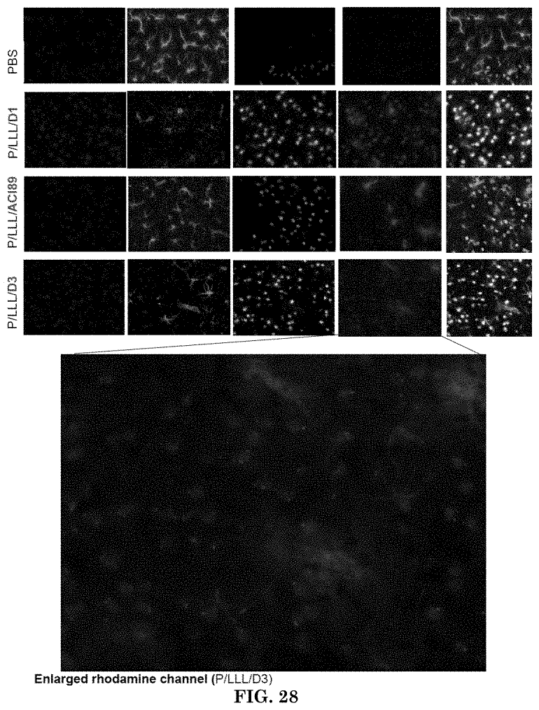

FIG. 28 is a set of photographs showing fluorescence uptake in the cortical layer II/III (B) neurons and astroglia in cortical layers II/III of animals that were injected with P/LLL/D1/rh, P/LLL/ACI89/rh, P/LLL/D3/rh and PBS. PBS or drug is shown in red, neurons are shown in yellow, nucleus in blue and astroglia are shown in green. In the bottom, an enlarged figure of the drug fluorescence only for P/LLL/D3 nanoconjugate.

Referring to FIGS. 27A-27C, stronger particle fluorescence was for D-peptide conjugates and also a notable fluorescent staining of the cellular matrix (P/LLL/D1/rh vs. P/LLL/AP2/rh, as was also shown in FIG. 24). Altogether, three regions of fluorescence were observed. First, diffuse staining not confined to cells, but in the form of soluble conjugates moving through parenchymal extracellular space (asterisks in FIG. 22A). Second, subcellular located diffuse fluorescence as discussed for D-peptide containing conjugates (grey-scale in FIG. 24, and red fluorescence in FIGS. 27A-27C). Third, particulate fluorescence in neurons and less in other cells, located proximal to the nuclei (arrows in FIGS. 24 and 27C).

### Example 21

### The Consideration of Brain Cell Uptake Forming Fluorescent Particles

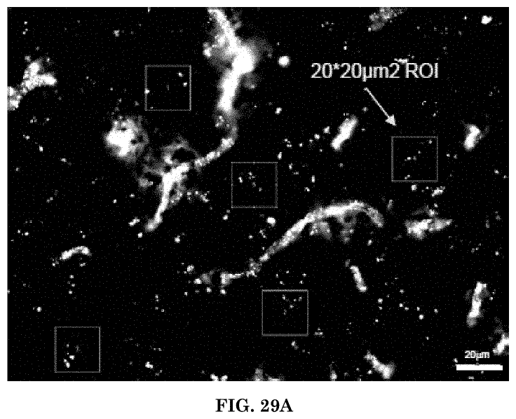

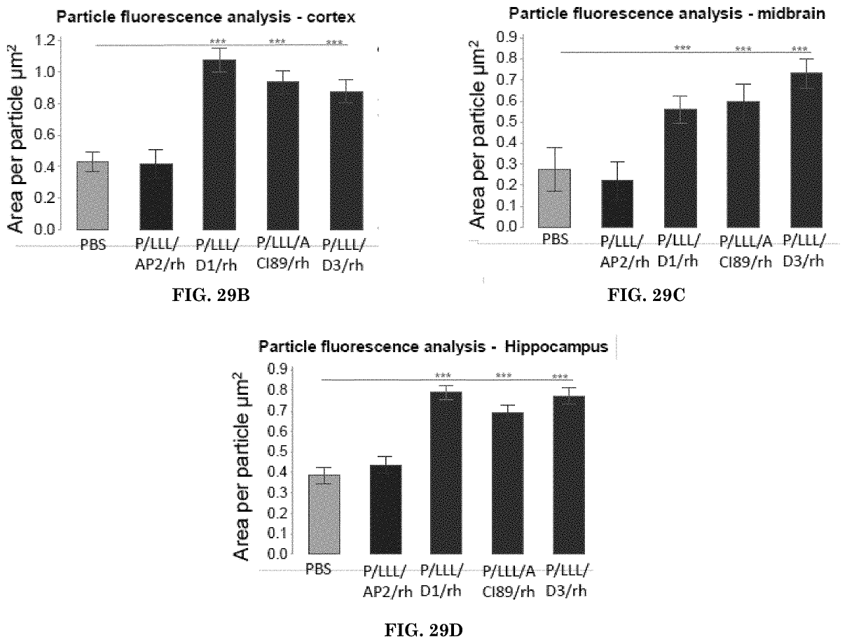

All particles detected for the D1-peptide conjugate were found in intracellular perinuclear locations, and all other D-peptide conjugates (meaning D3 and ACI89) showed consistent results. In order to strengthen that D-peptide conjugates vs. AP2-peptides were incorporated in higher amounts, we further compared the size of the particles. A more reliable method to separate the particulate from the cellular fluorescence was reasoned to be by adjusting the levels of the image and converting it to a binary image, allowing the detection of only particulates eliminating interference of the diffused background. FIGS. 29A-29D illustrate intracellular fluorescence of mini nanodrugs.

FIG. 29A is an image of the P/LLL/D1 conjugate which demonstrates the method: 20*20 μm² ROIs were placed randomly however away from vessels for each image. Each ROI was converted to binary (black and white) image and the area and number of particles were quantified. 3 images per brain area were tested for 3 mice per group.

FIGS. 29B-29D illustrate intracellular accumulation of measured ROI as average area per particle in samples of the brain following injections of mice with P/LLL/AP2/rh, P/LLL/D1/rh, P/LLL/AC189/rh, P/LLL/D3/rh, or PBS in cortex (FIG. 29B), midbrain (FIG. 29C) or hippocampus (FIG. 29C). Average area per particle is significantly higher for P/LLL/D1, P/LLL/ACI89 and P/LLL/D3±S.E.M compared to PBS and P/LLL/AP2±S.E.M in the cortex (FIG. 29B), midbrain (FIG. 29C), and hippocampus (FIG. 29D). The results are indicated with asterisks where *=p<0.01, **=p<0.001, and ***=p<0.0001. The lines at the top of the bars show the average area per particle of PBS and P/LLL/AP2/rh (correspondingly) against which each comparison was made.

The particle content (example of ROI in FIG. 29A) then was measured. The average size of the particulate fluorescence is a proxy to estimate the intracellular load of fluorescent nanoconjugates following their injections. An example of such an analysis is shown in FIG. 29A: the yellow squares indicate regions of interest that were used to quantify particulate fluorescence. FIGS. 29B-29C summarize the average size of the intracellular fluorescence particles in the cortex (FIG. 29A), midbrain (FIG. 29C), and the hippocampus (FIG. 29C). The particles after injection with P/LLL/AP2 have similar sizes as measured after injection of PBS and thus do not form particulates (e.g. cortex: P/LLL/AP2=0.42±0.72 and PBS=0.43±1.17 μm²; Tukey test: p=1.000). In contrast, nanoconjugates containing D-shuttle peptides P/LLL/D1, P/LLL/D3, and P/LLL/ACI89 were measured to form particles at a 2.5-fold increased diameter in comparison with PBS (One-way ANOVA: p=0.000; Tukey-tests: P/LLL/D1 t=6.4, p<0.0001; P/LLL/ACI89 t=5.13, p<0.0001 and P/LLL/D3 t=4.48, p<0.001). These data show that systemically injected D-peptide not only entered brain parenchyma at increased efficiency but also targeted and accumulated forming particulates in certain brain cells.

### Example 22

### Distribution of Fluorescent Particles in Neurons and Glia Cells

According to their specificity through selection by phage display, it was observed that D-peptides target amyloid peptides not only on plaques but also when located on neuron and glia cell surfaces. When the D3 peptide alone was injected in mice entorhinal cortex or infusion into the hippocampus, neuronal uptake was observed. That phenomenon was explained by the binding of D3 to APP or Aβ which are axonally transported, which then carries it into the cell. Accordingly, these conjugates would not only locate on the cell surface but also, should eventually internalize, into neuron and similarly but to less extant into glia cells, using the same mechanism. To this end, after systemic injection of our D-conjugates we counterstained brain tissue sections of hippocampus and cortex with anti-Neun to label neurons, and with anti-GFAP to label astroglia (FIGS. 27A-27C) and inspected the stained sections for red fluorescent particulates. All particulates were located next to cell nuclei and appeared to be part of the stained cells. Positive neurons and glia cells were counted after systemic injection of conjugates containing D1, D3, AC189 peptides, and of a control PBS (FIGS. 27A-27C). Cell were considered positive for drug when both particulate and diffused fluorescence were detected with visibly higher intensity than the PBS background (FIGS. 27A and 27B). There was almost no particulate fluorescence detected in glia in PBS mice (FIGS. 27A and 27B). The results are summarized in Table 4. For P/LLL/ACI89/rh, 93.9% of neurons were found to contain fluorescent particles as well as visible diffuse fluorescence around the nuclei (FIG. 27C, purple arrow). In contrast, only 8.7% of astroglia contained drug (FIG. 27C, yellow arrow for positive astroglia, white arrow for negative astroglia), indicating the preferential uptake of D-peptide conjugates by neurons. A similar GFAP and NEUN staining for P/LLL/AP2/rh (FIG. 28) showed no cell association. The numbers for PBS were insignificant and with much lower intensity, probably reflecting lipofuscin particles and compared with numbers after injection of P/LLL/AP2/rh.

TABLE 4
Percentage of cells containing fluorescent drug particulate
after injection of rhodamine labeled conjugates P/LLL/D1,
P/LLL/D3 and P/LLL/ACI89. The content of lipofuscin
labeled vesicles was negligible.

| | Mini nanodrug % of positive cells | | | |
|---|---|---|---|---|
| | [#Labeled/#total counted cells] | | | |
| | Cortex | | Hippocampus | |
| | astroglia | neurons | astroglia | neurons |
| P/LLL/D1 | 12.5 | 84.4 | 7.0 | 66.0 |
| | [5/40] | [119/141] | [4/57] | [286/433] |
| P/LLL/D3 | 15.2 | 95.3 | 29.8 | 92.45 |
| | [7/46] | [286/300] | [14/47] | [894/967] |
| P/LLL/ACI89 | 8.7 | 93.9 | 17.5 | 97.6 |
| | [4/46] | [199/212] | [7/40] | [887/909] |

### Example 23

### Fluorescence Distribution and D-Peptide Conjugate Uptake by Brain Cells

The causative correlation of the diffuse fluorescence in FIGS. 24 and 28A-28C with the efficacy of systemic injection of mini nanodrugs that carry D-peptide was examined. Since particulate and diffused fluorescence around neurons could not be measured separately, the combination of both to achieve fluorescence intensity of neurons was first measured and then compared with the average particulate fluorescence (FIG. 29B-29D).

FIGS. 30A-30B illustrate fluorescence in neurons following mice injection with mini nanodrugs. FIG. 30A is a set of photographs of neuron staining and optical imaging of the brain following injections of mice with 0.274 μmol/Kg of P/LLL/D3/rh: neuron nucleus (yellow, Neun) surrounded with ROIs (top left), drug (grey, rhodamine channel) and ROI's (yellow) (top right) and drug only (grey) (bottom). FIG. 30B are bar graphs illustrating average fluorescence per neuron nucleus, after PBS deduction of P/LLL/D3/rh (0.274 μmol/Kg), P/LLL/D1 (0.274 μmol/kg), and P/LLL/ACI89 (0274 μmol/Kg). All statistical tests were conducted as a one-way ANOVA with Tukey t-tests conducted between experimental conditions in each brain regions. Statistical significance is indicated as follows: *=p<0.01, **=p<0.001, and ***=p<0.0001. Referring to FIG. 30A, each cell, was regarded drawing a ROI containing the nucleus and the perinuclear particles. Referring to FIG. 30B, then the average fluorescence intensity was measured for D1, D3, AC189, and PBS (control). Fluorescence values for the peptide nanoconjugates were significantly higher than for PBS (One-way ANOVA: p=0.000; Tukey-tests: P/LLL/D1/rh t=8.53, p<0.0001; P/LLL/ACI89/rh t=4.14, p<0.0001 and P/LLL/D3/rh t=15.13, p<0.001) and outstanding for P/LLL/D3/rh. AP2.

In the case of glioblastoma, a brain tumor, systemically injected anti-transferrin receptor antibody-targeted P/LLL conjugates (1) accumulated via a receptor-mediated mechanism in endosomes identified microscopically as particulates in brain tumor cells, and (2) the P/LLL group promoted the release of the conjugates from the endosome into the tumor cell cytoplasm. It was assumed that the D-peptides function in targeting delivery through BBB and forming particle-like endosomal uptake in brain cells. The LLL could also mediate release of the conjugates from endosomes into cytoplasm explaining the occurrence of intraneuronal fluorescence.

### Example 24

### Distribution of Morpholino Antisense Oligo Nucleotide (AON) Attached to P/LLL/D-Peptides

BBB-penetration and brain cell internalization of mini nanodrugs is useful for drug delivery in the treatment of neurodegenerative diseases. The results in FIGS. 29B-29D and 30B favor the conjugates P/LLL/D1 and P/LLL/D3 as delivery platforms. Of particular interest was the delivery of Morpholino antisense oligo nucleotides (AONs). To provide experimental evidence for the delivery, P/LLL/D3/AON fluorescence labeled at AON or polymalic acid residues conjugates were synthesized, to follow their transgression through BBB and internalization into neurons after intravenous injection of healthy mice. The delivery has been demonstrated successful in the antibody targeted preclinical treatment of glioblastoma and of metastases in the brain. Accordingly, the P/LLL/D3 and P/LLL/D1 platforms were tested for AON delivery into brain cells. To unequivocally demonstrate the delivery of AONs, carboxy-fluorescein labeled mock AON (SEQ ID NO: 13) was used (AON-F), and P/LLL/AON-F chosen as a negative control. Each mouse was injected with a dose of 500 μg AON-F (60 nmol; 0.4 mmol/kg)

Mice were euthanized two hours post injection and optical imaging data were collected for cortex following the method described on FIG. 24 for diffuse fluorescence in cortex and in FIG. 29A for the fluorescence of particulates in brain cells.

FIGS. 31A-31C are optical imaging data following mice injections with mini nanodrugs that carry AONs.

FIG. 31A is a set of photographs showing optical imaging data in the samples of the brain cortex following mice injection with P/LLL/D1/AON-F, P/LLL/D3/AON-F and P/LLL/AON-F. Combined images on the left show lectin stained vessels in red, labeled nanoconjugate in green, and DAPI in blue. The correlating binary image used to calculate particulate fluorescence is shown to the right.

FIG. 31B are bar graphs showing data of the diffused fluorescence measurements in the cortex following mice injection with P/LLL/AON-F, P/LLL/D1/AON-F, and P/LLL/D3/AON-F.

FIG. 31C are bar graphs showing data of the particulate fluorescence analysis (area per particle, μm²) in the cortex following mice injection with P/LLL/AON-F, P/LLL/D1/AON-F, and P/LLL/D3/AON-F. All statistical tests were conducted as a one-way ANOVA with post-hoc Tukey t-tests. Statistical significance is indicated as follows: *=p<0.01, **=p<0.001, and ***=p<0.0001.

As shown on FIG. 31C, delivered P/LLL/D3/AON-F produced significantly larger particles than the PBS control (ANOVA: p=0.000, F=118.09) or the untargeted P/LLL/AON-F. A similar result was obtained for injected conjugates and PBS shown on FIG. 31B indicating the diffuse fluorescence in extracellular cortex (29.45 a.u. fluorescence; Tukey p<0.0001); next P/LLL/D3/AON-F with a value of 33.46 (P-value 0.000, T value 12.27). The fluorescence intensity for P/LLL/D3/AON-F was significantly higher than the intensities for PBS and P/LLL/AON-F (P-value 0.000, T-value 6.84) and indicates that this conjugate was an efficient deliverer of AON to the brain cells. The higher efficacy in comparison with P/LLL/D1/AON-F (FIG. 31C) is in agreement with the inverse situation shown on FIG. 31B, showing higher efficiency of accumulation in the extracellular cortex. It was hypothesized that the favorable cellular uptake of P/LLL/D3/AON-F causes the observed extracellular depletion which is not visible in the case of P/LLL/D1/AON-F which affords a minor decrease in the parenchyma due to less cellular uptake.

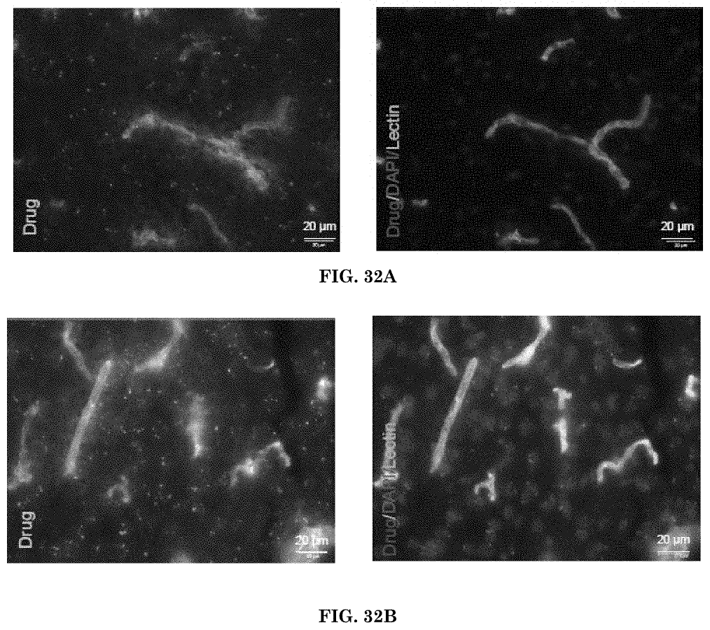

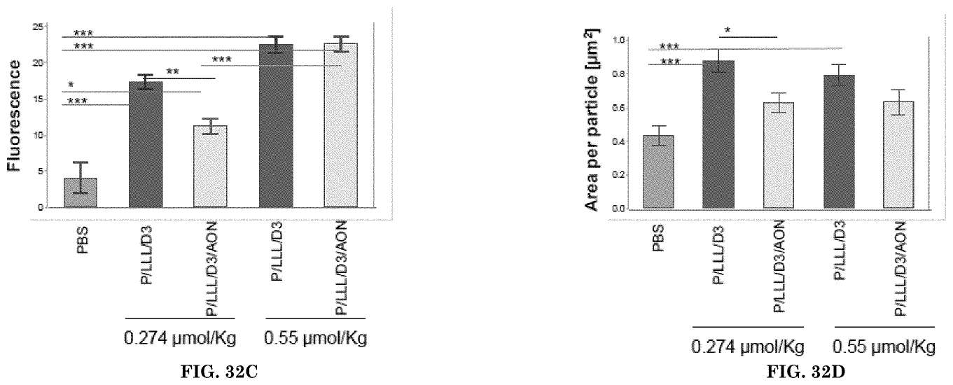

After having demonstrated the uptake of P/LLL/D-peptide/AON-F, the effect of doubling the injected dosage for P/LLL/D3/rh/AON on the level of fluorescence in the parenchyma (diffusible nanoconjugate) and the area of fluorescence emitted by the particles after internalization into the brain cells was tested. The results are shown on FIGS. 32A-32D. FIG. 32A is a set of photographs showing optical imaging data in the brain cortex following injection of the mice with 0.274 μmol/Kg of P/LLL/D3/AON/rh. FIG. 32B is a set of photographs showing optical imaging data in the brain cortex following injection of the mice with 0.55 μmol/Kg of P/LLL/D3/AON/rh. FIG. 32C are bar graphs showing data of the diffused fluorescence measurements in the cortex and dose dependence following injection of the mice with P/LLL/D3/AON/rh, P/LLL/D3/rh or PBS. FIG. 32D are bar graphs showing data of the particulate fluorescence analysis (area per particle, μm²) in the cortex following mice injection with P/LLL/D3/AON/rh, P/LLL/D3/rh or PBS.

While the fluorescence in the parenchyma doubled in agreement with the doubled dose, the particle size, after internalization, was unvaried (FIG. 32C) and suggested that the capacity of the “particles” was limited.

### Example 25

### Shuttle Peptide Structure Drives Efficacy of BBB Permeation, Parenchyma Penetration, Brain Cell Uptake and AON Delivery

The scope of the investigation was to test whether the shuttle peptide dependent penetration of BBB could be used as a pharmacological tool to deliver antisense oligonucleotides across the barrier into cells of healthy brain. This was achieved with D-configured peptides, but not with Angiopep-2 conjugated with the same polymeric platform. The D-peptides and Angiopep-2 however competitively used the LRP-1 transcytosis pathway for crossing BBB. In the parenchyma, the mini nanodrugs migrated many microns deeply into the brain through the intercellular matrix, the D-peptide conjugates further than the Angiopep-2 conjugates. Because of their selection by phage display to bind Aβ, the D-peptides recognized neurons and to a lesser extent glia cells and thus owned the activity to target these cells. The mechanism of targeting has not been resolved but the targeting specificity of the D-peptides suggest their bonding to cell surface-amyloid peptides or to unknown D-peptide recognizing receptors. The binding was followed by internalization and storage into particles. For the purpose of delivering antisense oligonucleotides (AON) active in the inhibition of protein synthesis they were reversibly conjugated to the polymer scaffold by disulfide bonding. The mini nanodrugs delivered the AONs efficiently over BBB into neurons and in a capacity limited amount into the particles. In analogy to endosome involved delivery in brain tumor treatment, AONs in neurons will inhibit mRNA dependent protein synthesis. The described findings could help establish cell targeted drug treatment of neurodegenerative diseases.

It was shown that Aβ-targeting D peptides can be conjugated to a polymeric nanocarrier and that they cross the intact BBB better than a nanoconjugate that is conjugated to an AP2 peptide. AP2 has been used increasingly as a ‘go-to’ peptide to shuttle a variety of nanoplatforms across the BBB, given that other peptides have, by comparison, shown less BBB penetrating ability. The data indicate that Aβ targeting D peptides, provided that they are conjugated to PMLA, enhance BBB penetration of nanocarriers. Also, these mini nanodrugs showed better penetration into the parenchyma when the distance was measured, as well as promoted neuron targeting and entry. Furthermore, it was shown that these mini nanodrugs can be modified with antisense oligo nucleotides, can cross the BBB and can be delivered into cells, preferably neurons. The same approach can be applied to include more Nano systems such as micelles, liposomes, polymeric nanoparticles, and crystalline nanoparticles like iron oxide and gold. This data can also help design better nanocarriers that can travel further into the parenchyma and get into neurons for therapeutic purposes such as gene mediated therapy.

### Example 26

### Mini Nanodrugs Containing IgG-rh Antibodies

There can be benefits of combining the antibodies and peptides in the synthetic nanoconjugates designed for targeting specific diseases. Antibodies can be obtained through immunization, and are readily available in the field. Antibodies bind with extreme affinities to targets in the brain. Additionally, very low concentrations of antibodies are needed for efficient targeting. With mini nano carriers capable of penetrating through BBB, the concentrations for efficient specific reactions of delivered antibodies within the brain after BBB crossing can be easily attained. These mini nanocarriers can be used for disease treatments as well as for scientific exploration of the targeted reactions in the brain that includes imaging.

The mini nanocarriers containing IgG antibody, peptide(s) and an additional payload were constructed for targeted transcytosis through BBB. The mini nanocarriers containing rhodamine-labeled IgG as follows.

Nanocarriers with additional payloads has been described to follow very similar synthetic routes as given above for single peptide nano carriers.

The payload in these mini nanocarriers can be additional peptides, an antibody and/or antisense oligonucleotides or other synthetic drugs used for delivery through BBB. A typical payload can be a peptide. The peptide dependent transport though BBB had been found to have an increased efficacy, if tri-leucine (LLL) was conjugated to the polymalic acid-peptide platform. It has been described herein that the polymalic acid/LLL (40%) itself permeated through the barrier. The peptides either serve targeting through BBB permeation pathways by binding to pathway specific receptors. Additional (payload) peptides or antibodies will target receptors on brain cells (microglia or neurons) or polypeptide plaques like in the treatment of Alzheimer's disease. Antibodies as payloads will have the same functions as payload peptides, however they are more easily available and have higher receptor binding affinities that is of advantage if their targeting is designed to function downstream of reaction cascades. The consecutive function of peptides and antibodies to pass a series of barriers is designed to target diseased cells such as tumor cells or aberrant microglioma or neuronal cells. These cells may receive drugs attached to the polymeric platform for treatment by driving these cells into apoptosis or necrosis.

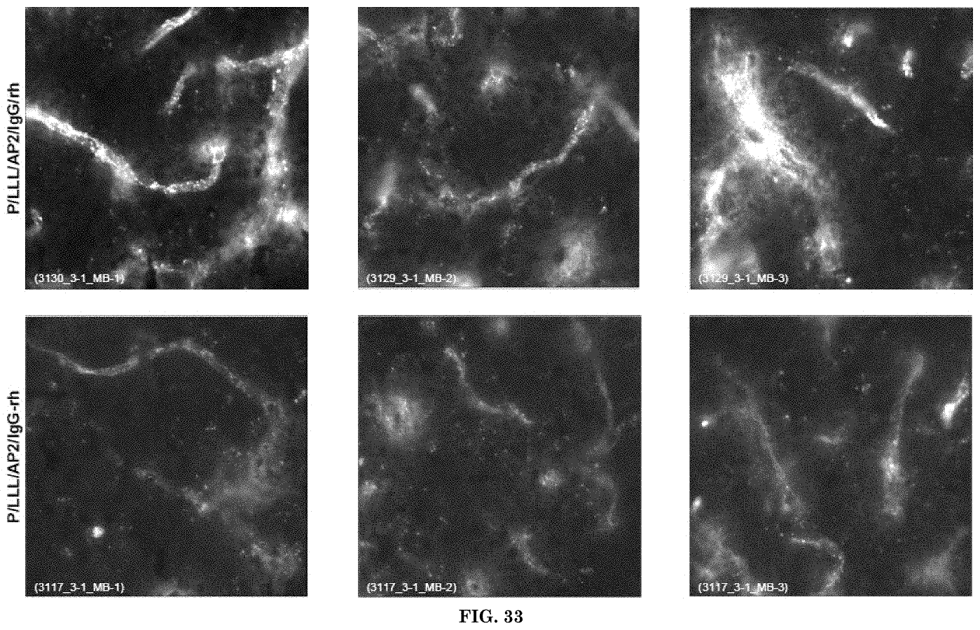

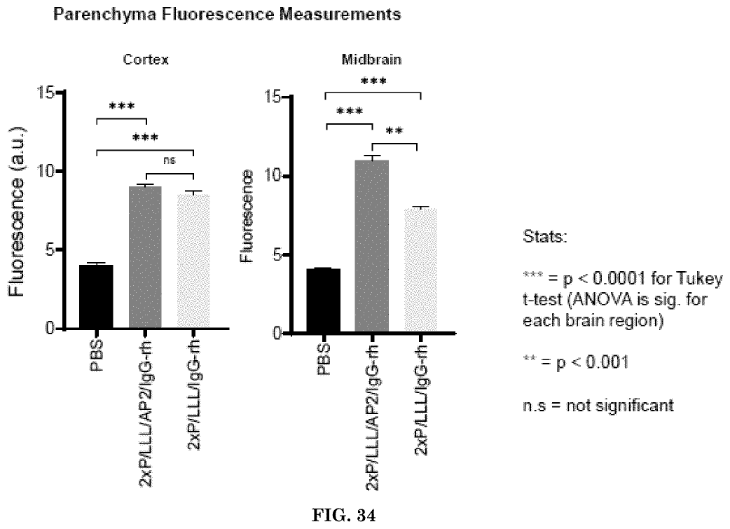

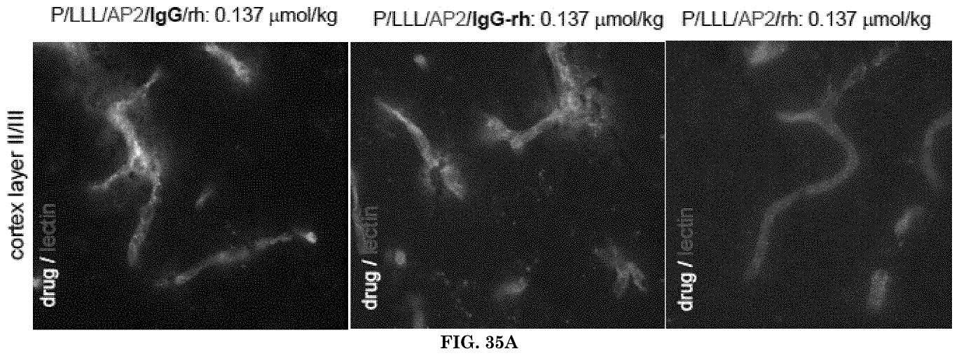

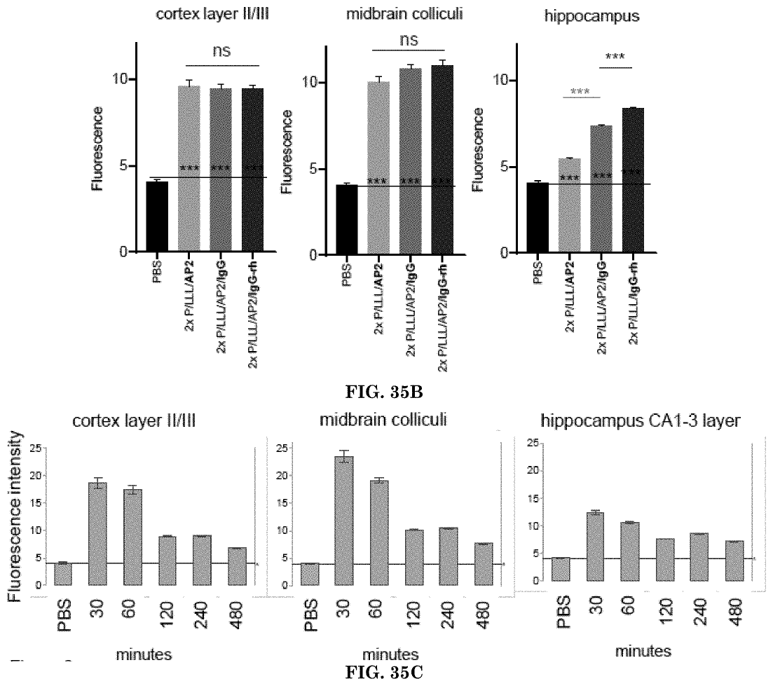

The BBB permeation of an antibody as payload is shown in FIGS. 34 and 35. FIG. 33 is set of photographs illustrating optical imaging data of midbrain following mice injected with P/LLL/AP-2/IgG, in which P (polymalic acid backbone) is labeled with rhodamine for fluorescence (top row) and P/LLL/AP-2/IgG-rh, in which IgG is labeled with rhodamine for fluorescence (bottom row). All panels (left, middle and right) of the top and bottom rows shows images taken from midbrain regions of the brain. Staining of these regions has been performed by identical batches for each agent indicated. Each panel shows blood capillaries filled with the nano agent. In addition, the images shows extruding nanoagents through the BBB walls of each vessel.

Referring to FIG. 33, the nanoconjugates were injected into the tail vein of different mice at a dose 0.137 μmol/kg. After 120 minutes following the injection, the mice were sacrificed, and the brain fixed and stained for vasculature as described. The microtome-sliced brain was visualized under fluorescence microscope as described in examples herein. It was observed that the BBB permeation efficacy of the nanoconjugates that carry the IgG labeled with rhodamine was lower (bottom row) compared to that for the nanoconjugates, in which the PMLA backbone was labeled with rhodamine (top row) as was assessed by visualizing the rhodamine fluorescence. Diffused staining observed on the images (FIG. 33, bottom row) indicates distribution of the nanoconjugate P/LLL/AP2/IgG-rh (IgG labeled with rhodamine) from vasculature into parenchyma by visualizing the fluorescence of rhodamine. The data shows that fluorescence in the midbrain resulted from the rhodamine-labeled antibody crossing the BBB. The experiment rules out an erroneous result, for example, provoked by hydrolysis of PMLA into fragments which would then be able to permeate as small compounds the blood-brain barrier.

Fluorescence data for the P/LLL/AP-2/IgG-rh nanoconjugate and P/LLL/IgG-rh, the simplified version of the nanoconjugate that did not contain AP-2, was compared. FIG. 34 are bar graphs illustrating the intensity of fluorescence in the samples of the brain following injections of mice with P/LLL (40%)/AP-2/IgG-rh (0.2%), P/LLL (40%)/IgG-rh (0.2%) or PBS buffer in cortex (left graph) and midbrain (right graph). Fluorescence data for P/LLL (40%)/IgG-rh were obtained in the same way for both these brain regions. It was demonstrated herein that both P/LLL (40%)/IgG and P/LLL (40%)/AP-2/IgG permeate the BBB. However, P/LLL (40%)/AP-2/IgG permeates the BBB with higher efficacy compared to P/LLL (40%)/IgG.

Additional PMLA-based nanoconjugates were synthesized following the chemical methods described herein. The nanoconjugates contained different peptides for targeting different pathway-receptors, for example, LPR-receptor-guided pathway (AP-2 peptide), Na/K ion channel pathway (MiniAp-4 peptide), and TfR-guided pathway (B6 peptide). The efficacy of transfer through BBB using other pathway targeting peptides is comparable with that of the peptide conjugates targeting a single pathway described herein. The results are surprising, since the efficacies when using different pathway peptides such as AP-2, MiniAp-4, B6 could have differed depending on the type of pathway using either LRP1, transferrin receptor (TfR) or a less well studied component of Na/K ion channel. The positive effect of P/LLL (40%) on permeation efficacy or “boosting” effect is an additional enhancement observed for all the three permeation pathways. To conclude, it was observed that AP-2, B6, and MiniAp-4 (M4) are actively chaperon the nanoagents described herein through the BBB using different transcytosis pathways. The antibodies can be valuable tools to target receptors downstream of reaction cascades. The receptors can be on neurons and other cells. After binding, the nanoagent would be internalized into the targeted brain cell and release its co-load into the cytoplasm or other cellular compartments as was previously described. The co-load then bind to mRNA in the case of AONs, antisense oligo nucleotides, to inhibit synthesis of proteins essential for cancer growth or performing other inhibitory functions as anti-tumor drugs. An IgG2a is a non-limiting example of the antibodies that can be used with the mini nanodrugs described herein. Different antibodies may also be used upon adjusting the chemistry, structure, and/or size of the mini nanodrugs.

### Example 27

### BBB Crossing of Nanoconjugates in Normal Brain: The IgG “Cargo”

To study whether an antibody as “cargo” can be brought into the brain of BALB/C mice, P/LLL/IgG2a conjugates with shuttle peptides were synthesized. A neutral antibody was selected that would not interfere having a specific targeting activity. It was attached via thiol formation to synthetic aminoethyl-SH linker of P/LLL/AP2/rh following as described (Israel et al., 2019, ACS Nano, 13, 1253-1271, which is incorporated herein by reference as if fully set forth). Two forms with high chemical purity were synthesized, one with the rhodamine label on the polymer and one with the label on the immune globulin and characterized to be consistent with antibody nanoconjugate by SEC-HPLC and chemical composition analysis. The version with the label on IgG was denoted as P/LLL/AP2/IgG-rh.

FIGS. 35A-35C illustrate optical imaging data of the brain tissue following mice injections with P/LLL/AP2/IgG/rh, P/LLL/AP2/IgG-rh, and P/LLL/AP2/rh mini nanodrugs. FIG. 35A is a set of photographs illustrating optical imaging data of the brain following the injection of mice with 2× (0.137 μmol/kg) of P/LLL/AP2/IgG/rh (left), P/LLL/AP2/IgG-rh (middle), P/LLL/AP2/rh (right). FIG. 35B are bar graphs illustrating the intensity of fluorescence in the cortex layer II/III, midbrain colliculi and hippocampus following 2 hours post injections of mice with P/LLL/AP2/IgG/rh, P/LLL/AP2/IgG-rh, P/LLL/AP2/rh, or PBS buffer. FIG. 35C are bar graphs illustrating the intensity of fluorescence in the cortex layer II/III, midbrain colliculi and hippocampus CA1-3 layer following 30, 60, 120, 240, or 480 minutes post injections of mice with P/LLL/AP2/IgG/rh, P/LLL/AP2/IgG-rh, P/LLL/AP2/rh, or PBS buffer.

FIGS. 35A and 35B show imaging of BBB permeation by the optical method with the rhodamine-labeling of the antibody, P/LLL/AP2/IgG-rh, and labeling of the platform P/LLL/AP2/rh (FIG. 23B). Referring to FIG. 35B, left and middle panels, per the results for cortex, midbrain, and the dose 2×, both versions cross BBB with almost the same efficacy (fluorescence intensity) as did P/LLL/AP2/rh in the absence of IgG. Referring to FIG. 35B, right panel, the permeation efficacies in the hippocampus are different, which exhibit stepwise rising levels for P/LLL/AP2/rh (the control), P/LLL/AP2/IgG/rh and P/LLL/AP2/IgG-rh.

The PK of P/LLL/AP2/IgG/rh in blood after tail vein injection of dose 2× (0.137 μmol/Kg) followed a half-life of 40 min. In comparison, the half-life for P/LLL/AP2/rh was 76.7 min (Israel et al., 2019, ACS Nano, 13, 1253-1271, which is incorporated herein by reference as if fully set forth). The time dependence in the brain parenchyma followed a fast-rising phase between injection and 30 min after injection, a decline with a half-life in the range of 80 to 100 min and a further slow decline extending into the >500 min region (FIG. 35C). The time dependence for P/LLL/AP2/rh followed also 3 phase; however the first decline followed a half-life of >120 min and a time independent plateau at >240 min (Israel et al., 2019, ACS Nano, 13, 1253-1271, which is incorporated herein by reference as if fully set forth). For both nanoconjugates, the overall residing period in the parenchyma extended beyond the half-life of blood clearing and can be described approximately by a steady state equilibrium between influx and efflux from blood to parenchyma and in the reverse from parenchyma to blood. The final retention >500 min for P/LLL/AP2/IgG/rh indicated significant amount of nanoconjugate adhering to cells or particulate in the presence of attached IgG.

Considering that the movement of the ferry platform through BBB encounters binding sites for the shuttle peptide recognition site and an allosteric regulatory allosteric site, the introduction of IgG cargo, raises the possibility of new interactions. While the similar fluorescence intensities of the conjugates (FIG. 35B) at dose 2× were not significantly affected for cortex and midbrain and suggested that such interactions did not exist, the changes seen for the hippocampus signaled significant permeation increase between P/LLL/AP2/IgG/rh and P/LLL/AP2/rh resulting from an IgG interaction.

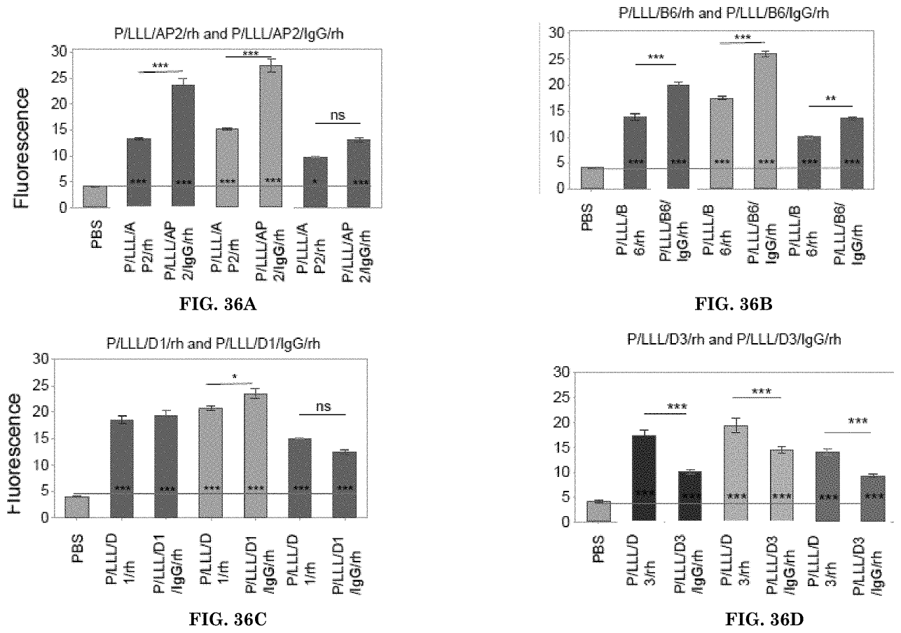

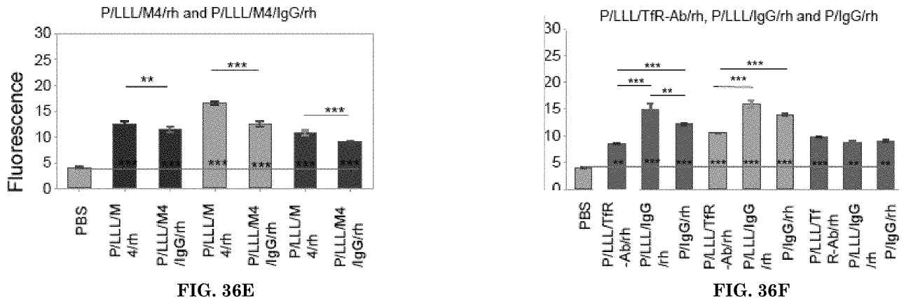

For obtaining further evidence, the dose was increased from 2× to dose 4×. FIGS. 36A-36F are bar graphs illustrating optical data quantification 2 hours post injection for IgG and non-IgG mini nanodrugs at 0.274 μmol/kg (4×). FIG. 36A are bar graphs illustrating the intensity of fluorescence in the brain following injections of mice with P/LLL/AP2/rh, P/LLL/AP2/IgG/rh, or PBS buffer.

FIG. 36B are bar graphs illustrating the intensity of fluorescence in the brain following injections of mice with P/LLL/B6/rh, P/LLL/B6/IgG/rh, or PBS buffer.

FIG. 36C are bar graphs illustrating the intensity of fluorescence in the brain following injections of mice with P/LLL/AD1/rh, P/LLL/D1/IgG/rh, or PBS buffer. FIG. 36D are bar graphs illustrating the intensity of fluorescence in the brain following injections of mice with P/LLL/D3/rh, P/LLL/D3/IgG/rh, or PBS buffer. FIG. 36E are bar graphs illustrating the intensity of fluorescence in the brain following injections of mice with P/LLL/M4/rh, P/LLL/M4/IgG/rh, or PBS buffer. FIG. 36F are bar graphs illustrating the intensity of fluorescence in the brain following injections of mice with P/LLL/TfR-ab/rh, P/LLL/IgG/rh, P/IgG/rh or PBS buffer.

Referring to FIG. 36A, this change increased the fluorescence intensity, and made it similar in all three regions of the brain. The degree of IgG-induced intensity varied with the kind of shuttle peptide. While the intensity increase for P/LLL/AP2/rh in cortex and midbrain was relatively modest, the intensities for the IgG-nano conjugate doubled. Referring to FIG. 36B, the intensities measured for P/LLL/B6/IgG/rh relative to control P/LLL/B6/rh were increased in a similar fashion. Referring to FIGS. 36C-36E, by the same criteria applied to D1, D3, M4 conjugates, changes were found which coupled the IgG effect either with a small increase (D1) or a significant decrease in fluorescence intensity.

Referring to FIG. 36F, the presence of IgG did not prevent BBB permeation in absence of shuttle peptides. Comparison of P/LLL/IgG/rh with control P/IgG/rh revealed an LLL-induced increase in permeation. In addition, competition with P/LLL (40%) at dose 20× did not reduced BBB permeation of IgG. This confirmed that IgG did not interfere with site (B) binding of tri-leucine (40%). In nanoconjugates containing anti-TfR antibody (aTfR) instead of IgG, BBB permeation was relatively inefficient because of the low degree of antibody-TfR dissociation at sub nmolar concentrations in agreement with the high affinity of the antibody-TfR complex (Yu et al. 2011, ScienceTranslationalMedicine.org, which is incorporated herein by reference as if fully set forth).

In summary, IgG may be regarded as nanoconjugate-integrated “reporter”, by effecting an increase in (FIGS. 36A and 36B), no increase (FIG. 36C) and a decrease (FIGS. 36D and 36E) of BBB permeation efficacy. The direction of the effect is coupled with the permeation efficacy owned by the nanoconjugates (“controls”) prior to conjugation with IgG. A possible explanation is that receptor binding of D1, D3 or M4 in contrast to AP2 or B6 interfered with receptor binding of IgG resulting in the observed reduced permeation efficacy. The structure dependence of BBB permeation in normal brain revealed binding of the PMLA nanoconjugates to regions that bind shuttle peptides, tri-leucine and the cargo IgG. The sites occupied by the peptides AP2, B6, D1, D3 are probably contiguous to a site interacting with IgG and to the P/LLL (40%) binding region, which is allosterically involved in shaping the shuttle peptides binding site(s). The sites as a unity participates in transcytosis through BBB and their efficacies appear to be modulated depending of their location in cortex, midbrain and hippocampus of normal brain.

The occupation of binding sites follows site-specific concentration dependences which can produce concentration dependent additive permeation or subtractive effects, reflecting competition when overlapping same sites, for example, displayed in the presence of IgG. It may be assumed that the D-peptide subsites, but not the AP2 and B6 sites, were positioned in the IgG specific binding site. Transcytosis routes could be at least partially colocalized as reported for LDL and Tf and LRP1 and Aβ (Ramanathan et al, (2015) Frontiers in Aging Neuroscience, 7, 136, which is incorporated by reference as if fully set forth).

For the design of medical treatment, the ligand specific design as well as the brain region modulation of delivery have to be taken into account. A few empirical statements may be proposed which could be useful for the selection of nanoconjugates: (1) In all brain regions the BBB permeation was highest for the conjugates containing the antibody and a shuttle peptide. (2) For M4, D1 and D3 or other nanoconjugates, a boosting effect by “cargo” may not be observed if maximum permeation efficacy is indicated in the absence of cargo. (3) The clearance of P/LLL/AP2/IgG/rh from parenchyma revealed an IgG-related prolonged time period which could results in accumulation of conjugate during repeated injections.

### Example 28

### BBB Crossing in Double Transgenic AD Mouse

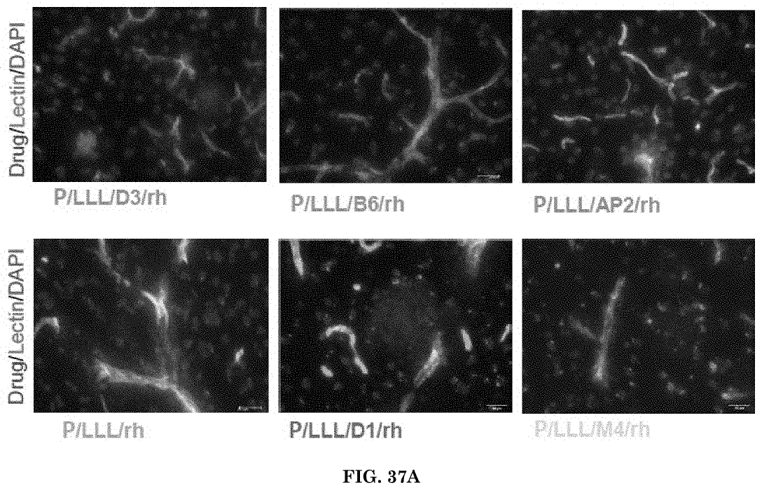

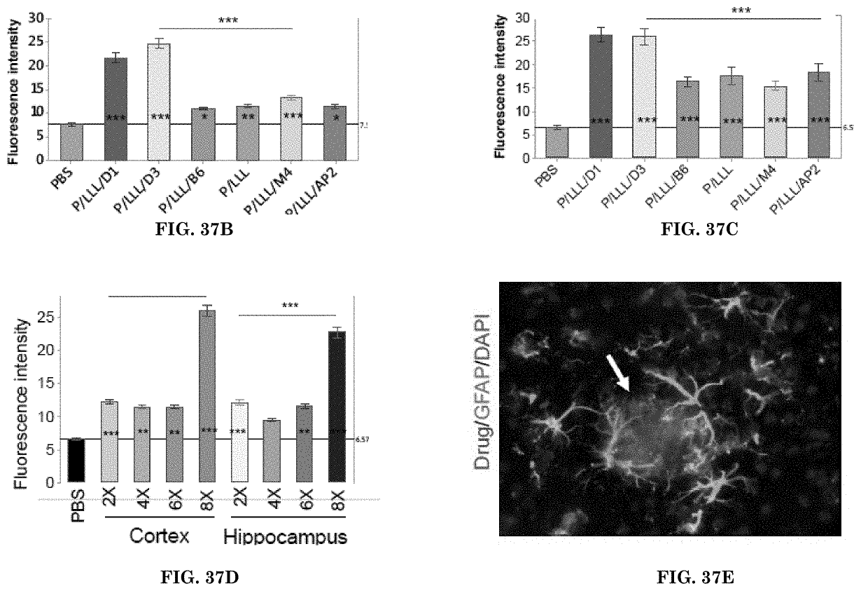

The BBB permeation efficacy of mini nanodrugs was tested in diseased brain. A comparison with normal brain was beneficial for understanding of the development of Alzheimer's disease. Because LRP1 receptor in Alzheimer's brain is downregulated while TfR is unaffected, a difference of BBB permeability between normal 1 BALB/C brain and a brain of the transgenic mouse [6-8 month old 5-FAD (B6.Cg-Tg (APPswe/PS1ΔE9) 85Dbo/J hemizygous)] was investigated (Ramanathan et al, (2015) Frontiers in Aging Neuroscience, 7, 136; and Bourassa et al., (2019) Molecular Pharmaceutics, 16, 583-594, both of which are incorporated by reference as if fully set forth). FIGS. 37A-37E illustrate the BBB permeation efficacies following injections of mice with P/LLL/D 1/rh, P/LLL/D3/rh, P/LLL/B6/rh, P/LLL/rh, P/LLL/M4/rh, P/LLL/AP2/rh

FIG. 37A is a set of photographs illustrating optical imaging data of the cortex of the AD brain following the injection of mice with 8× [0.548 μmol/kg] of P/LLL/D3/rh (top left), P/LLL/B6/rh (top middle), P/LLL/AP2/rh (top right), P/LLL/rh (bottom left), P/LLL/D 1/rh (bottom middle), and P/LLL/M4/rh (bottom right) in the tumor (left) and the other hemisphere (brain; right). FIG. 37B are bar graphs illustrating the intensity of fluorescence in the hippocampus of AD brain following injections of mice P/LLL/D1/rh, P/LLL/D3/rh, P/LLL/B6/rh, P/LLL/rh, P/LLL/M4/rh, P/LLL/AP2/rh or PBS buffer. FIG. 37C are bar graphs illustrating the intensity of fluorescence in the cortex of AD brain following injections of mice with nanodrug P/LLL/D1/rh, P/LLL/D3/rh, P/LLL/B6/rh, P/LLL/rh, P/LLL/M4/rh, P/LLL/AP2/rh or PBS buffer. FIG. 37D are bar graphs illustrating the intensity of fluorescence in AD brain parenchyma following injections of mice with P/LLL/D3/rh or PBS buffer at 2×, 4×, 6×, or 8× dose in the cortex or hippocampus. [1×=0.068 μmol/kg]

FIG. 37E is a photograph illustrating optical imaging data of Aβ plaque in the AD brain parenchyma following the injection of mice with P/LLL/D3/rh.

Referring to FIG. 37A, the results of the tail IV injections of P/LLL/D3/rh, P/LLL/B6/rh, P/LLL/AP2/rh, P/LLL (40%)/rh P/LLL/D1/rh, P/LLL/M4/rh into transgenic mice are shown in in the photographs following injections of mice with mini nanodrugs at dose 0.548 μmol/kg (8×). Referring to FIGS. 37B and 37C, the averaged BBB permeation efficacies (in terms of fluorescence intensities) shown as bars in these drawings (B6: hippocampus t-3.03 p-0.039 cortex t-6.22 p-0.000; M4: hippocampus t-5.08 p-0.000 cortex t-5.62 p-0.00; AP2: hippocampus t-3.44 p-0.011 cortex t-7.51 p-0.000; LLL: hippocampus t-3.56 p-0.007 cortex t-7.06 p-0.000); D3 hippocampus t-14.51 p-0.000; cortex t-13.72 p-0.000; D1: hippocampus t-11.94 p-0.000 and in cortex t-13.99 p-0.000) (t=degree of significance). Referring to FIG. 37D, the dose dependence is depicted for P/LLL/D3/rh in in cortex and hippocampus. The permeation efficacies were compared for normal mice (BALB/C data are illustrated on FIGS. 36A-36F) and the transgenic mice.

Permeation efficacies for transgenic mice were distributed below the level for normal mice. In dose range 0.137-0.274 μmol/kg (2×-6×) the efficacies were scattered rather than indicating an increase, except in the case of P/LLL/D3/rh, at dose 0.548 μmol/kg (8×), a steep rise was observed in both cortex and hippocampus.

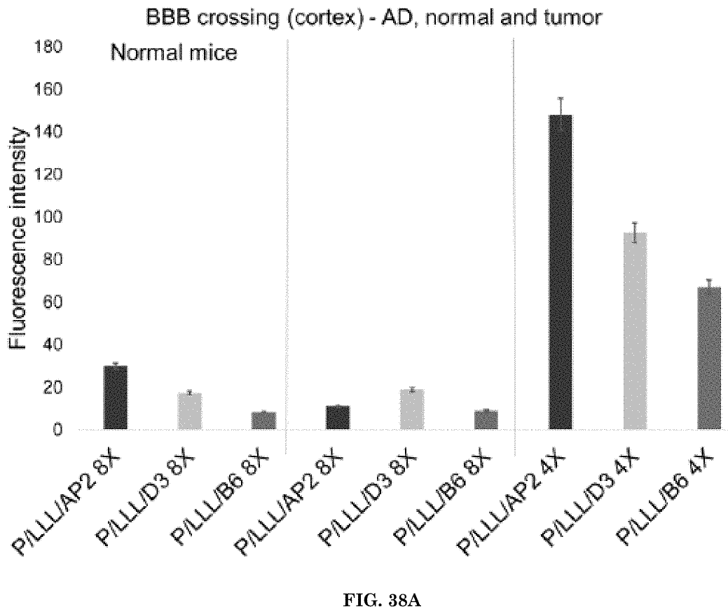

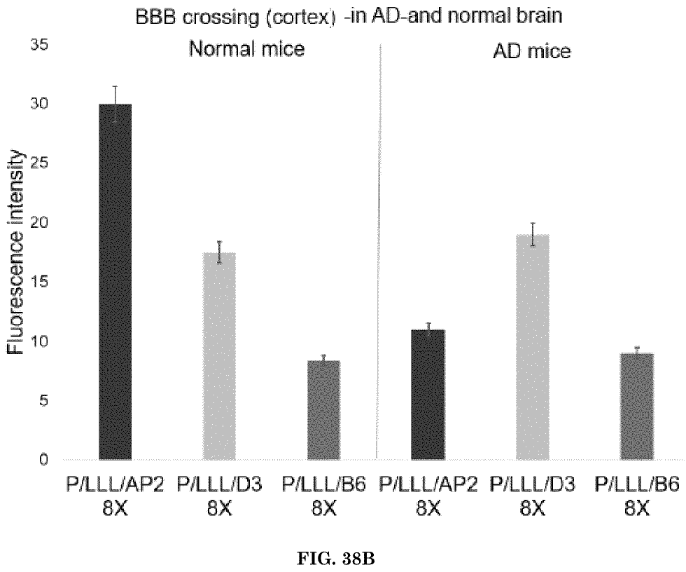

FIGS. 38A-38B are bar graphs illustrating the mean intensity of fluorescence (after PBS deduction) in the normal, AD and tumor (FIG. 38A) or normal and AD brain (FIG. 38B) following injections of mice with 8× of P/LLL/AP2/rh, P/LLL/D3/rh, P/LLL/B6/rh, P/LLL/AP2/rh, P/LLL/D3/rh, P/LLL/B6/rh P/LLL//rh and 4× of P/LLL/AP2/rh, P/LLL/D3/rh, P/LLL/B6/rh.

A decrease of permeation efficacy for P/LLL/AP2/rh in AD at dose 8× is indicated by the decrease in fluorescence intensity from 30 units in normal brain (FIG. 38A, left side) to 10 units in AD brain (FIG. 38B, right side) and corresponds with down regulation of LRP1 receptor. A decrease was not observed for P/LLL (40%), P/LLL/D3/rh and P/LLL/B6/rh. In the case of B6, the data can be explained by downregulation of a TfR in AD as well P/LLL (40%) which is not down regulated in AD brain. In the case of D3, the steep rise in the efficacy during the dose increase from 6× to 8× was unexpected. Because it was demonstrated by competition with AP2/LLL/AP2/rh in normal brain and by in vitro experiments that D-peptides bind with LRP1, its permeation efficacy should have been reduced in comparison with normal brain.

The steep rise was assumed to be connected with the increased level of amyloid-beta in the diseased AD brain due to overproduction of secretases and enhanced RAGE-mediated influx. Because D-peptides were phage-selected for binding Aβ, they would largely exist in the form of P/LLL/D1, D3/rh-Aβ complexes in the transgenic AD-brain. Assuming that the non-complexed D-nanoconjugates are the shuttle peptides moved by LRP1-mediated transcytosis into parenchyma, their Aβ-P/LLL/D1, D3/rh are inhibitors and block transcytosis. This effect of amyloid-beta was best illustrated regarding their BBB permeation in cortex (after subtraction of background intensity), reflected by fluorescence intensity of 6 units in AD brain at the dose 4× (FIG. 38D) while the fluorescence of 14 units is measured in normal brain (FIG. 36D). This mechanism alone does not explain why the BBB permeation efficacy (fluorescence intensity) at dose 8× steeply increased (FIG. 38D) to levels seen in normal brain (FIGS. 36C and 36D). While BBB permeation represents the flux in the direction blood to brain, LRP1 has also an important clearing function by moving Aβ in the direction brain to blood. If Aβ complexation with LRP1 interferes with binding of P/LLL/D1, D3/rh it would inhibit transcytosis of the D1, D3-nanoconjugates. The dilemma could be solved if the D-conjugates “neutralized” Aβ by complexation giving excess D-conjugates the opportunity for transcytosis. The abrupt rise from low to high BBB-permeation intensities at high dose (FIG. 38D) is reminiscent of an endpoint during amyloid-beta titration with D-nanoconjugate. Above the neutralization, the now free D1, D3 shuttle peptides complex with LRP1/amyloid-beta (holo)receptor and resume transcytosis.

TABLE 5
Percentage of total plaques labeled by mini nanodrugs

| | Percentage of total plaques |
|---|---|
| Conjugate | labeled by the reagents |
| P/LLL/D1 | 0% |
| P/LLL/D3 | 21.1% |
| P/LLL | 4.5% |
| P/LLL/B6 | 3.3% |
| P/LLL/AP2 | 1.23% |
| P/LLL/M4 | 0% |
| P/LLL/D3 | |
| Injected dose | |
| 2X | 0% all in a single box |
| 4X | 0% |
| 6X | 0% |
| 8X | 21.1% |

### Example 29

### Detailed Analysis for BBB Permeation in the Presence of IgG in the Supplementary Part.

Surprisingly, the increase in dose caused a boosting in BBB crossing in the cortex and midbrain for two of the tested nanoconjugates. P/LLL/B6/IgG levels at 4× were significantly higher than P/LLL/B6 levels also in the hippocampus, while an increase was visible for P/LLL/AP2/IgG in the cortex and midbrain. That effect was not visible at these brain regions for P/LLL/AP2/IgG/rh at a lower dose (FIG. 36B). For D3, M4 and D1, no increase was detected, however, all IgG conjugates were significantly higher than PBS level. For P/LLL/D1/IgG/rh, the levels in the midbrain and cortex remained the same as P/LLL/D1/rh, while a slight drop was seen at the hippocampus (FIG. 36C). P/LLL/M4/IgG/rh showed a slight drop in all brain regions (FIG. 36E), while P/LLL/D3/IgG/rh showed a larger drop compared to P/LLL/D3/rh (FIG. 36D), however, the BBB crossing was still significantly higher than PBS in all brain regions which were measured (cortex: t-6.18 p-0.000, midbrain: t-10.7 p-0.000 hippocampus: t-5.29 p-0.000).

When comparing the levels of P/LLL/peptide/IgG/rh for AP2, D1 and B6 (FIGS. 36B-36C), all IgG nanoconjugates with peptides performed better then carrier/IgG controls. However, despite the drop P/LLL/D1/IgG/rh showed in the hippocampus, the fluorescence levels remained similar for P/LLL/D1/IgG/rh, P/LLL/AP2/IgG/rh and P/LLL/B6/IgG/rh. At the midbrain, P/LLL/AP2/IgG/rh—had a small but significant increase (p-0.0031) over P/LLL/D1/IgG/rh while in the cortex P/LLL/AP2/IgG/rh showed a slightly increased level also in comparison to P/LLL/B6/IgG/rh.

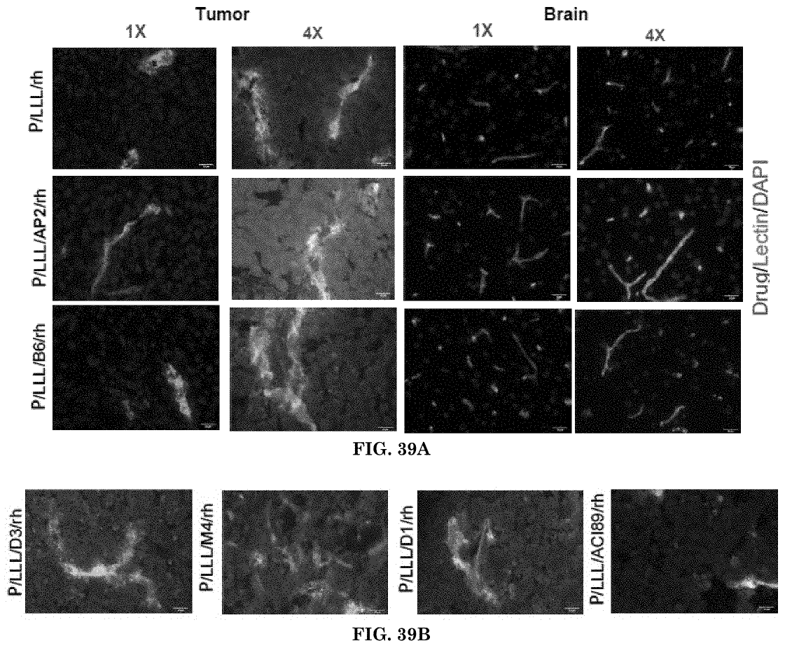

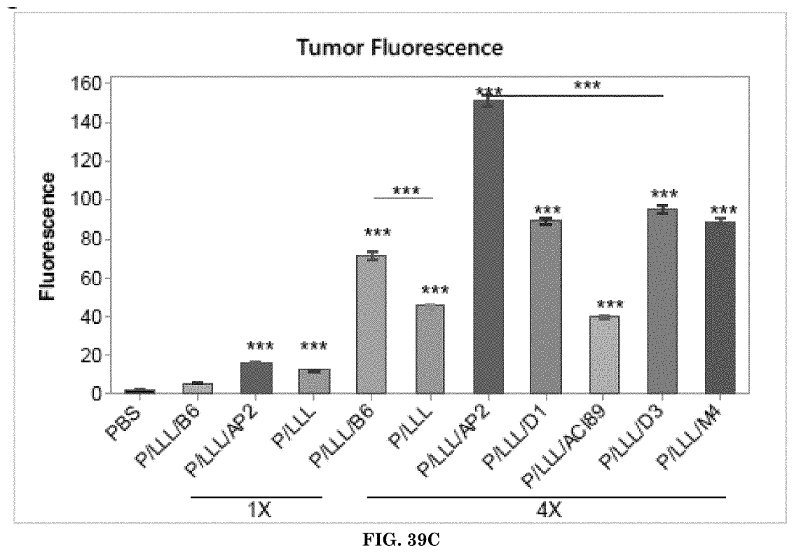

These results imply that the mechanism of BBB crossing for the P/LLL/peptides/IgG/rh is similar to those suggested for the tumor model (FIGS. 39A-39C). It can include more than one pathway which an increase in dose can saturate one pathway and force the nanoconjugate to cross via another pathway, or an allosteric model.

### Example 30

### Increased BBB Permeation Efficacy in Brain Tumor: The Example Human Xenogeneic Glioblastoma GL-261 Mouse Model

Glioblastoma BBB was tested as an example of a profoundly aberrant blood-brain barrier. Glioblastoma GL-261 represents an aggressive human brain tumor. Briefly, three weeks post inoculation, tumor bearing mice were injected with rhodamine labeled nanodrug and euthanized 2 h post injection. Brains were sliced, and microscopic images examined of both the tumor area and the corresponding non-tumor symmetrically positioned in the other brain hemisphere. FIGS. 39A-39C illustrate optical imaging data the tumor area and the corresponding non-tumor symmetrically positioned in the other brain hemisphere following mice injections with the mini nanodrugs.

FIG. 39A is a set of photographs illustrating optical imaging data in cortex of tumor bearing brain following the injection of mice with 1× (0.0685 μmol/kg) or 4× (0.274 μmol/kg) of P/LLL/B6/rh (bottom), P/LLL/AP2/rh (middle) and P/LLL/rh in the tumor (left) and the other hemisphere (brain; right). FIG. 39B is a set of photographs illustrating optical imaging data in cortex of tumor bearing brain following the injection of mice with 4× (0.274 μmol/kg) of P/LLL/D3/rh (left), P/LLL/M4/rh (middle left), P/LLL/D1/rh (middle right) and P/LLL/AC189/rh (right). FIG. 39C are bar graphs illustrating the intensity of fluorescence in the tumor following injections of mice with 1× of P/LLL/B6/rh, P/LLL/AP2/rh P/LLL/rh, and 4× of P/LLL/rh, P/LLL/AP2/rh, P/LLL/B6/rh, P/LLL/D1/rh, P/LLL/D1/rh, P/LLL/AC189/rh, P/LLL/D3/rh, P/LLL/M4/rh or PBS buffer.

The drug fluorescence intensity as a measure of drug quantity was assessed in twenty 10×10 μm² regions of interest (ROI) per image, 5 images per area per mouse, and three mice per group as described herein. Data in Table 6 are corrected by subtraction of background fluorescence measured after PBS injection.

As seen in both FIGS. 37A-37E and Table 6, the fluorescence intensity for P/LLL/AP2/rh at dose 4× (0.274 μmol/Kg) is the highest in comparison with the intensities for the other nanoconjugates. Except for P/LLL/rh, the fluorescence intensities at doses 1× and 4× are not proportional. In fact, the dose dependences appeared to be exponential except for P/LLL/rh with a linear dependence. For P/LLL/AP2/rh the dose-dependent change was 11.2-fold, and for P/LLL/B6/rh 23-fold between 1× and 4×.

The availability of the fluorescence intensity of the nanoconjugates in the other hemisphere allowed calculation of tumor selectivity over normal brain (T/B). For the control P/LLL/rh, the T/B for 1× and 4× doses was similar, 9.7 and 8.3 (Table 6). For P/LLL/AP2/rh, T/B(1×)=6.5 and T/B(4×)=15.7 showing a 2.4-fold increase in tumor selectivity with the increase of the dose. A similar trend was observed for P/LLL/B6/rh, however, the selectivity T/B(4×)=10.2 was less than for P/LLL/AP2/rh (Table 6). The most selective conjugate was P/LLL/M4/rh with a T/B(4×) of 22.8, followed by P/LLL/D1/rh with a T/B(4×)=16.4. The results of linear dose dependence, low permeation efficacy and absence of selectivity for P/LLL (40%) is consistent with passive penetration of BBB following the EPR-mechanism. This passive mechanism is in contrast with the observed supra linear dose dependence and the much higher efficacy when the shuttle peptide nanoconjugates were physically combined, suggesting cooperatively coupling via an allosteric mechanism. This mechanism was shown to function in normal brain and was found and confirmed and magnified in brain tumor. It was shown that P/LLL (40%) crosses the healthy BBB without any peptide attached, however, adding coupling to a BBB crossing peptide increased both uptake and selectivity for the tumor.

In this example, a glioblastoma model has been used which overexpresses both LRP1 and TfR [22]. Per our results, the nanoconjugate P/LLL/AP2/rh (4×) known to use the LRP1 pathway was the clear winner in terms of tumor uptake, followed by the other conjugates (P/LLL/D 1/rh and P/LLL/D3/rh), P/LLL/M4/rh and P/LLL/B6/rh which precedes conjugates P/LLL/ACI89/rh and P/LLL/rh. Thus the nanoconjugate of B6 not highly effective at Dose 4×, although using the TfR-transcytosis pathway.

TABLE 6
Fluorescence intensity in tumor and
brain based on mini nanodrug doses

| | | | Brain (other | |
|---|---|---|---|---|
| | | Tumor | hemisphere) | ratio |
| Conjugate | Dose | fluorescence | fluorescence | Tumor/Brain |
| P/LLL | 1X | 10.0 | 1.0 | 9.7 |
| | 4X | 42.7 | 5.1 | 8.3 |
| P/LLL/AP2 | 1X | 13.5 | 2.1 | 6.5 |
| | 4X | 148.2 | 9.4 | 15.7 |
| P/LLL/B6 | 1X | 3.0 | 1.2 | 2.5 |
| | 4X | 69.2 | 6.8 | 10.2 |
| P/LLL/D1 | 4X | 86.6 | 5.3 | 16.4 |
| P/LLL/ACI89 | 4X | 37.1 | 4.3 | 8.6 |
| P/LLL/D3 | 4X | 92.6 | 7.8 | 11.9 |
| P/LLL/M4 | 4X | 86.2 | 3.8 | 22.8 |

### Example 31

### Receptor Complexes, BBB-Permeation Efficacy. Tumor Selectivity and a Multifactorial Study

By interrogating a family of nanoconjugates with the chemical structure P/LLL/vector consisting of peptides with referenced functions, and brains of different disease status, an underlying consensus route for BBB crossing was found in normal brain, AD-brain and tumor-brain. The consensus involves conservative binding and auxiliary binding which in a coupled fashion contribute to BBB permeation.

Binding sites were assigned for basic vectors (AP2, B6), modified vectors (D1, D3, ACI 186), subsites involved in vector modification, and “cargo” (e.g., IgG). Basic vectors are of minimal design targeting transcytosis receptors such as LRP1 and TfR. Modified vectors elected subsites added to LRP1 for the addition of binding affinity and shaping new specificity. In the case that not all vector subsites sites can be occupied without steric or otherwise repulsive interaction, the ligand offering the least promotion of increased binding affinity is expelled and stays in competition with the optimal arrangement. It was found that the effector P/LLL (40%) induces via allosteric re-arrangement a pathway with maximum selectivity and efficacy of BBB permeation.

Because P/LLL-effector and vector are integrated portions of the mini nanodrugs, the situation is complicated showing besides the allosteric also a competitive component. FIGS. 40A-40B are schematic representations of the mini nanodrugs binding via two pathways mechanism (FIG. 40A) and via the allosteric mechanism (FIG. 40B)

Referring to FIG. 40A, variant I: site (A) binds P/LLL, but is unproductive in BBB-permeation and competes with peptide binding in site (B) which binds the peptide (vector) with low affinity but is productive. The concentration dependence for permeation activity is biphasic, especially if binding to the unproductive site is of higher affinity than for binding to site (B). FIG. 40B, variant II: site (A) is not very productive, but when occupied, site (A) allosterically gives rise to the exposure of site (B) which binds the vector peptide and is much more active than site (A) The dose dependence is biphasic. By inspecting the data in Table 6 for P/LLL (40%) and P/LLL (40%)/AP2 the evidence is in support of variant II indicated by the significantly higher effect on fluorescence in the tumor than the control (the healthy hemisphere) also reflected by the significantly increased selectivity for P/LLL/AP2.

In the investigation of shuttle peptides, the evidence was found for (partially) shared common and specific transcytosis pathways. In asking for support of the receptor model that involves a coupling between the non-identical binding sites (A) and (B), and coupling between LRP1- and B6 transcytosis pathways, a “multifactorial study” was launched (Design of Experiment, DoE) analysis (Kenett and Zacks, 1998; and Kenett, 2014, both of which are incorporated herein by reference as if fully set forth).

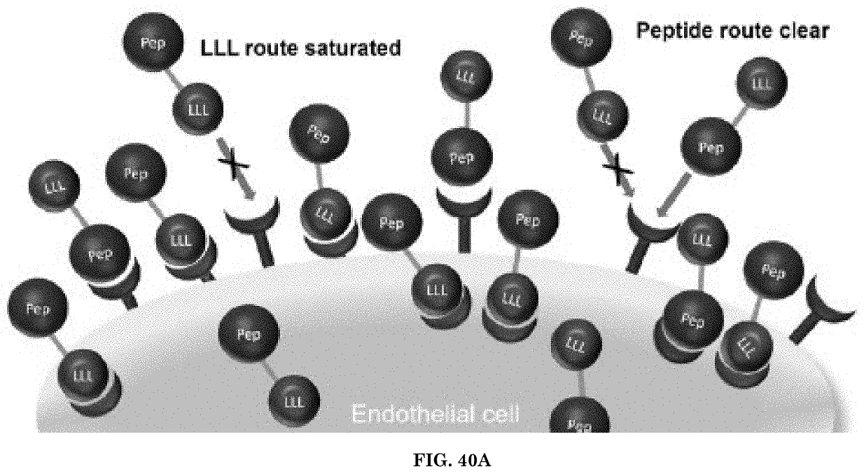

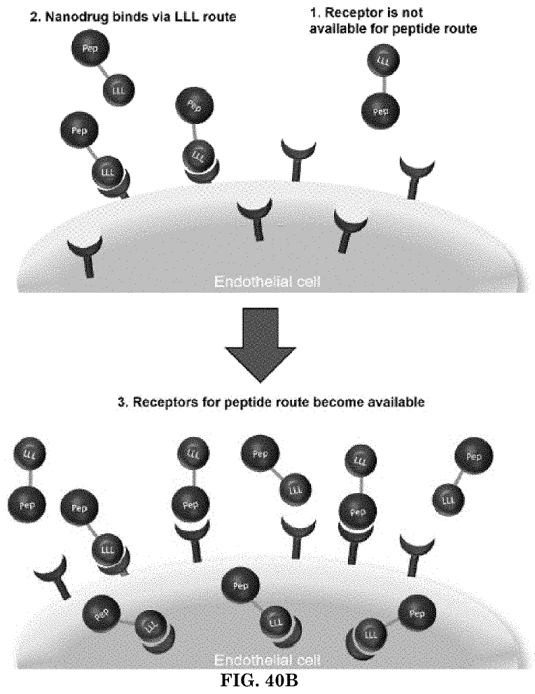

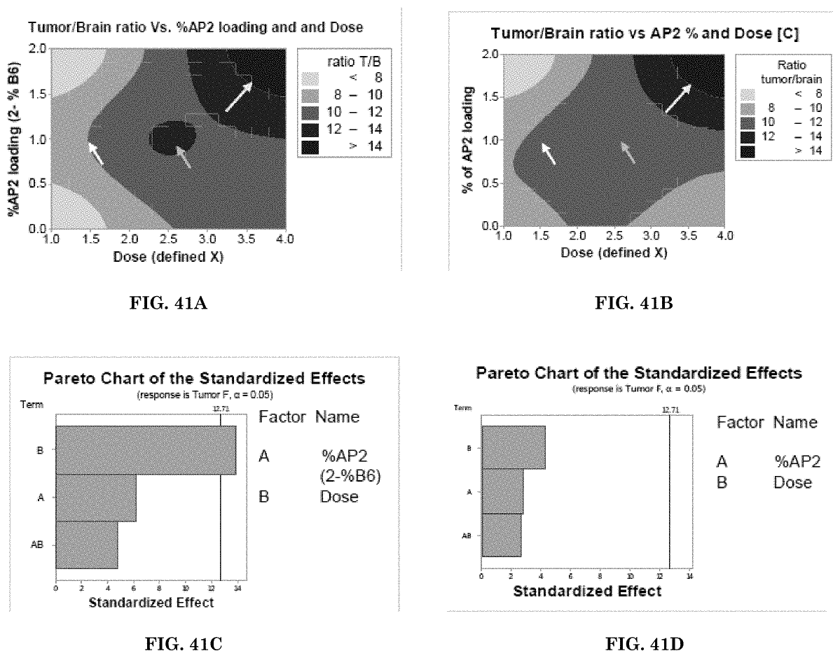

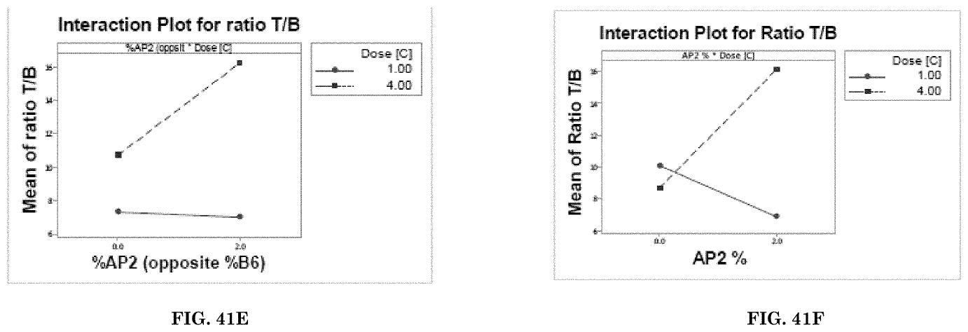

As an example, the permeation of glioblastoma-BBB by P/LLL/AP2/rh (FIG. 41B) and by P/LLL/AP2/B6/rh (FIG. 41A) was selected. P/LLL/AP2/rh shown on FIG. 41B involves a route across a single (LRP1) transcytosis pathway and P/LLL/AP2/B6/rh shown on FIG. 41A involves a route sharing two transcytosis pathways (LRP1 and TfR).

For each case a DoE-matrix which contained factor “Loading”, factor “DoseX”, the “Fluorescence Intensity” as response one, and the tumor “Selectivity” as the other response was built. The AP2-matrix for experiments with P/LLL/AP2/rh and the combined matrix for experiments with p/LLL/AP2/B6/rh are depicted in Table 7.

In Table 7, each matrix contained also a “centerpoint” which in the AP2-matrix contained the “Fluorescence Intensity”/“Selectivity” measured for P/LLL/AP2 (1%)/rh at dose of 2.5× (66.65 μmol/Kg), and in the combined-matrix the intensity measured for P/LLL/B6 (1%)/AP2 (1%)/rh at dose 2.5× (66.65 μmol/Kg).

TABLE 7
DoE Analysis Data
AP2-matrix*

| | | Response: | |
|---|---|---|---|
| | | Fluorescence uptake | |
| Factor: | Factor: | in tumor region | Response: |
| AP2 (%) | Dose | [arbitrary | Selectivity to the |
| loading | [X] | fluorescence units] | tumor, T/B ratio |
| 0 | 4 | 42.66 | 8.33 |
| 0 | 1 | 10.02 | 9.73 |
| 1 | 2.5 | 31.20 | 11.87 |
| 2 | 1 | 13.48 | 6.51 |
| 2 | 4 | 148.25 | 15.75 |

Combined-matrix**

| | | | Response: | |
|---|---|---|---|---|
| | | | Fluorescence | |
| | | | Uptake in | Response: |
| Factor: | Factor: | | tumor Region | Selectivity |
| AP2(%) | B6(%) | Factor: | [arbitrary | to the |
| loading | loading | Dose | fluorescence | tumor, |
| (2%-x%B6) | (2%-x%AP2) | [X] | units] | T/B |
| 2 | 0 | 1 | 13.48 | 6.55 |
| 1 | 1 | 2.5 | 50.39 | 12.17 |
| 0 | 2 | 4 | 69.20 | 10.25 |
| 2 | 0 | 4 | 148.25 | 15.75 |
| 0 | 2 | 1 | 2.97 | 6.84 |

*data presented at FIG. 41B
**data presented at FIG. 41A

FIGS. 41A-41F illustrate factorial study data for P/LLL/AP2/B6/rh matrix (FIGS. 41A, 41C and 41E)) and P/LLL/AP2/rh matrix (FIGS. 41B, 41D and 41F). FIGS. 41A and 41B illustrate 2D contour plots for the response tumor/brain (T/B) (axis: Z-T/B ratio, Y-% of AP2 (%) loading and X-dose).

FIG. 41A illustrates data for combined matrix. Referring to this figure, γ-axis indicates Factor AP2 (%) loading=(2%-x % B6) and Factor B6 (%) loading=(2%-x %AP2); x-axis indicates Factor Dose [X] and z-axis indicates Response fluorescence intensity measured in arbitrary units in tumor region and color-coded or T/B (fluorescence intensity ratio tumor divided by control).

FIG. 41B illustrates data for AP2 matrix. Referring to this figure, y-axis indicates Factor AP2 (%) loading; x-axis indicates Factor Dose [X] and z-axis indicates fluorescence intensity tumor or T/B. FIGS. 41C and 41D illustrate the pareto charts for standardized effects for tumor fluorescence intensity response. FIGS. 41E and 41F illustrate interaction plots for T/B ratio response.

Each matrix contained also the data for “end point” experiments. Referring to FIGS. 41A-41F, for each matrix, the DoE software generated a “contour”-plot (FIGS. 41A-41B), a “significance”-plot (“Parreto chart”) (FIGS. 41C and 41D), and an “interaction”-plot (FIGS. 41E and 41F). In the “Contour Plots” (FIGS. 41A and 41B), the “Selectivity” (T/B) of tumor (T) over normal brain (B) is coded by different shades of blue.

Inspection of the contour plots shows that the region of highest tumor selectivity is the one closest to the highest loading dose 4× (arrows pointing to the right corners in FIGS. 41A and 41B). Presence of B6-peptide (case 2) enhances selectivity close to the center point (FIG. 41B for 2.5×, 1% AP2, arrows in the center of the chart), while reduces selectivity at lower doses (FIG. 41B, left arrow at 1.5×, 1%). The plot predicts that in doses below 1.5×, the conjugation of B6 infers selectivity which is less (FIG. 41B) than observed for P/LLL/AP2 (1×) (FIG. 41A) at 1×, and less than the one for (P/LLL (1×) (FIG. 41A) i.e. in the absence of shuttle peptide B6 (Table 7). It is also evident that nanoconjugate P/LLL/AP2 (1%) cannot achieve selectivity R/T>12 at any injected dose 1×-4× (FIG. 41A, light grey). However, the selectivity can be achieved by combination of AP2 and B6 (P/LLL/B6 (1%)/AP2 (1%)) at a dose of 2.5×. These data support evidence for coupling between the LRP1- and TfR-transcytosis pathways. It is also seen that the threshold for selectivity is highest for nanoconjugate P/LLL/AP2 (2%) and out of reach for nanoconjugates that contain simultaneously AP2 and B6.

Referring to FIGS. 41B and 41C, Parreto charts present the calculated statistical significance of the response (R/T) inferred by the loading of single (AP2) and combined (AP2, B6) shuttle peptides on R/T, analogous to commonly used P-value. The vertical lines on the charts mark the calculated degree of significance. A factor is significant if the bar crosses the red line. For the AP2-matrix (P/LLL/AP2, FIG. 41C), both percent loading and dose are not significant factors (FIG. 41D). When B6-peptide is added to the system (combined-matrix, P/LLL/AP2/B6), the shuttle peptide loading is still not a significant factor, but the dose dependence is significant (crossing the vertical line on FIG. 41C). These observations bear on the reliability of observations made earlier on the basis of dose variations in the contour plots (FIGS. 41A-41B).

Additional evidence for interaction (coupling) resting on the peptide AP2 and B6 loading is obtained by the impact of different doses 1× and 4× in plot FIG. 41E (the AP2 matrix) and FIG. 41F (the combined matrix) achieving the selectivity towards tumor (T/B). The slopes of the lines in both graphs have opposite signs which indicates coupling between the “percent of peptide loading” and the “dose” in generating the T/B response. There would be no coupling if the lines run parallel. A coupling was also indicated in the different contour plot FIG. 41B where a singular maximum is found in the mid-point, not present in the dose dependence of AP2 alone (FIG. 41A). A coupling is supported by the allosteric interactions between vector binding at site (A) and P/LLL at site (B) of the two-site receptor model in FIG. 41B. Moreover, the data confirm a coupling between the LRP1- and TfR-transcytosis pathways through the combined loading of AP2 and B6 on the same platform molecule.

The coupling becomes evident in FIG. 41E where upon the increase from dose 1× to 4× for both P/LLL (40%) and the shuttle peptides P/LLL (40%)/pep (2%), the T/B values decrease from 9.7 to 8.3 for P/LLL (40%) and increased from 6.5 to 15.7 for P/LLL (40%)/AP2 (2%) and from 2.5 to 10.2 for P/LLL (40%)/B6 (2%).

With the help of DoE-analysis further data which predict uptake and tumor selectivity as function of the nanoconjugate ligand-loading and injected dose was obtained. For instance, the nanodrug for optimal glioblastoma uptake and selectivity at dose 4× is P/LLL/AP2 (2%)/rh. Based on the available data obtained with the shuttle peptides, no advantage was apparent by using combinations of peptides other than the ones tested here that exceeded the potency of AP2 accomplished in glioblastoma targeting. The result is specific for glioblastoma, but other optimal factors such as the influence of IgG (cargo) are expected for normal brain, AD-brain, and in dependence of the location in the brain as well of doses (FIGS. 24, 36A-36C, 37A-37E, and 38A-38B). Depending on the particular composition of the nanoconjugate and the molecular environment of the endothelial permeation pathway, additional receptor sites, competitive and allosteric coupling could be expected.

### Example 32

### BBB Permeation Pathways for Polymalic Acid/Tri-Leucine/Shuttle Peptide Nanoconjugates in Normal, AD, and Glioma Brain

Having investigated in detail the correspondence between chemical design and the permeation efficacy of polymalic acid-trileucine nanoconjugates through BBB, we found general and specific criteria in normal brain, AD brain and Glioblastoma. The structural outfit common to the binding of an internalizing nanoconjugate manages a conservative response to the binding of the polymalic acid-trileucine copolymer (P/LLL) moiety and specific responses of binding the shuttle peptides recognized by receptors, e.g., LDLR and TfR of transcytosis pathways. The extracellular domain is rich in ligand binding ligand-sites and include, e.g., 4 sites binding Aβ. The single intracellular domain is involved in docking in proteins mediating cellular endocytosis. LRP1 located on the abluminal membrane mediates removal of Aβ in maintenance of normal brain homeostasis. It has been hypothesized that binding of Aβ involves a conformational change which initiates phosphate inositol-binding clathrin assembly protein (PICALM) on the intracellular domain that could regulate Aβ-transcytosis into the blood stream and by association with Rab5 and Rab11 mediating biogenesis of early and late endosomes including and of vesicles controlling transcytosis. LRP1 located at the luminal membrane surface may involve a similar protein outfit to accomplish cell internalization and transcytosis towards the brain parenchyma; however the mechanism of transcytosis through endothelial cells distinguishing opposite directions is still elusive.

Before internalization, the LRP1-receptor poses a signal recognized by a clathrin-attachment protein which controls the “wrapping” into a vesicle. The highly negatively charged polymalic acid-trileucine copolymer can be regarded as the platform for the attachment of “shuttle peptide” vectors and additional cargo on LRP1-receptor. The platform, vectors and cargo elect specific conformational change-driven interactions with membrane and receptor proteins on the intracellular domain endothelia cell membrane similarly as has been proposed for the initial reactions of the internalization of LRP1-Aβ complex. These interactions regulating the uptake of shuttle peptides intensified in our case by the interaction with cargo (IgG) or by an intensified shuttle peptide-receptor binding as in the case of the Aβ-binding (D-peptide) vectors. The interacting P/LLL copolymer platform (40% of the malic acid carboxylates amidated to LLL corresponding to a stretch of backbone that consists of 170 of malyl-β-ester units) is understood as a structurally flexible membrane anchorage that relocates and intensifies the vector/cargo receptor interactions as well may promote “wrapping” by the clathrin-coated endothelial membrane surface at the onset of the transcytosis pathway. The assembly of P/LLL-vector/cargo receptor complex is similar in the investigated brain locations cortex, midbrain and hippocampus. In normal and in diseased brain the endothelial cell layers display different receptor status and structural compactness resulting in different trans-BBB efficacies. Thus, AD brain is down regulated in LRP1, and glioma brain is upregulated in LRP1 and TfR and is structurally less compact giving rise to facilitated uptake and release of trespassing solutes (EPR-effect). Indeed, these differences are paralleled by the measured permeation efficacies (FIGS. 38A and 38B).

Ordering the BBB permeation by efficacy shows the shuttle peptides AP2, D3, D1 in the first positions in tumor (glioblastoma) at dose 4×: AP2>D3>D1≈M4>B6>P/LLL (FIG. 38A and Table 6). This leadership is confirmed in the glioma-free hemisphere of the same animal at 4×: AP2>D3>B6>D1≈P/LLL>M4 (Table 6). For comparison the hierarchy in cortex of tumor-free normal BALB/C at 8× (FIG. 38B): AP2>D3>B6 shows very similar results; however, at dose 4×: AP2>B6>M4; also: D1>D3>AP2 (FIG. 23B) and also: D1>D3>B6≈M4≈AP2 (FIGS. 36A-36F) the leadership of AP2 has changed place with D1 and D2 and has become comparable with B6 and M4. For hippocampus in normal brain the hierarchy is at 4×: D1≈D3>AP2 (FIG. 23B); D1>D3>AP2≈B6≈M4 (FIGS. 36A-36F); and for mid brain 4×: D1>D3>B6≈M4>AP2 (FIGS. 36A-36F) the leadership has again been held by D1, D3. The discrepancy between tumor-free hemisphere in the nude mouse model and the normal brain in BALB/C mice that could suggest a reflection of the brain tumor on the glioma bearing hemisphere is interesting but requires confirmation. The hierarchy in cortex of transgenic AD mice, dose 8×, is: D1≈D3>AP2>P/LLL≈B6≈M4 (FIG. 38B), and for hippocampus D3>D1>M4≈P/LLL≈AP2≈B6 (FIG. 38B), which shows a strong lead of the D-peptides manifested only at this dose 8×. In conclusion, the D1 and D3 are almost consistently the shuttle peptides generating the highest permeation efficacy through non-tumor BBB.

The by far the highest BBB permeation efficacies are measured in glioblastoma are which much reduced in normal brain and AD brain. The nanoconjugate P/LLL/AP2 of the peptide AP2 yielded highest efficacies in glioblastoma. This is consistent with an upregulation of the gene for LRP1, which is not matched by upregulation of the gene for TfR. The outstanding efficacies for AP2, D1, D3, M4 and B6-nanoconjugates in glioblastoma are largely owned to their more than linear dependence on their concentration and less to the P/LLL-effect. This is expressed by the over-linear dose dependence while the P/LLL contribution is linear. Because of their favorable efficacy (FIGS. 39A-39C, Table 6) their application is recommended for drug delivery to glioblastoma. In AD brain however, the dominant efficacy noticed for AP2 in glioma delivery is replaced by D1 and D3 conjugates at dose 8× increasing significantly over the efficacies for AP2 and the over shuttle peptides (FIGS. 37A-37E and 38A-38B). The superiority of D1, D3-conjugates in AD brain despite the downregulation of LRP1 in AD brain is referred to their phage-selected specificity of binding M. It is hypothesized that this specially involves binding to an LRP1 amyloid-receptor complex. In normal brain, where LRP1 is involved in mediating the efflux of Aβ as part of maintenance of homeostasis, a subsite containing Aβ could contribute to the superior permeation efficacy (FIG. 23B).

An extensive study of in vivo blood-brain barrier (BBB) crossing for normal (healthy) brain, AD brain, and brain tumor (glioblastoma) employing a fluorescence optical method was performed. The in vivo conditions were essential to demonstrate the influence of full conditions, for example the importance of Aβ in the BBB permeation of D1-, D3-conjugates, and for the identifying the effects in AD- and glioma-brain.

The results obtained for a multi-site polymeric vehicle were estimated to be highly significant for drug delivery to the brain parenchyma. This comprises in the future a multitude of drugs targeting receptors of brain cells for the purpose of research and treatment. Highest effort has been devoted characterizing and comparing different peptides aimed at different transcytosis pathways through BBB (shuttle peptides), their dose, and brain location dependent permeation efficacy. Qualitatively reproducible hierarchy arrays of shuttle peptide efficacy showed dose-dependent patterns typical for different brain status and brain locations. The arrays could be used as sensitive indicators of receptors involved in permeation pathways and as a tool to reveal variations in endothelial structure and functional capacity according to brain status and location.

In vitro BBB studies have confirmed that polymalic acid-mini nanodrugs containing combinations of shuttle peptides, tri-leucine peptide efficiently cross human endothelial BBB, without a requirement for opening tight junctions of the endothelial cell layer. In vivo different pathologies called for different strategies regarding platform composition and dosing for iv injection. For Alzheimer's disease, downregulation of receptors efficient in normal brain cells were encountered by injection of shuttle peptides, which were phage-selected to bind amyloid-beta containing LRP1 receptors at high injected dose 0.548 μmol/Kg (8×). In glioblastoma, upregulation of transcytosis pathways allowed high permeability though BBB at intermediate dose 0.274 μmol/Kg (4×). Dose dependence was exponential-like rather than linear and achieved a 10-15-fold increase for AP2-conjugate, but less efficacies for other shuttle peptides.

The delivery of “cargo”, i.e., of molecules attached to the platform and intended to function inside brain parenchyma has been exemplified here for IgG devoid of brain-specific binding. After attachment of rhodamine, the antibody was demonstrated to access normal brain. Shuttle peptides were grouped by their efficacies, P/LLL/AP2/IgG and P/LLL/B6/IgG in the highest category followed by P/LLL/D1/IgG and less by P/LLL/D3/IgG. In AD brain the D1-, D3-peptide conjugates were favored at dose 8× due to a mechanistically unresolved amyloid-beta peptide-supported function of LRP1 receptor.

It was observed herein that the permeation efficacy tended to be elevated at high doses of nanoconjugates suggesting dose-dependent multisite interactions between conjugates and receptors along the BBB permeation pathway. Conducting multifactorial analysis of tumor (glioblastoma) brain confirmed coupling between ligands AP2 and B6 at the polymer platform and their interactions with receptor sites along the BBB permeation path. According to our similar findings of exponential-like dose dependency, the basic conclusions from brain tumor, although efficacies were significantly higher, can be extended to normal brain and AD brain. The multifactorial analysis resting on the optical measurement of free vector accumulation in parenchyma can be applied to brain of different status and brain locations. It is novel because it follows the in situ permeation from brain capillary into parenchyma of peptide vectors attached to a multi-functional platform, single or in array, and examine additional functional cargo (drugs and targets).

### Example 33

### Advantages of the Mini Nanodrugs for Trans-BBB Delivery

A biodegradable non-toxic β-poly(L-malic acid) (PMLA or P) was synthesized as a scaffold to chemically bind the BBB crossing peptides Angiopep-2 (AP2), Miniap-4 (M4), and the transferrin receptor directed ligands cTfRL and B6. In addition, a tri-leucine endosome escape unit (LLL) and a fluorescent marker (rhodamine) were attached to the PMLA backbone. The pharmacokinetics, BBB penetration and distribution of mini nanodrugs were examined in different brain regions and at multiple time points via optical imaging. The mini nanodrug containing P/LLL/AP-2 produced significant fluorescence in the parenchyma of the cortex, midbrain and hippocampus 30 minutes after a single intravenous injection; clearance was observed after four hours. The mini nanodrug variant P/LLL lacking AP-2, or the variant P/AP-2 lacking LLL, showed significantly less BBB penetration. The LLL moiety appeared to stabilize the nanoconjugate, while AP-2 enhanced BBB penetration. The mini nanodrug containing the peptide cTfRL displayed comparably little and/or inconsistent infiltration of brain parenchyma, likely due to reduced trans-BBB transport. P/LLL/AP-2 or the other peptides can now be functionalized with intra-brain targeting and drug treatment moieties that are aimed at molecular pathways implicated in neurological disorders.

A nanodrug platform for trans-BBB drug delivery was presented. The strategy builds on previously published peptides to shuttle a PMLA-based drug platform across the BBB. Surprisingly, PMLA/LLL/peptide interactions were observed to determine the BBB passage, and detailed investigation was performed to determine how the mini nanodrug was distributed in the brain. In addition, it was observed that moieties of inherent hydrophobic structure, such as LLL, influence and enhance brain delivery, especially in areas with high blood vessel density such as the midbrain. This effect may be due to inherent drug properties. The results indicate that the BBB, for the nanodrug s (P/LLL/AP-2, P/LLL/M4 or P/LLL/B6-conjugates) and under applied conditions, may not constitute an efficient barrier and that it can be open to deliver high amounts of covalently bound drug for pharmaceutical treatment.

Neurological disorders affect brain regions differently, and almost every disease can be attributed to specific malfunctions in a brain region. A detailed knowledge of nanodrug behavior in different brain regions is thus useful for drug development and such information is provided here. With P/LLL/AP-2, only 50% of the carboxylic acids was functionalized, leaving the construct with additional sites to further equip the nanodrug with targeting and drug treatment moieties.

Advantages of the system: (1) Easy and low cost synthesis of novel combination of peptides conjugates with polymalic acid. (2) Microscopic evidence is provided that demonstrates the nanodevices permeation across healthy and Alzheimer BBB. (3) Fast exit from vascular into targeted tissue with long-lived retention in tissue (PK and comparison with microscopic prevalence of nanodevices in parenchyma). (4) Therefore replacement of BBB transcytosis targeting antibodies by receptor affine specific peptides providing tuned affinity and rates of receptor-peptides association/dissociation. (5) Nano platform with multiplicity of sites for drugs and targeting groups. (6) Controlled site-responsive release of drugs (pH, enzymes, disulfide exchange). (7) Drug and targeting molecules freely accessible for immediate activity (linear array of ligands on nanocarrier platform, absence of occlusions by crowding antibodies). (8) Mini nanocarriers of <10 nm size and elongated shape (high axial ratio) i.e. absence of bulky proteins (antibodies) for fast diffusion through barriers and deep tissue penetration (Ding et al. (2016) Nanomedicine 13, 631-659, which is incorporated herein by reference as if fully set forth). (9) Macromolecular nanocarriers (all covalent bonds) to ensure tunable high chemical and physical stability. (10) Cleavage resistant peptides with exocyclic structure and lack of substrate properties for absence of enzymatic cleavage. (11) Fast systemic clearance of nanodevice to keep interference by degradation fragments at minimum. (12) Biodegradability, absence of uncontrolled systemic toxicity and antigenicity (e.g. by antibodies). (13) Manufacture as a powder. Soluble mini nanodrug by infusion at the time and place of application.

Sequences and conformation of targeting and functioning peptides provide high resistance to in vivo degradation (exocyclic or D-conformation). Values of dissociation constants at micro molar or below. Except for tau, nucleic acids sequences of genes/amino acid sequences for targeting malignant disease marker proteins β-secretase 1 (BACE1), presenilin 1, are available for targeting and the design of antisense oligo nucleotides.

Additionally, a mini nanodrug provides sufficient activity against homeostasis imbalancing body constituents during treatment of the recipients. Mini nanodrugs do not oversupply the recipient organism with drugs and delivering vehicles and the components they are built from. A mini drug eludes principles of carrying a close to minimal supply at maximum effective drug doses in the best officious physical make up for deep tissue penetration. The mini nanodrug is a receptor targeting construct of minimum surface, elongated form and moderately strong binding affinities in order to maximise receptor releasing kinetics and fast bio barrier penetration, minimum antigenetic content to minimise immune reaction and biodegradability to avoid long lasting in vivo depositions.

### REFERENCES

Abbott, N. J. Blood-brain barrier structure and function and the challenges for CNS drug delivery. Journal of inherited metabolic disease 2013, 36, 437-449.
Alyautdin, R.; Khalin, I.; Nafeeza, M. I.; Haron, M. H.; Kuznetsov, D. Nanoscale drug delivery systems and the blood-brain barrier. International journal of nanomedicine 2014, 9, 795.
Borlak, J.; Länger, F.; Spanel, R.; Schöndorfer, G.; Dittrich, C. Immune-mediated liver injury of the cancer therapeutic antibody catumaxomab targeting EpCAM, CD3 and Fcγ receptors. Oncotarget 2016, 7, 28059.
Bourassa, P., Alata, W., Tremblay, C , Paris-Robidas, S., Calon, F., Transferrin Receptor-Mediated Uptake at the Blood-Brain Barrier Is Not Impaired by Alzheimer's Disease Neuropathology, Molecular Pharmaceutics, 16 (2019) 583-594.
Chen C, Liang Z, Zhou B, Li X, Lui, Ip N Y, Qu J Y In Vivo Near-Infrared Two-Photon Imaging of Amyloid Plaques in Deep Brain of Alzheimer's Disease Mouse Model. ACS Chem Neurosci. 2018 Aug. 16. doi: 10.1021/acschemneuro.8b00306.
Dehouck, B.; Fenart, L.; Dehouck, M.-P.; Pierce, A.; Torpier, G.; Cecchelli, R. A new function for the LDL receptor: transcytosis of LDL across the blood-brain barrier. The Journal of cell biology 1997, 138, 877-889.
Demeule M, Beaudet N, Régina A, Besserer-Offroy E, Murza A, Tétreault P, et al. (2014) Conjugation of a brain-penetrant peptide with neurotensin provides antinociceptive properties. Clin Invest. 124, 1199-1213. doi:10.1172/JCI70647.
Demeule, M., Currie, J. C., Bertrand, Y., . Che, C., Nguyen, T., Regina, A., Gabathuler, R., Castaigne, J. P., Beliveau, R., Involvement of the low-density lipoprotein receptor-related protein in the transcytosis of the brain delivery vector Angiopep-2, Journal of neurochemistry, 106 (2008) 1534-1544.
Demeule M, Regina A, Che C, Poirier J, Nguyen T, Gabathuler R, Castaigne J-P, Beliveau R. (2008) Identification and Design of Peptides as a New Drug Delivery System for the Brain. J Pharm Exp Ther 324,1064-1072.
DeVos S L, Miller R L, Schoch K M, Holmes B B, Kebodeaux C S, Wegener A J, et al. (2017) Tau reduction prevents neuronal loss and reverses pathological tau deposition and seeding in mice with tauopathy. Sci. Transl. Med. 9, eaag0481.
Di Guardo, G. Lipofuscin, Lipofuscin-Like Pigments and Autofluorescence. European Journal of Histochemistry: EJH 2015, 59, 2485.
Ding H, Gangalum P R, Galstyan A, Fox I, Patil R, Hubbard P, Murali R, Ljubimova J Y, Holler E. (2016) HER2-positive breast cancer targeting and treatment by a peptide-conjugated mini nanodrug. Nanomedicine 13, 631-659.
Ding, H.; Inoue, S.; Ljubimov, A. V.; Patil, R.; Portilla-Arias, J.; Hu, J.; Konda, B.; Wawrowsky, K. A.; Fujita, M.; Karabalin, N. Inhibition of brain tumor growth by intravenous poly (β-L-malic acid) nanobioconjugate with pH-dependent drug release. Proceedings of the National Academy of Sciences 2010, 107, 18143-18148.
Drean, A.; Goldwirt, L.; Verreault, M.; Canney, M.; Schmitt, C.; Guehennec, J.; Delattre, J.-Y.; Carpentier, A.; Idbaih, A. Blood-brain barrier, cytotoxic chemotherapies and glioblastoma. Expert Review of Neurotherapeutics 2016, 16, 1285-1300.
Elnegaard, M. P.; List, M.; Christiansen, H.; Schmidt, S.; Mollenhauer, J.; Block, I. Protein-based nanotoxicology assessment strategy. Nanomedicine: Nanotechnology, Biology and Medicine 2017, 13, 1229-1233.
Funke S A, Bartnik D, Gluck J M, Piorkowska K, Wiesehan K, Weber U, Gulyas B, . . . et al. (2012) Development of a Small D-Enantiomeric Alzheimer's Amyloid-b Binding Peptide Ligand for Future In Vivo Imaging Applications. PLoS ONE 7 (7): e41457. doi:10.1371/journal.pone.0041457.
Gao S, Tian H, Xing Z, Zhang D, Guo Y, Gu Z, et al. (2016) A non-viral suicide gene delivery system traversing the blood brain barrier for non-invasive glioma targeting treatment. Journal of Controlled Release 243, 357-369.
Georgieva, J. V.; Hoekstra, D.; Zuhorn, I. S. Smuggling drugs into the brain: an overview of ligands targeting transcytosis for drug delivery across the blood-brain barrier. Pharmaceutics 2014, 6, 557-583.
Grimmer, T.; Goldhardt, O.; Guo, L.-H.; Yousefi, B. H.; Förster, S.; Drzezga, A.; Sorg, C.; Alexopoulos, P.; Förstl, H.; Kurz, A LRP-1 polymorphism is associated with global and regional amyloid load in Alzheimer's disease in humans in-vivo. NeuroImage: Clinical 2014, 4, 411-416.
Herr, U. M.; Strecker, P.; Storck, S. E.; Thomas, C.; Rabiej, V.; Junker, A.; Schilling, S.; Schmidt, N.; Dowds, C. M.; Eggert, S.; Pietrzik, C. U.; Kins, S., LRP1 Modulates APP Intraneuronal Transport and Processing in Its Monomeric and Dimeric State. Frontiers in molecular neuroscience 2017, 10, 118.
Israel, L. L., Braubach, O., Galstyan, A., Chiechi, A., Shatalova, E. S., Grodzinski, Z., Ding, H., Black, K. L., Ljubimova, J. Y., Holler, E., A Combination of Tri-Leucine and Angiopep-2 Drives a Poly-Anionic Polymalic Acid Nanodrug Platform Across the Blood-Brain Barrier, ACS Nano, 13, 2019, 1253-1271.
Jiang, N., Frenzel, D., Schartmann, E., van Groen, T., Kadish, I., Shah, N. J., Langen, K. J., Willbold, D., Willuweit, A., Blood-brain barrier penetration of an Abeta-targeted, arginine-rich, d-enantiomeric peptide, Biochimica et biophysica acta, 1858 (2016) 2717-2724.
Johnsen, K. B.; Burkhart, A.; Melander, F.; Kempen, P. J.; Vejlebo, J. B.; Siupka, P.; Nielsen, M. S.; Andresen, T. L.; Moos, T. Targeting transferrin receptors at the blood-brain barrier improves the uptake of immunoliposomes and subsequent cargo transport into the brain parenchyma. Scientific reports 2017, 7, 10396.
Kenett, with contributions by Amberti, Modern Industrial Statistics: with applications in R, MINITAB and JMP, 2^{nd} ed., John Wiley and Sons, Chichester 2014.
Kenett and Zacks, Modern industrial statistics: design and control of quality and reliability, Duxbury press San Francisco 1998.
Kung M P, Weng C C, Lin K J, Yen T H C, Wey S P. Amyloid Plaque Imaging from IMPY/SPECT to AV-45/PET. Journey of Aβimaging, Chang Gung Med J Vol. 35 No. 3. May-June 2012
Li Y, Zheng X , Gong M, Zhang J (2016) Delivery of a peptide-drug conjugate targeting the blood brain. Oncotarget 7, 79401-49407.
Liu, M.; Li, X.; Xie, Z.; Xie, C.; Zhan, C.; Hu, X.; Shen, Q.; Wei, X.; Su, B.; Wang, J.; Lu, W., D-Peptides as Recognition Molecules and Therapeutic Agents. Chemical record (New York, N.Y.) 2016, 16 (4), 1772-86.
Ljubimova J Y, Sun T, Mashouf L, Ljubimov A V, Israel L L, Ljubimov V A, Falahatian V, Holler E. (2017) Covalent nano delivery systems for selective imaging and treatment of brain tumors. Adv Drug Deliv Rev 113, 177-200.
Ljubimova J Y, Ding H, Portilla-Arias J, Patil R, Gangalum P R, Chesnokova A, Inoue S, Rekechenetskiy A, Nassoura T, Black K L, Holler E. Polymalic Acid-based Nano Biopolymers for Targeting of Multiple Tumor Markers: An Opportunity for Personalized Medicine? J Vis Exp. 13, e50668 (2014).
Lu F, Pang Z, Zhao J, Jin K, Li H, Pang Q, et al, (2017) Angiopep-2-conjugated poly(ethylene glycol)-copoly(ε-caprolactone) polymersomes for dual targeting drug delivery to glioma in rats. International Journal of Nanomedicine 12, 2117-2127.
Meng, J.; Agrahari, V.; Youm, I. Advances in targeted drug delivery approaches for the central nervous system tumors: the inspiration of nanobiotechnology. Journal of Neuroimmune Pharmacology 2017, 12, 84-98.
Miller, S. M.; Simon, R. J.; Ng, S.; Zuckermann, R. N.; Kerr, J. M.; Moos, W. H., Comparison of the proteolytic susceptibilities of homologous L-amino acid, D-amino acid, and N-substituted glycine peptide and peptoid oligomers. Drug Development Research 1995, 35 (1), 20-32.
Morales-Zavalaa F, Arriagadac H, Hassana N, Velascoa C, Riverosa A, R. Álvarez A R. (2017) Peptide multi functionalized gold nanorods decrease toxicity of β-amyloid peptide in a Caenorhabditis elegans model of Alzheimer's disease. Nanomedicine: Nanotechnology, Biology, and Medicine 13, 2341-2350.
Nordberg A, Amyloid plaque imaging in vivo: current achievement and future prospects. Eur J Nucl Med Mol Imaging. 2008 DOI 10.1007/s00259-007-0700-2.
Oller-Salvia B, Sanchez-Navarro M, Ciudad S, Guiu M, Arranz-Gibert P, Garcia C, Gomis R R, Cecchelli R, Garcia J, Giralt E, Teixid M (2010) MiniAp-4: A venom-Inspired Peptidomimetic for Brain Delivery. Angew Chem Int Ed 55, 572-575.
Oller-Salvia, B.; Sánchez-Navarro, M.; Ciudad, S.; Guiu, M.; Arranz-Gibert, P.; Garcia, C.; Gomis, R. R.; Cecchelli, R.; García, J.; Giralt, E. MiniAp-4: a venom-inspired peptidomimetic for brain delivery. Angewandte Chemie International Edition 2016, 55, 572-575.
O'Sullivan C C, Lindenberg M, Bryla C, Patronas N, Peer C J, Amiri-Kordestani L. (2016) ANG1005 for breast cancer brain metastases: correlation between 18F-FLT PET after first cycle and MRI in response assessment. Breast Cancer Res Treat 160, 51-59.
Patil, R.; Ljubimov, A. V.; Gangalum, P. R.; Ding, H.; Portilla-Arias, J.; Wagner, S.; Inoue, S.; Konda, B.; Rekechenetskiy, A.; Chesnokova, A.; et al. MRI Virtual Biopsy and Treatment of Brain Metastatic Tumors with Targeted Nanobioconjugates: Nanoclinic in the Brain. ACS Nano 2015, 9 (5), 5594-5608.
Ramanathan, A., Nelson, A. R., Sagare, A. P. , Zlokovic, B. V., Impaired vascular-mediated clearance of brain amyloid beta in Alzheimer's disease: the role, regulation and restoration of LRP1, Frontiers in Aging Neuroscience, 7 (2015) 136.
Regina A, Demeule M, Che C, Lavalle I, Poirier J, Gabathuler R, et al. (2008) Anti tumour activity of ANG1005, a conjugate between paclitaxel and the new brain delivery vector Angiopep-2. British Journal of Pharmacology 155, 185-197.
Rozema, D. B.; Ekena, K.; Lewis, D. L.; Loomis, A. G.; Wolff, J. A. Endosomolysis by Masking of a Membrane-Active Agent (EMMA) for Cytop
Schindelin, J.; Arganda-Carreras, I.; Frise, E.; Kaynig, V.; Longair, M.; Pietzsch, T.; Preibisch, S.; Rueden, C.; Saalfeld, S.; Schmid, B.; et al. Fiji: an open-source platform for biological-image analysis. Nature methods 2012, 9, 676-82.
Scot J D., Li S W., Brunskill J P, . . . , et al. Discovery of the 3-Imino-1,2,4-thiadiazine 1,1-Dioxide Derivative Verubecestat (MK-8931)—A β—Site Amyloid Precursor Protein Cleaving Enzyme 1 Inhibitor for the Treatment of Alzheimer's Disease, J Med Chem, 2016, 59, 10435-10450.
Sevigny, J.; Chiao, P.; Bussiere, T.; Weinreb, P. H.; Williams, L.; Maier, M.; Dunstan, R.; Salloway, S.; Chen, T.; Ling, Y. The antibody aducanumab reduces Aβ plaques in Alzheimer's disease. Nature 2016, 537, 50-56.
Staquicini F I, Ozawa M G, Moya C A, Driessen W H P, Barbu E M, Nishimori H, et al. (2011) Systemic combinatorial peptide selection yields a non-canonical iron-mimicry mechanism for targeting tumors in a mouse model of human glioblastoma. J Clin Invest 121, 161-173. doi:10.1172/JCI44798.
Van Groen T, Kadish I, Wiesehan K, Funke S A, and Willbol D. (2009) In vitro and in vivo Staining Characteristics of Small, Fluorescent, Ab42-Binding d-Enantiomeric Peptides inTransgenic AD Mouse Models. ChemMedChem, 4, 276-282.
Vassar R. (2014) BACE1 inhibitor drugs in clinical trials for Alzheimer's disease Alzheimer's Research & Therapy 6:89. DOI 10.1186/s13195-014-0089-7.
Wiesehan K, Buder K, Linke R P, Patt S, Stoldt M, Unger E, et al. (2003) Selection of Diamino-acid peptides that bind to Alzheimer's disease amyloid peptide abeta1-42 by mirror image phage display. Chembiochem 4, 748e53.
Xin H, Sha X, Jiang X, Zhang W, Chen L, Fang X. (2012) Anti-glioblastoma efficacy and safety of paclitaxel-loading Angiopep-conjugated dual targeting PEG-PCL nanoparticles. Biomaterials 33, 8167-8176
Yanick Bertrand Y, Currie J-C, Demeule M, Régina A, Ché C, Abulrob A, Fatehi D, Sartelet H, Gabathuler R, Castaigne J-P, Stanimirovic D, Beliveau R. (2010) Transport characteristics of a novel peptide platform for CNS therapeutics. J Cell Mol Med 14, 2827-2839.
Yan H, Wang L, Wang J, Weng X, Lei H, Wang X, et al. (2012) Two-Order Targeted Brain Tumor Imaging by Using an Optical/Paramagnetic Nanoprobe across the Blood Brain Barrier. ACS Nano 6, 410-420.
Yin, T.; Yang, L.; Liu, Y.; Zhou, X.; Sun, J.; Liu, J. Sialic acid (SA)-modified selenium nanoparticles coated with a high blood-brain barrier permeability peptide-B6 peptide for potential use in Alzheimer's disease. Acta biomaterialia 2015, 25, 172-183.
Yi Wang, Xiaoying Ying, Liying Chen, Yao Liu, Ying Wang, Jiao Liang, et al. (2016) Electro responsive Nanoparticles Improve Anti seizure Effect of Phenytoin in Generalized Tonic-Clonic Seizures. Neurotherapeutics 13, 603-613.
Yu, Y. J., Zhang, Y., Kenrick, M., Hoyte, K., Luk, W. , Lu, Y., Atwal, J., Elliott, J. M., Prabhu, S., Watts, R. J., Dennis, M. S., Boosting Brain Uptake of a Therapeutic Antibody by Reducing Its Affinity for a Transcytosis Target. ScienceTranslationalMedicine.org 25 May 2011 Vol 3 Issue 84 84ra44.
Zeidman, P.; Maguire, E. A. Anterior hippocampus: the anatomy of perception, imagination and episodic memory. Nature Reviews Neuroscience 2016, 17, 173.
Zhang C, Zheng X, Wan X, Shao X, Liu Q, Zhang Z, Zhang Q. (2014) The potential use of H102 peptide-loaded dual-functional nanoparticles in the treatment of Alzheimer's disease. J Controlled Release 192, 317-324.
Zheng X, Pang X, Yang P, Wan X, Wei Y, Guo G, et al. (2017) A hybrid siRNA delivery complex for enhanced brain penetration and precise amyloid plaque targeting in Alzheimer's disease mice. Acta Biomaterialia 49 (2017) 388-401.

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

What is claimed is:

1. A mini nanodrug comprising:
a polymalic acid-based molecular scaffold;
at least one peptide capable of crossing the blood-brain barrier;
at least one plaque-binding peptide; and
an endosomolytic ligand; wherein
each of the at least one peptide capable of crossing the blood-brain barrier, the at least one plaque-binding peptide and the endosomolytic ligand are covalently linked to the polymalic acid-based molecular scaffold, and
the mini nanodrug ranges in size from 1 nm to 10 nm.

2. The mini nanodrug of claim 1, wherein the at least one peptide capable of crossing the blood-brain barrier is an LRP-1 ligand, or a transferrin receptor ligand.

3. The mini nanodrug of claim 1, wherein the at least one peptide capable of crossing the blood-brain barrier is a peptide selected from the group consisting of Angiopep-2, Fe mimetic peptide, B6 peptide, and Miniap-4 peptide, or variants thereof.

4. The mini nanodrug of claim 3, wherein the at least one peptide capable of crossing the blood-brain barrier is Angiopep-2 comprising a sequence of SEQ ID NO: 1, or a variant thereof.

5. The mini nanodrug of claim 3, wherein the at least one peptide capable of crossing the blood-brain barrier is Fe mimetic peptide comprising an amino acid sequence of SEQ ID NO: 2, or a variant thereof.

6. The mini nanodrug of claim 3, wherein the at least one peptide crossing the blood-brain barrier is B6 peptide comprising an amino acid sequence of SEQ ID NO: 8, or a variant thereof.

7. The mini nanodrug of claim 3, wherein the at least one peptide crossing the blood-brain barrier is a Miniap-4 peptide comprising an amino acid sequence of SEQ ID NO: 3, or a variant thereof.

8. The mini nanodrug of claim 1, wherein the at least one peptide capable of crossing the blood-brain barrier comprises at least two peptides capable of crossing the blood-brain barrier.

9. The mini nanodrug of claim 8, wherein each of the at least two peptides capable of crossing the blood-brain barrier is selected independently.

10. The mini nanodrug of claim 1, wherein each of the at least one peptide capable of crossing the blood-brain barrier and the plaque-binding peptide is conjugated to the polymalic acid-based molecular scaffold by a linker.

11. The mini nanodrug of claim 10, wherein the linker comprises a polyethylene glycol (PEG).

12. The mini nanodrug of claim 1, wherein the endosomolytic ligand comprises Trp-Trp-Trp (WWW), Phe-Phe-Phe (FFF), Leu-Leu-Leu (LLL), or Ile-Ile-Ile (I-I-I).

13. The mini nanodrug of claim 1, wherein the mini nanodrug further comprises a therapeutic agent

14. The mini nanodrug of claim 13, wherein the therapeutic agent is selected from the group consisting of: an antisense oligonucleotide, an RNA oligonucleotide, a peptide, and a low molecular weight drug.

15. The mini nanodrug of claim 14, wherein the therapeutic agent is an antisense oligonucleotide complementary to a β-secretase mRNA sequence or a γ-secretase mRNA sequence.

16. The mini nanodrug of claim 15, wherein the antisense oligonucleotide comprises a nucleic acid sequence with at least 90% identity to SEQ ID NO: 4.

17. The mini nanodrug of claim 13, wherein the therapeutic agent is an oligonucleotide capable of targeting a messenger RNA transcribed from a target gene.

18. The mini nanodrug of claim 17, wherein the target gene encodes BACE1, and the oligonucleotide comprises a sequence with at least 90% identity to SEQ ID NO: 14.

19. The mini nanodrug of claim 14, wherein the therapeutic agent is a peptide.

20. The mini nanodrug of claim 19, wherein the peptide is a β-sheet breaker peptide comprising a sequence of SEQ ID NO: 6, or a variant thereof.

21. The mini nanodrug of claim 1, wherein the plaque-binding peptide is a D-enantiomeric peptide.

22. The mini nanodrug of claim 21, wherein the D-enantiomeric peptide is selected from the group consisting of: a D 1-peptide, a D3-peptide and an ACI-89-peptide, or variants thereof.

23. The mini nanodrug of claim 22, wherein the D-enantiomeric peptide is the D1-peptide comprising an amino acid sequence of SEQ ID NO: 9, or a variant thereof.

24. The mini nanodrug of claim 22, wherein the D-enantiomeric peptide is the D3-peptide comprising an amino acid sequence of SEQ ID NO: 10, or a variant thereof.

25. The mini nanodrug of claim 22, wherein the D-enantiomeric peptide is the ACI-89-peptide comprising an amino acid sequence of SEQ ID NO: 11, or a variant thereof.

26. The mini nanodrug of claim 1, wherein the plaque-binding peptide comprises at least two plaque-binding peptides.

27. The mini nanodrug of claim 1, wherein the at least one peptide capable of crossing the blood brain barrier is selected from the group consisting of: Angiopep-2, Fe mimetic peptide, B6 peptide, Miniap-4 peptide, and variants thereof, the at least one plaque-binding peptide is selected from the group consisting of: a D1-peptide, a D3-peptide and an ACI-89-peptide, or variants thereof, and the endosomolytic ligand comprises Trp-Trp-Trp (WWW), Phe-Phe-Phe (FFF), Leu-Leu-Leu (LLL), or Ile-Ile-Ile (I-I-I).

28. The mini nanodrug of claim 1, wherein the nanodrug further comprises an imaging agent covalently linked with the polymalic acid-based molecular scaffold.

29. The mini nanodrug of claim 28, wherein the imaging agent comprises a fluorescence moiety, a radioisotope moiety, or a magnetic resonance imaging moiety.

30. The mini nanodrug of claim 28 comprising the at least one peptide capable of crossing the blood brain barrier selected from the group consisting of: Angiopep-2, Fe mimetic peptide, B6 peptide, Miniap-4 peptide, and variants thereof, the plaque-binding peptide selected from the group consisting of: a D1-peptide, a D3-peptide and an ACI-89-peptide, or variants thereof, the endosomolytic ligand comprising Trp-Trp-Trp (WWW), Phe-Phe-Phe (FFF), Leu-Leu-Leu (LLL), or Ile-Ile-Ile (I-I-I), and the imaging agent comprising a fluorescence moiety, a radioisotope moiety, or a magnetic resonance imaging moiety.

31. The mini nanodrug of claim 1, wherein the polymalic acid-based molecular scaffold comprises poly(β-L-malic acid).

32. The mini nanodrug of claim 1, wherein the mini nanodrug further comprises an antibody.

33. The mini nanodrug of claim 32, wherein the antibody is an IgG antibody, or fragment thereof.

34. A mini nanodrug comprising:
a polymalic acid-based molecular scaffold;
at least one peptide capable of crossing the blood-brain barrier;
an endosomolytic ligand; and
a therapeutic agent, wherein
each of the at least peptide capable of crossing the blood-brain barrier, the endosomolytic ligand and the therapeutic agent are covalently linked to the polymalic acid-based molecular scaffold, and
the mini nanodrug ranges in size from 1 nm to 10 nm.

35. The mini nanodrug of claim 34, wherein the at least one peptide capable of crossing the blood-brain barrier is a peptide selected from the group consisting of Angiopep-2, Fe mimetic peptide, B6 peptide, and Miniap-4 peptide, or variants thereof.

36. The mini nanodrug of claim 35, wherein the at least one peptide capable of crossing the blood-brain barrier is Angiopep-2 comprising an amino acid sequence of SEQ ID NO: 1, or a variant thereof.

37. The mini nanodrug of claim 35, wherein the at least one peptide capable of crossing the blood-brain barrier is Fe mimetic peptide comprising an amino acid sequence of SEQ ID NO: 2, or a variant thereof.

38. The mini nanodrug of claim 35, wherein the at least one peptide capable of crossing the blood-brain barrier is B6 peptide comprising an amino acid sequence of SEQ ID NO: 8, or a variant thereof.

39. The mini nanodrug of claim 35, wherein the at least one peptide capable of crossing the blood-brain barrier is a Miniap-4 peptide comprising an amino acid sequence of SEQ ID NO: 3, or a variant thereof.

40. The mini nanodrug of claim 34, wherein the at least one peptide capable of crossing the blood-brain barrier comprises at least two peptides capable of crossing the blood-brain-barrier.

41. The mini nanodrug of claim 40, wherein each of the at least two peptides is selected independently.

42. The mini nanodrug of claim 34, wherein each of the at least one peptide capable of crossing the blood-brain barrier is conjugated to the polymalic acid-based molecular scaffold by a linker.

43. The mini nanodrug of claim 34, wherein the endosomolytic ligand comprises Trp-Trp-Trp (WWW), Phe-Phe-Phe (FFF), Leu-Leu-Leu (LLL), or Ile-Ile-Ile (I-I-I).

44. The mini nanodrug of claim 34, wherein the therapeutic agent is selected from the group consisting of: an antisense oligonucleotide, an siRNA oligonucleotide, a peptide, and a low molecular weight drug.

45. The mini nanodrug of claim 44, wherein the therapeutic agent comprises an antisense oligonucleotide complementary to a β-secretase mRNA sequence or a γ-secretase mRNA sequence.

46. The mini nanodrug of claim 44, wherein the antisense oligonucleotide comprises a nucleic acid sequence with at least 90% identity to SEQ ID NO: 4.

47. The mini nanodrug of claim 44, wherein the therapeutic agent is an oligonucleotide capable of targeting a messenger RNA transcribed from a target gene.

48. The mini nanodrug of claim 47, wherein the target gene encodes BACE1, and the oligonucleotide comprises a sequence with at least 90% identity to SEQ ID NO: 14.

49. The mini nanodrug of claim 34, wherein the at least one peptide capable of crossing the blood-brain barrier is a peptide selected from the group consisting of Angiopep-2, Fe mimetic peptide, B6 peptide, and Miniap-4 peptide, or variants thereof, the therapeutic agent comprises an antisense oligonucleotide complementary to a β-secretase mRNA sequence or a γ-secretase mRNA sequence, and the endosomolytic ligand comprises Trp-Trp-Trp (WWW), Phe-Phe-Phe (FFF), Leu-Leu-Leu (LLL), or Ile-Ile-Ile (I-I-I).

50. The mini nanodrug of claim 44, wherein the therapeutic agent is a peptide comprising a β-sheet breaker peptide.

51. The mini nanodrug of claim 50, wherein the β-sheet breaker peptide comprises an amino acid sequence of SEQ ID NO: 6, or a variant thereof.

52. The mini nanodrug of claim 34, wherein the at least one peptide capable of crossing the blood-brain barrier is a peptide selected from the group consisting of Angiopep-2, Fe mimetic peptide, B6 peptide, and Miniap-4 peptide, or variants thereof, the therapeutic agent comprises a β-sheet breaker peptide, and the endosomolytic ligand comprises Trp-Trp-Trp (WWW), Phe-Phe-Phe (FFF), Leu-Leu-Leu (LLL), or Ile-Ile-Ile (I-I-I).

53. The mini nanodrug of claim 34, wherein the mini nanodrug further comprises a plaque-binding peptide.

54. The mini nanodrug of claim 53, wherein the plaque-binding peptide is a D-enantiomeric peptide selected from the group consisting of: a D1-peptide, a D3-peptide and an ACI-89-peptide, or variants thereof.

55. The mini nanodrug of claim 53, wherein the D-enantiomeric peptide is a peptide comprising an amino acid sequence of SEQ ID NO: 9, 10 or 11, or variants thereof.

56. The mini nanodrug of claims 34, wherein the mini nanodrug further comprises an imaging agent covalently linked with the polymalic acid-based molecular scaffold.

57. The mini nanodrug of claim 56, wherein the imaging agent comprises a fluorescence moiety, a radioisotope moiety, or a magnetic resonance imaging moiety.

58. The mini nanodrug of claim 56, wherein the at least one peptide capable of crossing the blood-brain barrier is a peptide selected from the group consisting of Angiopep-2, Fe mimetic peptide, B6 peptide, and Miniap-4 peptide, or variants thereof, the therapeutic agent comprises an antisense oligonucleotide complementary to a β-secretase mRNA sequence or a γ-secretase mRNA sequence, the endosomolytic ligand comprises Trp-Trp-Trp (WWW), Phe-Phe-Phe (FFF), Leu-Leu-Leu (LLL), or Ile-Ile-Ile (I-I-I), and the imaging agent comprising a fluorescence moiety, a radioisotope moiety, or a magnetic resonance imaging moiety.

59. The mini nanodrug of claim 34, wherein the at least one peptide capable of crossing the blood-brain barrier is a peptide selected from the group consisting of Angiopep-2, Fe mimetic peptide, B6 peptide, and Miniap-4 peptide, or variants thereof, the therapeutic agent comprises a β-sheet breaker peptide, the endosomolytic ligand comprises Trp-Trp-Trp (WWW), Phe-Phe-Phe (FFF), Leu-Leu-Leu (LLL), or Ile-Ile-Ile (I-I-I), and the imaging agent comprising a fluorescence moiety, a radioisotope moiety, or a magnetic resonance imaging moiety.

60. The mini nanodrug of claim 34, wherein the mini nanodrug further comprises an antibody.

61. The mini nanodrug of claim 67, wherein the antibody is an IgG antibody or fragment thereof.

62. A pharmaceutically acceptable composition comprising a mini nanodrug of claim 1 or 34, and a pharmaceutically acceptable carrier or excipient.

63. A method for treating a brain disease or abnormal condition in a subject, comprising: administering a therapeutically effective amount of a mini nanodrug of claim 1 or 34, or a pharmaceutically acceptable composition of claim 62 to a subject in need thereof.

64. The method of claim 63, wherein the brain disease or abnormal condition is selected from the group consisting of Alzheimer's disease, Multiple sclerosis, Parkinson's disease, Huntington's disease, schizophrenia, anxiety, dementia, mental retardation, and anxiety

65. The method of claim 64, wherein the brain disease is Alzheimer's disease.

66. The method of claim 65, wherein the Alzheimer's disease is treated, prevented or ameliorated after administering the mini nanodrug.

67. The method of claim 63, wherein administration is performed at least once a week, at least once a day, or at least twice a day for at least one month.

68. The method of claim 63, wherein the subject is a mammal.

69. The method of claim 68, wherein the mammal is selected from the group consisting of: a rodent, a canine, a primate, an equine, an experimental human-breast tumor-bearing nude mouse, and a human.

70. A method for reducing formation of amyloid plaques in the brain of a subject, comprising administering the mini nanodrug of claim 1 or 34, or composition of claim 62 to a subject in need thereof.

71. A method for treating a proliferative disease in a subject, comprising:
administering a therapeutically effective amount of a mini nanodrug comprising a polymalic acid-based molecular scaffold, at least one peptide capable of crossing the blood-brain barrier, an endosomolytic ligand and an therapeutic agent to a subject in need thereof,
wherein each of the at least peptide, the endosomolytic ligand and the therapeutic agent are covalently linked to the polymalic acid-based molecular scaffold, and the mini nanodrug ranges in size from 1 nm to 10 nm.

72. The method of claim 71, wherein the proliferative disease is a cancer.

73. The method of claim 72, wherein the cancer is selected from the group consisting of: glioma, glioblastoma, breast cancer metastasized to the brain and lung cancer metastasized to the brain.

74. The method of claim 71, wherein the therapeutic agent is an anti-cancer agent selected from the group consisting of: an antisense oligonucleotide, an siRNA oligonucleotide, a peptide, and a low molecular weight drug.


SEQUENCE LISTING (15 sequences)

SEQ ID NO: 1 - protein, 20 aa, Artificial Sequence
  Synthetic construct, Angiopep-2-C
TFFYGGSRGKRNNFKTEEYC

SEQ ID NO: 2 - protein, 9 aa, Artificial Sequence
  Synthetic construct, Fe mimetic peptide
CRTIGPSVC

SEQ ID NO: 3 - protein, 8 aa, Apis mellifera
  MISC_FEATURE (1)..(8): Miniap-4 peptide
KAPETALD

SEQ ID NO: 4 - DNA, 21 bp, Artificial Sequence
  Synthetic construct, AON_antisense_B-secretase mRNA
uccaccaucuccacaaagctt

SEQ ID NO: 5 - DNA, 21 bp, Artificial Sequence
  Synthetic construct, control  sequence
acgugacacguucggagaatt

SEQ ID NO: 6 - protein, 10 aa, Artificial Sequence
  Synthetic construct, H102 peptide
HKQLPFFEED

SEQ ID NO: 7 - protein, 20 aa, Artificial Sequence
  Synthetic construct, Angiopep-7C peptide
TFFYGGSRGRRNNFRTEEYC

SEQ ID NO: 8 - protein, 10 aa, Artificial Sequence
  Synthetic construct, B6 peptide
CGHKAKGPRK

SEQ ID NO: 9 - protein, 13 aa, Artificial Sequence
  Syntehtic construct, D1 peptide
QSHYRHISPAQVC

SEQ ID NO: 10 - protein, 13 aa, Artificial Sequence
  Synthetic construct, D3 peptide
RPRTRLHTHRNRC

SEQ ID NO: 11 - protein, 13 aa, Artificial Sequence
  Synthetic construct, ACI-89 peptide
PSHYRHISPAQKC

SEQ ID NO: 12 - protein, 13 aa, Artificial Sequence
  Synthetic construct, ACI-89 peptide
PSHYRHISPAQKC

SEQ ID NO: 13 - DNA, 22 bp, Artificial Sequence
  Synthetic construct, Ms-EGFR AON
ctgagggtcgcatctctgaccg

SEQ ID NO: 14 - DNA, 22 bp, Artificial Sequence
  Synthetic construct, miR-186
caaagaattctccttttgggct

SEQ ID NO: 15 - protein, 19 aa, Artificial Sequence
  Synthetic construct, Angiopep-2
TFFYGGSRGKRNNFKTEEY